(19) United States
(12) Patent Application Publication
(10) Pub. No.: US 2014/0121371 A1
(43) Pub. Date: May 1, 2014

(54) PROCESSES AND REAGENTS FOR MAKING DIARYLIODONIUM SALTS

(71) Applicant: Ground Fluor Pharmaceuticals, Inc., Lincoln, NE (US)

(72) Inventor: Stephen G. DiMagno, Lincoln, NE (US)

(73) Assignee: Ground Fluor Pharmaceuticals, Inc., Lincoln, NE (US)

(21) Appl. No.: 14/063,261

(22) Filed: Oct. 25, 2013

Related U.S. Application Data
(60) Provisional application No. 61/719,387, filed on Oct. 27, 2012.

(51) Int. Cl. C07D 265/30; C07D 239/94; C07D 417/04; C07C 229/36; C07D 277/66; C07D 207/46; C07D 207/448; C07D 475/04; C07D 405/04

(52) U.S. Cl. CPC C07D 265/30; C07D 475/04; C07D 239/94; C07D 417/04; C07D 405/04; C07D 277/66; C07D 207/46; C07D 207/448; C07C 229/36
USPC 544/174; 544/261; 544/293; 546/270.1; 546/284.1; 548/178; 548/542; 548/548; 562/442

(57) ABSTRACT

This disclosure relates to processes and reagents for making diaryliodonium salts, which are useful for the preparation of fluorinated, iodinated, astatinated and radiofluorinated aromatic compounds.

### RELATED APPLICATIONS

This patent application claims priority to U.S. provisional patent application Ser. No. 61/719,387, filed Oct. 27, 2012, the content of which is incorporated herein by reference in its entirety.

### TECHNICAL FIELD

This invention relates to processes and reagents for making diaryliodonium salts, which are useful for the preparation of fluorinated, iodinated, astatinated and radiofluorinated aromatic compounds.

### BACKGROUND

Diaryliodonium salts are useful as arylating agents for a large variety of organic and inorganic nucleophiles. They have also been applied in metal-catalyzed cross-coupling reactions (Ryan, J. H. and P. J. Stang, Tetrahedron Lett. 1997, 38, 5061-5064; Zhang, B.-X., et al., Heterocycles 2004, 64, 199-206; Kang, S.-K., et al., J. Org. Chem. 1996, 61, 4720-4724; Al-Qahtani, M. H. and V. W. Pike, Perkin 1 2000, 1033-1036; Kang, S.-K., et al., Tetrahedron Lett. 1997, 38, 1947-1950) due to the excellent leaving-group ability of the aryl iodide moiety (Okuyama, T., et al, J. Am. Chem. Soc. 1995, 117, 3360-7). Other than these applications, diaryliodonium salts were found to play a role as oxidants for dearomatization of phenols (Moriarty, R. M. and O. Prakash, Org. React. (N. Y.) 2001, 57, 327-415; Moore, J. D. and P. R. Hanson, Chemtracts 2002, 15, 74-80; Ciufolini, M. A., et al., Synthesis 2007, 3759-3772) and as cationic photoinitiators in photochemistry (Toba, Y., J. Photopolym. Sci. Technol. 2003, 16, 115-118; Crivello, J. V., J. Polym. Sci., Part A: Polym. Chem. 2009, 47, 866-875; Crivello, J. V., Polym. Prepr. (Am. Chem. Soc., Div. Polym. Chem.) 2006, 47, 208-209).

Diaryliodonium salts are also useful for the synthesis of aryl fluorides, for example, in the preparation of ¹⁸F labeled radiotracers. Aryl fluorides are structural moieties in natural products as well as a number of therapeutically important compounds, including pharmaceuticals and positron emission tomography (PET) tracers. Diaryliodonium salts are particularly useful for the nucleophilic fluorination of electron-rich arenes, a class of compounds that is inaccessible using conventional nucleophilic fluorination methods.

For at least these reasons, there is a need to develop new routes in diaryliodonium salts, particularly those having a broad range of functional groups. This application addresses this need and others.

### SUMMARY

The present application provides, inter alia, a process for making a compound of Formula I:

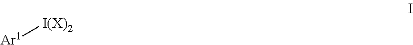

comprising treating a compound of Formula II:

Ar¹—I II

with a tetravalent silicon moiety having at least one X group bound to Si; and (1-chloromethyl-4-fluoro-1,4-diazoniabicyclo[2.2.2]octane)bis(tetrafluoroborate) (SelectFluor™), (1-fluoro-4-methyl-1,4-diazoniabicyclo[2.2.2]octane)bis(tetrafluoroborate) (SelectFluorII™), or optionally substituted N-fluoropyridinium tetrafluoroborate;

wherein:

each X is, independently, a ligand that is a conjugate base of an acid HX, wherein HX has a pKa of less than or equal to 12; and

Ar¹ is optionally substituted aryl or heteroaryl, wherein Ar¹ does not have unprotected protic groups.

The present application further provides a process of converting the compound of Formula I to a compound of Formula III:

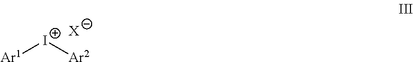

wherein Ar² is an optionally substituted aryl or heteroaryl.

The compound of Formula I can be isolated and then used to prepare the compound of Formula III or the two steps can be carried out in an efficient one-pot synthesis.

This process allows the preparation of iodine (III) precursors of Formula I without the use of acidic conditions or the use of reagents that must be prepared in acidic media as in other synthetic procedures. Acidic conditions are not compatible with substrates featuring acid sensitive moieties or heteroatoms that are prone to protonation or oxidation. Hence, the current process allows the synthesis of a broad range of diaryliodonium salts, which were previously inaccessible. For example, the process has been shown to be applicable to both electron-rich and electron-deficient arenes and is tolerant of molecules featuring acid sensitive moieties and protected L-amino acid groups. Further, this process is also more economical in that less than 2 equivalents of the oxidation agent may be utilized to achieve the oxidation, unlike other processes which use a high excess of the oxidation agent.

The present application also provides certain new compounds of Formulas I, II, III, and V.

### DETAILED DESCRIPTION

The present application provides, inter alia, a process for making a compound of Formula I:

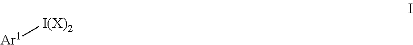

comprising treating a compound of Formula II:

Ar¹—I II

with a tetravalent silicon moiety having at least one X group bound to Si; and (1-chloromethyl-4-fluoro-1,4-diazoniabicyclo[2.2.2]octane)bis(tetrafluoroborate), (1-fluoro-4-methyl-1,4-diazoniabicyclo[2.2.2]octane)bis(tetrafluoroborate), or optionally substituted N-fluoropyridinium tetrafluoroborate;

wherein:

each X is, independently, a ligand that is a conjugate base of an acid HX, wherein HX has a pKa of less than or equal to 12; and

Ar¹ is optionally substituted aryl or heteroaryl.

In some embodiments, Ar¹ does not have any iodo groups (e.g., Ar¹—I has only the single iodo group).

In some embodiments, Ar¹ is optionally substituted aryl or heteroaryl, wherein Ar¹ does not have unprotected protic groups. As used herein, “protic groups” means groups having a hydrogen atom directly attached to an oxygen, nitrogen or sulfur atom (non-limiting examples of these groups include alcohols, primary and secondary amines, carbamates, ureas, amides, sulfonic acids, thiols, hydrazines, hydrazides, and semicarbazides).

As described above, the current process allows the synthesis of a broad range of diaryliodonium salts, including both electron-rich and electron-deficient arenes and is tolerant of molecules featuring acid sensitive moieties and protected L-amino acid groups. Without wishing to be bound by any theory, the process is believed to operate by the process shown in the example below. It is thought that the highly activated I(III) intermediate aryl-IF+, formed from two-electron oxidation of an aryl iodide by F-TEDA-BF₄, is sufficiently Lewis acidic to remove a fluoride from BF₄— to form the aryl-IF₂ trifluoroborane complex. Aryl-IF₂ reacts subsequently with TMS-X to give 1a and TMSF, while boron trifluoride is coordinated by the free amine of reduced Selectfluor to form the zwitterionic adduct, which is able to exchange fluoride with excess TMS-X (e.g., TMSOAc). The aryl-IF₂ compound undergoes a fast ligand exchange process with X—. The premixed TMSOAc therefore converted aryl-IF₂ to corresponding ArI(OAc)₂ immediately upon formation of ArIF₂.

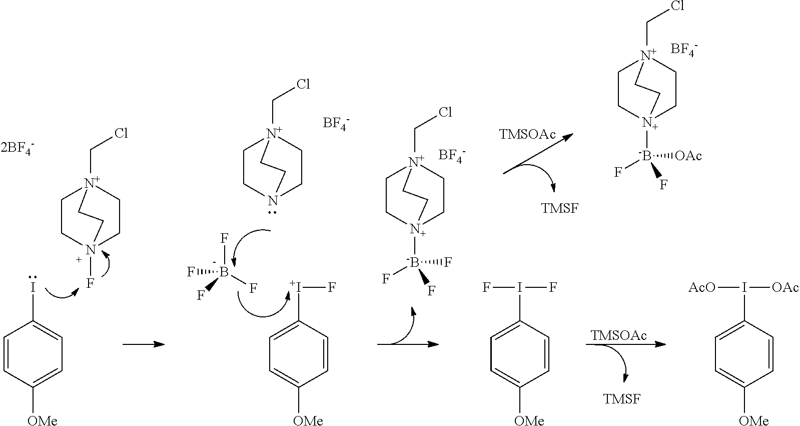

In some embodiments, the process is carried out in the absence of added acid (e.g., protic acid).

In some embodiments, the process utilizes (1-chloromethyl-4-fluoro-1,4-diazoniabicyclo[2.2.2]octane)bis(tetrafluoroborate).

In some embodiments, the process utilizes (1-fluoro-4-methyl-1,4-diazoniabicyclo[2.2.2]octane)bis(tetrafluoroborate).

In some embodiments, the process utilizes N-fluoropyridinium tetrafluoroborate, wherein the pyridine ring is optionally substituted by 1, 2, 3, 4, or 5 groups independently selected from halo, cyano, nitro, C₁₋₆ alkyl, C₁₋₆ haloalkyl, C₂₋₆ alkenyl, C₂₋₆ alkynyl, C₃₋₁₀ cycloalkyl, C₃₋₁₀ cycloalkyl-C₁₋₄-alkyl, C₂₋₁₀ heterocycloalkyl, C₂₋₁₀ heterocycloalkyl-C₁₋₄-alkyl, C₆₋₁₀ aryl, C₆₋₁₀ aryl-C₁₋₄-alkyl, C₁₋₁₀ heteroaryl, hydroxy, C₁₋₆ alkoxy, C₁₋₆ haloalkoxy, C₁₋₆ alkylthio, C₁₋₆ alkylsulfinyl, C₁₋₆ alkylsulfonyl, carbamyl, C₁₋₆ alkylcarbamyl, di(C₁₋₆ alkyl)carbamyl, carboxy, amino, C₁₋₆ alkylamino, di-C₁₋₆ alkylamino, C₁₋₆ alkylcarbonyl, C₁₋₆ alkoxycarbonyl, C₁₋₆ alkylcarbonyloxy, C₁₋₆ alkylcarbonylamino, C₁₋₆ alkylsulfonylamino, amino sulfonyl, C₁₋₆ alkylamino sulfonyl, di(C₁₋₆ alkyl) amino sulfonyl, amino sulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino; wherein said C₁₋₆ alkyl, C₃₋₁₀ cycloalkyl-C₁₋₄-alkyl, C₂₋₁₀ heterocycloalkyl, C₂₋₁₀ heterocycloalkyl-C₁₋₄-alkyl, C₆₋₁₀ aryl, C₆₋₁₀ aryl-C₁₋₄-alkyl, C₁₋₁₀ heteroaryl are each optionally substituted by one or more groups selected from halo, cyano, nitro, C₁₋₆ alkyl, C₁₋₆ haloalkyl, C₂₋₆ alkenyl, C₂₋₆ alkynyl, C₁₋₆ alkoxy, hydroxy, C₁₋₆ alkoxy, C₁₋₆ haloalkoxy, C₁₋₆ alkylthio, C₁₋₆ alkylsulfinyl, C₁₋₆ alkylsulfonyl, carbamyl, C₁₋₆ alkylcarbamyl, di(C₁₋₆ alkyl)carbamyl, carboxy, amino, C₁₋₆ alkylamino, di-C₁₋₆ alkylamino, C₁₋₆ alkylcarbonyl, C₁₋₆ alkoxycarbonyl, C₁₋₆ alkylcarbonyloxy, C₁₋₆ alkylcarbonylamino, C₁₋₆ alkylsulfonylamino, amino sulfonyl, C₁₋₆ alkylamino sulfonyl, di(C₁₋₆ alkyl) amino sulfonyl, amino sulfonylamino, C₁₋₆ alkylaminosulfonylamino, di(C₁₋₆ alkyl)aminosulfonylamino, aminocarbonylamino, C₁₋₆ alkylaminocarbonylamino, di(C₁₋₆ alkyl)aminocarbonylamino, and C₃₋₁₀ cycloalkyl-C₁₋₄-alkyl, C₂₋₁₀ heterocycloalkyl, C₂₋₁₀ heterocycloalkyl-C₁₋₄-alkyl, C₆₋₁₀ aryl, C₆₋₁₀ aryl-C₁₋₄-alkyl, C₁₋₁₀ heteroaryl.

In some embodiments, the process utilizes N-fluoropyridinium tetrafluoroborate, wherein the pyridine ring is optionally substituted by 1, 2, 3, 4, or 5 groups independently selected halo groups.

In some embodiments, the process utilizes N-fluoropyridinium tetrafluoroborate, wherein the pyridine ring is optionally substituted by 1, 2, 3, 4, or 5 groups independently selected halo groups.

In some embodiments, the process utilizes N-fluoro-2,3,4,5,6-pentachloropyridinium tetrafluoroborate.

In some embodiments, the process utilizes less than 2 equivalents of (1-chloromethyl-4-fluoro-1,4-diazoniabicyclo[2.2.2]octane)bis(tetrafluoroborate), (1-fluoro-4-methyl-1,4-diazoniabicyclo[2.2.2]octane)bis(tetrafluoroborate), or optionally substituted N-fluoropyridinium tetrafluoroborate for 1 equivalent of the compound of Formula II. In some embodiments, the process utilizes less than 1.5 equivalents of (1-chloromethyl-4-fluoro-1,4-diazoniabicyclo[2.2.2]octane)bis(tetrafluoroborate), (1-fluoro-4-methyl-1,4-diazoniabicyclo[2.2.2]octane)bis(tetrafluoroborate), or optionally substituted N-fluoropyridinium tetrafluoroborate for 1 equivalent of the compound of Formula II.

In some embodiments, each X is, independently, a ligand that is a conjugate base of an acid HX, wherein HX has a pKa of less than or equal to 5.

In some embodiments, X can be chosen from halide, aryl carboxylate, alkyl carboxylate, phosphate, phosphonate, phosphonite, azide, thiocyanate, cyanate, phenoxide, triflate, thiolates, and stabilized enolates.

In some embodiments, X is O(C═O)CH₃.

In some embodiments, the tetravalent silicon moiety is (R¹)₃Si—X, (R¹)₂Si—(X)₂, R¹Si—(X)₃, and Si(X)₄; wherein each R¹ is, independently, C₁₋₁₂ alkyl or aryl.

In some embodiments, the tetravalent silicon moiety is (R¹)₃Si—X, wherein each R¹ is, independently, C₁₋₁₂ alkyl or aryl.

In some embodiments, each R¹ is, independently, C₁₋₁₂ alkyl.

In some embodiments, each R¹ is, independently, C₁₋₄ alkyl.

In some embodiments, each R¹ is independently, methyl.

In some embodiments, (R¹)₃Si—X is (CH₃)₃Si—X.

In some embodiments, (R¹)₃Si—X is (CH₃)₃Si—O(C═O)CH₃.

At various points, the process utilizes protecting groups. Appropriate protecting groups for various functional groups include, but are not limited to the protecting groups delineated in Wuts and Greene, Protective Groups in Organic Synthesis, 4th ed., John Wiley & Sons: New Jersey, which is incorporated herein by reference in its entirety. For example, protecting groups for amines include, but are not limited to, t-butoxycarbonyl (BOC), benzyloxycarbonyl (Cbz), 2,2,2-trichloroethoxycarbonyl (Troc), 2-(4-trifluoromethylphenylsulfonyl)ethoxycarbonyl (Tsc), 1-adamantyloxycarbonyl (Adoc), 2-adamantylcarbonyl (2-Adoc), 2,4-dimethylpent-3-yloxycarbonyl (Doc), cyclohexyloxycarbonyl (Hoc), 1,1-dimethyl-2,2,2-trichloroethoxycarbonyl (TcBOC), vinyl, 2-chloroethyl, 2-phenylsulfonylethyl, allyl, benzyl, 2-nitrobenzyl, 4-nitrobenzyl, diphenyl-4-pyridylmethyl, N′,N′-dimethylhydrazinyl, methoxymethyl, t-butoxymethyl (Bum), benzyloxymethyl (BOM), or 2-tetrahydropyranyl (THP).

Carboxylic acids can be protected as their alkyl, allyl, or benzyl esters, among other groups.

Alcohols can be protected as esters, such as acetyl, benzoyl, or pivaloyl, or as ethers. Examples of ether protecting groups for alcohols include, but are not limited to alkyl, allyl, benzyl, methoxymethyl (MOM), t-butoxymethyl, tetrahydropyranyl (THP), p-methoxybenzyl (PMB), trityl, and methoxyethoxymethyl (MEM).

In some embodiments, the protecting groups are acid labile protecting groups.

In some embodiments, the protecting groups are base labile protecting groups.

In some embodiments, the protecting group are acid labile protecting groups, which can be easily be removed at the end of all synthetic steps under acidic deprotection conditions.

In general, the methods described herein are not compatible with compounds having N—H or O—H bonds.

In some embodiments, the process utilizes 2 equivalents or more of the tetravalent silicon moiety for 1 equivalent of the compound of Formula II. As used herein, the equivalents are per X group bound to the Si atom of the tetravalent silicon moiety (e.g., when 2× groups are bound to the Si atom, then only 1 equivalent or more of the tetravalent silicon moiety are needed for 1 equivalent of the compound of Formula II). In some embodiments, the process utilizes 2.5 equivalents to 3 equivalents of the tetravalent silicon moiety for 1 equivalent of the compound of Formula II. In some embodiments, the process utilizes 2 equivalents or more of (R¹)₃Si—X for 1 equivalent of the compound of Formula II. In some embodiments, the process utilizes 2.5 equivalents to 3 equivalents of (R¹)₃Si—X for 1 equivalent of the compound of Formula II.

In some embodiments, the processes comprises treating a compound of Formula II with (CH₃)₃Si—O(C═O)CH₃; and (1-chloromethyl-4-fluoro-1,4-diazoniabicyclo[2.2.2]octane) bis(tetrafluoroborate). In some embodiments, the processes comprises treating a compound of Formula II with 2.5 equivalents to 3 equivalents of (CH₃)₃Si—O(C═O)CH₃; and less than 1.5 equivalents of (1-chloromethyl-4-fluoro-1,4-diazoniabicyclo[2.2.2]octane)bis(tetrafluoroborate).

In some embodiments:

Ar¹ is aryl or heteroaryl, which is optionally substituted by one or more groups independently selected from halo, cyano, nitro, C₁₋₆ alkyl, C₁₋₁₆ alkyl, C₁₋₆ haloalkyl, C₂₋₁₆ alkenyl, C₂₋₁₆ alkynyl, C₁₋₆ alkoxy, C₃₋₁₄ cycloalkyl, C₃₋₁₄ cycloalkyl-C₁₋₄-alkyl, C₂₋₁₄ heterocycloalkyl, C₂₋₁₄ heterocycloalkyl-C₁₋₄-alkyl, C₆₋₁₄ aryl, C₆₋₁₄ aryl-C₁₋₄-alkyl, C₁₋₁₄ heteroaryl, C₁₋₁₄ heteroaryl-C₁₋₄-alkyl, —S(═O)R^{a}, —S(═O)₂R^{a}, —S(═O)₂NR^{g}R^{h}, —C(C═O)R^{b}, —C(C═O)NR^{g}R^{h}, —OC(═O)R^{a}, —OC(═O)NR^{g}R^{h}, —NR^{k}C(═O)R^{a}, —NR^{k}C(═O)OR^{b}, —NR^{k}C(═O)NR^{g}NR^{h}, —NR^{k}S(═O)₂R^{a}, —NR^{k}S(═O)₂NR^{g}R^{h}, C(═NRⁱ)NR^{g}R^{h}, NR^{k}C(═NRⁱ)NR^{g}R^{h}, —OR^{c}, —SR^{d}, —S(═O)₂OR^{e}, —C(C═O)OR^{f}, and —NR^{g}R^{h}; wherein said C₁₋₆ alkyl, C₁₋₆ haloalkyl, C₂₋₆ alkenyl, C₂₋₆ alkynyl, C₁₋₆ alkoxy, C₃₋₁₄ cycloalkyl, C₃₋₁₄ cycloalkyl-C₁₋₄-alkyl, C₂₋₁₄ heterocycloalkyl, C₂₋₁₄ heterocycloalkyl-C₁₋₄-alkyl, C₆₋₁₄ aryl, C₆₋₁₄ aryl-C₁₋₄-alkyl, C₁₋₁₄ heteroaryl, and C₁₋₁₄ heteroaryl-C₁₋₄-alkyl are each optionally substituted by one or more independently selected R² groups;

each Rⁱ is independently selected from H, C₁₋₆ alkyl, CN, C₁₋₆ alkoxy, or C(O)C₁₋₆ alkyl; each R^{a} is independently selected from H, C₁₋₆ alkyl, C₁₋₆ haloalkyl, C₂₋₆ alkenyl, C₂₋₆ alkynyl, aryl, C₃₋₁₀ cycloalkyl, C₃₋₁₀ cycloalkyl-C₁₋₄-alkyl, C₂₋₁₀heterocycloalkyl, C₂₋₁₀ heterocycloalkyl-C₁₋₄-alkyl, C₆₋₁₀ aryl, C₆₋₁₀ aryl-C₁₋₄-alkyl, C₁₋₁₀heteroaryl, wherein said C₁₋₆ alkyl, C₁₋₆ haloalkyl, C₂₋₆ alkenyl, C₂₋₆ alkynyl, C₃₋₁₀ cycloalkyl, C₃₋₁₀ cycloalkyl-C₁₋₄-alkyl, C₂₋₁₀ heterocycloalkyl, C₂₋₁₀ heterocycloalkyl-C₁₋₄-alkyl, C₆₋₁₀ aryl, C₆₋₁₀ aryl-C₁₋₄-alkyl, C₁₋₁₀ heteroaryl, and C₁₋₁₀ heteroaryl-C₁₋₄-alkyl are each optionally substituted by one or more independently selected R² groups;

each R^{b} is independently selected from C₁₋₆ alkyl, C₁₋₆ haloalkyl, C₂₋₆ alkenyl, C₂₋₆ alkynyl, C₃₋₁₀ cycloalkyl, C₃₋₁₀ cycloalkyl-C₁₋₄-alkyl, C₂₋₁₀ heterocycloalkyl, C₂₋₁₀ heterocycloalkyl-C₁₋₄-alkyl, C₆₋₁₀ aryl, C₆₋₁₀ aryl-C₁₋₄-alkyl, C₁₋₁₀ heteroaryl, wherein said C₁₋₆ alkyl, C₁₋₆ haloalkyl, C₂₋₆ alkenyl, C₂₋₆ alkynyl, aryl, C₃₋₁₀ cycloalkyl, C₃₋₁₀ cycloalkyl-C₁₋₄-alkyl, C₂₋₁₀ heterocycloalkyl, C₂₋₁₀ heterocycloalkyl-C₁₋₄-alkyl, C₆₋₁₀ aryl, C₆₋₁₀ aryl-C₁₋₄-alkyl, C₁₋₁₀ heteroaryl, and C₁₋₁₀ heteroaryl-C₁₋₄-alkyl are each optionally substituted by one or more independently selected R² groups;

each R^{e} is independently selected from a protecting group, C₁₋₆ alkyl, C₁₋₆haloalkyl, C₂₋₆ alkenyl, C₂₋₆ alkynyl, C₃₋₁₀ cyclo alkyl, C₃₋₁₀ cycloalkyl-C₁₋₄-alkyl, C₂₋₁₀ heterocycloalkyl, C₂₋₁₀ heterocycloalkyl-C₁₋₄-alkyl, C₆₋₁₀ aryl, C₆₋₁₀ aryl-C₁₋₄-alkyl, C₁₋₁₀ heteroaryl, wherein said C₁₋₆ alkyl, C₁₋₆haloalkyl, C₂₋₆ alkenyl, C₂₋₆ alkynyl, aryl, C₃₋₁₀ cycloalkyl, C₃₋₁₀ cycloalkyl-C₁₋₄-alkyl, C₂₋₁₀ heterocycloalkyl, C₂₋₁₀ heterocycloalkyl-C₁₋₄-alkyl, C₆₋₁₀ aryl, C₆₋₁₀ aryl-C₁₋₄-alkyl, C₁₋₁₀ heteroaryl, and C₁₋₁₀ heteroaryl-C₁₋₄-alkyl are each optionally substituted by one or more independently selected R² groups;

each R^{d} is independently selected from a protecting group, C₁₋₆ alkyl, C₁₋₆haloalkyl, C₂₋₆ alkenyl, C₂₋₆ alkynyl, C₃₋₁₀ cyclo alkyl, C₃₋₁₀ cycloalkyl-C₁₋₄-alkyl, C₂₋₁₀ heterocycloalkyl, C₂₋₁₀ heterocycloalkyl-C₁₋₄-alkyl, C₆₋₁₀ aryl, C₆₋₁₀ aryl-C₁₋₄-alkyl, C₁₋₁₀ heteroaryl, wherein said C₁₋₆ alkyl, C₁₋₆haloalkyl, C₂₋₆ alkenyl, C₂₋₆ alkynyl, aryl, C₃₋₁₀ cycloalkyl, C₃₋₁₀ cycloalkyl-C₁₋₄-alkyl, C₂₋₁₀ heterocycloalkyl, C₂₋₁₀ heterocycloalkyl-C₁₋₄-alkyl, C₆₋₁₀ aryl, C₆₋₁₀ aryl-C₁₋₄-alkyl, C₁₋₁₀ heteroaryl, and C₁₋₁₀ heteroaryl-C₁₋₄-alkyl are each optionally substituted by one or more independently selected R² groups;

each R^{e} is independently selected from a protecting group, C₁₋₆ alkyl, C₁₋₆haloalkyl, C₂₋₆ alkenyl, C₂₋₆ alkynyl, C₃₋₁₀ cyclo alkyl, C₃₋₁₀ cycloalkyl-C₁₋₄-alkyl, C₂₋₁₀ heterocycloalkyl, C₂₋₁₀ heterocycloalkyl-C₁₋₄-alkyl, C₆₋₁₀ aryl, C₆₋₁₀ aryl-C₁₋₄-alkyl, C₁₋₁₀ heteroaryl, wherein said C₁₋₆ alkyl, C₁₋₆haloalkyl, C₂₋₆ alkenyl, C₂₋₆ alkynyl, aryl, C₃₋₁₀ cycloalkyl, C₃₋₁₀ cycloalkyl-C₁₋₄-alkyl, C₂₋₁₀ heterocycloalkyl, C₂₋₁₀ heterocycloalkyl-C₁₋₄-alkyl, C₆₋₁₀ aryl, C₆₋₁₀ aryl-C₁₋₄-alkyl, C₁₋₁₀ heteroaryl, and C₁₋₁₀ heteroaryl-C₁₋₄-alkyl are each optionally substituted by one or more independently selected R² groups;

each R^{f} is independently selected from a protecting group, C₁₋₆ alkyl, C₁₋₆haloalkyl, C₂₋₆ alkenyl, C₂₋₆ alkynyl, C₃₋₁₀ cyclo alkyl, C₃₋₁₀ cycloalkyl-C₁₋₄-alkyl, C₂₋₁₀ heterocycloalkyl, C₂₋₁₀ heterocycloalkyl-C₁₋₄-alkyl, C₆₋₁₀ aryl, C₆₋₁₀ aryl-C₁₋₄-alkyl, C₁₋₁₀ heteroaryl, wherein said C₁₋₆ alkyl, C₁₋₆haloalkyl, C₂₋₆ alkenyl, C₂₋₆ alkynyl, aryl, C₃₋₁₀ cycloalkyl, C₃₋₁₀ cycloalkyl-C₁₋₄-alkyl, C₂₋₁₀ heterocycloalkyl, C₂₋₁₀ heterocycloalkyl-C₁₋₄-alkyl, C₆₋₁₀ aryl, C₆₋₁₀ aryl-C₁₋₄-alkyl, C₁₋₁₀ heteroaryl, and C₁₋₁₀ heteroaryl-C₁₋₄-alkyl are each optionally substituted by one or more independently selected R² groups;

each R^{k}, R^{g} and R^{h} is independently selected from a protecting group, C₁₋₆ alkyl, C₁₋₆ haloalkyl, C₂₋₆ alkenyl, C₂₋₆ alkynyl, C₃₋₁₀ cycloalkyl, C₃₋₁₀ cycloalkyl-C₁₋₄-alkyl, C₂₋₁₀ heterocycloalkyl, C₂₋₁₀ heterocycloalkyl-C₁₋₄-alkyl, C₆₋₁₀ aryl, C₆₋₁₀ aryl-C₁₋₄-alkyl, C₁₋₁₀heteroaryl, wherein said C₁₋₆ alkyl, C₁₋₆ haloalkyl, C₂₋₆ alkenyl, C₂₋₆ alkynyl, aryl, C₃₋₁₀ cycloalkyl, C₃₋₁₀ cycloalkyl-C₁₋₄-alkyl, C₂₋₁₀ heterocycloalkyl, C₂₋₁₀ heterocycloalkyl-C₁₋₄-alkyl, C₆₋₁₀ aryl, C₆₋₁₀ aryl-C₁₋₄-alkyl, C₁₋₁₀ heteroaryl, and C₁₋₁₀ heteroaryl-C₁₋₄-alkyl are each optionally substituted by one or more independently selected R² groups;

or alternatively, R^{k} and R^{a}, taken together with the atoms to which they are attached, form a heterocycloalkyl or heteroaryl ring, which is optionally substituted by one or more R² groups;

or alternatively, R^{k} and R^{b}, taken together with the atoms to which they are attached, form a heterocycloalkyl or heteroaryl ring, which is optionally substituted by one or more R² groups;

or alternatively, R^{k} and R^{g}, taken together with the atoms to which they are attached, form a heterocycloalkyl or heteroaryl ring, which is optionally substituted by one or more R² groups;

or alternatively, R^{g} and R^{h}, taken together with the nitrogen atom to which they are attached, form a heterocycloalkyl or heteroaryl ring, which is optionally substituted by one or more R⁴ groups;

each R² is independently selected from halo, cyano, nitro, C₁₋₆ alkyl, C₁₋₆ haloalkyl, C₁₋₆ haloalkyl, C₂₋₆ alkenyl, C₂₋₆ alkynyl, C₁₋₆ alkoxy, C₃₋₁₀ cycloalkyl, C₃₋₁₀ cycloalkyl-C₁₋₄-alkyl, C₂₋₁₀ heterocycloalkyl, C₂₋₁₀ heterocycloalkyl-C₁₋₄-alkyl, C₆₋₁₀ aryl, C₆₋₁₀ aryl-C₁₋₄-alkyl, C₁₋₁₀ heteroaryl, C₁₋₁₀ heteroaryl-C₁₋₄-alkyl, —S(═O)R^{a1}, —S(═O)₂R^{a1}, —S(═O)₂NR^{g1}R^{h1}, —C(C═O)R^{h1}, —C(C═O)NR^{g1}R^{h1}, —OC(═O)R^{a1}, —OC(═O)NR^{g1}R^{h1}, —NR^{k1}C(═O)R^{a1}, —NR^{k1}C(═O)OR^{b1}, —NR^{k1}C(═O)NR^{g1}NR^{h1}, —NR^{k1}S(═O)₂R^{a1}, —NR^{k1}S(═O)₂NR^{g1}R^{h1}C(═NRⁱ)NR^{g1}R^{h1}, NR^{k1}C(═NRⁱ)NR^{g1}R^{h1}, —NR^{k1}S(═O)₂OR^{e1}, —C(C═O)OR¹, and —NR^{g1}R^{h1}; wherein said C₁₋₆ alkyl, C₁₋₆ haloalkyl, C₂₋₆ alkenyl, C₂₋₆ alkynyl, C₁₋₆ alkoxy, C₃₋₁₀ cycloalkyl, C₃₋₁₀ cycloalkyl-C₁₋₄-alkyl, C₂₋₁₀ heterocycloalkyl, C₂₋₁₀ heterocycloalkyl-C₁₋₄-alkyl, C₆₋₁₀ aryl, C₆₋₁₀ aryl-C₁₋₄-alkyl, C₁₋₁₀ heteroaryl, and C₁₋₁₀ heteroaryl-C₁₋₄-alkyl are each optionally substituted by one or more independently selected R³ groups;

each R^{a1} is independently selected from H, C₁₋₆ alkyl, C₁₋₆ haloalkyl, C₂₋₆ alkenyl, C₂₋₆ alkynyl, C₃₋₁₀ cycloalkyl, C₃₋₁₀ cycloalkyl-C₁₋₄-alkyl, C₂₋₁₀ heterocycloalkyl, C₂₋₁₀ heterocycloalkyl-C₁₋₄-alkyl, C₆₋₁₀ aryl, C₆₋₁₀ aryl-C₁₋₄-alkyl, C₁₋₁₀ heteroaryl, wherein said C₁₋₆ alkyl, C₁₋₆ haloalkyl, C₂₋₆ alkenyl, C₂₋₆ alkynyl, C₃₋₁₀ cycloalkyl, C₃₋₁₀ cycloalkyl-C₁₋₄-alkyl, C₂₋₁₀ heterocycloalkyl, C₂₋₁₀ heterocycloalkyl-C₁₋₄-alkyl, C₆₋₁₀ aryl, C₆₋₁₀ aryl-C₁₋₄-alkyl, C₁₋₁₀ heteroaryl, and C₁₋₁₀ heteroaryl-C₁₋₄-alkyl are each optionally substituted by one or more independently selected R³ groups;

each R^{b1} is independently selected from C₁₋₆ alkyl, C₁₋₆ haloalkyl, C₂₋₆ alkenyl, C₂₋₆ alkynyl, C₃₋₁₀ cycloalkyl, C₃₋₁₀ cycloalkyl-C₁₋₄-alkyl, C₂₋₁₀ heterocycloalkyl, C₂₋₁₀ heterocycloalkyl-C₁₋₄-alkyl, C₆₋₁₀ aryl, C₆₋₁₀ aryl-C₁₋₄-alkyl, C₁₋₁₀ heteroaryl, wherein said C₁₋₆ alkyl, C₁₋₆ haloalkyl, C₂₋₆ alkenyl, C₂₋₆ alkynyl, C₃₋₁₀ cycloalkyl, C₃₋₁₀ cycloalkyl-C₁₋₄-alkyl, C₂₋₁₀ heterocycloalkyl, C₂₋₁₀ heterocycloalkyl-C₁₋₄-alkyl, C₆₋₁₀ aryl, C₆₋₁₀ aryl-C₁₋₄-alkyl, C₁₋₁₀ heteroaryl, and C₁₋₁₀ heteroaryl-C₁₋₄-alkyl are each optionally substituted by one or more independently selected R³ groups;

each R^{c1} is independently selected from a protecting group, C₁₋₆ alkyl, C₁₋₆ haloalkyl, C₂₋₆ alkenyl, C₂₋₆ alkynyl, C₃₋₁₀ cyclo alkyl, C₃₋₁₀ cycloalkyl-C₁₋₄-alkyl, C₂₋₁₀ heterocycloalkyl, C₂₋₁₀ heterocycloalkyl-C₁₋₄-alkyl, C₆₋₁₀ aryl, C₆₋₁₀ aryl-C₁₋₄-alkyl, C₁₋₁₀ heteroaryl, wherein said C₁₋₆ alkyl, C₁₋₆ haloalkyl, C₂₋₆ alkenyl, C₂₋₆ alkynyl, C₃₋₁₀ cycloalkyl, C₃₋₁₀ cycloalkyl-C₁₋₄-alkyl, C₂₋₁₀ heterocycloalkyl, C₂₋₁₀ heterocycloalkyl-C₁₋₄-alkyl, C₆₋₁₀ aryl, C₆₋₁₀ aryl-C₁₋₄-alkyl, C₁₋₁₀ heteroaryl, and C₁₋₁₀ heteroaryl-C₁₋₄-alkyl are each optionally substituted by one or more independently selected R³ groups;

each R^{d1} is independently selected from a protecting group, C₁₋₆ alkyl, C₁₋₆ haloalkyl, C₂₋₆ alkenyl, C₂₋₆ alkynyl, C₃₋₁₀ cyclo alkyl, C₃₋₁₀ cycloalkyl-C₁₋₄-alkyl, C₂₋₁₀ heterocycloalkyl, C₂₋₁₀ heterocycloalkyl-C₁₋₄-alkyl, C₆₋₁₀ aryl, C₆₋₁₀ aryl-C₁₋₄-alkyl, C₁₋₁₀ heteroaryl, wherein said C₁₋₆ alkyl, C₁₋₆ haloalkyl, C₂₋₆ alkenyl, C₂₋₆ alkynyl, C₃₋₁₀ cycloalkyl, C₃₋₁₀ cycloalkyl-C₁₋₄-alkyl, C₂₋₁₀ heterocycloalkyl, C₂₋₁₀ heterocycloalkyl-C₁₋₄-alkyl, C₆₋₁₀ aryl, C₆₋₁₀ aryl-C₁₋₄-alkyl, C₁₋₁₀ heteroaryl, and C₁₋₁₀ heteroaryl-C₁₋₄-alkyl are each optionally substituted by one or more independently selected R³ groups;

each R^{e1} is independently selected from a protecting group, C₁₋₆ alkyl, C₁₋₆ haloalkyl, C₂₋₆ alkenyl, C₂₋₆ alkynyl, C₃₋₁₀ cyclo alkyl, C₃₋₁₀ cycloalkyl-C₁₋₄-alkyl, C₂₋₁₀ heterocycloalkyl, C₂₋₁₀ heterocycloalkyl-C₁₋₄-alkyl, C₆₋₁₀ aryl, C₆₋₁₀ aryl-C₁₋₄-alkyl, C₁₋₁₀ heteroaryl, wherein said C₁₋₆ alkyl, C₁₋₆ haloalkyl, C₂₋₆ alkenyl, C₂₋₆ alkynyl, C₃₋₁₀ cycloalkyl, C₃₋₁₀ cycloalkyl-C₁₋₄-alkyl, C₂₋₁₀ heterocycloalkyl, C₂₋₁₀ heterocycloalkyl-C₁₋₄-alkyl, C₆₋₁₀ aryl, C₆₋₁₀ aryl-C₁₋₄-alkyl, C₁₋₁₀ heteroaryl, and C₁₋₁₀ heteroaryl-C₁₋₄-alkyl are each optionally substituted by one or more independently selected R³ groups;

each R^{f1} is independently selected from a protecting group, C₁₋₆ alkyl, C₁₋₆ haloalkyl, C₂₋₆ alkenyl, C₂₋₆ alkynyl, C₃₋₁₀ cyclo alkyl, C₃₋₁₀ cycloalkyl-C₁₋₄-alkyl, C₂₋₁₀ heterocycloalkyl, C₂₋₁₀ heterocycloalkyl-C₁₋₄-alkyl, C₆₋₁₀ aryl, C₆₋₁₀ aryl-C₁₋₄-alkyl, C₁₋₁₀ heteroaryl, wherein said C₁₋₆ alkyl, C₁₋₆ haloalkyl, C₂₋₆ alkenyl, C₂₋₆ alkynyl, C₃₋₁₀ cycloalkyl, C₃₋₁₀ cycloalkyl-C₁₋₄-alkyl, C₂₋₁₀ heterocycloalkyl, C₂₋₁₀ heterocycloalkyl-C₁₋₄-alkyl, C₆₋₁₀ aryl, C₆₋₁₀ aryl-C₁₋₄-alkyl, C₁₋₁₀ heteroaryl, and C₁₋₁₀ heteroaryl-C₁₋₄-alkyl are each optionally substituted by one or more independently selected R³ groups;

each R^{k1}, R^{g1} and R^{h2} is independently selected from a protecting group, C₁₋₆ alkyl, C₁₋₆ haloalkyl, C₂₋₆ alkenyl, C₂₋₆ alkynyl, C₃₋₁₀ cycloalkyl, C₃₋₁₀ cycloalkyl-C₁₋₄-alkyl, C₂₋₁₀ heterocycloalkyl, C₂₋₁₀ heterocycloalkyl-C₁₋₄-alkyl, C₆₋₁₀ aryl, C₆₋₁₀ aryl-C₁₋₄-alkyl, C₁₋₁₀ heteroaryl, wherein said C₁₋₆ alkyl, C₁₋₆ haloalkyl, C₂₋₆ alkenyl, C₂₋₆ alkynyl, C₃₋₁₀ cycloalkyl, C₃₋₁₀ cycloalkyl-C₁₋₄-alkyl, C₂₋₁₀ heterocycloalkyl, C₂₋₁₀ heterocycloalkyl-C₁₋₄-alkyl, C₆₋₁₀ aryl, C₆₋₁₀ aryl-C₁₋₄-alkyl, C₁₋₁₀ heteroaryl, and C₁₋₁₀ heteroaryl-C₁₋₄-alkyl are each optionally substituted by one or more independently selected R³ groups;

or alternatively, R^{k1} and R^{a1}, taken together with the atoms to which they are attached, form a heterocycloalkyl or heteroaryl ring, which is optionally substituted by one or more R³ groups;

or alternatively, R^{k1} and R^{b1}, taken together with the atoms to which they are attached, form a heterocycloalkyl or heteroaryl ring, which is optionally substituted by one or more R³ groups;

or alternatively, R^{k1} and R^{g1}, taken together with the atoms to which they are attached, form a heterocycloalkyl or heteroaryl ring, which is optionally substituted by one or more R³ groups;

or alternatively, R^{g1} and R^{h1}, taken together with the nitrogen atom to which they are attached, form a heterocycloalkyl or heteroaryl ring, which is optionally substituted by one or more R³ groups;

each R³ is independently selected from halo, cyano, nitro, C₁₋₆ alkyl, C₁₋₆ haloalkyl, C₁₋₆ haloalkyl, C₂₋₆ alkenyl, C₂₋₆ alkynyl, C₁₋₆ alkoxy, C₃₋₁₀ cycloalkyl, C₃₋₁₀ cycloalkyl-C₁₋₄-alkyl, C₂₋₁₀ heterocycloalkyl, C₂₋₁₀ heterocycloalkyl-C₁₋₄-alkyl, C₆₋₁₀ aryl, C₆₋₁₀ aryl-C₁₋₄-alkyl, C₁₋₁₀ heteroaryl, C₁₋₁₀heteroaryl-C₁₋₄-alkyl, —S(═O)R^{a2}, —S(═O)₂R^{a2}, —S(═O)₂NR^{g2}R^{h2}, —C(═O)R^{b2}, —C(C═O)NR^{g2}R^{h2}, —OC(═O)R^{a2}, —OC(═O)NR^{g2}R^{h2}, —NR^{k2}C(═O)R^{a2}, —NR^{k2}C(═O)OR^{b2}, —NR^{k2}C(═O)NR^{g2}NR^{h2}, —NR^{k2}S(═O)₂R^{a2}, —NR^{k2}S(═O)₂NR^{g2}R^{h2}, C(═NRⁱ)NR^{g2}R^{h2}, NR^{k2}C(═NRⁱ)NR^{g2}R^{h2}, —OR^{e2}, —SR^{d2}, —S(═O)₂OR^{e2}, —C(C═O)OR^{f2}, and —NR^{g2}R^{h2}; wherein said C₁₋₆ alkyl, C₁₋₆ haloalkyl, C₂₋₆ alkenyl, C₂₋₆ alkynyl, C₁₋₆ alkoxy, C₃₋₁₀ cycloalkyl, C₃₋₁₀ cycloalkyl-C₁₋₄-alkyl, C₂₋₁₀ heterocycloalkyl, C₂₋₁₀ heterocycloalkyl-C₁₋₄-alkyl, C₆₋₁₀ aryl, C₆₋₁₀ aryl-C₁₋₄-alkyl, C₁₋₁₀heteroaryl, and C₁₋₁₀ heteroaryl-C₁₋₄-alkyl are each optionally substituted by one or more independently selected R⁴ groups;

each R^{a2} is independently selected from H, C₁₋₆ alkyl, C₁₋₆ haloalkyl, C₂₋₆ alkenyl, C₂₋₆ alkynyl, C₃₋₁₀ cycloalkyl, C₃₋₁₀ cycloalkyl-C₁₋₄-alkyl, C₂₋₁₀ heterocycloalkyl, C₂₋₁₀ heterocycloalkyl-C₁₋₄-alkyl, C₆₋₁₀ aryl, C₆₋₁₀ aryl-C₁₋₄-alkyl, C₁₋₁₀ heteroaryl, wherein said C₁₋₆ alkyl, C₁₋₆ haloalkyl, C₂₋₆ alkenyl, C₂₋₆ alkynyl, C₃₋₁₀ cycloalkyl, C₃₋₁₀ cycloalkyl-C₁₋₄-alkyl, C₂₋₁₀ heterocycloalkyl, C₂₋₁₀ heterocycloalkyl-C₁₋₄-alkyl, C₆₋₁₀ aryl, C₆₋₁₀ aryl-C₁₋₄-alkyl, C₁₋₁₀ heteroaryl, and C₁₋₁₀ heteroaryl-C₁₋₄-alkyl are each optionally substituted by one or more independently selected R⁴ groups;

each R^{b2} is independently selected from C₁₋₆ alkyl, C₁₋₆ haloalkyl, C₂₋₆ alkenyl, C₂₋₆ alkynyl, C₃₋₁₀ cycloalkyl, C₃₋₁₀ cycloalkyl-C₁₋₄-alkyl, C₂₋₁₀ heterocycloalkyl, C₂₋₁₀ heterocycloalkyl-C₁₋₄-alkyl, C₆₋₁₀ aryl, C₆₋₁₀ aryl-C₁₋₄-alkyl, C₁₋₁₀ heteroaryl, wherein said C₁₋₆ alkyl, C₁₋₆ haloalkyl, C₂₋₆ alkenyl, C₂₋₆ alkynyl, C₃₋₁₀ cycloalkyl, C₃₋₁₀ cycloalkyl-C₁₋₄-alkyl, C₂₋₁₀ heterocycloalkyl, C₂₋₁₀ heterocycloalkyl-C₁₋₄-alkyl, C₆₋₁₀ aryl, C₆₋₁₀ aryl-C₁₋₄-alkyl, C₁₋₁₀ heteroaryl, and C₁₋₁₀ heteroaryl-C₁₋₄-alkyl are each optionally substituted by one or more independently selected R⁴ groups;

each R^{c2} is independently selected from a protecting group, C₁₋₆ alkyl, C₁₋₆ haloalkyl, C₂₋₆ alkenyl, C₂₋₆ alkynyl, C₃₋₁₀ cyclo alkyl, C₃₋₁₀ cycloalkyl-C₁₋₄-alkyl, C₂₋₁₀ heterocycloalkyl, C₂₋₁₀ heterocycloalkyl-C₁₋₄-alkyl, C₆₋₁₀ aryl, C₆₋₁₀ aryl-C₁₋₄-alkyl, C₁₋₁₀ heteroaryl, wherein said C₁₋₆ alkyl, C₁₋₆ haloalkyl, C₂₋₆ alkenyl, C₂₋₆ alkynyl, C₃₋₁₀ cycloalkyl, C₃₋₁₀ cycloalkyl-C₁₋₄-alkyl, C₂₋₁₀ heterocycloalkyl, C₂₋₁₀ heterocycloalkyl-C₁₋₄-alkyl, C₆₋₁₀ aryl, C₆₋₁₀ aryl-C₁₋₄-alkyl, C₁₋₁₀ heteroaryl, and C₁₋₁₀ heteroaryl-C₁₋₄-alkyl are each optionally substituted by one or more independently selected R⁴ groups;

each R^{d2} is independently selected from a protecting group, C₁₋₆ alkyl, C₁₋₆ haloalkyl, C₂₋₆ alkenyl, C₂₋₆ alkynyl, C₃₋₁₀ cyclo alkyl, C₃₋₁₀ cycloalkyl-C₁₋₄-alkyl, C₂₋₁₀ heterocycloalkyl, C₂₋₁₀ heterocycloalkyl-C₁₋₄-alkyl, C₆₋₁₀ aryl, C₆₋₁₀ aryl-C₁₋₄-alkyl, C₁₋₁₀ heteroaryl, wherein said C₁₋₆ alkyl, C₁₋₆ haloalkyl, C₂₋₆ alkenyl, C₂₋₆ alkynyl, C₃₋₁₀ cycloalkyl, C₃₋₁₀ cycloalkyl-C₁₋₄-alkyl, C₂₋₁₀heterocycloalkyl, C₂₋₁₀ heterocycloalkyl-C₁₋₄-alkyl, C₆₋₁₀ aryl, C₆₋₁₀ aryl-C₁₋₄-alkyl, C₁₋₁₀ heteroaryl, and C₁₋₁₀ heteroaryl-C₁₋₄-alkyl are each optionally substituted by one or more independently selected R⁴ groups;

each R^{e2} is independently selected from a protecting group, C₁₋₆ alkyl, C₁₋₆ haloalkyl, C₂₋₆ alkenyl, C₂₋₆ alkynyl, C₃₋₁₀ cyclo alkyl, C₃₋₁₀ cycloalkyl-C₁₋₄-alkyl, C₂₋₁₀ heterocycloalkyl, C₂₋₁₀ heterocycloalkyl-C₁₋₄-alkyl, C₆₋₁₀ aryl, C₆₋₁₀ aryl-C₁₋₄-alkyl, C₁₋₁₀ heteroaryl, wherein said C₁₋₆ alkyl, C₁₋₆ haloalkyl, C₂₋₆ alkenyl, C₂₋₆ alkynyl, C₃₋₁₀ cycloalkyl, C₃₋₁₀ cycloalkyl-C₁₋₄-alkyl, C₂₋₁₀ heterocycloalkyl, C₂₋₁₀ heterocycloalkyl-C₁₋₄-alkyl, C₆₋₁₀ aryl, C₆₋₁₀ aryl-C₁₋₄-alkyl, C₁₋₁₀ heteroaryl, and C₁₋₁₀ heteroaryl-C₁₋₄-alkyl are each optionally substituted by one or more independently selected R⁴ groups;

each R^{f2} is independently selected from a protecting group, C₁₋₆ alkyl, C₁₋₆ haloalkyl, C₂₋₆ alkenyl, C₂₋₆ alkynyl, C₃₋₁₀ cyclo alkyl, C₃₋₁₀ cycloalkyl-C₁₋₄-alkyl, C₂₋₁₀ heterocycloalkyl, C₂₋₁₀ heterocycloalkyl-C₁₋₄-alkyl, C₆₋₁₀ aryl, C₆₋₁₀ aryl-C₁₋₄-alkyl, C₁₋₁₀ heteroaryl, wherein said C₁₋₆ alkyl, C₁₋₆ haloalkyl, C₂₋₆ alkenyl, C₂₋₆ alkynyl, C₃₋₁₀ cycloalkyl, C₃₋₁₀ cycloalkyl-C₁₋₄-alkyl, C₂₋₁₀ heterocycloalkyl, C₂₋₁₀ heterocycloalkyl-C₁₋₄-alkyl, C₆₋₁₀ aryl, C₆₋₁₀ aryl-C₁₋₄-alkyl, C₁₋₁₀ heteroaryl, and C₁₋₁₀ heteroaryl-C₁₋₄-alkyl are each optionally substituted by one or more independently selected R⁴ groups;

each R^{k2}, R^{g2} and R^{h2} is independently selected from a protecting group, C₁₋₆ alkyl, C₁₋₆ haloalkyl, C₂₋₆ alkenyl, C₂₋₆ alkynyl, C₃₋₁₀ cycloalkyl, C₃₋₁₀ cycloalkyl-C₁₋₄-alkyl, C₂₋₁₀ heterocycloalkyl, C₂₋₁₀ heterocycloalkyl-C₁₋₄-alkyl, C₆₋₁₀ aryl, C₆₋₁₀ aryl-C₁₋₄-alkyl, C₁₋₁₀ heteroaryl, wherein said C₁₋₆ alkyl, C₁₋₆ haloalkyl, C₂₋₆ alkenyl, C₂₋₆ alkynyl, C₃₋₁₀ cycloalkyl, C₃₋₁₀ cycloalkyl-C₁₋₄-alkyl, C₂₋₁₀ heterocycloalkyl, C₂₋₁₀ heterocycloalkyl-C₁₋₄-alkyl, C₆₋₁₀ aryl, C₆₋₁₀ aryl-C₁₋₄-alkyl, C₁₋₁₀ heteroaryl, and C₁₋₁₀ heteroaryl-C₁₋₄-alkyl are each optionally substituted by one or more independently selected R⁴ groups;

or alternatively, R^{k2} and R^{a2}, taken together with the atoms to which they are attached, form a heterocycloalkyl or heteroaryl ring, which is optionally substituted by one or more R⁴ groups;

or alternatively, R^{k2} and R^{b2}, taken together with the atoms to which they are attached, form a heterocycloalkyl or heteroaryl ring, which is optionally substituted by one or more R⁴ groups;

or alternatively, R^{k2} and R^{g2}, taken together with the atoms to which they are attached, form a heterocycloalkyl or heteroaryl ring, which is optionally substituted by one or more R⁴ groups;

or alternatively, R^{g2} and R^{h2}, taken together with the nitrogen atom to which they are attached, form a heterocycloalkyl or heteroaryl ring, which is optionally substituted by one or more R⁴ groups;

each R⁴ is independently selected from halo, cyano, nitro, C₁₋₆ alkyl, C₁₋₆ haloalkyl, C₁₋₆ alkyl-NR^{4a}—C₁₋₆ alkylene, C₁₋₆ alkyl-O—C₁₋₆ alkylene, C₂₋₆ alkenyl, C₂₋₆ alkynyl, C₃₋₁₀ cycloalkyl, C₃₋₁₀ cycloalkyl-C₁₋₄-alkyl, C₂₋₁₀ heterocycloalkyl, C₂₋₁₀ heterocycloalkyl-C₁₋₄-alkyl, C₆₋₁₀ aryl, C₆₋₁₀ aryl-C₁₋₄-alkyl, C₁₋₁₀ heteroaryl, hydroxy, C₁₋₆ alkoxy, C₁₋₆haloalkoxy, C₁₋₆ alkylthio, C₁₋₆ alkylsulfinyl, C₁₋₆ alkylsulfonyl, carbamyl, C₁₋₆ alkylcarbamyl, di(C₁₋₆ alkyl)carbamyl, carboxy, amino, C₁₋₆ alkylamino, di-C₁₋₆ alkylamino, C₁₋₆ alkylcarbonyl, C₁₋₆ alkoxycarbonyl, C₁₋₆ alkylcarbonyloxy, C₁₋₆ alkylcarbonylamino, C₁₋₆ alkylsulfonylamino, amino sulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino; wherein said C₁₋₆ alkyl, C₁₋₆ haloalkyl, C₂₋₆ alkenyl, C₂₋₆ alkynyl, C₁₋₆ alkoxy, C₁₋₆ alkyl-NR^{4a}—C₁₋₆ alkylene, C₁₋₆ alkyl-O—C₁₋₆ alkylene, C₃₋₁₀ cyclo alkyl, C₃₋₁₀ cycloalkyl-C₁₋₄-alkyl, C₂₋₁₀ heterocycloalkyl, C₂₋₁₀ heterocycloalkyl-C₁₋₄-alkyl, C₆₋₁₀ aryl, C₆₋₁₀ aryl-C₁₋₄-alkyl, C₁₋₁₀ heteroaryl are each optionally substituted by one or more groups selected from halo, cyano, nitro, C₁₋₆ alkyl, C₁₋₆ haloalkyl, C₂₋₆ alkenyl, C₂₋₆ alkynyl, C₁₋₆ alkoxy, hydroxy, C₁₋₆ alkoxy, C₁₋₆ haloalkoxy, C₁₋₆ alkylthio, C₁₋₆ alkylsulfinyl, C₁₋₆ alkylsulfonyl, carbamyl, C₁₋₆ alkylcarbamyl, di(C₁₋₆ alkyl)carbamyl, carboxy, amino, C₁₋₆ alkylamino, di-C₁₋₆ alkylamino, C₁₋₆ alkylcarbonyl, C₁₋₆ alkoxycarbonyl, C₁₋₆ alkylcarbonyloxy, C₁₋₆ alkylcarbonylamino, C₁₋₆ alkylsulfonylamino, amino sulfonyl, C₁₋₆ alkylamino sulfonyl, di(C₁₋₆ alkyl)aminosulfonyl, amino sulfonylamino, C₁₋₆ alkylaminosulfonylamino, di(C₁₋₆ alkyl)aminosulfonylamino, aminocarbonylamino, C₁₋₆ alkylaminocarbonylamino, di(C₁₋₆ alkyl)aminocarbonylamino, and C₃₋₁₀ cycloalkyl-C₁₋₄-alkyl, C₂₋₁₀ heterocycloalkyl, C₂₋₁₀ heterocycloalkyl-C₁₋₄-alkyl, C₆₋₁₀ aryl, C₆₋₁₀ aryl-C₁₋₄-alkyl, C₁₋₁₀ heteroaryl; and

each R^{4a} is independently selected from H and C₁₋₆ alkyl.

In one embodiments of the aforementioned embodiment, it is provided that each hydrogen atom in which is directly attached to a nitrogen atom, sulfur atom, or oxygen atom in any of the aforementioned groups (e.g., heteroaryl, heterocycloalkyl, C₁₋₆ alkyl-NR^{4a}—C₁₋₆ alkylene, hydroxy, carbamyl, carboxy, amino, C₁₋₆ alkylamino, C₁₋₆ alkylsulfonylamino, aminosulfonyl, C₁₋₆ alkylaminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino) is replaced by a protecting group.

Starting materials of Formula II can be obtained by reacting the aryl or heteroaryl substrate with a N-iodosuccinamide (NIS) in an appropriate solvent such as dry acetonitrile to give a compound of Formula II. Protecting groups can added if necessary as described in Wuts and Greene, Protective Groups in Organic Synthesis, 4th ed., John Wiley & Sons: New Jersey, which is incorporated herein by reference in its entirety. For example, amine groups can be protected by reacting di-tert-butyl dicarbonate (BOC anhydride in the presence of a tertiary amine (e.g, 4-dimethylpyridine and triethylamine) to form a BOC (tert-butylcarbonyl) protected amine.

In some embodiments, the present application provides a process of converting the compound of Formula I to a compound of Formula III:

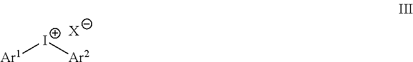

wherein Ar² is an optionally substituted aryl or heteroaryl.

In some embodiments, the conversion of the compound of Formula I to a compound of Formula III is done in the same pot as the reaction of the compound of Formula II to form the compound of Formula I.

In some embodiments, the converting comprises reacting the compound of Formula I with a compound of Formula IV:

Ar²-M¹ IV

wherein M¹ is a borate, stannane, silane, or zinc moiety.

In some embodiments, M¹ is Sn(R^{x})₃, Si(R^{y})₃, B(OR^{z})₂, or B(X²)₃M²; wherein:

each R^{x} is, independently, C₁₋₆ alkyl;

each R^{y} is, independently, C₁₋₆ alkyl;

each R^{z} is, independently, OH or C₁₋₆ alkoxy; or

two R^{z} groups, taken together with the oxygen atoms to which they are attached and the boron atom to which the oxygen atoms are attached, form a 5- to 6-membered heterocyclic ring, which is optionally substituted with 1, 2, 3, or 4 C₁₋₄ alkyl groups;

each X² is, independently, halo; and

M² is a counterion.

In some embodiments, the zinc moiety is an zinc halide (Zn-halo). In some embodiments, the arylzinc halide is zinc chloride.

In some embodiments, the compound of Formula IV is Ar²BF₃M².

In some embodiments, the compound of Formula IV is Ar²

BF₃K.

In some embodiments, the process is carried out in the presence of a catalyst.

In some embodiments, the catalyst is trimethylsilyl trifluoroacetate.

The use of Ar²BF₃M² is preferred over the other reagents. Compared to organostannanes, organoboranes are relatively straightforward to handle and are quite reactive toward I(III) compounds. However, organoboranes themselves are limited by the inherent characteristics of the in situ hydroboration reaction used to create them. They also suffer from high sensitivity to air and poor functional-group compatibility in some cases. In contrast, aryltrifluoroborates are stable, crystalline compounds that have been shown to overcome these limitations. Organotrifluoroborates can be easily prepared from inexpensive materials. They are stable to air and moisture, features that allow shipping and storage of these reagents for long periods of time without noticeable degradation. Their versatility and stability has made them excellent reagents in many organic reactions. Further, trifluoroborates have the ability to resist chemical oxidation. This feature offers aryltrifluoroborates a unique opportunity to preserve the carbon-boron bond during the oxidation of remote functionality within the same molecule. Organoboron compounds are generally incompatible with oxidants, which readily cleave the labile carbon-boron bond. Organotrifluoroborates can be utilized to overcome this limitation in an important way; since the organometallic reagent needs to be stable to excess Selectfluor reagent that is present in one-pot synthetic approach. The oxidative strength of Selectfluor reagent is well tolerated by aryltrifluoroborates; they are unaffected by residual Selectfluor.

In one embodiment (a), Ar¹ and Ar² are each, independently, aryl or heteroaryl. In some embodiments, Ar¹ and Ar² are unsubstituted. In some embodiments, Ar¹ and Ar² are independently substituted by one or more groups independently selected from halo, cyano, nitro, C₁₋₆ alkyl, C₁₋₁₆ alkyl, C₁₋₆ haloalkyl, C₂₋₁₆ alkenyl, C₂₋₁₆ alkynyl, C₁₋₆ alkoxy, C₃₋₁₄ cycloalkyl, C₃₋₁₄ cycloalkyl-C₁₋₄-alkyl, C₂₋₁₄ heterocycloalkyl, C₂₋₁₄ heterocycloalkyl-C₁₋₄-alkyl, C₆₋₁₄ aryl, C₆₋₁₄ aryl-C₁₋₄-alkyl, C₁₋₁₄ heteroaryl, C₁₋₁₄ heteroaryl-C₁₋₄-alkyl, —S(═O)R^{a}, —S(═O)₂R^{a}, —S(═O)₂NR^{g}R^{h}, —C(C═O)R^{b}, —C(C═O)NR^{g}R^{h}, —OC(═O)R^{a}, —OC(═O)NR^{g}R^{h}, —NR^{k}C(═O)R^{a}, —NR^{k}C(═O)OR^{b}, —NR^{k}C(═O)NR^{g}NR^{h}, —NR^{k}S(═O)₂R^{a}, —NR^{k}S(═O)₂NR^{g}R^{h}, C(═NRⁱ)NR^{g}R^{h}, NR^{k}C(═NRⁱ)NR^{g}R^{h}, —OR^{c}, —SR^{d}, —S(═O)₂OR^{e}, —C(C═O)OR^{f}, and —NR^{g}R^{h}; wherein said C₁₋₆ alkyl, C₁₋₆ haloalkyl, C₂₋₆ alkenyl, C₂₋₆ alkynyl, C₁₋₆ alkoxy, C₃₋₁₄ cycloalkyl, C₃₋₁₄ cycloalkyl-C₁₋₄-alkyl, C₂₋₁₄ heterocycloalkyl, C₂₋₁₄ heterocycloalkyl-C₁₋₄-alkyl, C₆₋₁₄ aryl, C₆₋₁₄ aryl-C₁₋₄-alkyl, C₁₋₁₄ heteroaryl, and C₁₋₁₄ heteroaryl-C₁₋₄-alkyl are each optionally substituted by one or more independently selected R² groups;

each Rⁱ is independently selected from H, C₁₋₆ alkyl, CN, C₁₋₆ alkoxy, or C(O)C₁₋₆ alkyl; each R^{a} is independently selected from H, C₁₋₆ alkyl, C₁₋₆ haloalkyl, C₂₋₆ alkenyl, C₂₋₆ alkynyl, aryl, C₃₋₁₀ cycloalkyl, C₃₋₁₀ cycloalkyl-C₁₋₄-alkyl, C₂₋₁₀ heterocycloalkyl, C₂₋₁₀ heterocycloalkyl-C₁₋₄-alkyl, C₆₋₁₀ aryl, C₆₋₁₀ aryl-C₁₋₄-alkyl, C₁₋₁₀ heteroaryl, wherein said C₁₋₆ alkyl, C₁₋₆ haloalkyl, C₂₋₆ alkenyl, C₂₋₆ alkynyl, C₃₋₁₀ cycloalkyl, C₃₋₁₀ cycloalkyl-C₁₋₄-alkyl, C₂₋₁₀ heterocycloalkyl, C₂₋₁₀ heterocycloalkyl-C₁₋₄-alkyl, C₆₋₁₀ aryl, C₆₋₁₀ aryl-C₁₋₄-alkyl, C₁₋₁₀ heteroaryl, and C₁₋₁₀ heteroaryl-C₁₋₄-alkyl are each optionally substituted by one or more independently selected R² groups;

each R^{b} is independently selected from C₁₋₆ alkyl, C₁₋₆ haloalkyl, C₂₋₆ alkenyl, C₂₋₆ alkynyl, C₃₋₁₀ cycloalkyl, C₃₋₁₀ cycloalkyl-C₁₋₄-alkyl, C₂₋₁₀ heterocycloalkyl, C₂₋₁₀ heterocycloalkyl-C₁₋₄-alkyl, C₆₋₁₀ aryl, C₆₋₁₀ aryl-C₁₋₄-alkyl, C₁₋₁₀ heteroaryl, wherein said C₁₋₆ alkyl, C₁₋₆ haloalkyl, C₂₋₆ alkenyl, C₂₋₆ alkynyl, aryl, C₃₋₁₀ cycloalkyl, C₃₋₁₀ cycloalkyl-C₁₋₄-alkyl, C₂₋₁₀ heterocycloalkyl, C₂₋₁₀ heterocycloalkyl-C₁₋₄-alkyl, C₆₋₁₀ aryl, C₆₋₁₀ aryl-C₁₋₄-alkyl, C₁₋₁₀ heteroaryl, and C₁₋₁₀ heteroaryl-C₁₋₄-alkyl are each optionally substituted by one or more independently selected R² groups;

each R^{c} is independently selected from a protecting group, C₁₋₆ alkyl, C₁₋₆ haloalkyl, C₂₋₆ alkenyl, C₂₋₆ alkynyl, C₃₋₁₀ cyclo alkyl, C₃₋₁₀ cycloalkyl-C₁₋₄-alkyl, C₂₋₁₀ heterocycloalkyl, C₂₋₁₀ heterocycloalkyl-C₁₋₄-alkyl, C₆₋₁₀ aryl, C₆₋₁₀ aryl-C₁₋₄-alkyl, C₁₋₁₀ heteroaryl, wherein said C₁₋₆alkyl, C₁₋₆ haloalkyl, C₂₋₆ alkenyl, C₂₋₆ alkynyl, aryl, C₃₋₁₀ cycloalkyl, C₃₋₁₀ cycloalkyl-C₁₋₄-alkyl, C₂₋₁₀ heterocycloalkyl, C₂₋₁₀ heterocycloalkyl-C₁₋₄-alkyl, C₆₋₁₀ aryl, C₆₋₁₀ aryl-C₁₋₄-alkyl, C₁₋₁₀ heteroaryl, and C₁₋₁₀ heteroaryl-C₁₋₄-alkyl are each optionally substituted by one or more independently selected R² groups;

each R^{d} is independently selected from a protecting group, C₁₋₆ alkyl, C₁₋₆ haloalkyl, C₂₋₆ alkenyl, C₂₋₆ alkynyl, C₃₋₁₀ cyclo alkyl, C₃₋₁₀ cycloalkyl-C₁₋₄-alkyl, C₂₋₁₀ heterocycloalkyl, C₂₋₁₀ heterocycloalkyl-C₁₋₄-alkyl, C₆₋₁₀ aryl, C₆₋₁₀ aryl-C₁₋₄-alkyl, C₁₋₁₀ heteroaryl, wherein said C₁₋₆ alkyl, C₁₋₆ haloalkyl, C₂₋₆ alkenyl, C₂₋₆ alkynyl, aryl, C₃₋₁₀ cycloalkyl, C₃₋₁₀ cycloalkyl-C₁₋₄-alkyl, C₂₋₁₀ heterocycloalkyl, C₂₋₁₀ heterocycloalkyl-C₁₋₄-alkyl, C₆₋₁₀ aryl, C₆₋₁₀ aryl-C₁₋₄-alkyl, C₁₋₁₀ heteroaryl, and C₁₋₁₀ heteroaryl-C₁₋₄-alkyl are each optionally substituted by one or more independently selected R² groups;

each R^{e} is independently selected from a protecting group, C₁₋₆ alkyl, C₁₋₆ haloalkyl, C₂₋₆ alkenyl, C₂₋₆ alkynyl, C₃₋₁₀ cyclo alkyl, C₃₋₁₀ cycloalkyl-C₁₋₄-alkyl, C₂₋₁₀ heterocycloalkyl, C₂₋₁₀ heterocycloalkyl-C₁₋₄-alkyl, C₆₋₁₀ aryl, C₆₋₁₀ aryl-C₁₋₄-alkyl, C₁₋₁₀ heteroaryl, wherein said C₁₋₆ alkyl, C₁₋₆ haloalkyl, C₂₋₆ alkenyl, C₂₋₆ alkynyl, aryl, C₃₋₁₀ cycloalkyl, C₃₋₁₀ cycloalkyl-C₁₋₄-alkyl, C₂₋₁₀ heterocycloalkyl, C₂₋₁₀ heterocycloalkyl-C₁₋₄-alkyl, C₆₋₁₀ aryl, C₆₋₁₀ aryl-C₁₋₄-alkyl, C₁₋₁₀ heteroaryl, and C₁₋₁₀ heteroaryl-C₁₋₄-alkyl are each optionally substituted by one or more independently selected R² groups;

each R^{f} is independently selected from a protecting group, C₁₋₆ alkyl, C₁₋₆ haloalkyl, C₂₋₆ alkenyl, C₂₋₆ alkynyl, C₃₋₁₀ cyclo alkyl, C₃₋₁₀ cycloalkyl-C₁₋₄-alkyl, C₂₋₁₀ heterocycloalkyl, C₂₋₁₀ heterocycloalkyl-C₁₋₄-alkyl, C₆₋₁₀ aryl, C₆₋₁₀ aryl-C₁₋₄-alkyl, C₁₋₁₀ heteroaryl, wherein said C₁₋₆ alkyl, C₁₋₆ haloalkyl, C₂₋₆ alkenyl, C₂₋₆ alkynyl, aryl, C₃₋₁₀ cycloalkyl, C₃₋₁₀ cycloalkyl-C₁₋₄-alkyl, C₂₋₁₀ heterocycloalkyl, C₂₋₁₀ heterocycloalkyl-C₁₋₄-alkyl, C₆₋₁₀ aryl, C₆₋₁₀ aryl-C₁₋₄-alkyl, C₁₋₁₀ heteroaryl, and C₁₋₁₀ heteroaryl-C₁₋₄-alkyl are each optionally substituted by one or more independently selected R² groups;

each R^{k}, R^{g} and R^{h} is independently selected from a protecting group, C₁₋₆ alkyl, C₁₋₆ haloalkyl, C₂₋₆ alkenyl, C₂₋₆ alkynyl, C₃₋₁₀ cycloalkyl, C₃₋₁₀ cycloalkyl-C₁₋₄-alkyl, C₂₋₁₀ heterocycloalkyl, C₂₋₁₀ heterocycloalkyl-C₁₋₄-alkyl, C₆₋₁₀ aryl, C₆₋₁₀ aryl-C₁₋₄-alkyl, C₁₋₁₀ heteroaryl, wherein said C₁₋₆ alkyl, C₁₋₆ haloalkyl, C₂₋₆ alkenyl, C₂₋₆ alkynyl, aryl, C₃₋₁₀ cycloalkyl, C₃₋₁₀ cycloalkyl-C₁₋₄-alkyl, C₂₋₁₀ heterocycloalkyl, C₂₋₁₀ heterocycloalkyl-C₁₋₄-alkyl, C₆₋₁₀ aryl, C₆₋₁₀ aryl-C₁₋₄-alkyl, C₁₋₁₀heteroaryl, and C₁₋₁₀heteroaryl-C₁₋₄-alkyl are each optionally substituted by one or more independently selected R² groups;

or alternatively, R^{k} and R^{a}, taken together with the atoms to which they are attached, form a heterocycloalkyl or heteroaryl ring, which is optionally substituted by one or more R² groups;

or alternatively, R^{k} and R^{b}, taken together with the atoms to which they are attached, form a heterocycloalkyl or heteroaryl ring, which is optionally substituted by one or more R² groups;

or alternatively, R^{k} and R^{g}, taken together with the atoms to which they are attached, form a heterocycloalkyl or heteroaryl ring, which is optionally substituted by one or more R² groups;

or alternatively, R^{g} and R^{h}, taken together with the nitrogen atom to which they are attached, form a heterocycloalkyl or heteroaryl ring, which is optionally substituted by one or more R⁴ groups;

each R² is independently selected from halo, cyano, nitro, C₁₋₆ alkyl, C₁₋₆haloalkyl, C₁₋₆ haloalkyl, C₂₋₆ alkenyl, C₂₋₆ alkynyl, C₁₋₆ alkoxy, C₃₋₁₀ cycloalkyl, C₃₋₁₀ cycloalkyl-C₁₋₄-alkyl, C₂₋₁₀ heterocycloalkyl, C₂₋₁₀ heterocycloalkyl-C₁₋₄-alkyl, C₆₋₁₀ aryl, C₆₋₁₀ aryl-C₁₋₄-alkyl, C₁₋₁₀ heteroaryl, C₁₋₁₀heteroaryl-C₁₋₄-alkyl, —S(═O)R^{a1}, —S(═O)₂R^{a1}, —S(═O)₂NR^{g1}R^{h1}, —C(—O)R^{b1}, —C(C═O)NR^{g1}R^{h1}, —OC(═O)R^{a1}, —OC(═O)NR^{g1}R^{h1}, —NR^{k1}C(O)R^{a1}, —NR^{k1}C(O)OR^{b1}, —NR^{k1}C(═O)NR^{g1}NR^{h1}, —NR^{k1}S(O)₂R^{a1}, —NR^{k1}S(O)₂NR^{g1}R^{h1}, C(NRⁱ)NR^{g1}R^{h1}, NR^{k1}C(═NRⁱ)NR^{g1}R^{h1}, —OR^{c1}, —SR^{d1}, —S(═O)₂OR^{e1}, —C(C═O)OR^{f1}, and —NR^{g1}R^{h1}; wherein said C₁₋₆ alkyl, C₁₋₆haloalkyl, C₂₋₆ alkenyl, C₂₋₆ alkynyl, C₁₋₆ alkoxy, C₃₋₁₀ cycloalkyl, C₃₋₁₀ cycloalkyl-C₁-4-alkyl, C₂₋₁₀ heterocycloalkyl, C₂₋₁₀ heterocycloalkyl-C₁₋₄-alkyl, C₆₋₁₀ aryl, C₆₋₁₀ aryl-C₁₋₄-alkyl, C₁₋₁₀heteroaryl, and C₁₋₁₀ heteroaryl-C₁₋₄-alkyl are each optionally substituted by one or more independently selected R³ groups;

each R^{a1} is independently selected from H, C₁₋₆ alkyl, C₁₋₆haloalkyl, C₂₋₆ alkenyl, C₂₋₆ alkynyl, C₃₋₁₀ cycloalkyl, C₃₋₁₀ cycloalkyl-C₁₋₄-alkyl, C₂₋₁₀ heterocycloalkyl, C₂₋₁₀ heterocycloalkyl-C₁₋₄-alkyl, C₆₋₁₀ aryl, C₆₋₁₀ aryl-C₁₋₄-alkyl, C₁₋₁₀ heteroaryl, wherein said C₁₋₆ alkyl, C₁₋₆haloalkyl, C₂₋₆ alkenyl, C₂₋₆ alkynyl, C₃₋₁₀ cycloalkyl, C₃₋₁₀ cycloalkyl-C₁₋₄-alkyl, C₂₋₁₀ heterocycloalkyl, C₂₋₁₀ heterocycloalkyl-C₁₋₄-alkyl, C₆₋₁₀ aryl, C₆₋₁₀ aryl-C₁₋₄-alkyl, Cᵢᵣₒ heteroaryl, and C₁₋₁₀ heteroaryl-C₁₋₄-alkyl are each optionally substituted by one or more independently selected R³ groups;

each R^{b1} is independently selected from C₁₋₆ alkyl, C₁₋₆ haloalkyl, C₂₋₆ alkenyl, C₂₋₆ alkynyl, C₃₋₁₀ cycloalkyl, C₃₋₁₀ cycloalkyl-C₁₋₄-alkyl, C₂₋₁₀ heterocycloalkyl, C₂₋₁₀ heterocycloalkyl-C₁₋₄-alkyl, C₆₋₁₀ aryl, C₆₋₁₀ aryl-C₁₋₄-alkyl, C₁₋₁₀heteroaryl, wherein said C₁₋₆ alkyl, C₁₋₆ haloalkyl, C₂₋₆ alkenyl, C₂₋₆ alkynyl, C₃₋₁₀ cycloalkyl, C₃₋₁₀ cycloalkyl-C₁₋₄-alkyl, C₂₋₁₀ heterocycloalkyl, C₂₋₁₀ heterocycloalkyl-C₁₋₄-alkyl, C₆₋₁₀ aryl, C₆₋₁₀ aryl-C₁₋₄-alkyl, C₁₋₁₀ heteroaryl, and C₁₋₁₀ heteroaryl-C₁₋₄-alkyl are each optionally substituted by one or more independently selected R³ groups;

each R^{c1} is independently selected from a protecting group, C₁₋₆ alkyl, C₁₋₆ haloalkyl, C₂₋₆ alkenyl, C₂₋₆ alkynyl, C₃₋₁₀ cyclo alkyl, C₃₋₁₀ cycloalkyl-C₁₋₄-alkyl, C₂₋₁₀ heterocycloalkyl, C₂₋₁₀ heterocycloalkyl-C₁₋₄-alkyl, C₆₋₁₀ aryl, C₆₋₁₀ aryl-C₁₋₄-alkyl, C₁₋₁₀ heteroaryl, wherein said C₁₋₆ alkyl, C₁₋₆ haloalkyl, C₂₋₆ alkenyl, C₂₋₆ alkynyl, C₃₋₁₀ cycloalkyl, C₃₋₁₀ cycloalkyl-C₁₋₄-alkyl, C₂₋₁₀ heterocycloalkyl, C₂₋₁₀ heterocycloalkyl-C₁₋₄-alkyl, C₆₋₁₀ aryl, C₆₋₁₀ aryl-C₁₋₄-alkyl, C₁₋₁₀ heteroaryl, and C₁₋₁₀ heteroaryl-C₁₋₄-alkyl are each optionally substituted by one or more independently selected R³ groups;

each R^{d1} is independently selected from a protecting group, C₁₋₆ alkyl, C₁₋₆ haloalkyl, C₂₋₆ alkenyl, C₂₋₆ alkynyl, C₃₋₁₀ cyclo alkyl, C₃₋₁₀ cycloalkyl-C₁₋₄-alkyl, C₂₋₁₀ heterocycloalkyl, C₂₋₁₀ heterocycloalkyl-C₁₋₄-alkyl, C₆₋₁₀ aryl, C₆₋₁₀ aryl-C₁₋₄-alkyl, C₁₋₁₀heteroaryl, wherein said C₁₋₆ alkyl, C₁₋₆ haloalkyl, C₂₋₆ alkenyl, C₂₋₆ alkynyl, C₃₋₁₀ cycloalkyl, C₃₋₁₀ cycloalkyl-C₁₋₄-alkyl, C₂₋₁₀ heterocycloalkyl, C₂₋₁₀ heterocycloalkyl-C₁₋₄-alkyl, C₆₋₁₀ aryl, C₆₋₁₀ aryl-C₁₋₄-alkyl, C₁₋₁₀ heteroaryl, and C₁₋₁₀ heteroaryl-C₁₋₄-alkyl are each optionally substituted by one or more independently selected R³ groups;

each R^{e1} is independently selected from a protecting group, C₁₋₆ alkyl, C₁₋₆ haloalkyl, C₂₋₆ alkenyl, C₂₋₆ alkynyl, C₃₋₁₀ cyclo alkyl, C₃₋₁₀ cycloalkyl-C₁₋₄-alkyl, C₂₋₁₀ heterocycloalkyl, C₂₋₁₀ heterocycloalkyl-C₁₋₄-alkyl, C₆₋₁₀ aryl, C₆₋₁₀ aryl-C₁₋₄-alkyl, C₁₋₁₀ heteroaryl, wherein said C₁₋₆ alkyl, C₁₋₆ haloalkyl, C₂₋₆ alkenyl, C₂₋₆ alkynyl, C₃₋₁₀ cycloalkyl, C₃₋₁₀ cycloalkyl-C₁₋₄-alkyl, C₂₋₁₀ heterocycloalkyl, C₂₋₁₀ heterocycloalkyl-C₁₋₄-alkyl, C₆₋₁₀ aryl, C₆₋₁₀ aryl-C₁₋₄-alkyl, C₁₋₁₀ heteroaryl, and C₁₋₁₀ heteroaryl-C₁₋₄-alkyl are each optionally substituted by one or more independently selected R³ groups;

each R^{f1} is independently selected from a protecting group, C₁₋₆ alkyl, C₁₋₆ halo alkyl, C₂₋₆ alkenyl, C₂₋₆ alkynyl, C₃₋₁₀ cyclo alkyl, C₃₋₁₀ cycloalkyl-C₁₋₄-alkyl, C₂₋₁₀ heterocycloalkyl, C₂₋₁₀ heterocycloalkyl-C₁₋₄-alkyl, C₆₋₁₀ aryl, C₆₋₁₀ aryl-C₁₋₄-alkyl, C₁₋₁₀heteroaryl, wherein said C₁₋₆alkyl, C₁₋₆ haloalkyl, C₂₋₆ alkenyl, C₂₋₆ alkynyl, C₃₋₁₀ cycloalkyl, C₃₋₁₀ cycloalkyl-C₁₋₄-alkyl, C₂₋₁₀ heterocycloalkyl, C₂₋₁₀ heterocycloalkyl-C₁₋₄-alkyl, C₆₋₁₀ aryl, C₆₋₁₀ aryl-C₁₋₄-alkyl, C₁₋₁₀ heteroaryl, and C₁₋₁₀ heteroaryl-C₁₋₄-alkyl are each optionally substituted by one or more independently selected R³ groups;

each R^{k1}, R^{g1} and R^{h2} is independently selected from a protecting group, C₁₋₆ alkyl, C₁₋₆ haloalkyl, C₂₋₆ alkenyl, C₂₋₆ alkynyl, C₃₋₁₀ cycloalkyl, C₃₋₁₀ cycloalkyl-C₁₋₄-alkyl, C₂₋₁₀ heterocycloalkyl, C₂₋₁₀ heterocycloalkyl-C₁₋₄-alkyl, C₆₋₁₀ aryl, C₆₋₁₀ aryl-C₁₋₄-alkyl, C₁₋₁₀ heteroaryl, wherein said C₁₋₆ alkyl, C₁₋₆ haloalkyl, C₂₋₆ alkenyl, C₂₋₆ alkynyl, C₃₋₁₀ cycloalkyl, C₃₋₁₀ cycloalkyl-C₁₋₄-alkyl, C₂₋₁₀ heterocycloalkyl, C₂₋₁₀ heterocycloalkyl-C₁₋₄-alkyl, C₆₋₁₀ aryl, C₆₋₁₀ aryl-C₁₋₄-alkyl, C₁₋₁₀ heteroaryl, and C₁₋₁₀ heteroaryl-C₁₋₄-alkyl are each optionally substituted by one or more independently selected R³ groups;

or alternatively, R^{k1} and R^{a1}, taken together with the atoms to which they are attached, form a heterocycloalkyl or heteroaryl ring, which is optionally substituted by one or more R³ groups;

or alternatively, R^{k1} and R^{b1}, taken together with the atoms to which they are attached, form a heterocycloalkyl or heteroaryl ring, which is optionally substituted by one or more R³ groups;

or alternatively, R^{k1} and R^{g1}, taken together with the atoms to which they are attached, form a heterocycloalkyl or heteroaryl ring, which is optionally substituted by one or more R³ groups;

or alternatively, R^{g1} and R^{h1}, taken together with the nitrogen atom to which they are attached, form a heterocycloalkyl or heteroaryl ring, which is optionally substituted by one or more R³ groups;

each R³ is independently selected from halo, cyano, nitro, C₁₋₆ alkyl, C₁₋₆ haloalkyl, C₁₋₆ haloalkyl, C₂₋₆ alkenyl, C₂₋₆ alkynyl, C₁₋₆ alkoxy, C₃₋₁₀ cycloalkyl, C₃₋₁₀ cycloalkyl-C₁₋₄-alkyl, C₂₋₁₀ heterocycloalkyl, C₂₋₁₀ heterocycloalkyl-C₁₋₄-alkyl, C₆₋₁₀ aryl, C₆₋₁₀ aryl-C₁₋₄-alkyl, C₁₋₁₀ heteroaryl, C₁₋₁₀heteroaryl-C₁₋₄-alkyl, —S(═O)R^{a2}, —S(═O)₂R^{a2}, —S(═O)₂NR^{g2}R^{h2}, —C(C═O)R^{b2}, —C(C═O)NR^{g2}R^{h2}, —OC(═O)R^{a2}, —OC(═O)NR^{g2}R^{h2}, —NR^{k2}C(═O)R^{a2}, —NR^{k2}C(═O)OR^{b2}, —NR^{k2}C(═O)NR^{g2}NR^{h2}, —NR^{k2}S(═O)₂R^{a2}, —NR^{k2}S(═O)₂NR^{g2}R^{h2}, C(═NRⁱ)NR^{g2}R^{h2}, NR^{k2}C(═NRⁱ)NR^{g2}R^{h2}, —OR^{e2}, —SR^{d2}, —S(═O)₂OR^{e2}, —C(C═O)OR^{f2}, and —NR^{g2}R^{h2}; wherein said C₁₋₆ alkyl, C₁₋₆ haloalkyl, C₂₋₆ alkenyl, C₂₋₆ alkynyl, C₁₋₆ alkoxy, C₃₋₁₀ cycloalkyl, C₃₋₁₀ cycloalkyl-C₁₋₄-alkyl, C₂₋₁₀ heterocycloalkyl, C₂₋₁₀ heterocycloalkyl-C₁₋₄-alkyl, C₆₋₁₀ aryl, C₆₋₁₀ aryl-C₁₋₄-alkyl, C₁₋₁₀heteroaryl, and C₁₋₁₀ heteroaryl-C₁₋₄-alkyl are each optionally substituted by one or more independently selected R⁴ groups;

each R^{a2} is independently selected from H, C₁₋₆ alkyl, C₁₋₆ haloalkyl, C₂₋₆ alkenyl, C₂₋₆ alkynyl, C₃₋₁₀ cycloalkyl, C₃₋₁₀ cycloalkyl-C₁₋₄-alkyl, C₂₋₁₀ heterocycloalkyl, C₂₋₁₀ heterocycloalkyl-C₁₋₄-alkyl, C₆₋₁₀ aryl, C₆₋₁₀ aryl-C₁₋₄-alkyl, C₁₋₁₀heteroaryl, wherein said C₁₋₆ alkyl, C₁₋₆ haloalkyl, C₂₋₆ alkenyl, C₂₋₆ alkynyl, C₃₋₁₀ cycloalkyl, C₃₋₁₀ cycloalkyl-C₁₋₄-alkyl, C₂₋₁₀ heterocycloalkyl, C₂₋₁₀ heterocycloalkyl-C₁₋₄-alkyl, C₆₋₁₀ aryl, C₆₋₁₀ aryl-C₁₋₄-alkyl, C₁₋₁₀ heteroaryl, and C₁₋₁₀ heteroaryl-C₁₋₄-alkyl are each optionally substituted by one or more independently selected R⁴ groups;

each R^{b2} is independently selected from C₁₋₆ alkyl, C₁₋₆ haloalkyl, C₂₋₆ alkenyl, C₂₋₆ alkynyl, C₃₋₁₀ cycloalkyl, C₃₋₁₀ cycloalkyl-C₁₋₄-alkyl, C₂₋₁₀ heterocycloalkyl, C₂₋₁₀ heterocycloalkyl-C₁₋₄-alkyl, C₆₋₁₀ aryl, C₆₋₁₀ aryl-C₁₋₄-alkyl, C₁₋₁₀heteroaryl, wherein said C₁₋₆ alkyl, C₁₋₆ haloalkyl, C₂₋₆ alkenyl, C₂₋₆ alkynyl, C₃₋₁₀ cycloalkyl, C₃₋₁₀ cycloalkyl-C₁₋₄-alkyl, C₂₋₁₀ heterocycloalkyl, C₂₋₁₀ heterocycloalkyl-C₁₋₄-alkyl, C₆₋₁₀ aryl, C₆₋₁₀ aryl-C₁₋₄-alkyl, C₁₋₁₀ heteroaryl, and C₁₋₁₀ heteroaryl-C₁₋₄-alkyl are each optionally substituted by one or more independently selected R⁴ groups;

each R^{c2} is independently selected from a protecting group, C₁₋₆ alkyl, C₁₋₆ haloalkyl, C₂₋₆ alkenyl, C₂₋₆ alkynyl, C₃₋₁₀ cyclo alkyl, C₃₋₁₀ cycloalkyl-C₁₋₄-alkyl, C₂₋₁₀ heterocycloalkyl, C₂₋₁₀ heterocycloalkyl-C₁₋₄-alkyl, C₆₋₁₀ aryl, C₆₋₁₀ aryl-C₁₋₄-alkyl, C₁₋₁₀ heteroaryl, wherein said C₁₋₆ alkyl, C₁₋₆ haloalkyl, C₂₋₆ alkenyl, C₂₋₆ alkynyl, C₃₋₁₀ cycloalkyl, C₃₋₁₀ cycloalkyl-C₁₋₄-alkyl, C₂₋₁₀ heterocycloalkyl, C₂₋₁₀ heterocycloalkyl-C₁₋₄-alkyl, C₆₋₁₀ aryl, C₆₋₁₀ aryl-C₁₋₄-alkyl, C₁₋₁₀ heteroaryl, and C₁₋₁₀ heteroaryl-C₁₋₄-alkyl are each optionally substituted by one or more independently selected R⁴ groups;

each R^{d2} is independently selected from a protecting group, C₁₋₆ alkyl, C₁₋₆ haloalkyl, C₂₋₆ alkenyl, C₂₋₆ alkynyl, C₃₋₁₀ cyclo alkyl, C₃₋₁₀ cycloalkyl-C₁₋₄-alkyl, C₂₋₁₀ heterocycloalkyl, C₂₋₁₀ heterocycloalkyl-C₁₋₄-alkyl, C₆₋₁₀ aryl, C₆₋₁₀ aryl-C₁₋₄-alkyl, C₁₋₁₀heteroaryl, wherein said C₁₋₆ alkyl, C₁₋₆ haloalkyl, C₂₋₆ alkenyl, C₂₋₆ alkynyl, C₃₋₁₀ cycloalkyl, C₃₋₁₀ cycloalkyl-C₁₋₄-alkyl, C₂₋₁₀ heterocycloalkyl, C₂₋₁₀ heterocycloalkyl-C₁₋₄-alkyl, C₆₋₁₀ aryl, C₆₋₁₀ aryl-C₁₋₄-alkyl, C₁₋₁₀ heteroaryl, and C₁₋₁₀ heteroaryl-C₁₋₄-alkyl are each optionally substituted by one or more independently selected R⁴ groups;

each R^{e2} is independently selected from a protecting group, C₁₋₆ alkyl, C₁₋₆haloalkyl, C₂₋₆ alkenyl, C₂₋₆ alkynyl, C₃₋₁₀ cyclo alkyl, C₃₋₁₀ cycloalkyl-C₁₋₄-alkyl, C₂₋₁₀ heterocycloalkyl, C₂₋₁₀ heterocycloalkyl-C₁₋₄-alkyl, C₆₋₁₀ aryl, C₆₋₁₀ aryl-C₁₋₄-alkyl, C₁₋₁₀heteroaryl, wherein said C₁₋₆ alkyl, C₁₋₆haloalkyl, C₂₋₆ alkenyl, C₂₋₆ alkynyl, C₃₋₁₀ cycloalkyl, C₃₋₁₀ cycloalkyl-C₁₋₄-alkyl, C₂₋₁₀ heterocycloalkyl, C₂₋₁₀ heterocycloalkyl-C₁₋₄-alkyl, C₆₋₁₀ aryl, C₆₋₁₀ aryl-C₁₋₄-alkyl, C₁₋₁₀ heteroaryl, and C₁₋₁₀ heteroaryl-C₁₋₄-alkyl are each optionally substituted by one or more independently selected R⁴ groups;

each R^{f2} is independently selected from a protecting group, C₁₋₆ alkyl, C₁₋₆haloalkyl, C₂₋₆ alkenyl, C₂₋₆ alkynyl, C₃₋₁₀ cyclo alkyl, C₃₋₁₀ cycloalkyl-C₁₋₄-alkyl, C₂₋₁₀ heterocycloalkyl, C₂₋₁₀ heterocycloalkyl-C₁₋₄-alkyl, C₆₋₁₀ aryl, C₆₋₁₀ aryl-C₁₋₄-alkyl, C₁₋₁₀ heteroaryl, wherein said C₁₋₆ alkyl, C₁₋₆haloalkyl, C₂₋₆ alkenyl, C₂₋₆ alkynyl, C₃₋₁₀ cycloalkyl, C₃₋₁₀ cycloalkyl-C₁₋₄-alkyl, C₂₋₁₀ heterocycloalkyl, C₂₋₁₀ heterocycloalkyl-C₁₋₄-alkyl, C₆₋₁₀ aryl, C₆₋₁₀ aryl-C₁₋₄-alkyl, C₁₋₁₀ heteroaryl, and C₁₋₁₀ heteroaryl-C₁₋₄-alkyl are each optionally substituted by one or more independently selected R⁴ groups;

each R^{k2}, R^{g2} and R^{h2} is independently selected from a protecting group, C₁₋₆ alkyl, C₁₋₆ haloalkyl, C₂₋₆ alkenyl, C₂₋₆ alkynyl, C₃₋₁₀ cycloalkyl, C₃₋₁₀ cycloalkyl-C₁₋₄-alkyl, C₂₋₁₀ heterocycloalkyl, C₂₋₁₀ heterocycloalkyl-C₁₋₄-alkyl, C₆₋₁₀ aryl, C₆₋₁₀ aryl-C₁₋₄-alkyl, C₁₋₁₀ heteroaryl, wherein said C₁₋₆ alkyl, C₁₋₆haloalkyl, C₂₋₆ alkenyl, C₂₋₆ alkynyl, C₃₋₁₀ cycloalkyl, C₃₋₁₀ cycloalkyl-C₁₋₄-alkyl, C₂₋₁₀ heterocycloalkyl, C₂₋₁₀ heterocycloalkyl-C₁₋₄-alkyl, C₆₋₁₀ aryl, C₆₋₁₀ aryl-C₁₋₄-alkyl, C₁₋₁₀ heteroaryl, and C₁₋₁₀ heteroaryl-C₁₋₄-alkyl are each optionally substituted by one or more independently selected R⁴ groups;

or alternatively, R^{k2} and R^{a2}, taken together with the atoms to which they are attached, form a heterocycloalkyl or heteroaryl ring, which is optionally substituted by one or more R⁴ groups;

or alternatively, R^{k2} and R^{b2}, taken together with the atoms to which they are attached, form a heterocycloalkyl or heteroaryl ring, which is optionally substituted by one or more R⁴ groups;

or alternatively, R^{k2} and R^{g2}, taken together with the atoms to which they are attached, form a heterocycloalkyl or heteroaryl ring, which is optionally substituted by one or more R⁴ groups;

or alternatively, R^{g2} and R^{h2}, taken together with the nitrogen atom to which they are attached, form a heterocycloalkyl or heteroaryl ring, which is optionally substituted by one or more R⁴ groups;

each R⁴ is independently selected from halo, cyano, nitro, C₁₋₆ alkyl, C₁₋₆ haloalkyl, C₁₋₆ alkyl-NR^{4a}—C₁₋₆ alkylene, C₁₋₆ alkyl-O—C₁₋₆ alkylene, C₂₋₆ alkenyl, C₂₋₆ alkynyl, C₃₋₁₀ cycloalkyl, C₃₋₁₀ cycloalkyl-C₁₋₄-alkyl, C₂₋₁₀ heterocycloalkyl, C₂₋₁₀ heterocycloalkyl-C₁₋₄-alkyl, C₆₋₁₀ aryl, C₆₋₁₀ aryl-C₁₋₄-alkyl, C₁₋₁₀ heteroaryl, hydroxy, C₁₋₆ alkoxy, C₁₋₆ haloalkoxy, C₁₋₆ alkylthio, C₁₋₆ alkylsulfinyl, C₁₋₆ alkylsulfonyl, carbamyl, C₁₋₆ alkylcarbamyl, di(C₁₋₆ alkyl)carbamyl, carboxy, amino, C₁₋₆ alkylamino, di-C₁₋₆ alkylamino, C₁₋₆ alkylcarbonyl, C₁₋₆ alkoxycarbonyl, C₁₋₆ alkylcarbonyloxy, C₁₋₆ alkylcarbonylamino, C₁₋₆ alkylsulfonylamino, amino sulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino; wherein said C₁₋₆ alkyl, C₁₋₆ haloalkyl, C₂₋₆ alkenyl, C₂₋₆ alkynyl, C₁₋₆ alkoxy, C₁₋₆ alkyl-NR^{4a}—C₁₋₆ alkylene, C₁₋₆ alkyl-O—C₁₋₆ alkylene, C₃₋₁₀ cycloalkyl, C₃₋₁₀ cycloalkyl-C₁₋₄-alkyl, C₂₋₁₀ heterocycloalkyl, C₂₋₁₀ heterocycloalkyl-C₁₋₄-alkyl, C₆₋₁₀ aryl, C₆₋₁₀ aryl-C₁₋₄-alkyl, C₁₋₁₀ heteroaryl are each optionally substituted by one or more groups selected from halo, cyano, nitro, C₁₋₆ alkyl, C₁₋₆ haloalkyl, C₂₋₆ alkenyl, C₂₋₆ alkynyl, C₁₋₆ alkoxy, hydroxy, C₁₋₆ alkoxy, C₁₋₆ haloalkoxy, C₁₋₆ alkylthio, C₁₋₆ alkylsulfinyl, C₁₋₆ alkylsulfonyl, carbamyl, C₁₋₆ alkylcarbamyl, di(C₁₋₆ alkyl)carbamyl, carboxy, amino, C₁₋₆ alkylamino, di-C₁₋₆ alkylamino, C₁₋₆ alkylcarbonyl, C₁₋₆ alkoxycarbonyl, C₁₋₆ alkylcarbonyloxy, C₁₋₆ alkylcarbonylamino, C₁₋₆ alkylsulfonylamino, amino sulfonyl, C₁₋₆ alkylamino sulfonyl, di(C₁₋₆ alkyl)aminosulfonyl, amino sulfonylamino, C₁₋₆ alkylaminosulfonylamino, di(C₁₋₆ alkyl)aminosulfonylamino, aminocarbonylamino, C₁₋₆ alkylaminocarbonylamino, di(C₁₋₆ alkyl)aminocarbonylamino, and C₃₋₁₀ cycloalkyl-C₁₋₄-alkyl, C₂₋₁₀ heterocycloalkyl, C₂₋₁₀ heterocycloalkyl-C₁₋₄-alkyl, C₆₋₁₀ aryl, C₆₋₁₀ aryl-C₁₋₄-alkyl, C₁₋₁₀ heteroaryl; and

each R^{4a} is independently selected from H and C₁₋₆ alkyl;

provided that each hydrogen atom in which is directly attached to a nitrogen atom, sulfur atom, or oxygen atom in any of the aforementioned groups (e.g., heteroaryl, heterocycloalkyl, C₁₋₆ alkyl-NR^{4a}—C₁₋₆ alkylene, hydroxy, carbamyl, carboxy, amino, C₁₋₆ alkylamino, C₁₋₆ alkylsulfonylamino, amino sulfonyl, C₁₋₆ alkylamino sulfonyl, amino sulfonylamino, C₁₋₆alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino) is replaced by a protecting group.

In some embodiments, Ar¹ is defined as in embodiment (a).

In some embodiments, Ar¹ is:

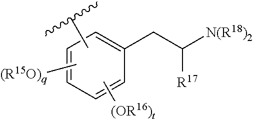

wherein;

q is 0 or 1;

t is 0 or 1;

R¹⁵ and R¹⁶ are each, independently, an acid labile protecting group;

R¹⁷ is selected from hydrogen and C(O)₂R¹⁹;

R¹⁸ in each occurrence is independently selected from hydrogen and t-butoxycarbonyl;

and

R¹⁹ is selected from hydrogen, methyl, and t-butyl.

In some embodiments, Ar¹ is:

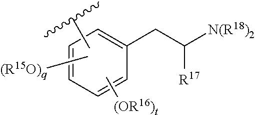

wherein;

q is 0 or 1;

t is 0 or 1;

R¹⁵ and R¹⁶ are each, independently, alkoxy;

R¹⁷ is selected from hydrogen and C(O)₂R¹⁹;

R¹⁸ in each occurrence is independently selected from hydrogen and t-butoxycarbonyl;

and

R¹⁹ is selected from hydrogen, methyl, and t-butyl.

It is to be understood that in all instances where a phenyl ring shows one or more dangling substituents, it is intended to mean that the particular substitutent(s) may be attached to any suitable carbon of the phenyl ring. This intended to apply as well to dangling points of attachment. For example, the following structure:

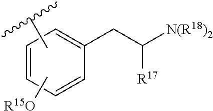

is intended to include at least the following structures:

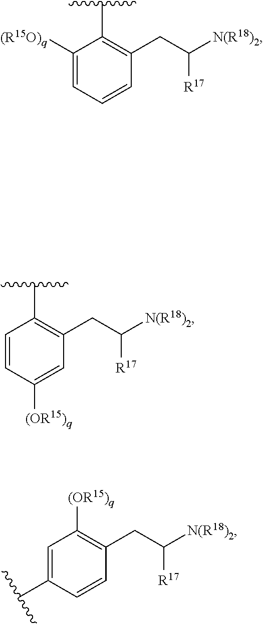

etc.

In some embodiments, Ar¹ is:

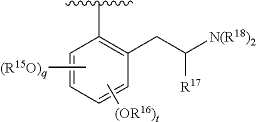

wherein;

q is 0 or 1;

t is 0 or 1;

R¹⁵ and R¹⁶ are each, independently, an acid labile protecting group;

R¹⁷ is selected from hydrogen and C(O)₂R¹⁹;

R¹⁸ in each occurrence is independently selected from hydrogen and t-butoxycarbonyl;

and

R¹⁹ is selected from hydrogen, methyl, and t-butyl.

In some embodiments, Ar¹ is:

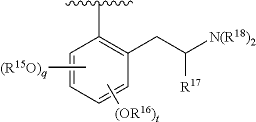

wherein;

q is 0 or 1;

t is 0 or 1;

R¹⁵ and R¹⁶ are each, independently, alkoxymethyl;

R¹⁷ is selected from hydrogen and C(O)₂R¹⁹;

R¹⁸ in each occurrence is independently selected from hydrogen and t-butoxycarbonyl;

and

R¹⁹ is selected from hydrogen, methyl, and t-butyl.

In some embodiments, Ar¹ is:

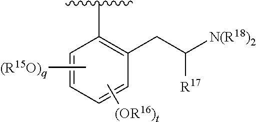

wherein;

q is 0 or 1;

t is 0 or 1;

R¹⁵ and R¹⁶ are each, independently, selected from benzyloxymethyl, ethoxymethyl, methoxyethoxymethyl, and methoxymethyl;

R¹⁷ is selected from hydrogen and C(O)₂R¹⁹;

R¹⁸ in each occurrence is independently selected from hydrogen and t-butoxycarbonyl;

and

R¹⁹ is selected from hydrogen, methyl, and t-butyl.

In some embodiments, Ar¹ is:

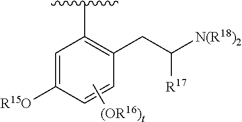

wherein;

t is 0 or 1;

R¹⁵ and R¹⁶ are each, independently alkoxymethyl;

R¹⁷ is selected from hydrogen and C(O)₂R¹⁹;

R¹⁸ in each occurrence is independently selected from hydrogen and t-butoxycarbonyl;

and

R¹⁹ is selected from hydrogen, methyl, and t-butyl.

In some embodiments, Ar¹ is:

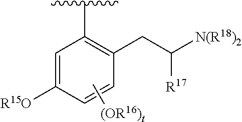

wherein;

t is 0 or 1;

R¹⁵ and R¹⁶ are each, independently, selected from benzyloxymethyl, ethoxymethyl, methoxyethoxymethyl, and methoxymethyl;

R¹⁷ is selected from hydrogen and C(O)₂R¹⁹;

R¹⁸ in each occurrence is independently selected from hydrogen and t-butoxycarbonyl;

and

R¹⁹ is selected from hydrogen, methyl, and t-butyl.

In some embodiments, Ar¹ is:

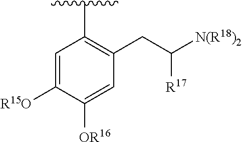

wherein;

R¹⁵ and R¹⁶ are each, independently, an acid labile protecting group;

R¹⁷ is selected from hydrogen and C(O)₂R¹⁹;

R¹⁸ in each occurrence is independently selected from hydrogen and t-butoxycarbonyl; and

R¹⁹ is selected from hydrogen, methyl, and t-butyl.

In some embodiments, Ar¹ is:

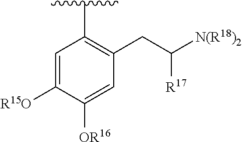

wherein;

R¹⁵ and R¹⁶ are each, independently, alkoxymethyl;

R¹⁷ is selected from hydrogen and C(O)₂R¹⁹;

R¹⁸ in each occurrence is independently selected from hydrogen and t-butoxycarbonyl; and

R¹⁹ is selected from hydrogen, methyl, and t-butyl.

In some embodiments, Ar¹ is:

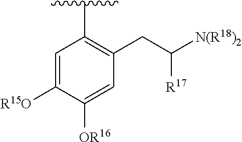

wherein;

R¹⁵ and R¹⁶ are each, independently, selected from benzyloxymethyl, ethoxymethyl, methoxyethoxymethyl, and methoxymethyl;

R¹⁷ is selected from hydrogen and C(O)₂R¹⁹;

R¹⁸ in each occurrence is independently selected from hydrogen and t-butoxycarbonyl; and

R¹⁹ is selected from hydrogen, methyl, and t-butyl.

In some embodiments, Ar¹ is:

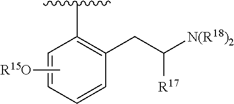

wherein;

R¹⁵ is an acid labile protecting group;

R¹⁷ is selected from hydrogen and C(O)₂R¹⁹;

R¹⁸ in each occurrence is independently selected from hydrogen and t-butoxycarbonyl; and

R¹⁹ is selected from hydrogen, methyl, and t-butyl.

In some embodiments, Ar¹ is:

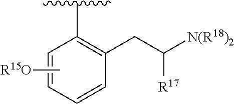

wherein;

R¹⁵ is alkoxymethyl;

R¹⁷ is selected from hydrogen and C(O)₂R¹⁹;

R¹⁸ in each occurrence is independently selected from hydrogen and t-butoxycarbonyl; and

R¹⁹ is selected from hydrogen, methyl, and t-butyl.

In some embodiments, R¹⁵ and R¹⁶ are alkoxy.

In some embodiments, R¹⁵ and R¹⁶ are ethoxymethyl.

In some embodiments, R¹⁵ is ethoxymethyl.

The exceptionally mild oxidation protocol is compatible with a wide range of acid labile hydroxyl protecting groups. The hydroxyl protecting groups may be easily cleaved under mild conditions, to provide, for example, radiotracer compounds. In general, crystallinity of the final product is desired; thus, lipophilic embodiments of R¹⁵ and R¹⁶ are generally to be avoided.

In some embodiments, Ar2 is defined as in embodiment (a).

In some embodiments, Ar² is aryl substituted by 1, 2, 3, 4, or 5 C₁₋₆ alkoxy groups.

In some embodiments, Ar² is aryl substituted by 1, 2, 3, 4, or 5 methoxy groups.

In some embodiments, Ar² is aryl substituted by 1 or 2 C₁₋₆ alkoxy groups.

In some embodiments, Ar² is aryl substituted by 1 or 2 methoxy groups.

In some embodiments, Ar² is aryl substituted by 1 C₁₋₆ alkoxy group.

In some embodiments, Ar² is aryl substituted by 1 methoxy group.

In some embodiments, Ar² is phenyl substituted by 1, 2, 3, 4, or 5 C₁₋₆ alkoxy groups.

In some embodiments, Ar² is phenyl substituted by 1, 2, 3, 4, or 5 methoxy groups.

In some embodiments, Ar² is phenyl substituted by 1 or 2 C₁₋₆ alkoxy groups.

In some embodiments, Ar² is phenyl substituted by 1 or 2 methoxy groups.

In some embodiments, Ar² is phenyl substituted by 1 C₁₋₆ alkoxy group.

In some embodiments, Ar² is phenyl substituted by 1 methoxy group.

In some embodiments, Ar² is p-methoxyphenyl.

In some embodiments, Ar² is 3,4-dimethoxyphenyl.

In some embodiments, Ar² is Formula (1):

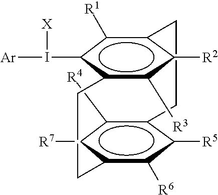

or Formula (4):

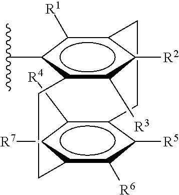

wherein:

R¹ is hydrogen or a substituent having a Hammett σₚ value of less than zero; and

R², R³, R⁴, R⁵, R⁶, and R⁷ are independently selected from the group consisting of: H, CF₃, OCF₃, CN, hydroxyl, amino, aminoalkyl, (CH₂)ₙN(CH₂)ₘ, —SR⁸, —SOR⁸, halo, SO₂R⁸, (CH₂)ₙOR⁸, C(═O)NR⁸R⁹, SO₂NR⁸R⁹, NR⁸SO₂R⁹, COOR⁸, NR⁸C(═O)R⁹, NR⁸C(═O)NR⁹, SO₂R⁸, (CH₂)ₙC(═O)NR⁸R⁹, (CH₂)ₙSO₂NR⁸R⁹, (CH₂)ₙNR⁸SO₂R⁹, (CH₂)ₙCOOR⁸, (CH₂)ₙNR⁸C(═O)R⁹, (CH₂)ₙNR⁸C(═O)NR⁹, alkoxy, substituted or unsubstituted alkyl, substituted or unsubstituted alkenyl, substituted or unsubstituted alkynyl, substituted or unsubstituted cycloalkyl, substituted or unsubstituted heterocycloalkyl, substituted or unsubstituted aryl, substituted or unsubstituted heteroaryl, and (L)ₚ-Z, or one or more of R² and R³, R⁴ and R⁷, and R⁵ and R⁶ come together to form a fused cycloalkyl, heterocycloalkyl, aryl, or heteroaryl ring system;

each m, n, and p are independently an integer from 0 to 10;

each R⁸ and R⁹ are independently chosen from H, substituted or unsubstituted alkyl, substituted or unsubstituted alkenyl, substituted or unsubstituted alkynyl, substituted or unsubstituted cycloalkyl, substituted or unsubstituted heterocycloalkyl, substituted or unsubstituted aryl, and substituted or unsubstituted heteroaryl;

L is a linker; and

Z is a solid support.

The aryl rings on the cyclophane moiety can be substituted or unsubstituted. In some embodiments, R¹ is selected from the group consisting of: —(C₁-C₁₀)alkyl, —(C₁-C₁₀)haloalkyl, (C₂-C₁₀)alkenyl, (C₂-C₁₀)alkynyl, —O—(C₁-C₁₀)alkyl, —C(O)—O—(C₁-C₁₀)alkyl, aryl, and heteroaryl. For example, R¹ can be —O—(C₁-C₁₀)alkyl (e.g., OCH₃). In some embodiments, R² is —O—(C₁-C₁₀)alkyl (e.g., OCH₃). For example, a compound of Formula (1) can be chosen from:

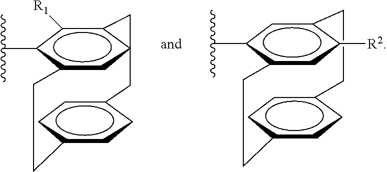

In some embodiments, R¹ is methoxy.

In some embodiments, one or more of R²-R⁷ is (L)ₚ-Z. L and Z can be covalently or noncovalently bound to one another.

In some embodiments, Ar² is any of the cyclophanes in US 2011/0190505, which is incorporated herein by reference in its entirety.

In some embodiments, Ar¹ is defined as in embodiment (a); and Ar² is one of the specific embodiments above.

In some embodiments, the process further comprises subjecting the compound of Formula III to ion-exchange in order to form a compound of Formula V:

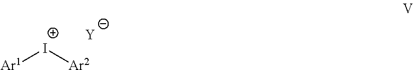

wherein Y is a counterion that is different than X.

In some embodiments, Y is a weakly coordinating anion (i.e., an anion that coordinates only weakly with iodine). For example, Y can be the conjugate base of a strong acid, for example, any anion for which the pKa of the conjugate acid (H—Y) is less than about 1. For example, Y can be triflate, mesylate, nonaflate, hexaflate, toluene sulfonate (tosylate), nitrophenyl sulfonate (nosylate), bromophenyl sulfonate (brosylate), perfluoroalkyl sulfonate (e.g., perfluoro C₂₋₁₀ alkyl sulfonate), tetraphenylborate, hexafluorophosphate, trifluoro acetate, perfluoroalkylcarboxylate, tetrafluoroborate, perchlorate, hexafluorostibate, hexachlorostibate, chloride, bromide, or iodide. In some embodiments, a slightly more basic leaving group such as acetate or benzoate may be used.

In some embodiments, the ion-exchange comprises treating the compound of Formula III with an aqueous solution of hexaflurophosphate ion, wherein Y is PF₆—. In some embodiments, the ion-exchange comprises treating the compound of Formula III with an aqueous solution of sodium hexaflurophosphate ion, wherein Y is PF₆—.

The present application further provides a process of forming a compound of Formula III:

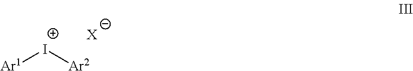

comprising:

(a) treating a compound of Formula II:

Ar¹—I II

with more than 2 equivalents of (R¹)₃Si—X; and less than 2 equivalents of (1-chloromethyl-4-fluoro-1,4-diazoniabicyclo[2.2.2]octane)bis(tetrafluoroborate) or (1-fluoro-4-methyl-1,4-diazoniabicyclo[2.2.2]octane)bis(tetrafluoroborate) in the absence of added acid to form a compound of Formula I: and

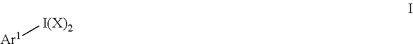

(b) reacting the compound of Formula I with Ar²BF₄M² in the presence of a catalyst to form a compound of Formula III: wherein:

each X is, independently, a ligand, wherein HX, the conjugate acid of X, has a pKₐ of less than or equal to 5;

Ar¹ is optionally substituted aryl or heteroaryl, wherein Ar¹ does not have unprotected protic groups;

Ar² is an optionally substituted aryl or heteroaryl;

each R¹ is, independently, C₁₋₄ alkyl; and

M² is a cation.

In some embodiments, the process utilizes (1-chloromethyl-4-fluoro-1,4-diazoniabicyclo[2.2.2]octane)bis(tetrafluoroborate); and (R¹)₃Si—X is (CH₃)₃Si—O(C═O)CH₃.

In some embodiments, steps (a) and (b) are carried out in a single pot.

In some embodiments, the present application provides compounds of Formula II and processes utilizing compounds of Formula II (e.g., a process of making a compound of Formula I, III, V, or VI), wherein the compounds of Formula II are selected from any of the following:

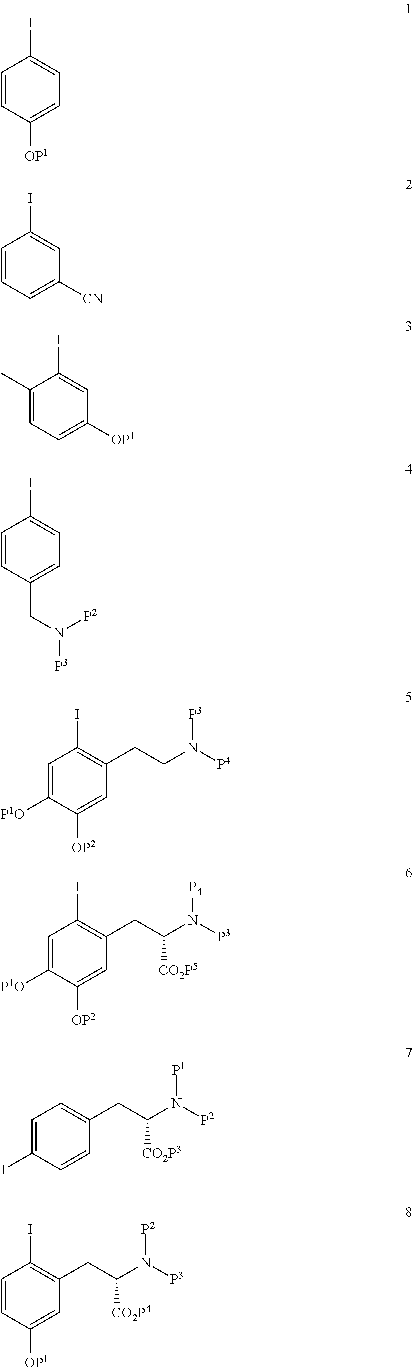

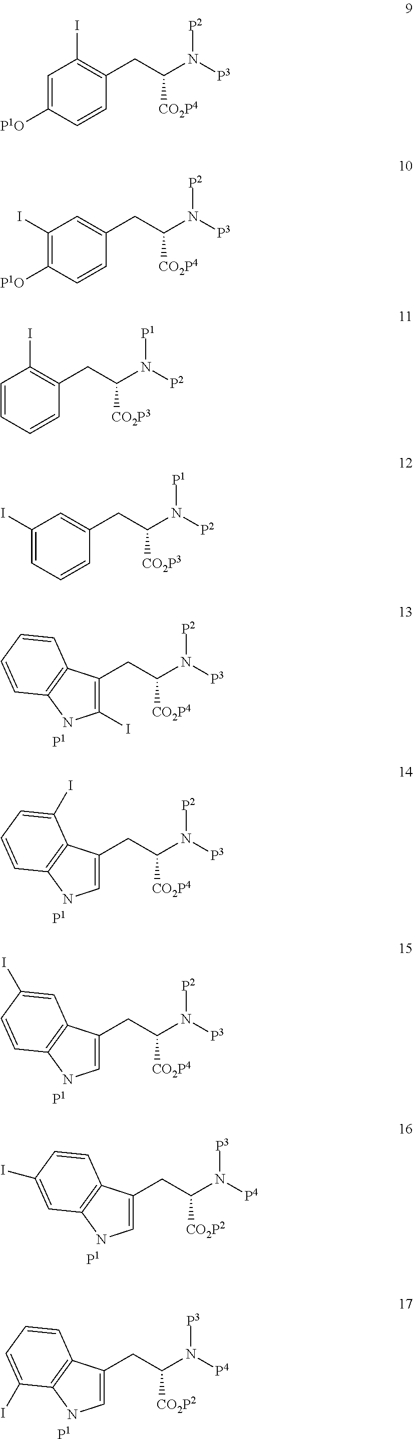

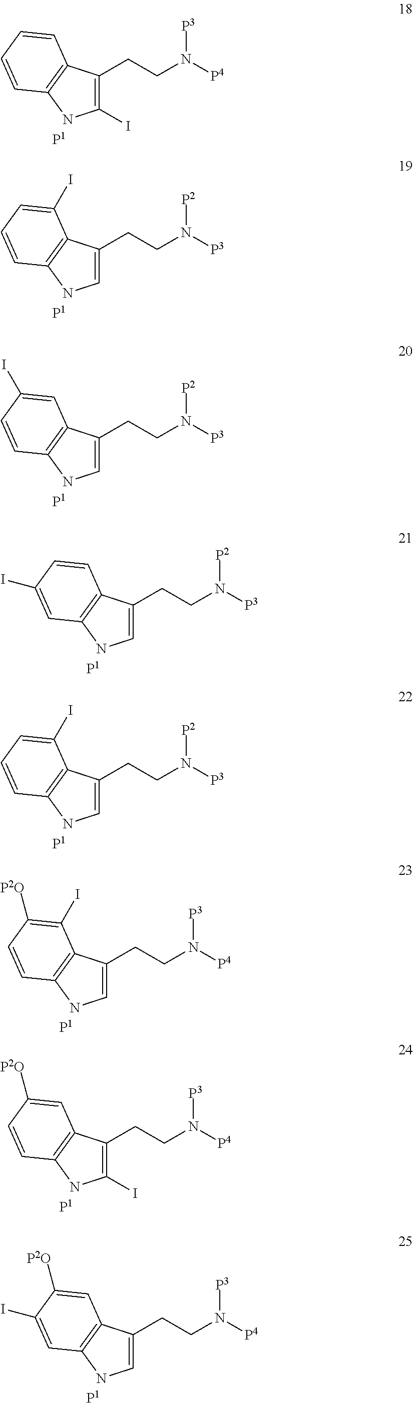

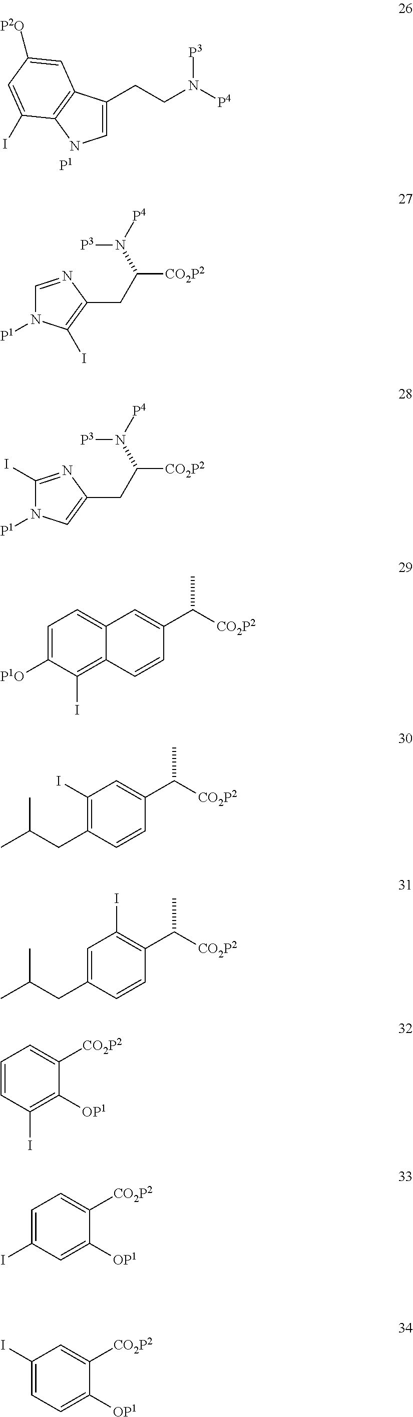

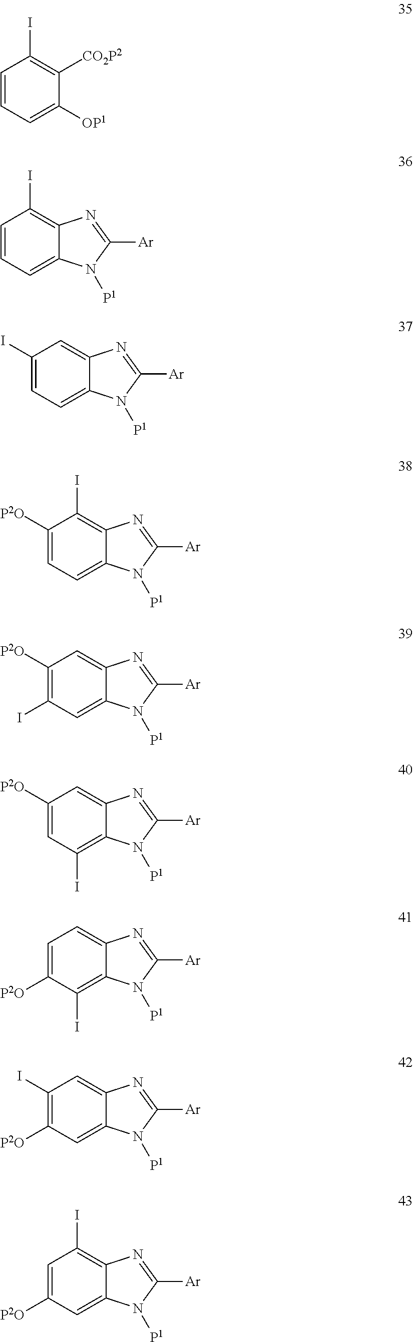

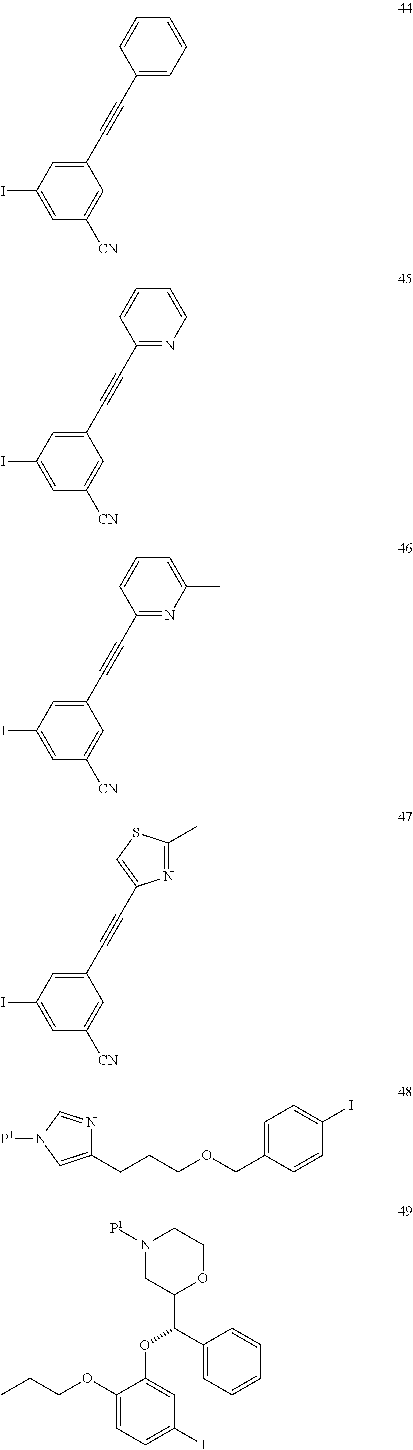

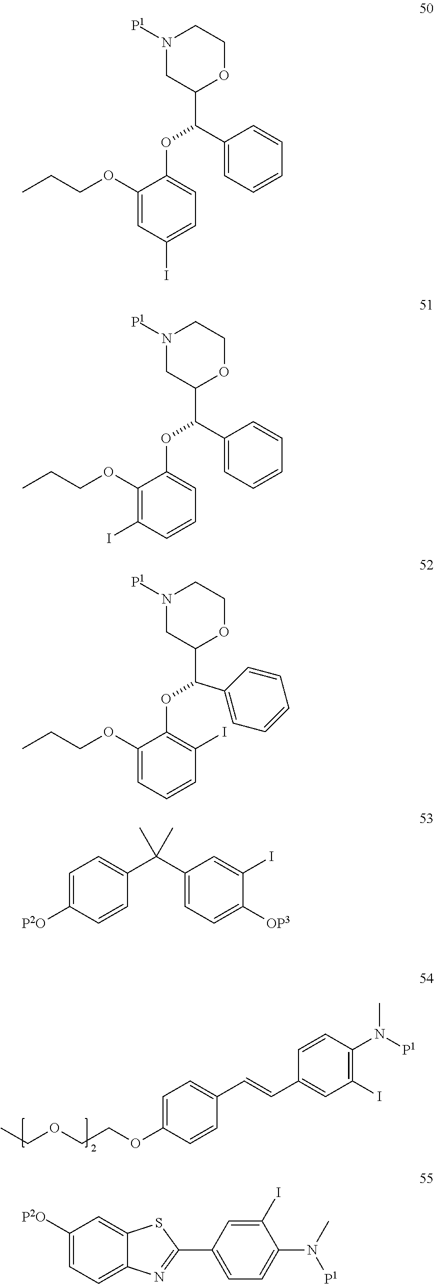

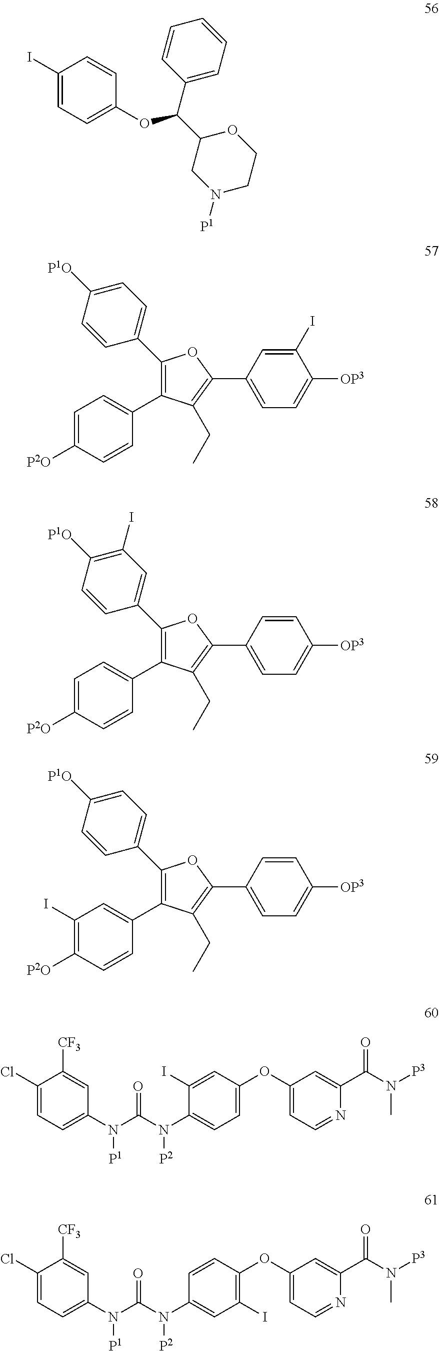

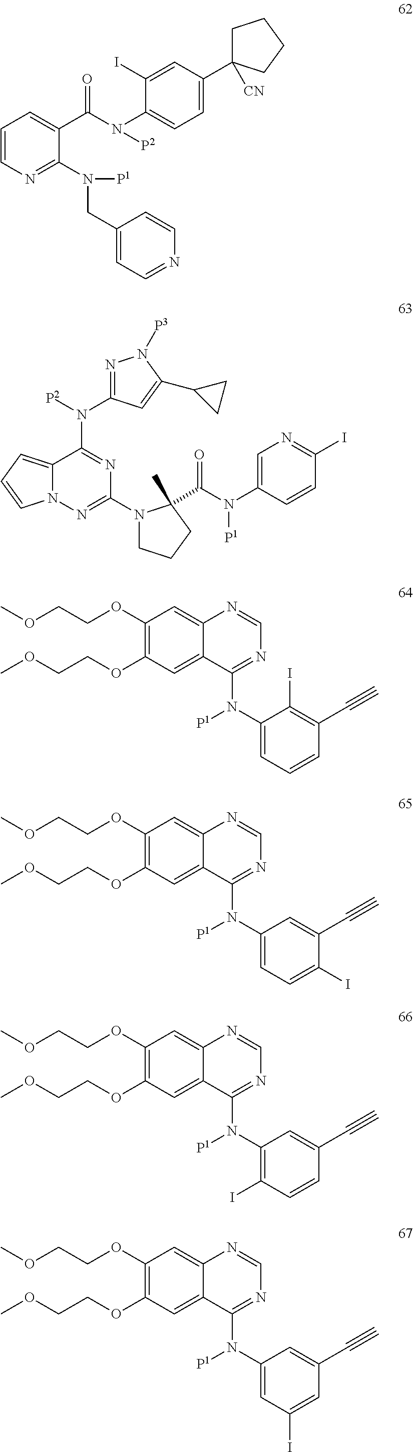

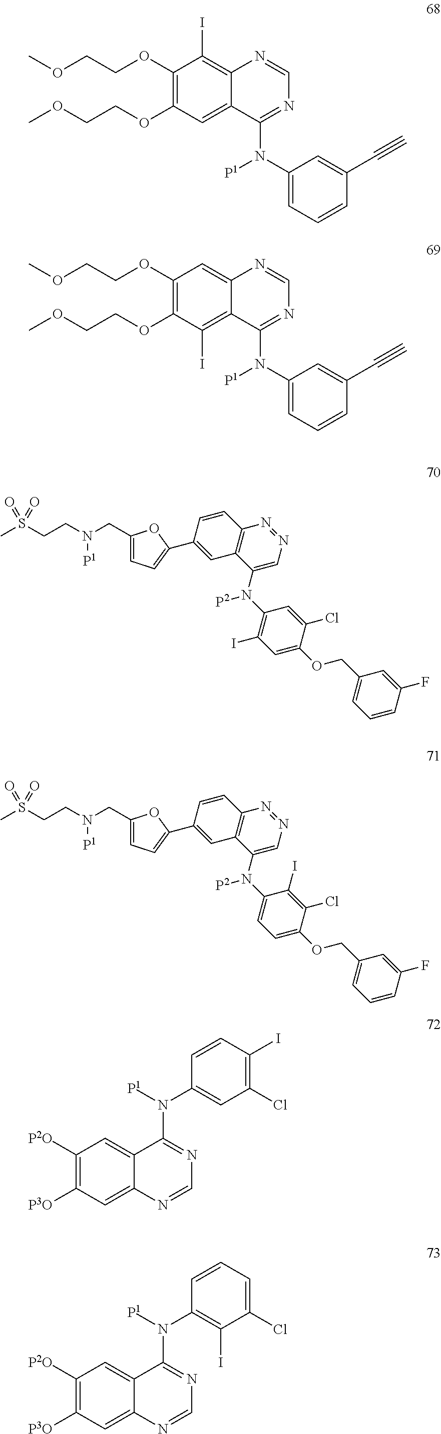

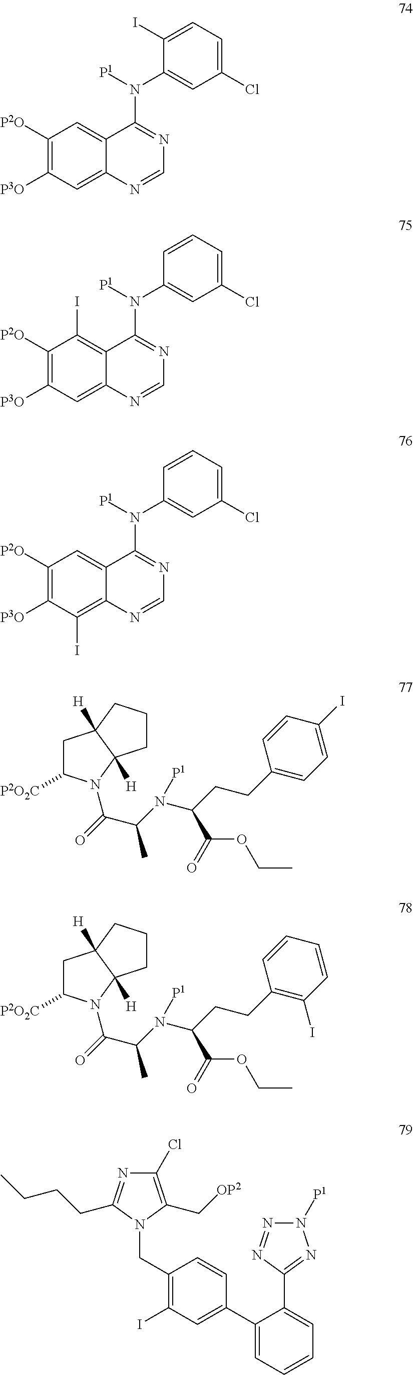

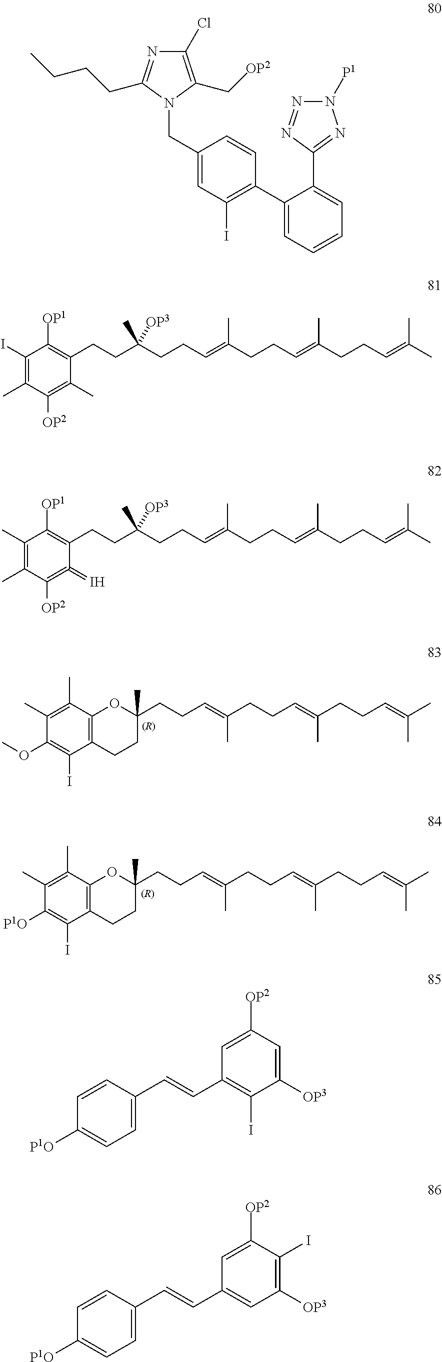

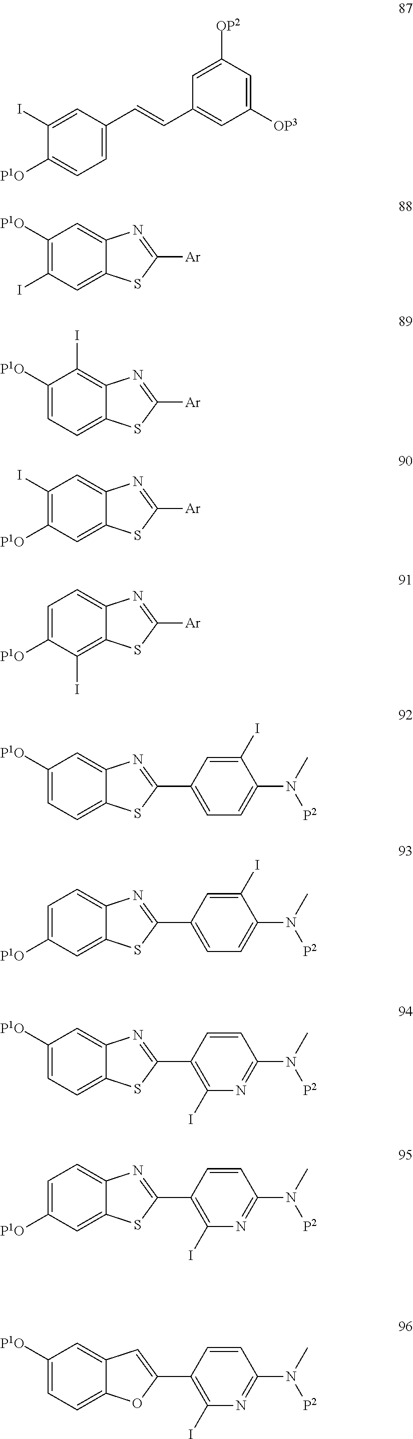

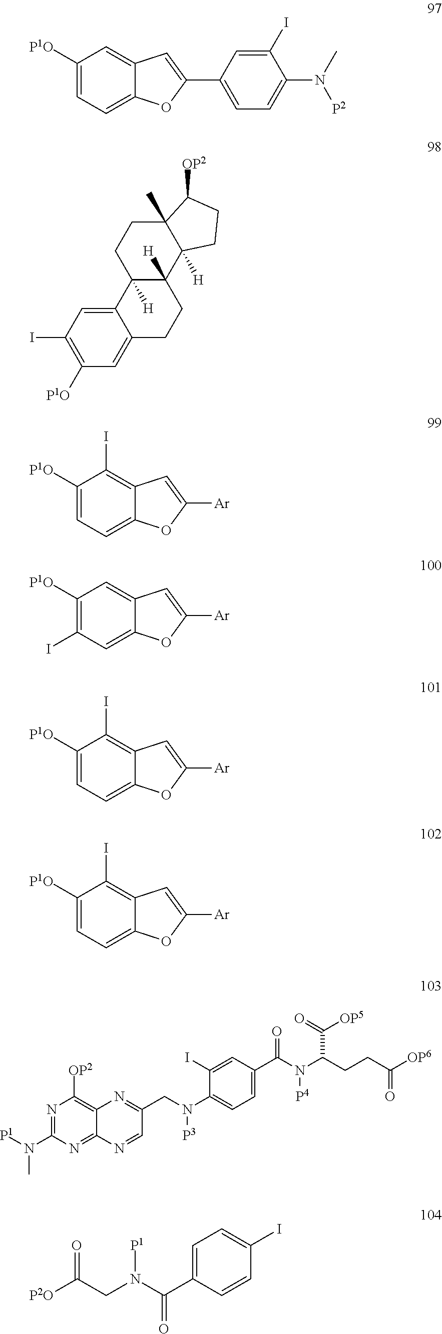

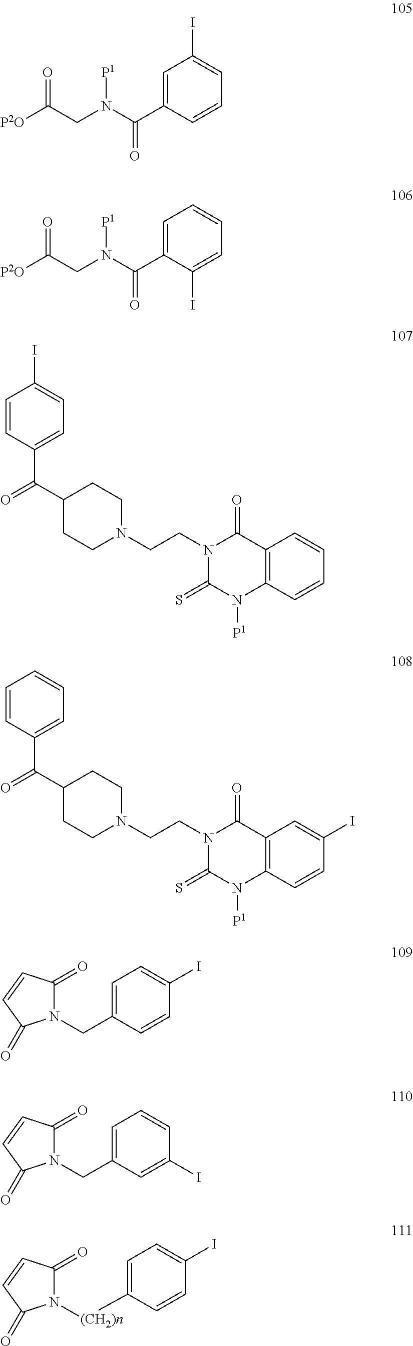

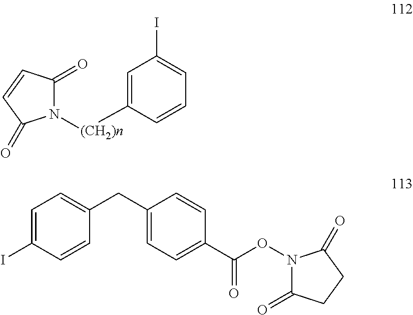

wherein Ar is an optionally substituted aryl or heteroaryl, wherein Ar does not have unprotected protic groups; and P¹, P², P³, P⁴, P⁵ and P⁶ are each, independently, protecting groups. In some embodiments, each X is acetate.

In certain preferred embodiments, the compound of Formula II is selected from the group consisting of compounds 109-113. In one preferred embodiment, the compound of Formula II is the compound 109. In another preferred embodiment, the compound of Formula II is the compound 113.

In some embodiments, the present application provides a compound of Formula I or a process utilizing a compound of Formula I (e.g., a process of making a compound of Formula III, V or VI starting from a compound of Formula I; or a process of making a compound of Formula I), wherein the compound of Formula I is selected from any of the following:

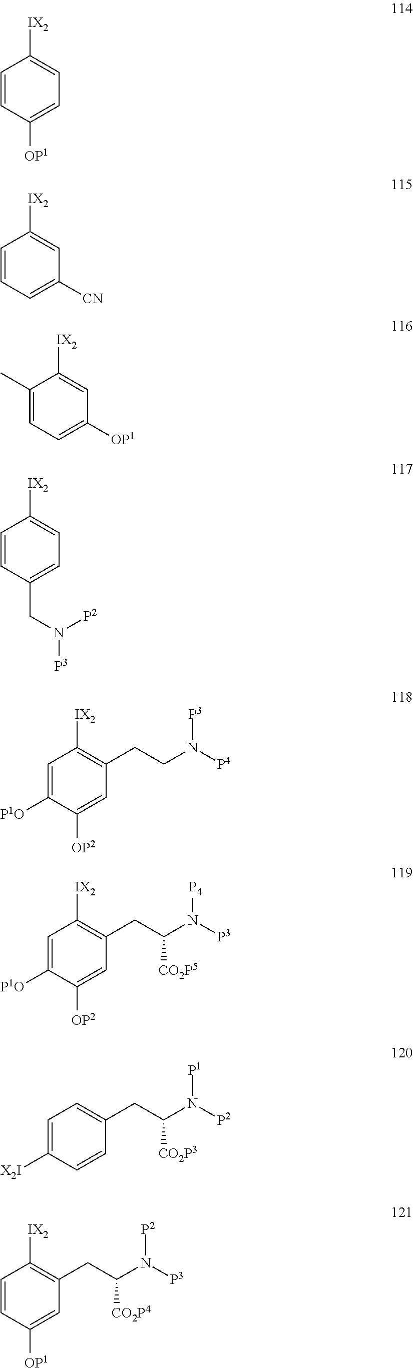

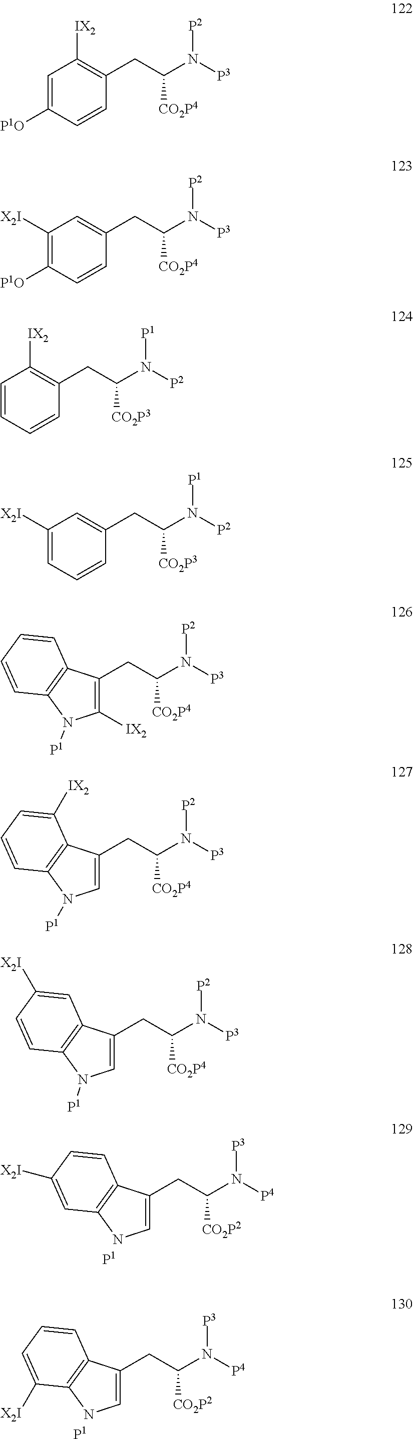

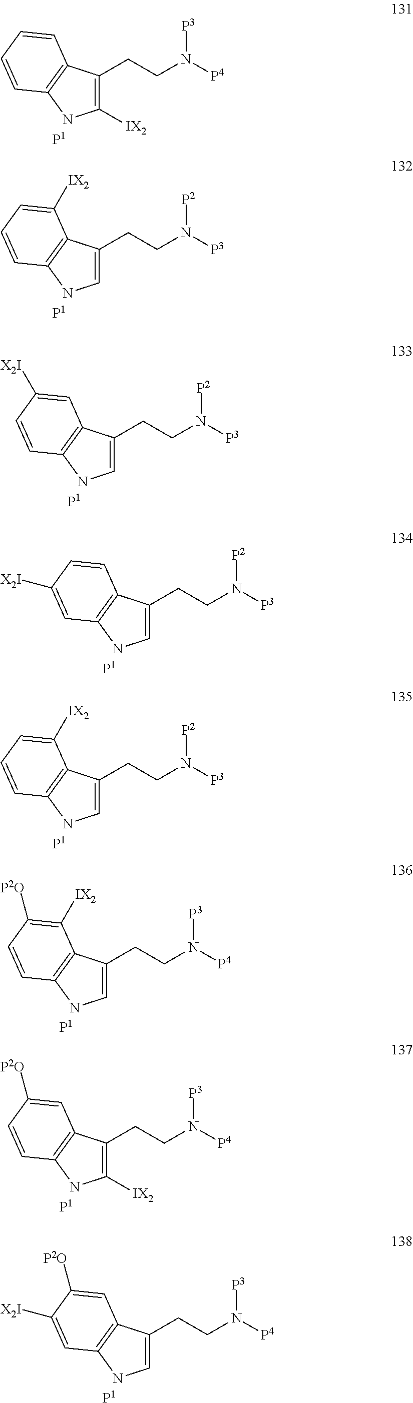

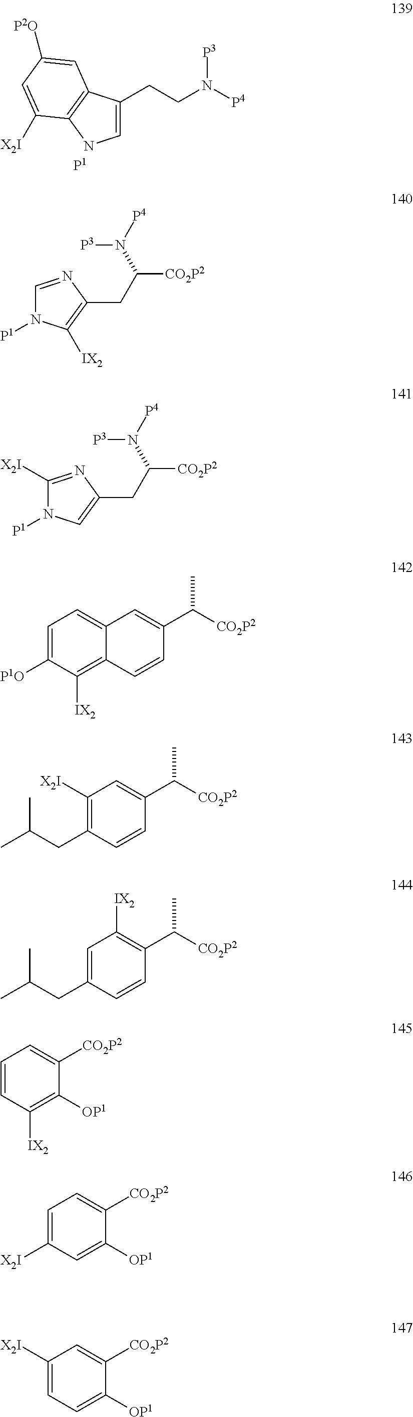

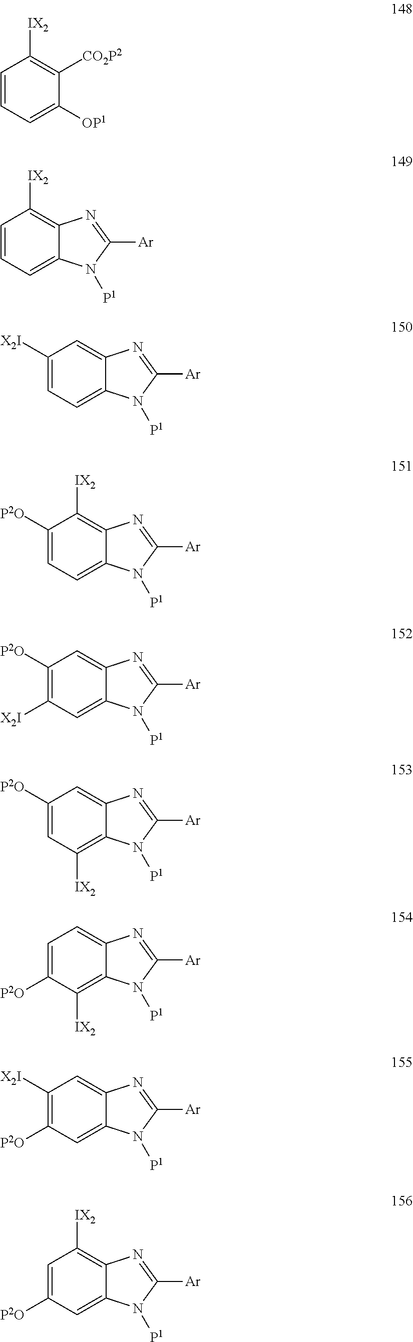

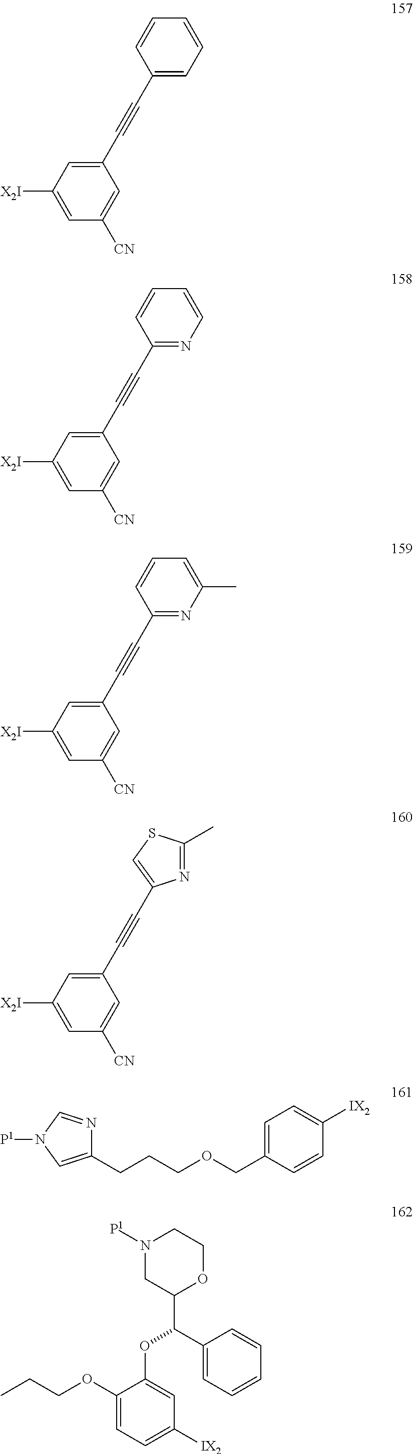

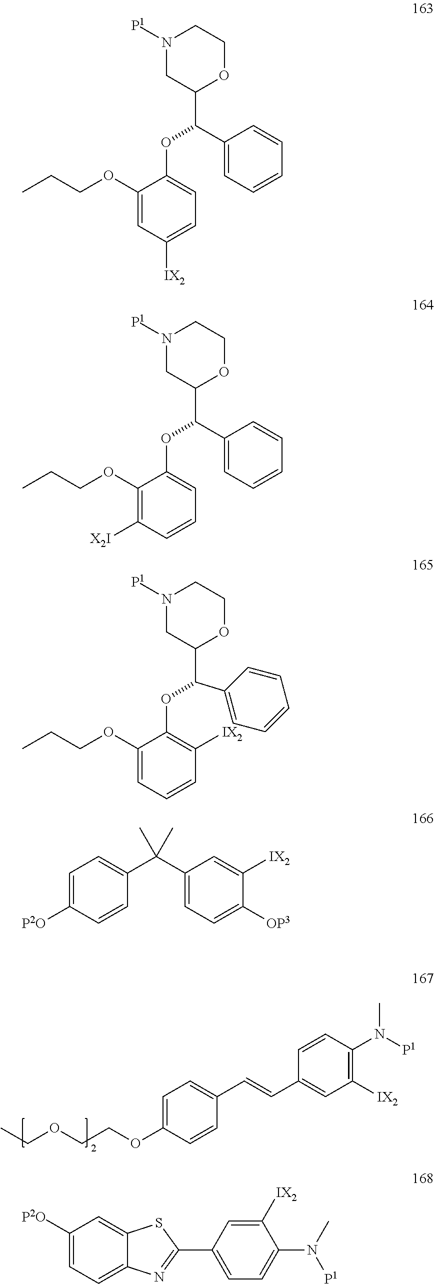

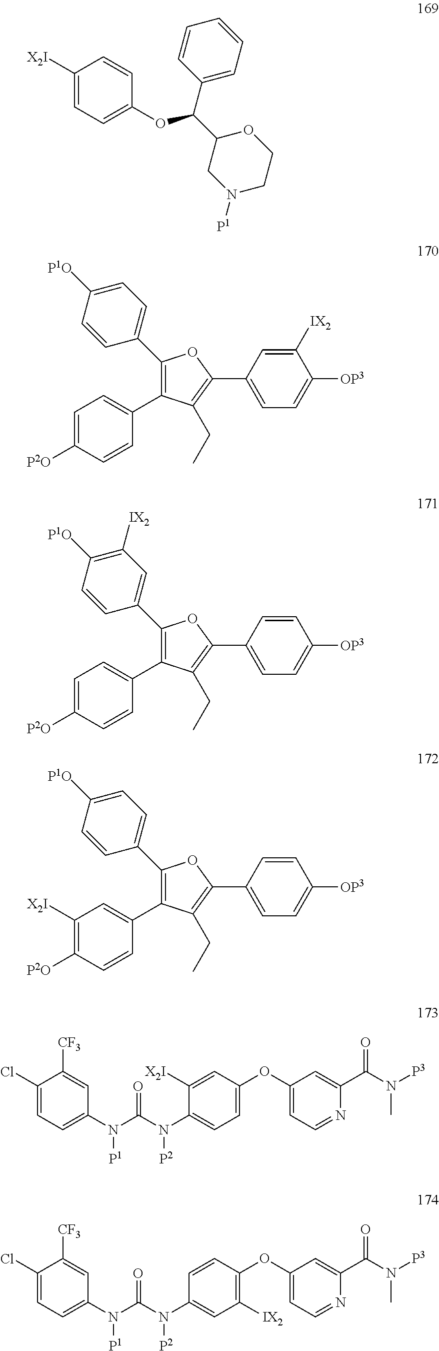

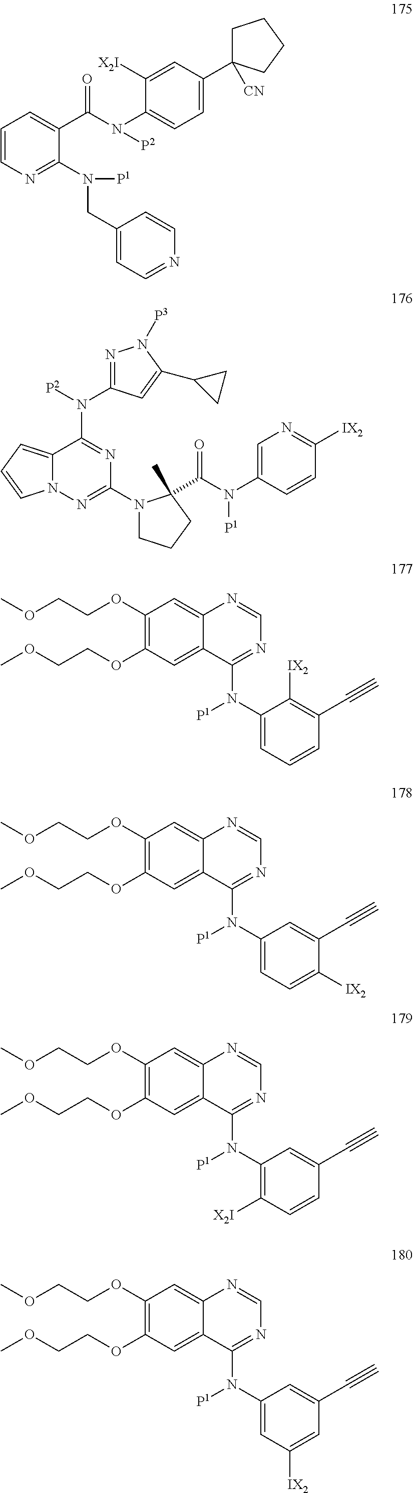

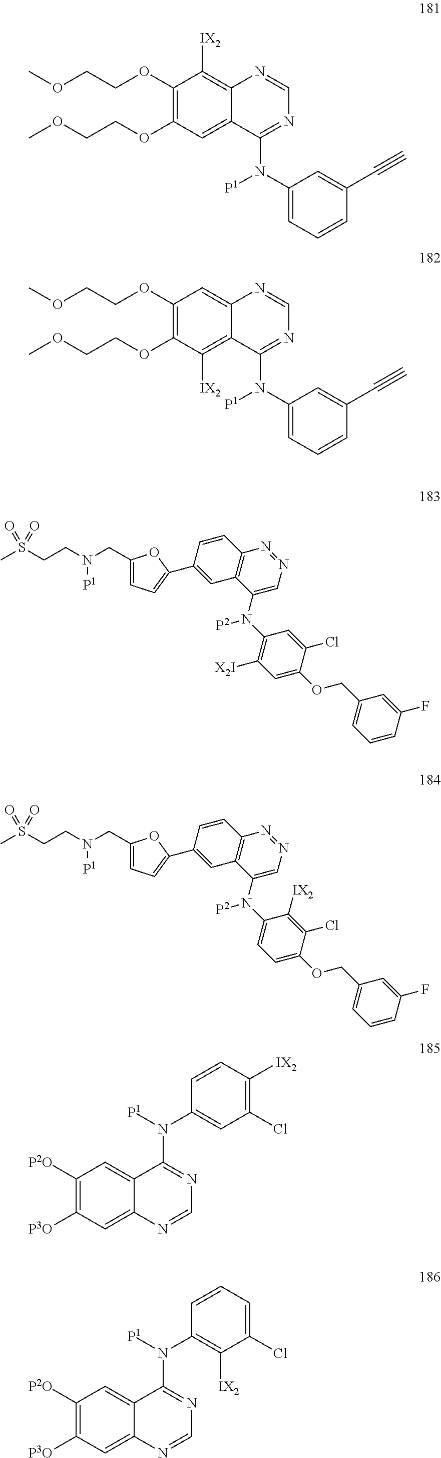

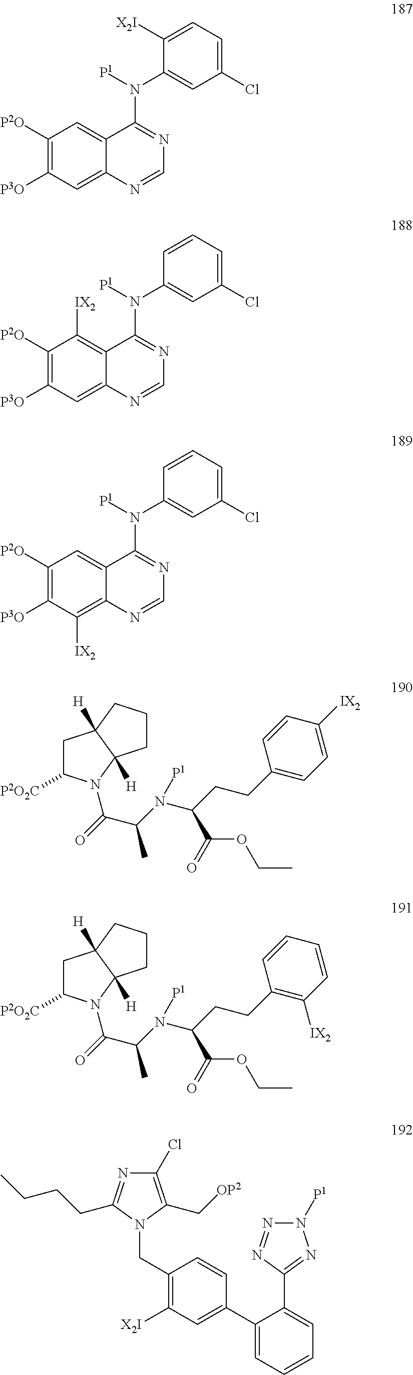

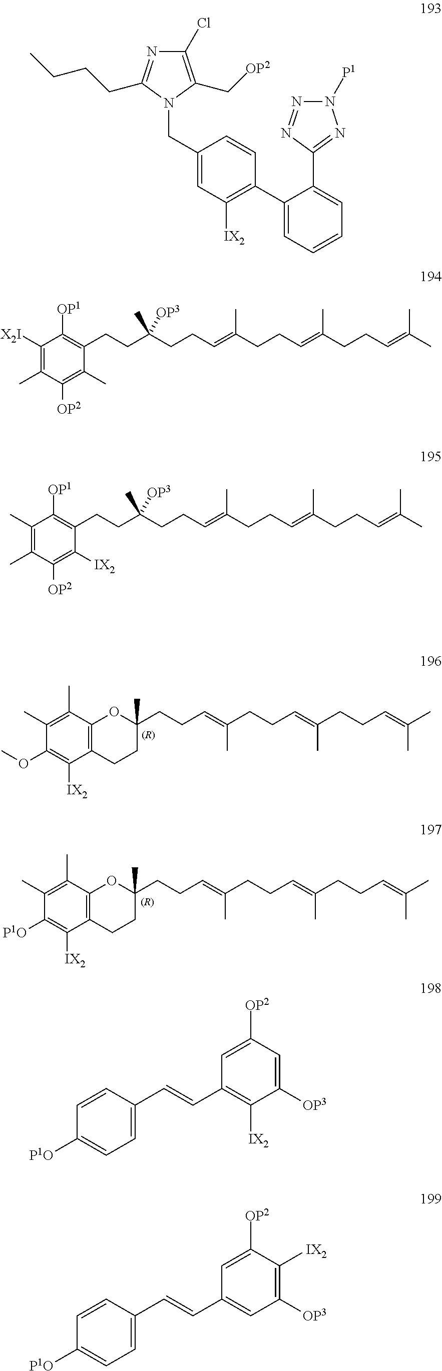

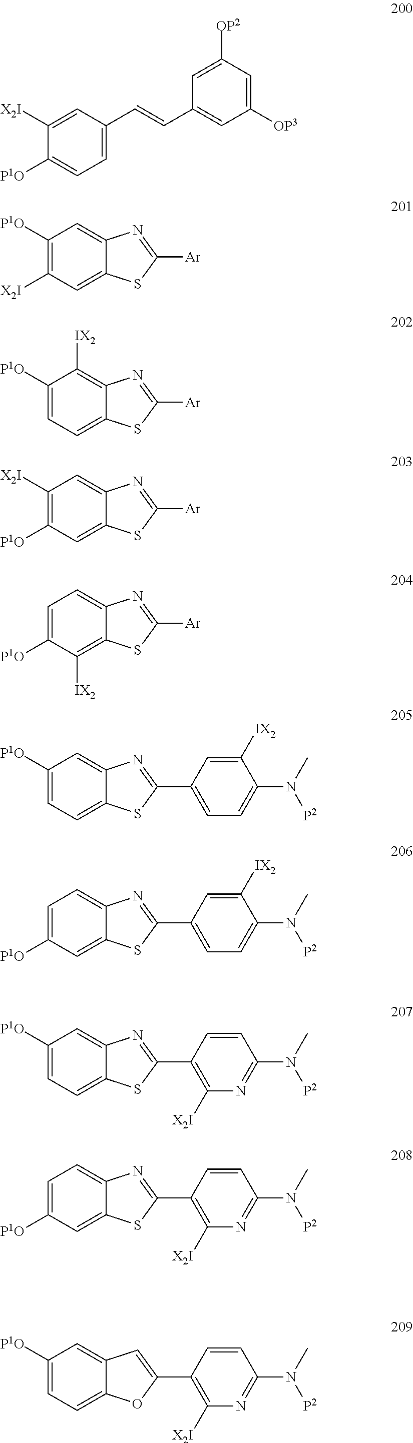

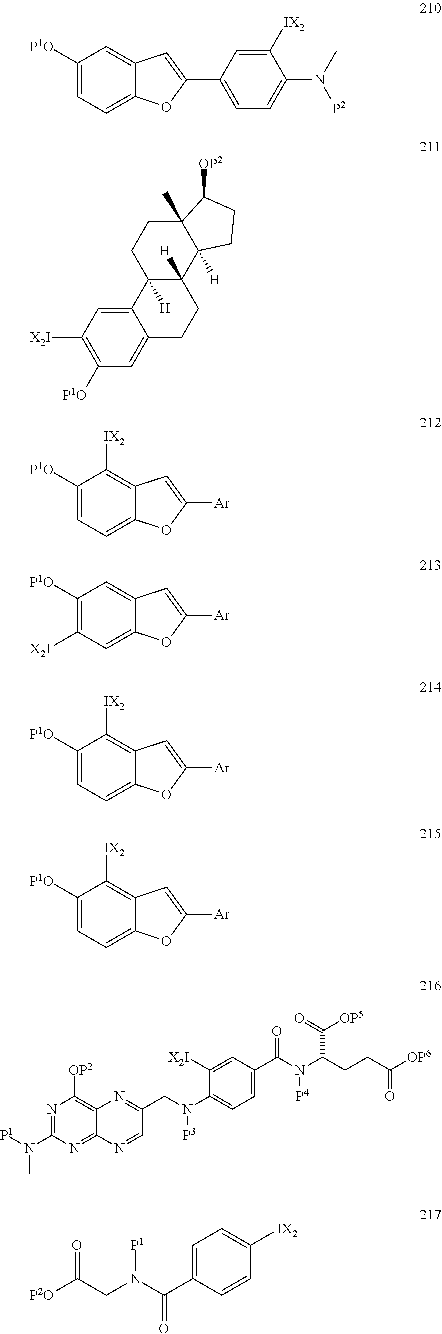

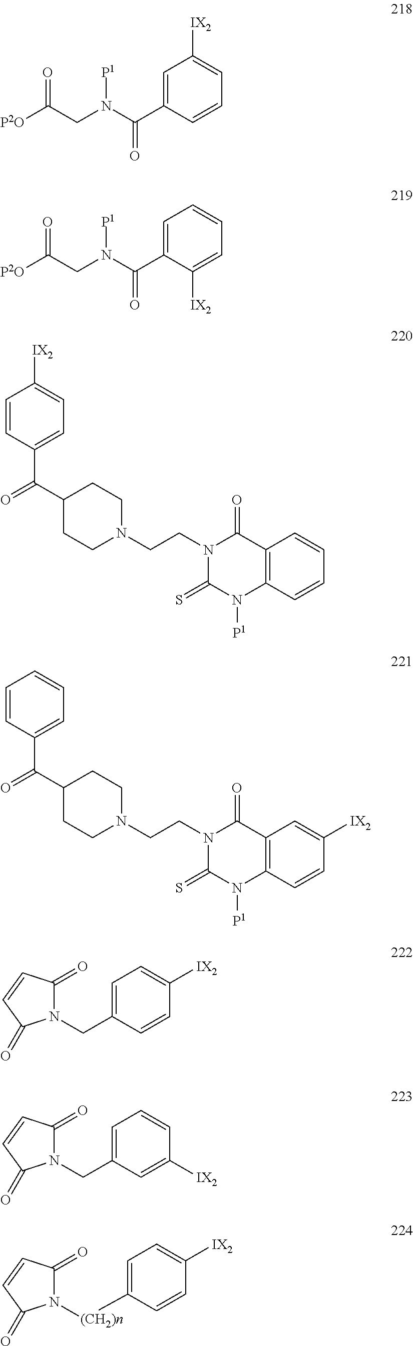

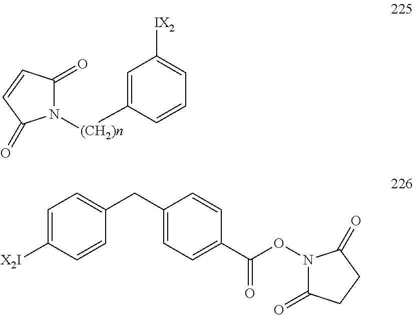

wherein Ar is an optionally substituted aryl or heteroaryl, wherein Ar does not have unprotected protic groups; and P¹, P², P³, P⁴, P⁵, and P⁶ are each, independently, protecting groups; and X is defined above. In some embodiments, each X is acetate.

In one preferred embodiment, the compound of Formula I is selected from the group consisting of compounds 118-122. In another preferred embodiment, the compound of Formula I is selected from the group consisting of compounds 177-182. In a particular embodiment, the compound of Formula I is compound 178. In another preferred embodiment, the compound of Formula I is selected from the group consisting of compounds 205-210. In another preferred embodiment, the compound of Formula I is selected from the group consisting of compounds 216, 222 and 226.

In some embodiments, the present application provides a compound of Formula III or a process involving a compound of Formula III (e.g., a process of making a compound of Formula III or a process of making a compound of Formula V or VI):

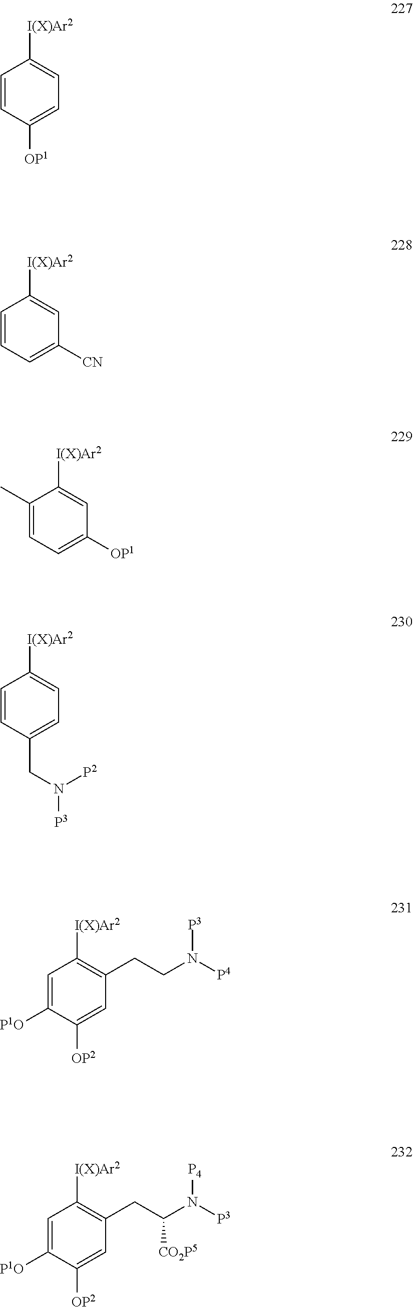

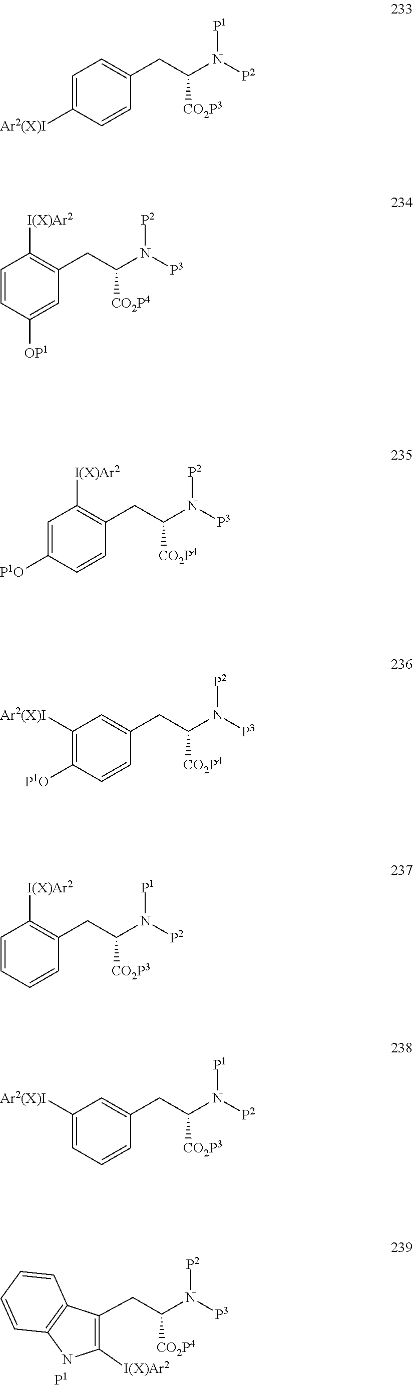

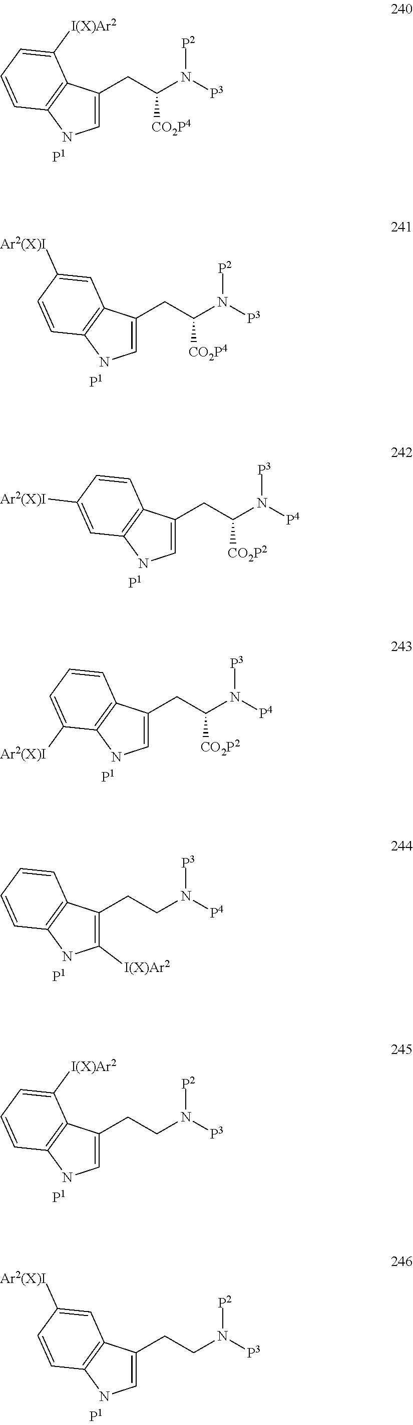

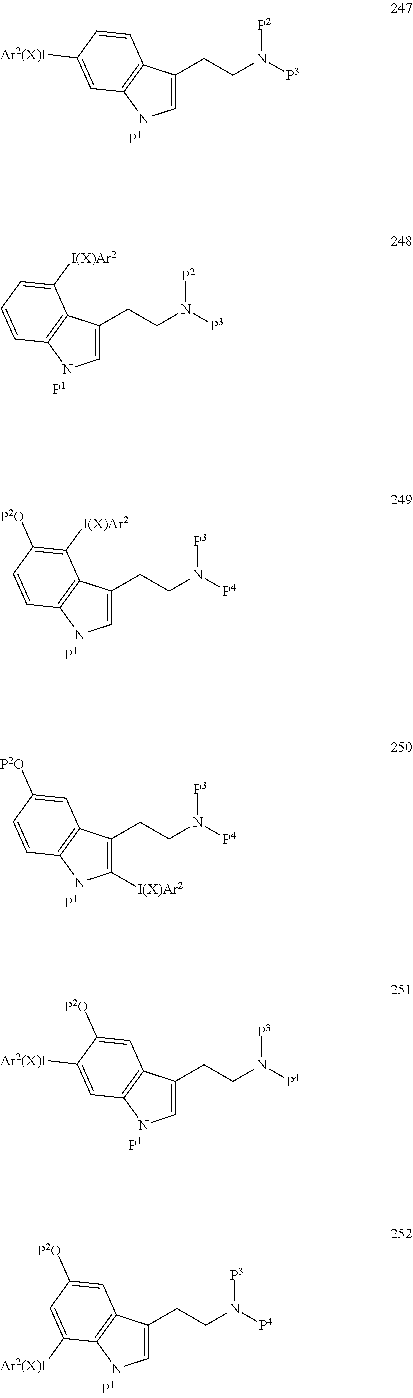

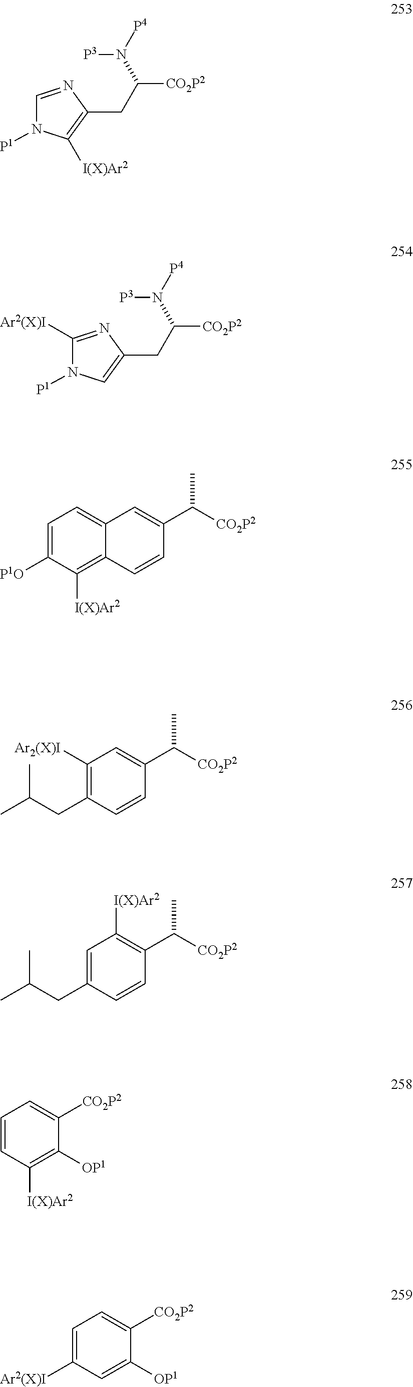

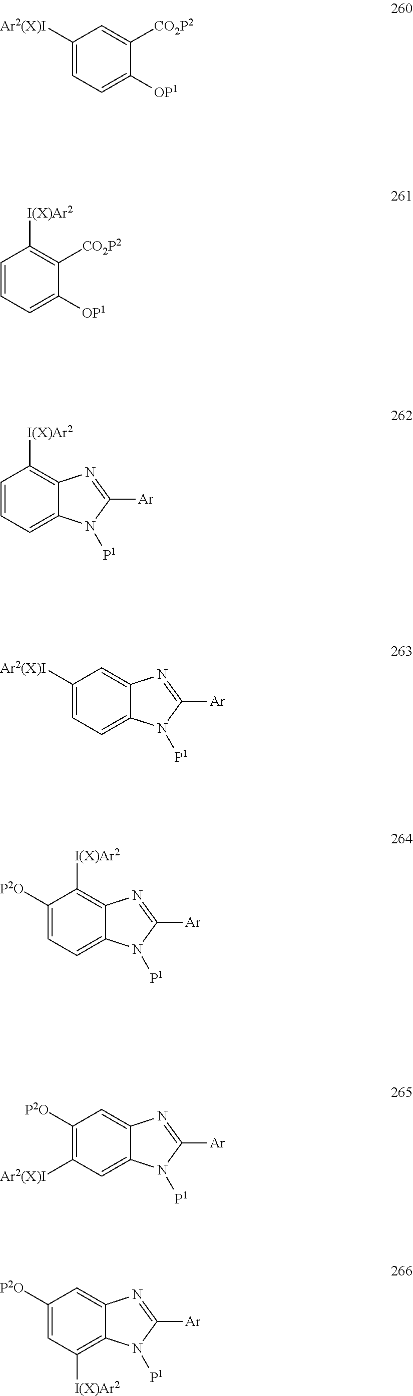

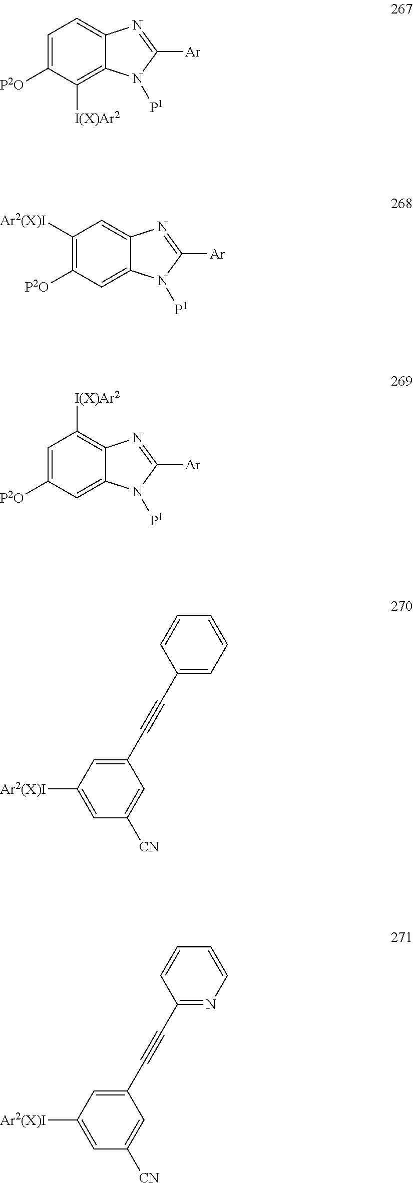

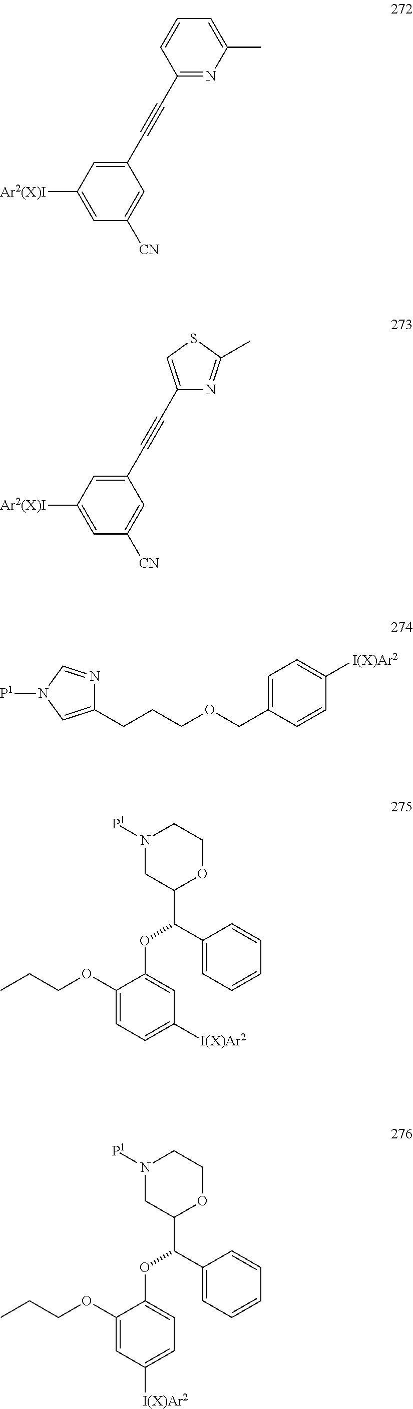

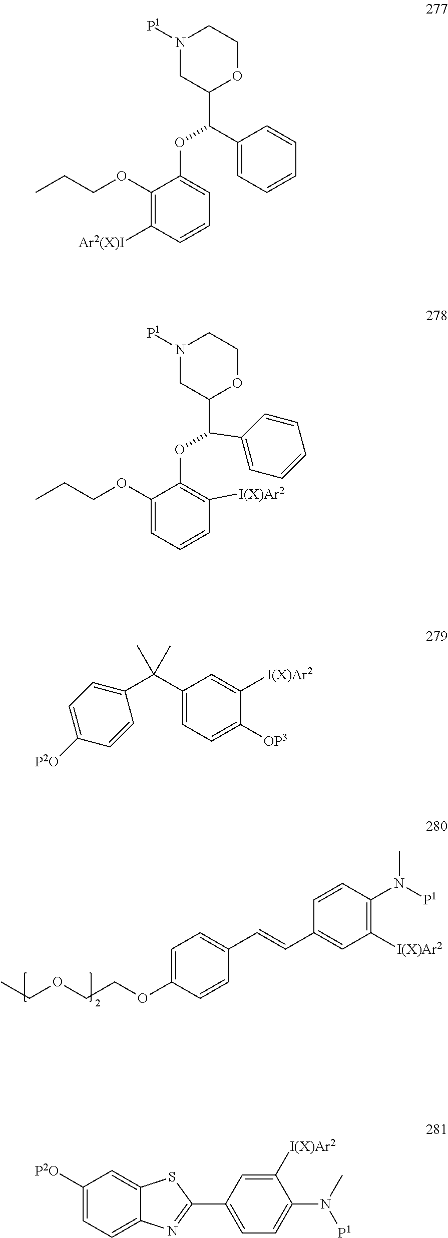

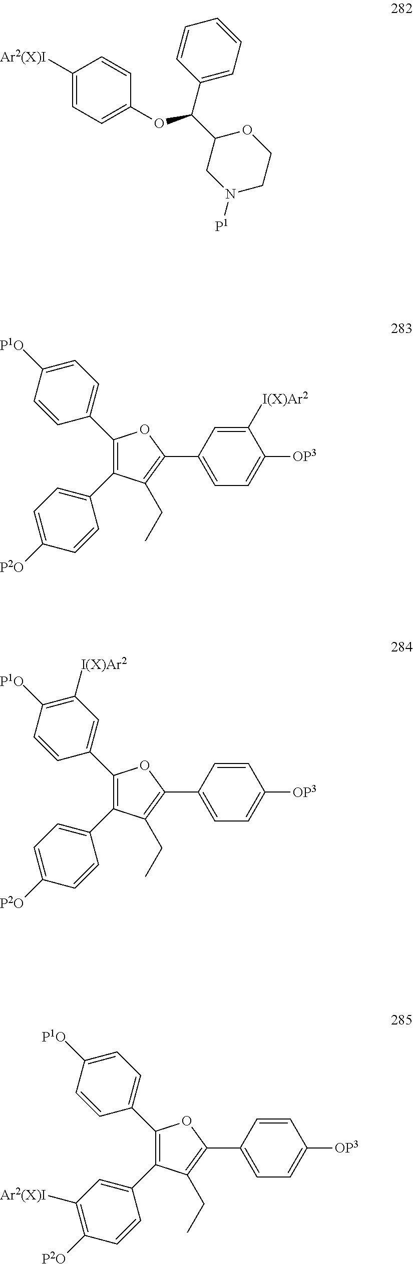

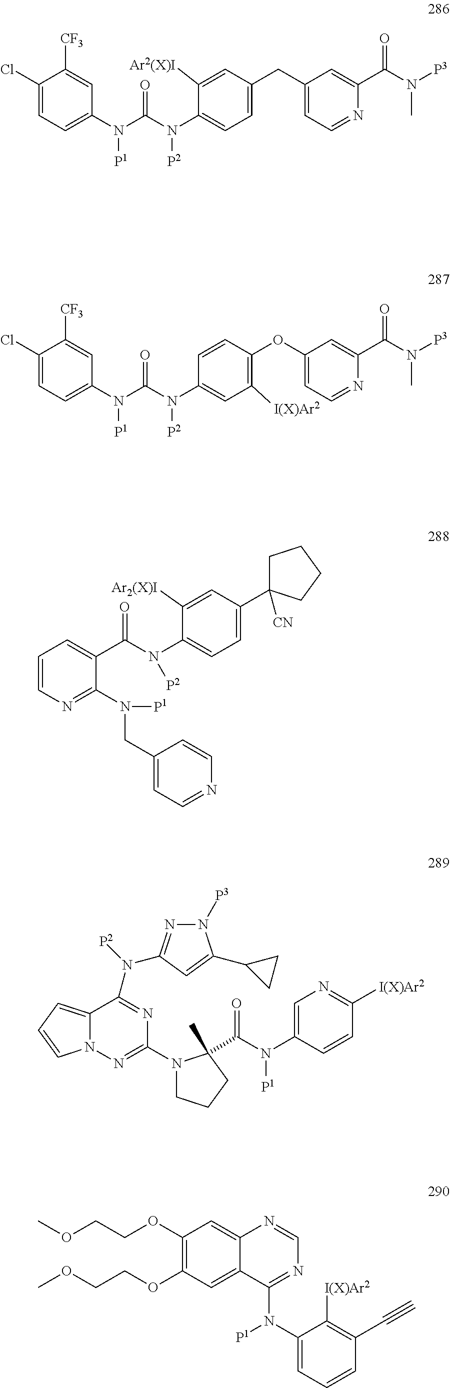

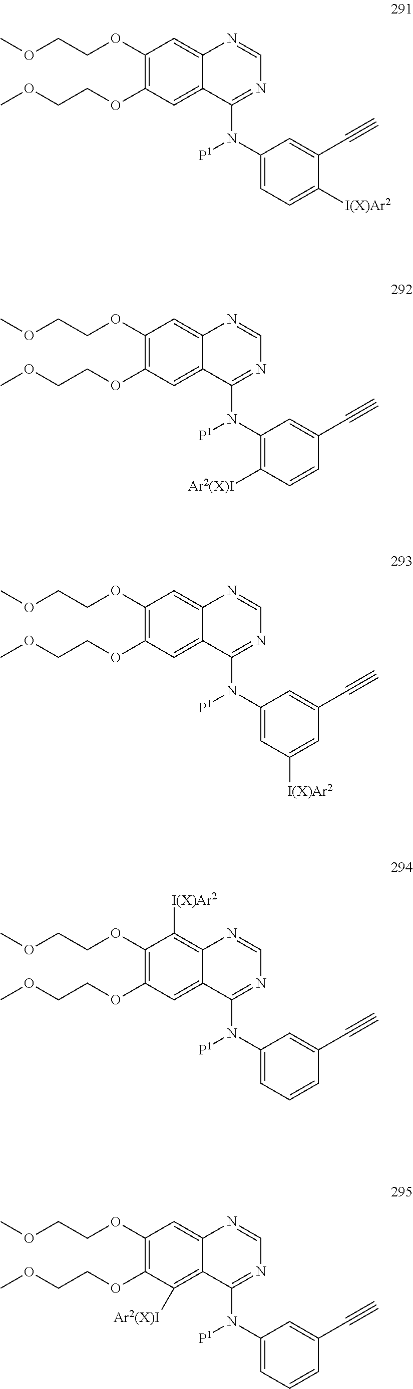

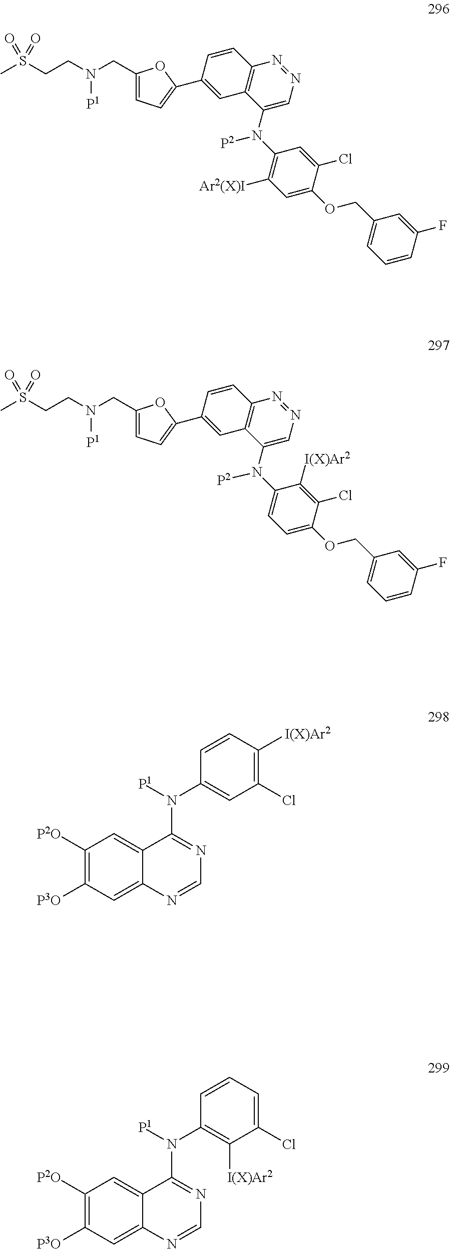

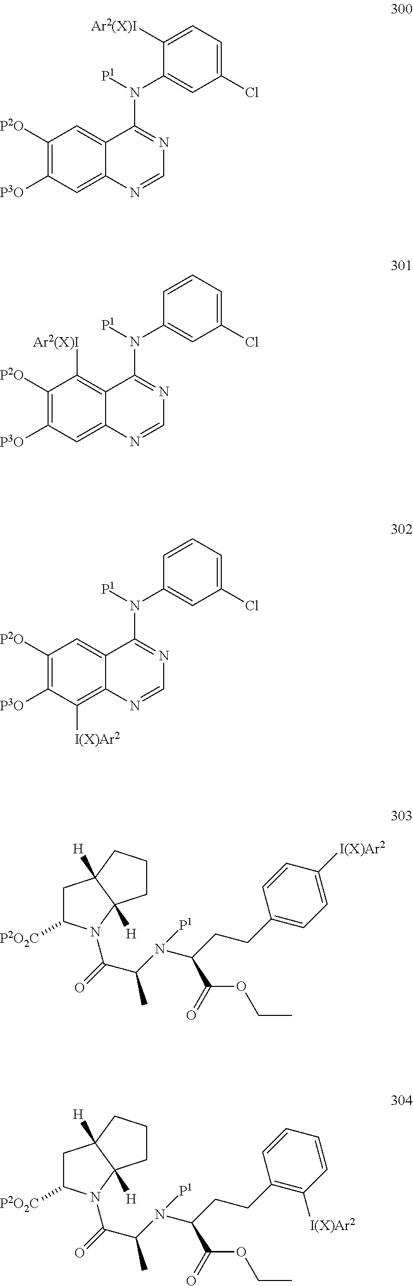

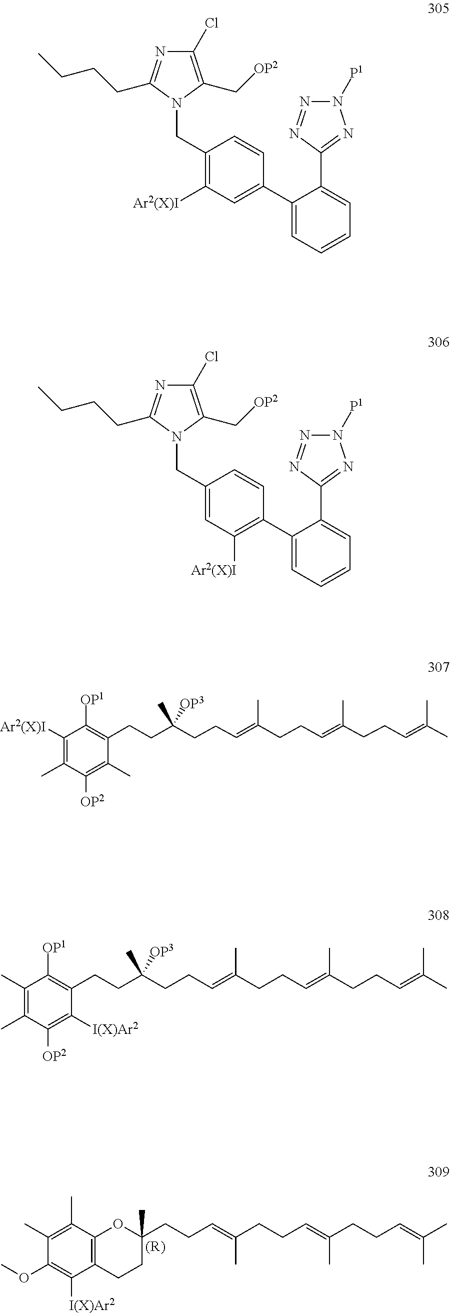

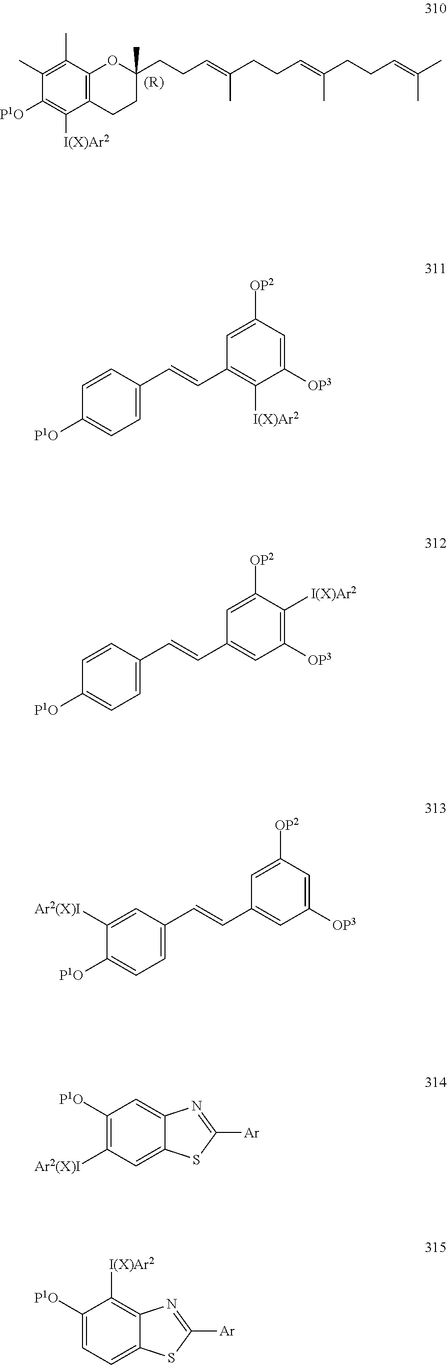

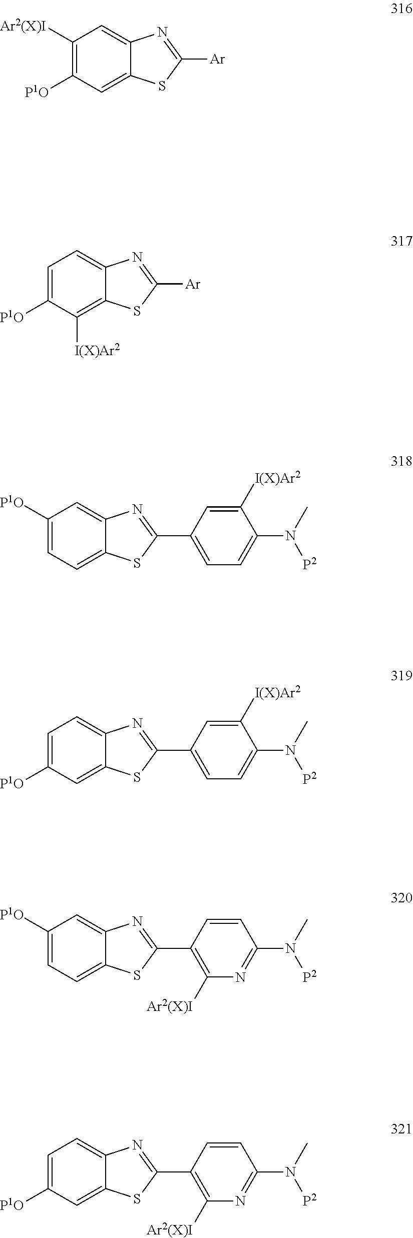

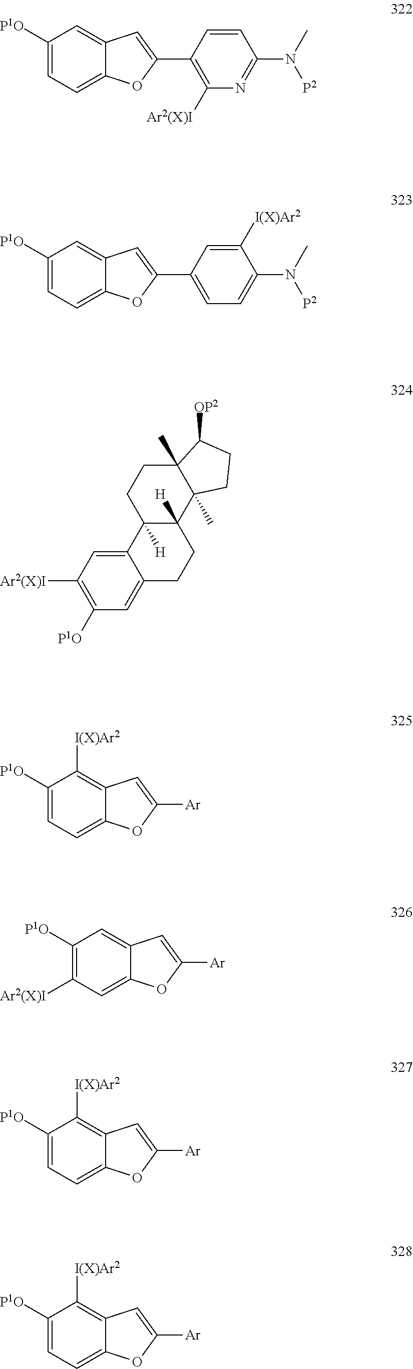

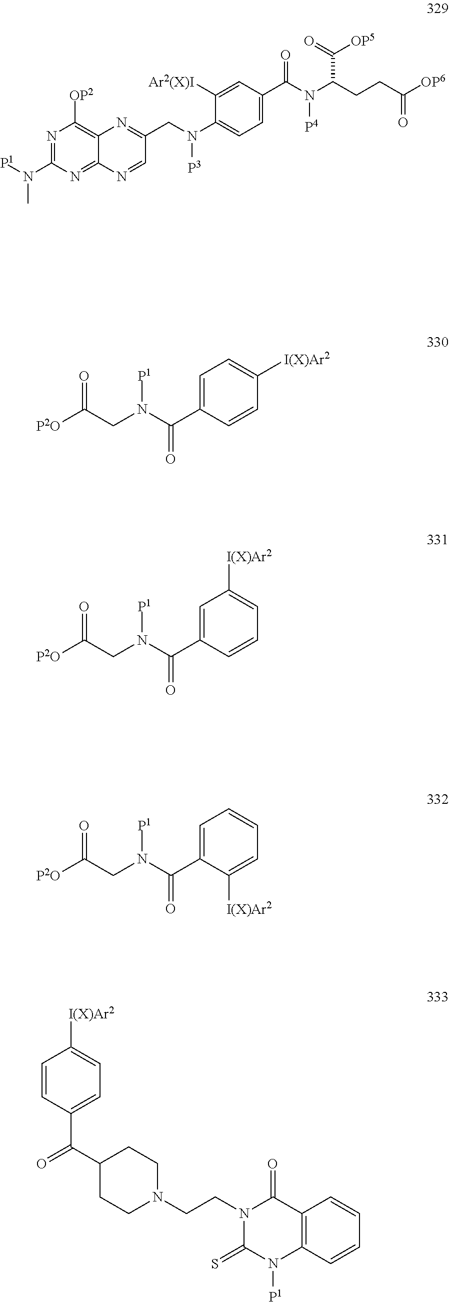

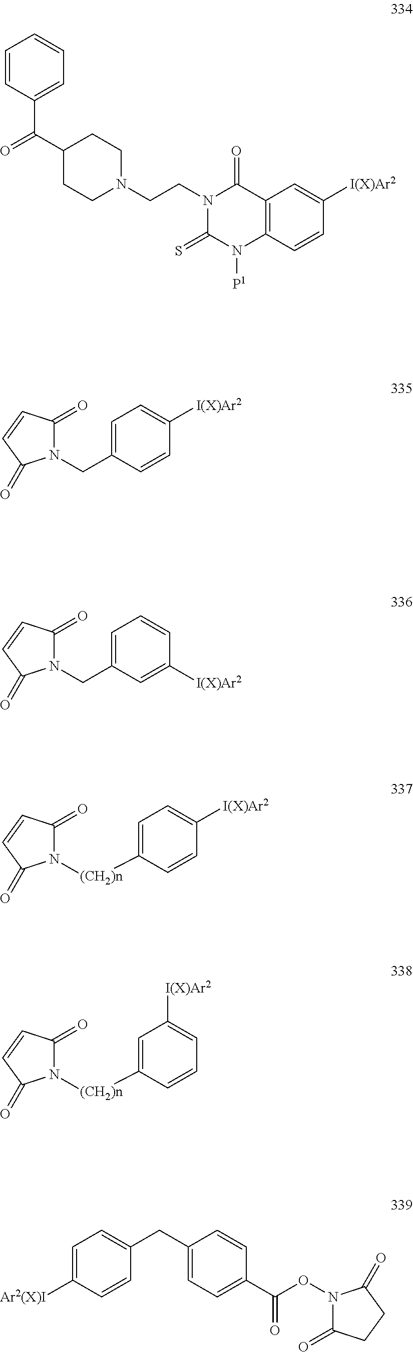

wherein Ar is an optionally substituted aryl or heteroaryl, wherein Ar does not have unprotected protic groups; and P¹, P², P³, P⁴, P⁵, and P⁶ are each, independently, protecting groups; and Ar² and X are defined above. In some embodiments, each X is acetate. In some embodiments, Ar² is p-methoxyphenyl.

In certain preferred embodiments, the compound of Formula III is selected from compounds 231-233. In other preferred embodiments, the compound of Formula III is selected from compounds 290-295. In other preferred embodiments, the compound of Formula III is selected from compounds 318-323. In one preferred embodiments, the compound of Formula III is compound 291. In another preferred embodiments, the compound of Formula III is compound 329. In another preferred embodiments, the compound of Formula III is compound 335. In another preferred embodiments, the compound of Formula III is compound 339.

In some embodiments, the present invention provides the compound of Formula V corresponding to compounds 227-329, wherein X is replaced by Y. In some embodiments, Y is PF₆- or triflate.

In some embodiments, the present application provides any of the individual compounds 1-339 disclosed herein. In some embodiments, the present invention provides any process described herein utilizing any of compounds 1-339. In some embodiments, the present invention provides a compound of Formula VI derived from compounds 227-339.

The compounds of Formula III or V can be used to make fluorinated compounds, including ¹⁸F labeled compounds as described in US 2011/0313170 and US 2012/0004417, which are incorporated herein by reference in its entirety.

For example, the compounds of Formula III or V can be utilized to prepare compounds of Formula VI:

Ar¹—W VI

wherein Ar¹ is as defined above; and W is a moiety wherein the pKa of the acid H—W is less than 12. In one embodiment, the method includes reacting in a polar solvent a compound MW, wherein M is a counter ion and W is as defined in Formula VI and a compound of Formula V:

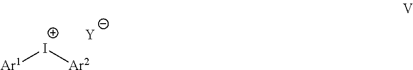

wherein Ar¹ and Ar² are as defined above; Y is a leaving group; and

W is as defined above.

The polar solvent can then be removed from the reaction mixture. The remaining mixture can then be combined with a nonpolar solvent and heated to produce a compound of Formula VI. In some embodiments, the method can include heating a mixture comprising a nonpolar solvent, a compound MW, and a compound of Formula V.

In some embodiments, the nonpolar solution of the reaction mixture of MW and a compound of Formula V can be filtered prior to heating. The filtration step can remove any insoluble material (e.g., insoluble salts) that remain in the reaction mixture. In some embodiments, the solvent can be removed from the filtrate prior to heating (i.e., the residue can be heated neat).

In further embodiments, the nonpolar solution of the reaction mixture of MW and a compound of Formula V can be filtered prior to heating, the nonpolar solvent can be removed (e.g., by evaporation), and the heating of the sample can be performed in a different solvent. In some embodiments, contaminant salts are removed from the solution of the reaction mixture of MW and a compound of Formula V in the polar or nonpolar solution by chromatography. For example, the contaminant salts can be removed by size exclusion, gel filtration, reverse phase, or other chromatographic method prior to heating.

Substituted aryls and heteroaryls which are prepared using the methods described herein can have a W moiety which includes any moiety in which the pKa of H—W (i.e., the conjugate acid of X) is less than about 12. In some cases, W is a radioactive isotope (e.g., ¹⁸F, ¹²³I, ¹³¹I, and compounds having ³²P and ³³P). In some embodiments, W can be chosen from halide, aryl carboxylate, alkyl carboxylate, phosphate, phosphonate, phosphonite, azide, thiocyanate, cyanate, phenoxide, triflate, trifluoroethoxide, thiolates, and stabilized enolates. For example, W can be fluoride, chloride, bromide, iodide, trifluoroacetate, benzoate, and acetate. In some embodiments, X is fluoride. In some embodiments, is a radioactive isotope of fluoride (e.g., ¹⁸F).

Y can be any suitable leaving group. In some embodiments, Y is a weakly coordinating anion (i.e., an anion that coordinates only weakly with iodine). For example, Y can be the conjugate base of a strong acid, for example, any anion for which the pKa of the conjugate acid (H—Y) is less than about 1. For example, Y can be triflate, mesylate, nonaflate, hexaflate, toluene sulfonate (tosylate), nitrophenyl sulfonate (nosylate), bromophenyl sulfonate (brosylate), perfluoroalkyl sulfonate (e.g., perfluoro C₂₋₁₀ alkyl sulfonate), tetraphenylborate, hexafluorophosphate, trifluoroacetate, perfluoroalkylcarboxylate, tetrafluoroborate, perchlorate, hexafluorostibate, hexachlorostibate, chloride, bromide, or iodide. In some embodiments, a slightly more basic leaving group such as acetate or benzoate may be used.

The counter ion M can be any suitable cation for the desired W. The choice of the source of W, and accordingly M, is readily within the knowledge of one of ordinary skill in the art. For example, M can be chosen from an alkali metal, alkaline earth metal and transition metal salts such as, for example, calcium, magnesium, potassium, sodium and zinc salts. Metal cations may also be complexed to cryptands or crown ethers to enhance their solubility and to labilize the W moiety. M can also include organic salts made from quaternized amines derived from, for example, N,N′ dibenzylethylenediamine, chloroprocaine, choline, diethanolamine, ethylenediamine, meglumine (N-methylglucamine) and procaine. In some embodiments, M can be a lithium, sodium, potassium, or cesium with cryptands or crown ethers, a tetrasubstituted ammonium cation, or phosphonium cation. When W is fluoride, the choice of fluoride source is also readily within the knowledge of one of ordinary skill in the art. A variety of fluoride sources can be used in the preparation of the fluorinated aryl and heteroaryl compounds as provided herein, including but not limited to NaF, KF, CsF, tetrabutylammonium fluoride, and tetramethylammonium fluoride. In certain instances the choice of fluoride source will depend on the functionality present on the compound of Formula V.

Accordingly, provided herein is the use of a compound of Formula III for the preparation of a compound of Formula VI, wherein Ar¹ and Ar² are independently, optionally substituted aryl or heteroaryl; X is a ligand that is a conjugate base of an acid HX, wherein HX has a pKa of less than or equal to 5; and W is selected from the group consisting of fluorine, iodine and radioactive isotopes thereof, and astatine. In one embodiment, W is selected from F, ¹⁸F, I, ¹²³I and ¹³¹I. In another embodiment, the compound of Formula III is selected from the group consisting of compounds 227-339. In another embodiment, the compound of Formula III is selected from the group consisting of compounds 231-233, 318-323, 329, 335 and 339.

The methods described above can be useful in the preparation of fluorinated aryl and heteroaryl ring systems. For example, the methods can be used to prepare a compound of Formula VII:

Ar¹—F VII

wherein Ar¹ is an aryl or heteroaryl ring system. In particular, the methods can be used to prepare radio labeled fluorinated aryl and heteroaryl ring systems (e.g., PET radiotracers). In some embodiments, the method can include reacting in a polar solvent a compound MF and a compound of Formula V. The polar solvent can then be removed from the reaction mixture. The remaining mixture can then be combined with a nonpolar solvent and heated to produce a compound of Formula VII.

In some embodiments, the method can include heating a mixture comprising a nonpolar solvent, a compound MF, and a compound of Formula V.

In some embodiments, the nonpolar solution of the reaction mixture of MF and a compound of Formula V can be filtered prior to heating. The filtration step can remove any insoluble material (e.g., insoluble salts) that remain in the reaction mixture. In some embodiments, the solvent can be removed from the filtrate prior to heating (i.e., the residue can be heated neat).

In some embodiments, the nonpolar solution of the reaction mixture of MF and a compound of Formula V can be filtered prior to heating, the nonpolar solvent can be removed (e.g., by evaporation), and the heating of the sample can be performed in a different solvent.

In some embodiments, contaminant salts are removed from the nonpolar solution of the reaction mixture of MF and a compound of Formula V by chromatography. For example, the contaminant salts can be removed by size exclusion, gel filtration, reverse phase, or other chromatographic method prior to heating.

In general, the methods described herein are not compatible with aryl iodides having N—H or O—H bonds.

### DEFINITIONS

It is appreciated that certain features of the invention, which are, for clarity, described in the context of separate embodiments, can also be provided in combination in a single embodiment. Conversely, various features of the invention which are, for brevity, described in the context of a single embodiment, can also be provided separately or in any suitable subcombination.

As used herein, the phrase “optionally substituted” means unsubstituted or substituted. As used herein, the term “substituted” means that a hydrogen atom is removed and replaced by a substituent. It is to be understood that substitution at a given atom is limited by valency. Throughout the definitions, the term “Cₙ₋ₘ” indicates a range which includes the endpoints, wherein n and m are integers and indicate the number of carbons. Examples include C₁₋₄, C₁₋₆, and the like.

The term “n-membered” where n is an integer typically describes the number of ring-forming atoms in a moiety where the number of ring-forming atoms is n. For example, piperidinyl is an example of a 6-membered heterocycloalkyl ring, pyrazolyl is an example of a 5-membered heteroaryl ring, pyridyl is an example of a 6-membered heteroaryl ring, and 1,2,3,4-tetrahydro-naphthalene is an example of a 10-membered cycloalkyl group. As used herein, the term “Cₙ₋ₘ alkyl”, employed alone or in combination with other terms, refers to a saturated hydrocarbon group that may be straight-chain or branched, having n to m carbons. In some embodiments, the alkyl group contains from 1 to 3 carbon atoms. Examples of alkyl moieties include, but are not limited to, chemical groups such as methyl, ethyl, n-propyl, and isopropyl.

As used herein, the term “Cₙ₋ₘ alkoxy”, employed alone or in combination with other terms, refers to a group of formula —O-alkyl, wherein the alkyl group has n to m carbons. Example alkoxy groups include methoxy, ethoxy, and propoxy (e.g., n-propoxy and isopropoxy). In some embodiments, the alkyl group has 1 to 3 carbon atoms.

As used herein, the term “alkylene”, employed alone or in combination with other terms, refers to a divalent alkyl linking group. Examples of alkylene groups include, but are not limited to, ethan-1,2-diyl, propan-1,3-diyl, propan-1,2-diyl, butan-1,4-diyl, butan-1,3-diyl, butan-1,2-diyl, 2-methyl-propan-1,3-diyl, and the like.

As used herein, “Cₙ₋ₘ alkenyl” refers to an alkyl group having one or more double carbon-carbon bonds and having n to m carbons. In some embodiments, the alkenyl moiety contains 2 to 6 or to 2 to 4 carbon atoms. Example alkenyl groups include, but are not limited to, ethenyl, n-propenyl, isopropenyl, n-butenyl, sec-butenyl, and the like.

As used herein, “Cₙ₋ₘ alkynyl” refers to an alkyl group having one or more triple carbon-carbon bonds and having n to m carbons. Example alkynyl groups include, but are not limited to, ethynyl, propyn-1-yl, propyn-2-yl, and the like. In some embodiments, the alkynyl moiety contains 2 to 6 or 2 to 4 carbon atoms.

As used herein, the term “Cₙ₋ₘ alkylamino” refers to a group of formula —NH(alkyl), wherein the alkyl group has n to m carbon atoms. In some embodiments, the alkyl group has 1 to 6 or 1 to 4 carbon atoms.

As used herein, the term “di-Cₙ₋ₘ-alkylamino” refers to a group of formula —)₂, wherein the two alkyl groups each has, independently, n to m carbon atoms. In some embodiments, each alkyl group independently has 1 to 6 or 1 to 4 carbon atoms.

As used herein, the term “Cₙ₋ₘ alkoxycarbonyl” refers to a group of formula —C(O)O-alkyl, wherein the alkyl group has n to m carbon atoms. In some embodiments, the alkyl group has 1 to 6 or 1 to 4 carbon atoms.

As used herein, the term “Cₙ₋ₘ alkylcarbonyl” refers to a group of formula —C(O)-alkyl, wherein the alkyl group has n to m carbon atoms. In some embodiments, the alkyl group has 1 to 6 or 1 to 4 carbon atoms.

As used herein, the term “Cₙ₋ₘ alkylcarbonylamino” refers to a group of formula —NHC(O)-alkyl, wherein the alkyl group has n to m carbon atoms. In some embodiments, the alkyl group has 1 to 6 or 1 to 4 carbon atoms.

As used herein, the term “Cₙ₋ₘ alkylsulfonylamino” refers to a group of formula —NHS(O)₂-alkyl, wherein the alkyl group has n to m carbon atoms. In some embodiments, the alkyl group has 1 to 6 or 1 to 4 carbon atoms.

As used herein, the term “aminosulfonyl”, employed alone or in combination with other terms, refers to a group of formula —S(O)₂NH₂.

As used herein, the term “Cₙ₋ₘ alkylaminosulfonyl” refers to a group of formula —S(O)₂NH(alkyl), wherein the alkyl group has n to m carbon atoms. In some embodiments, the alkyl group has 1 to 6 or 1 to 4 carbon atoms.

As used herein, the term “di(Cₙ₋ₘ alkyl)aminosulfonyl” refers to a group of formula —S(O)₂N(alkyl)₂, wherein each alkyl group independently has n to m carbon atoms. In some embodiments, each alkyl group has, independently, 1 to 6 or 1 to 4 carbon atoms. As used herein, the term “aminosulfonylamino” refers to a group of formula —NHS(O)₂NH₂. As used herein, the term “Cₙ₋ₘ alkylaminosulfonylamino” refers to a group of formula —NHS(O)₂NH(alkyl), wherein the alkyl group has n to m carbon atoms. In some embodiments, the alkyl group has 1 to 6 or 1 to 4 carbon atoms.

As used herein, the term “di(Cₙ₋ₘ alkyl)aminosulfonylamino” refers to a group of formula —NHS(O)₂N(alkyl)₂, wherein each alkyl group independently has n to m carbon atoms. In some embodiments, each alkyl group has, independently, 1 to 6 or 1 to 4 carbon atoms. As used herein, the term “aminocarbonylamino” refers to a group of formula —NHC(O)NH₂.

As used herein, the term “Cₙ₋ₘ alkylaminocarbonylamino” refers to a group of formula —NHC(O)NH(alkyl), wherein the alkyl group has n to m carbon atoms. In some embodiments, the alkyl group has 1 to 6 or 1 to 4 carbon atoms.

As used herein, the term “di(Cₙ₋ₘ alkyl)aminocarbonylamino” refers to a group of formula —NHC(O)N(alkyl)₂, wherein each alkyl group independently has n to m carbon atoms. In some embodiments, each alkyl group has, independently, 1 to 6 or 1 to 4 carbon atoms.

As used herein, the term “Cₙ₋ₘ alkylcarbamyl” refers to a group of formula —C(O)—NH(alkyl), wherein the alkyl group has n to m carbon atoms. In some embodiments, the alkyl group has 1 to 6 or 1 to 4 carbon atoms.

As used herein, the term “di(Cₙ₋ₘ₋alkyl)carbamyl” refers to a group of formula —C(O)N(alkyl)₂, wherein the two alkyl groups each has, independently, n to m carbon atoms. In some embodiments, each alkyl group independently has 1 to 6 or 1 to 4 carbon atoms. As used herein, the term “Cₙ₋ₘ alkylthio” refers to a group of formula —S-alkyl, wherein the alkyl group has n to m carbon atoms. In some embodiments, the alkyl group has 1 to 6 or 1 to 4 carbon atoms.

As used herein, the term “Cₙ₋ₘ alkylsulfinyl” refers to a group of formula —S(O)-alkyl, wherein the alkyl group has n to m carbon atoms. In some embodiments, the alkyl group has 1 to 6 or 1 to 4 carbon atoms.

As used herein, the term “Cₙ₋ₘ alkylsulfonyl” refers to a group of formula —S(O)₂-alkyl, wherein the alkyl group has n to m carbon atoms. In some embodiments, the alkyl group has 1 to 6 or 1 to 4 carbon atoms.

As used herein, the term “amino” refers to a group of formula NH₂.

As used herein, the term “C₁₋₆ alkyl-O—C₁₋₆ alkylene” refers to a group of formula C₁₋₆ alkylene-O—C₁₋₆ alkyl.

As used herein, the term “C₁₋₆ alkyl-NR^{4a}—C₁₋₆ alkylene” refers to a group of formula C₁₋₆ alkylene-NR^{4a}—C₁₋₆ alkyl.

As used herein, the term “aryl”, employed alone or in combination with other terms, refers to a monocyclic or polycyclic (e.g., having 2, 3 or 4 fused rings) aromatic hydrocarbon, such as, but not limited to, phenyl, 1-naphthyl, 2-naphthyl, anthracenyl, phenanthrenyl, and the like. In some embodiments, aryl is C₆₋₁₀ aryl. In some embodiments, the aryl group is a naphthalene ring or phenyl ring. In some embodiments, the aryl group is phenyl.

As used herein, the term “arylalkyl” refers to a group of formula -alkylenearyl. In some embodiments, arylalkyl is C₆₋₁₀ aryl-C₁₋₃ alkyl. In some embodiments, arylalkyl is C₆₋₁₀ aryl-C₁₄ alkyl. In some embodiments, arylalkyl is benzyl.
As used herein, the term “carbamyl” refers to a group of formula C(O)NH₂.
As used herein, the term “carbonyl”, employed alone or in combination with other terms, refers to a —C(O)— group.

As used herein, the term “carboxy” refers to a group of formula —C(O)OH. As used herein, the term “cycloalkyl”, employed alone or in combination with other terms, refers to a non-aromatic cyclic hydrocarbon moiety, which may optionally contain one or more alkenylene groups as part of the ring structure. Cycloalkyl groups can include mono- or polycyclic (e.g., having 2, 3 or 4 fused, bridged or spiro rings) ring systems. Also included in the definition of cycloalkyl are moieties that have one or more aromatic rings fused (i.e., having a bond in common with) to the cycloalkyl ring, for example, benzo derivatives of cyclopentane, cyclopentene, cyclohexane, and the like. One or more ring-forming carbon atoms of a cycloalkyl group can be oxidized to form C═O or C═S linkages. In some embodiments, cycloalkyl is C₃₋₁₂ cycloalkyl, which is monocyclic or bicyclic. Exemplary cycloalkyl groups include 1,2,3,4-tetrahydro-naphthalene, cyclopropyl, cyclobutyl, cyclopentyl, cyclohexyl, cycloheptyl, cyclopentenyl, cyclohexenyl, cyclohexadienyl, cycloheptatrienyl, norbornyl, norpinyl, norcarnyl, adamantyl, and the like. In some embodiments, the cycloalkyl group is cyclopropyl, cyclobutyl, cyclopentyl, or cyclohexyl.

As used herein, the term “cycloalkylalkyl” refers to a group of formula -alkylene-cycloalkyl. In some embodiments, cycloalkylalkyl is C₃₋₁₂ cycloalkyl-C₁₋₃ alkyl, wherein the cycloalkyl portion is monocyclic or bicyclic. In some embodiments, cycloalkylalkyl is C₃₋₁₂ cycloalkyl-C₁₋₄ alkyl, wherein the cycloalkyl portion is monocyclic or bicyclic. As used herein, “Cₙ₋ₘ haloalkoxy” refers to a group of formula —O-haloalkyl having n to m carbon atoms. An example haloalkoxy group is OCF₃. In some embodiments, the haloalkoxy group is fluorinated only. In some embodiments, the alkyl group has 1 to 6 or 1 to 4 carbon atoms.

As used herein, the term “halo” refers to a halogen atom selected from F, Cl, I or Br. As used herein, the term “Cₙ₋ₘ haloalkyl”, employed alone or in combination with other terms, refers to an alkyl group having from one halogen atom to 2s+1 halogen atoms which may be the same or different, where “s” is the number of carbon atoms in the alkyl group, wherein the alkyl group has n to m carbon atoms. In some embodiments, the haloalkyl group is fluorinated only. In some embodiments, the haloalkyl group is fluoromethyl, difluoromethyl, or trifluoromethyl. In some embodiments, the haloalkyl group is trifluoromethyl. In some embodiments, the alkyl group has 1 to 6 or 1 to 4 carbon atoms.

As used herein, the term “heteroaryl”, employed alone or in combination with other terms, refers to a monocyclic or polycyclic (e.g., having 2, 3 or 4 fused rings) aromatic hydrocarbon moiety, having one or more heteroatom ring members selected from nitrogen, sulfur and oxygen. In some embodiments, heteroaryl is 5- to 10-membered C₁₋₉ heteroaryl, which is monocyclic or bicyclic and which has 1, 2, 3, or 4 heteroatom ring members independently selected from nitrogen, sulfur and oxygen. The heteroaryl may have one or more C═O or C═S linkages. When the heteroaryl group contains more than one heteroatom ring member, the heteroatoms may be the same or different. Example heteroaryl groups include, but are not limited to, pyridine, pyrimidine, pyrazine, pyridazine, pyrrole, pyrazole, azolyl, oxazole, thiazole, imidazole, furan, thiophene, quinoline, isoquinoline, indole, benzothiophene, benzofuran, benzisoxazole, imidazo[1,2-b]thiazole, purine, or the like.

A five-membered ring heteroaryl is a heteroaryl with a ring having five ring atoms wherein one or more (e.g., 1, 2, or 3) ring atoms are independently selected from N, O, and S. Exemplary five-membered ring heteroaryls are thienyl, furyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, and 1,3,4-oxadiazolyl.

A six-membered ring heteroaryl is a heteroaryl with a ring having six ring atoms wherein one or more (e.g., 1, 2, or 3) ring atoms are independently selected from N, O, and S. Exemplary six-membered ring heteroaryls are pyridyl, pyrazinyl, pyrimidinyl, triazinyl and pyridazinyl. As used herein, the term “heteroarylalkyl” refers to a group of formula alkylene-heteroaryl. In some embodiments, heteroarylalkyl is C₁₋₉ heteroaryl-C₁₋₃ alkyl, wherein the heteroaryl portion is monocyclic or bicyclic and has 1, 2, 3, or 4 heteroatom ring members independently selected from nitrogen, sulfur and oxygen. In some embodiments, heteroarylalkyl is C₁₋₉ heteroaryl-C₁₄ alkyl, wherein the heteroaryl portion is monocyclic or bicyclic and has 1, 2, 3, or 4 heteroatom ring members independently selected from nitrogen, sulfur and oxygen.

As used herein, the term “heterocycloalkyl”, employed alone or in combination with other terms, refers to non-aromatic ring system, which may optionally contain one or more alkenylene or alkynylene groups as part of the ring structure, and which has at least one heteroatom ring member independently selected from nitrogen, sulfur and oxygen. When the heterocycloalkyl groups contains more than one heteroatom, the heteroatoms may be the same or different. Heterocycloalkyl groups can include mono- or polycyclic (e.g., having 2, 3 or 4 fused, bridged, or spiro rings) ring systems, including spiro systems. Also included in the definition of heterocycloalkyl are moieties that have one or more aromatic rings fused (i.e., having a bond in common with) to the non-aromatic ring, for example, 1,2,3,4-tetrahydro-quinoline and the like. The carbon atoms or heteroatoms in the ring(s) of the heterocycloalkyl group can be oxidized to form a C═O, C═S, S═O, or S(═O)₂ group (or other oxidized linkage) or a nitrogen atom can be quaternized. In some embodiments, heterocycloalkyl is 5- to 10-membered C₂₋₉ heterocycloalkyl, which is monocyclic or bicyclic and which has 1, 2, 3, or 4 heteroatom ring members independently selected from nitrogen, sulfur and oxygen. Examples of heterocycloalkyl groups include 1,2,3,4-tetrahydro-quinoline, azetidine, azepane, pyrrolidine, piperidine, piperazine, morpholine, thiomorpholine, pyran, and a 2-oxo-1,3-oxazolidine ring. As used herein, the term “heterocycloalkylalkyl” refers to a group of formula -alkylene-heterocycloalkyl. In some embodiments, heterocycloalkylalkyl is C₂₋₉ heterocycloalkyl-C₁₋₃ alkyl, wherein the heterocycloalkyl portion is monocyclic or bicyclic and has 1, 2, 3, or 4 heteroatom ring members independently selected from nitrogen, sulfur and oxygen. In some embodiments, heterocycloalkylalkyl is C₂₋₉ heterocycloalkyl-C₁₄ alkyl, wherein the heterocycloalkyl portion is monocyclic or bicyclic and has 1, 2, 3, or 4 heteroatom ring members independently selected from nitrogen, sulfur and oxygen.

The compounds described herein can be asymmetric (e.g., having one or more stereocenters). All stereoisomers, such as enantiomers and diastereomers, are intended unless otherwise indicated. Compounds of the present invention that contain asymmetrically substituted carbon atoms can be isolated in optically active or racemic forms. Methods on how to prepare optically active forms from optically inactive starting materials are known in the art, such as by resolution of racemic mixtures or by stereoselective synthesis. Many geometric isomers of olefins, C═N double bonds, and the like can also be present in the compounds described herein, and all such stable isomers are contemplated in the present invention. Cis and trans geometric isomers of the compounds of the present invention are described and may be isolated as a mixture of isomers or as separated isomeric forms.

Resolution of racemic mixtures of compounds can be carried out by any of numerous methods known in the art. An example method includes fractional recrystallization using a chiral resolving acid which is an optically active, salt-forming organic acid. Suitable resolving agents for fractional recrystallization methods are, for example, optically active acids, such as the D and L forms of tartaric acid, diacetyltartaric acid, dibenzoyltartaric acid, mandelic acid, malic acid, lactic acid or the various optically active camphorsulfonic acids such as -camphorsulfonic acid. Other resolving agents suitable for fractional crystallization methods include stereoisomerically pure forms of -methylbenzylamine (e.g., S and R forms, or diastereomerically pure forms), 2-phenylglycinol, norephedrine, ephedrine, N-methylephedrine, cyclohexylethylamine, 1,2-diaminocyclohexane, and the like.

Resolution of racemic mixtures can also be carried out by elution on a column packed with an optically active resolving agent (e.g., dinitrobenzoylphenylglycine). Suitable elution solvent composition can be determined by one skilled in the art.

Compounds of the invention also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone enol pairs, amide-imidic acid pairs, lactam-lactim pairs, amide-imidic acid pairs, enamine imine pairs, and annular forms where a proton can occupy two or more positions of a heterocyclic system, for example, 1H- and 3H-imidazole, 1H-, 2H- and 4H-1,2,4-triazole, 1H- and 2H-isoindole, and 1H- and 2H-pyrazole. Tautomeric forms can be in equilibrium or sterically locked into one form by appropriate substitution.

Compounds of the invention can also include all isotopes of atoms occurring in the intermediates or final compounds. Isotopes include those atoms having the same atomic number but different mass numbers. For example, isotopes of hydrogen include tritium and deuterium.

The term, “compound,” as used herein is meant to include all stereoisomers, geometric isomers, tautomers, and isotopes of the structures depicted. Compounds herein identified by name or structure as one particular tautomeric form are intended to include other tautomeric forms unless otherwise specified.

### EXAMPLES

The invention will be described in greater detail by way of specific examples. The following examples are offered for illustrative purposes, and are not intended to limit the invention in any manner. Those of skill in the art will readily recognize a variety of noncritical parameters which can be changed or modified to yield essentially the same results.

General Procedure for Oxidation of an Iodoarene with F-TEDA-BF₄/TMSOAc

Under a dry atmosphere of N₂, 0.5 mmol of the aryl iodide (1-113) was dissolved in 3 mL of dry acetonitrile. Trimethylsilyl acetate (165 mg, 1.25 mmol) was added to the solution followed by a solution of F-TEDA-BF₄ (220 mg, 0.65 mmol) in an additional 3 mL of dry acetonitrile. The reaction mixture was allowed to stand at room temperature for 3-8 h. Acetonitrile was then removed in vacuo and 3×3 mL dichloromethane were used to extract the remaining mixture. The combined dichloromethane solutions were washed with 4×6 mL aqueous acetate buffer (NaOAc: HOAc=0.5 M: 0.5 M, pH=5) and dried over sodium sulfate. The dichloromethane was removed in vacuo to yield the crude product, which was dissolved in 3 mL of dichloromethane and dripped into 150 mL pentane to precipitate the aryliodonium diacetate products, which were collected by vacuum filtration.

### Example 1

### 1-(Diacetoxyiodo)-4-methoxybenzene (1a)

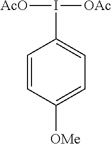 [SMILES: COc1ccc(I(OC(C)=O)OC(C)=O)cc1]

(70%) ¹H NMR (CD₃CN, 400 MHz, 25° C.): δ 8.055 (d, J=9.1 Hz, 2H), 7.053 (d, J=9.1 Hz, 2H), 3.861 (s, 3H), 1.905 (s, 6H); ¹³C NMR (CD₃CN, 100 MHz, 25° C.) δ 177.73, 163.73, 138.75, 118.00, 111.97, 56.85, 20.76; HRMS: (HRFAB) calcd. for C₁₋₄H₁₃NO₄I⁺ [M−2OAc+3-NBA]⁺ 385.9889 found 385.9885. This compound has been prepared previously: Cerioni, G. and G. Uccheddu, “Solution structure of bis(acetoxy)iodoarenes as observed by 170 NMR spectroscopy”, Tetrahedron Lett. 2004, 45, 505-507. Characterization data were consistent with the previous literature.

### Example 2

### 3-(Diacetoxyiodo)benzonitrile

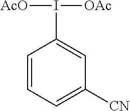 [SMILES: CC(=O)OI(OC(C)=O)c1cccc(C#N)c1]

¹H NMR (CD₃CN, 400 MHz, 25° C.): δ 8.515 (s, 1H, H2), 8.406 (d, J=8.1 Hz, 1H, H6), 7.866 (d, J=8.1 Hz, 1H, H4), 7.711 (t, J=8.1 Hz, 1H, H5), 1.954 (s, 6H, (OCOCH₃)₂); ¹³C NMR (CD₃CN, 100 MHz, 25° C.) δ 178.25 (CO), 140.65 (C6), 139.69 (C2), 136.88 (C5), 132.95 (C4), 121.84 (C3), 115.82 (CN), 109.99 (C1); HRMS (HRFAB): calcd. For C₁₋₄H₁₀N₂O₃I [M 2Oac+3-NBA]⁺ 380.9736 found 380.9722. (Kazmierczak, P. and L. Skulski, “A simple, two-step conversion of various iodo arenes to (diacetoxyiodo) arenes with chromium(VI) oxide as the oxidant”, Synthesis 1998, 1721-1723): ¹H NMR (CDCl₃, 200 MHz) δ 7.61-8.39 (4H, m, ArH), 2.02 (6H, s, MeCO₂).)

### Example 3

### 2-[2-[(Di-tert-butoxycarbonyl)amino]ethyl]-4,5-dimethoxyiodobenzene

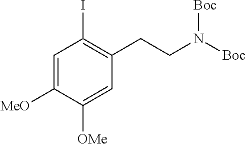 [SMILES: COc1cc(I)c(CCN(C(=O)OC(C)(C)C)C(=O)OC(C)(C)C)cc1OC]

To a solution of N-iodosuccinamide (NIS) (4.95 g, 22 mmol) in dry acetonitrile (50 mL) was added 2-(3,4-dimethoxyphenyl)ethanamine (3.32 mL, 20 mmol) and trifluoroacetic acid (3.85 mL, 50 mmol) with stirring. The mixture was stirred at room temperature in a 250 mL round bottom flask for two hours. The acetonitrile was removed and the remaining solid was taken up in water. The water solution was treated with saturated sodium bisulfite aqueous solution until the purple color disappeared. The pH was adjusted to 8 and the aqueous solution was extracted with dichloromethane (3×50 mL). The organic layers were combined and dried over sodium sulfate. The solvent was evaporated to yield 2-(2-iodo-4,5-dimethoxyphenyl)ethanamine (4.3 g, 70%). The crude product was dried under dynamic vacuum overnight and was sufficiently pure for subsequent steps.

2-(2-iodo-4,5-dimethoxyphenyl)ethanamine (4.3 g) was dissolved in a dry acetonitrile (30 mL) solution containing BOC anhydride (4.84 g, 22 mmol), 4-dimethylpyridine (195 mg, 1.6 mmol), and triethylamine (3.1 mL, 22 mmol). The reaction was stirred overnight at room temperature before being concentrated under reduced pressure. The concentrate was diluted with 30 mL ethyl acetate and washed with saturated NH₄Cl solution, water, and brine. The organic layer was dried over sodium sulfate and concentrated under reduced pressure. The crude product was purified by column chromatography (60 Å silica, 20% ethyl acetate in hexanes, R_{f}=0.3) before subjected to a second round of BOC protection. The purified, BOC-protected 2-(2-iodo-4,5-dimethoxyphenyl)ethanamine was dissolved in 30 mL of an acetonitrile solution containing BOC anhydride (4.36 g, 20 mmol), DMAP (195 mg, 1.6 mmol), and triethylamine (2.78 mL, 20 mmol) and stirred at room temperature for 20 h. The reaction mixture was concentrated in vacuo, diluted with 30 mL ethyl acetate and washed with saturated NH₄Cl solution, water, and brine. The organic layer was dried over sodium sulfate and concentrated under reduced pressure. The crude product was purified by column chromatography (60 Å silica, 15% ethyl acetate in hexanes, Rf=0.3) to yield 8.8 g (90%) 2-[2-[(di-tert-butoxycarbonyl)amino]ethyl]-4,5-dimethoxyiodobenzene. ¹H NMR (CD₃CN, 400 MHz, 25° C.): δ 7.25 (s, 1H), 6.72 (s, 1H), 3.77 (t, J=6.60 Hz, 1H), 3.76 (s, 3H), 3.74 (s, 3H), 2.93 (t, J=6.60 Hz, 1H), 1.41 (s, 18H); ¹³C NMR (CD₃CN, 400 MHz, 25° C.): δ 170.9, 153.3, 150.6, 149.6, 135.3, 122.9, 114.7, 88.9, 82.8, 56.8, 56.4, 47.0, 40.1, 28.3; HRMS (HREI): calcd. for C₂₀H₃₀INO₆ M⁺ 507.1118 found 507.1122; calcd. for C₂₀H₃₀INO₆ [M+Na]⁺ 530.1016 found 530.1036.

### Example 4

### 2-[(2S)-2-[(Di-tert-butoxycarbonyl)amino]-3-methoxy-3-oxopropyl]-4,5-dimethoxyiodobenzene

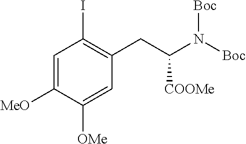 [SMILES: COC(=O)[C@H](Cc1cc(OC)c(OC)cc1I)N(C(=O)OC(C)(C)C)C(=O)OC(C)(C)C]

To a solution of N-iodosuccinamide (8.3 g, 37 mmol) in 80 mL of dry acetonitrile were added (S)-3-(3,4-dimethoxyphenyl)1-methoxy-1-oxopropan-2-amine hydrochloride (4.63 g, 16.8 mmol) and trifluoroacetic acid (2.7 mL, 37 mmol) with stirring. The reaction mixture was stirred at room temperature in a 250 mL round bottom flask protected from light for 2 and half hours. The acetonitrile was removed and the remaining solid was taken up into water. The water solution was treated with saturated sodium bisulfite aqueous solution until the purple color disappeared. The pH was adjusted to 8 using saturated sodium bicarbonate solution. The neutralized aqueous solution was extracted with dichloromethane (3×50 mL). The organic layers were combined and dried over sodium sulfate. The solvent was evaporated to yield (S)-3-(2-Iodo-4,5-dimethoxyphenyl)-1-methoxy-1-oxopropan-2-amine (5.17 g, 98%) as a pale yellow oil. The crude product was dried over dynamic vacuum overnight and was sufficiently pure for subsequent steps.

(S)-3-(2-Iodo-4,5-dimethoxyphenyl)-1-methoxy-1-oxopropan-2-amine (5.17 g) was dissolved in a dry acetonitrile (40 mL) solution containing BOC anhydride (7.17 g, 32.9 mmol) and 4-dimethylpyridine (320 mg, 2.63 mmol), triethylamine (4.57 mL, 32.9 mmol). The reaction was stirred overnight at room temperature before being concentrated under reduced pressure. The concentrate was diluted with 40 mL ethyl acetate and washed with saturated NH₄Cl solution, water, and brine. The organic layer was dried over sodium sulfate and concentrated under reduced pressure. The crude product was purified by column chromatography (60 Å silica, 20% ethyl acetate in hexanes, R_{f}=0.3) before being subjected to a second round of BOC protection. The product was dissolved in 40 mL of an acetonitrile solution containing BOC anhydride (7.17 g, 32.9 mmol), 4-dimethylpyridine (320 mg, 2.63 mmol), triethylamine (4.57 mL, 32.9 mmol) and stirred at room temperature for 20 h. The reaction mixture was concentrated in vacuo, diluted with 40 mL ethyl acetate, and washed with saturated NH₄Cl solution, water, and brine. The organic layer was dried over sodium sulfate and concentrated under reduced pressure. Chromatographic purification (60 Å silica, 15% ethyl acetate in hexanes, Rf=0.3) afforded 2-[(2S)-2-[(Di-tert-butoxycarbonyl)amino]-3-methoxy-3-oxopropyl]-4,5-dimethoxyiodobenzene (7.63 g, 82%). ¹H NMR (CD₂Cl₂, 400 MHz, 25° C.): δ 7.19 (s, 1H), 6.62 (s, 1H), 5.13 (dd, J₁=11.2 Hz, J₂=4.3 Hz, 1H), 3.77 (s, 3H), 3.76 (s, 3H), 3.74 (s, 3H), 3.44 (dd, J₁=14.1 Hz, J₂=4.3 Hz, 1H), 3.30 (dd, J₁=14.1 Hz, J₂=11.2 Hz, 1H), 1.36 (s, 18H); ¹³C NMR (CD₂Cl₂, 400 MHz, 25° C.): δ 170.9, 152.3, 149.9, 149.1, 133.1, 122.3, 114.5, 89.2, 83.4, 58.3, 56.6, 56.2, 52.7, 40.6, 28.1; HRMS (HRFAB): calcd. for C₂₂H₃₂INO₈ M⁺ 565.1173 found 565.1168, calcd. for C₂₂H₃₃INO₈ [M+H]⁺ 566.1251 found 566.1230.

### Example 4

### 2-[(2S)-2-[(Di-tert-butoxycarbonyl)amino]-3-methoxy-3-oxopropyl]-4,5-dimethoxyiodobenzene (Alternative Procedure)

(S)-3-(2-Iodo-4,5-dimethoxyphenyl)-1-methoxy-1-oxopropan-2-amine (70.0 g, 0.192 mol) was dissolved in 300 mL of tetrahydrofuran, 230 mL of saturated aqueous sodium bicarbonate was added and the mixture was stirred vigorously to avoid bi-layer formation. A 1 M solution of BOC anhydride in tetrahydrofuran (230 mL) was added slowly to the reaction mixture and the mixture was allowed to stir for 2 hours. After 2 hours the organic layer was separated, and the aqueous layer was extracted twice with 200 mL of ethyl acetate. The organic layers were combined and dried with sodium sulfate. Removal of the solvent by rotary evaporation gave a light yellow solid. This solid was dissolved in 2 L of acetonitrile and triethylamine (215 mL, 1.5 mol), Boc anhydride (58.7 g, 0.269 mol), and 4-(dimethylamino)pyridine (4.7 g, 0.038 mol) were added to the reaction mixture. The reaction mixture was allowed to stir for 20 hours. After 20 hours, acetonitrile was removed by rotary evaporation to give a deep red oily residue. The residue was purified by silica gel chromatography using a gradient of 5/10/20% ethyl acetate/hexanes (R_{f}=0.3) to afford 2-[(2S)-2-[(di-tert-butoxycarbonyl)amino]-3-methoxy-3-oxopropyl]-4,5-dimethoxyiodobenzene as a light yellow oil which spontaneously crystallized under vacuum. (The silica gel was deactivated by treating it with 1% trimethylamine in hexanes prior to chromatography in order to prevent loss of the amine Boc groups.)

### Example 5

### 2-(Diacetoxyiodo)-1-[2-[(di-tert-butoxycarbonyl)amino]ethyl]-4,5-dimethoxybenzene (5a)

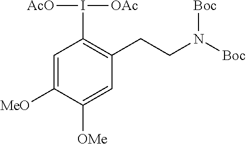 [SMILES: COc1cc(CCN(C(=O)OC(C)(C)C)C(=O)OC(C)(C)C)c(I(OC(C)=O)OC(C)=O)cc1OC]

In a N₂ charged glove box, 1 mmol (507 mg) of 2-[2-[(Di-tert-butoxycarbonyl)amino]ethyl]-4,5-dimethoxyiodobenzene was dissolved in 5 mL dry acetonitrile and transferred to a 20 mL high density polyethylene vial. Trimethylsilyl acetate (330 mg, 2.5 mmol) and a solution of F-TEDA-BF₄ (439 mg, 1.30 mmol) in 8 mL dry acetonitrile were dropwisely added sequentially. The reaction mixture was allowed to stand at room temperature for 8 h. The reaction solution was placed in a 100 mL Schlenk flask, sealed and removed from the glove box. Acetonitrile was removed by vacuum transfer and the remaining yellow oil was treated with 3 aliquots (5 mL each) of dichloromethane and the aliquots were decanted off of the colorless precipitated salts that remained in the flask. The combined dichloromethane extracts were washed (4×15 mL) with aqueous acetate buffer (NaOAc: HOAc=0.5 M: 0.5 M, pH=5) and dried over sodium sulfate. The dichloromethane was removed in vacuo to yield a pale yellow oil. Pentane (8 mL) was added to the oil and mixture was placed in an ultrasonic bath and sonicated until the salt solidified until. The pentane was decanted away and the remaining light yellow solid was dried under dynamic vacuum for overnight to yield 381 mg (0.61 mmol, 61%) 2-(Diacetoxyiodo)-1-[2-[(di-tert-butoxycarbonyl)amino]ethyl]-4,5-dimethoxybenzene. ¹H NMR (CD₃CN, 400 MHz, 25° C.): δ 7.732 (s, 1H), 7.047 (s, 1H), 3.882 (s, 3H), 3.848 (t, J=7.6 Hz, 2H), 3.830 (s, 3H), 3.120 (t, J=7.6 Hz, 2H), 1.899 (s, 6H), 1.451 (s, 9H); ¹³C NMR (CD₃CN, 100 MHz, 25° C.) δ 177.6, 153.8, 153.3, 149.8, 136.5, 121.6, 115.9, 113.9, 83.1, 57.1, 56.6, 48.2, 39.1, 28.3, 20.6; HRMS: (HRFAB) calcd. for C₂₆H₃₄IN₂O₉⁺ [M−2OAc+3-NBA]⁺ 645.1304 found 645.1312.

### Example 6

### 2-(Diacetoxyiodo)-1-[(2S)-2-[(di-tert-butoxycarbonyl)amino]-3-methoxy-3-oxopropyl]-4,5-dimethoxybenzene (6a)

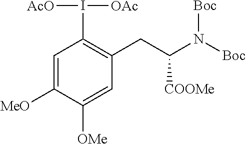 [SMILES: COC(=O)[C@H](Cc1cc(OC)c(OC)cc1I(OC(C)=O)OC(C)=O)N(C(=O)OC(C)(C)C)C(=O)OC(C)(C)C]

In a N₂ charged glove box, 1 mmol (565 mg) of 2-[(2S)-2-[(Di-tert-butoxycarbonyl)amino]-3-methoxy-3-oxopropyl]-4,5-dimethoxyiodobenzene was dissolved in 5 mL dry acetonitrile and transferred to a 20 mL high density polyethylene vial. Trimethylsilyl acetate (330 mg, 2.5 mmol) and a solution of F-TEDA-BF₄ (439 mg, 1.30 mmol) in 8 mL dry acetonitrile were dropwisely added sequentially. The reaction mixture was allowed to stand at room temperature for 8 h. The reaction solution was placed in a 100 mL Schlenk flask, sealed and removed from the glove box. Acetonitrile was removed by vacuum transfer and the remaining yellow oil was treated with 3 aliquotes (5 mL) of dichloromethane and the aliquots were decanted off of the colorless precipitated salts that remained in the flask. The combined dichloromethane extracts were washed (4×15 mL) with aqueous acetate buffer (NaOAc: HOAc=0.5 M: 0.5 M, pH=5) and dried over sodium sulfate. The dichloromethane was removed in vacuo to yield a pale yellow oil. Pentane (8 mL) was added to the oil and mixture was placed in an ultrasonic bath and sonicated until the salt solidified until. The pentane was decanted away and the remaining light yellow solid was dried under dynamic vacuum for overnight to yield 246 mg (0.36 mmol, 36%) 2-(Diacetoxyiodo)-1-[(2S)-2-[(di-tert-butoxycarbonyl)amino]-3-methoxy-3-oxopropyl]-4,5-dimethoxybenzene. ¹H NMR (CD₃CN, 400 MHz, 25° C.): δ 7.720 (s, 1H), 7.011 (s, 1H), 5.236 (dd, J₁=10.4 Hz, J₂=3.2 Hz, 1H), 3.864 (s, 3H), 3.821 (s, 3H), 3.728 (s, 3H), 3.676 (dd, J₁=14.8 Hz, J₂=3.2 Hz, 1H), 3.446 (dd, J₁=14.8 Hz, J₂=10.4 Hz, 1H), 1.898 (s, 6H), 1.352 (s, 9H); ¹³C NMR (CD₃CN, 100 MHz, 25° C.) δ 171.3, 153.3, 152.7, 149.9, 134.5, 121.6, 114.3, 84.2, 60.8, 57.2, 56.6, 53.3, 39.5, 28.1, 20.5; HRMS: (HRFAB) calcd. for C₂₈H₃₆IN₂O₁₁⁺ [M−2OAc+3-NBA]⁺ 703.1358 found 703. 1365.

### Example 7

### [2-[2-[(Di-tert-butoxycarbonyl)amino]ethyl]-4,5-dimethoxyphenyl]-(4′-methoxyphenyl)iodonium triflate

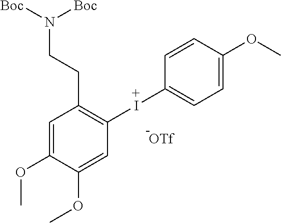

In a N₂ charged glove box, 381 mg (0.61 mmol) 2-(diacetoxyiodo)-1-[2-[(di-tert-butoxycarbonyl)amino]ethyl]-4,5-dimethoxybenzene was dissolved in 2 mL dry acetonitrile. A saturated solution of potassium (4-methoxylphenyl)trifluoroborate (130 mg, 0.61 mmol) in 5 mL dry acetonitrile was added to the reaction mixture followed by trimethylsilyl trifluoroacetate (113 mg, 0.61 mmol) solution in 2.5 mL dry acetonitrile. Acetonitrile was then removed in vacuo and dichloromethane (3×4 mL) were used to extract the remaining yellow oil. The combined dichloromethane solutions were washed (3×10 mL) with aqueous acetate buffer (NaOAc:HOAc=0.5 M:0.5 M, pH=5) and dried over sodium sulfate. Dichloromethane was removed in vacuo to yield a pale yellow oil. The oil was dissolved in 2 mL dry acetonitrile and poured into a 4 mL aqueous solution of sodium hexafluorophosphate (587 mg, 3.5 mmol) precipitating the diaryliodonium hexafluorophosphate salt. The mixture was extracted with dichloromethane (3×5 mL) and the combined organic layers were dried over sodium sulfate, and the solvent was removed under reduced pressure. The crude product was purified by column chromatography (60 Å silica, 40% acetone in hexanes, Rf=0.3) to yield 250 mg [2-[2-[(Di-tert-butoxycarbonyl)amino]ethyl]-4,5-dimethoxyphenyl]-(4′-methoxyphenyl)iodonium hexafluorophosphate (250 mg, 0.33 mmol). This compound was dissolved in 1 mL acetonitrile/water (9:1 by volume) solution and slowly passed down an Amberlite IRA-400 ion exchange column (triflate counterion). (The column was prepared for ion exchange by treating the commercially obtained Amberlite IRA-400 (Cl) resin with saturated sodium triflate solution and washing with 10 column volumes of distilled water.) [2-[2-[(Di-tert-butoxycarbonyl)amino]ethyl]-4,5-dimethoxyphenyl]-(4′-methoxyphenyl)iodonium triflate (250 mg, 0.33 mmol) was collected and dried under dynamic vacuum for 20 h. The salt was dissolved dichloromethane (2 mL) and transferred to a 20 mL borosilicate glass vial. Pentane (18 mL) was carefully layered on top of the previous dichloromethane solution. The vial was capped and the sealed container was shielded from ambient light with aluminum foil. Colorless needles formed at the solution interface; these were collected after 20 h. The needles were subjected to a second round of recrystallization using the identical conditions (dichloromethane (2 mL), pentane (18 mL) layering, 20 h in dark) to yield colorless needles of [2-[2-[(di-tert-butoxycarbonyl)amino]ethyl]-4,5-dimethoxyphenyl]-(4′-methoxyphenyl)iodonium triflate (180 mg, 0.24 mmol). The crystals were dried under vacuum and stored in a −40° C. freezer under N₂. ¹H NMR (CD₃CN, 400 MHz, 25° C.): δ 8.01 (d, J=9.01 Hz, 2H), 7.56 (s, 1H), 7.04 (d, J=9.01 Hz, 2H), 6.95 (s, 1H), 3.84 (s, 3H), 3.83 (s, 3H), 3.82 (s, 3H), 3.80 (t, J=7.16 Hz, 2H), 3.10 (t, J=7.16 Hz, 2H), 1.44 (s, 18H); ¹³C NMR (CD₃CN, 100 MHz, 25° C.) δ 164.3, 154.2, 153.8, 151.0, 138.2, 136.6, 120.3, 119.1, 115.2, 107.0, 83.8, 57.3, 56.9, 56.8, 47.4, 38.3, 28.3; ¹⁹F NMR (CD₃CN, 400 MHz, 25° C.): δ −79.3 (s, 3F). HRMS: (HREI) calcd. for C₂₇H₃₇O₇NI [M−OTf]⁺ 614.9165. found 614.1627.

### Example 8

### [2-[(2S)-2-[(Di-tert-butoxycarbonyl)amino]-3-methoxy-3-oxopropyl]-4,5-dimethoxyphenyl]-(4% methoxyphenyl)iodonium triflate (6b)

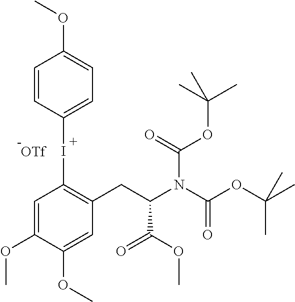

In a N₂ charged glove box, 492 mg (0.72 mmol) 2-(diacetoxyiodo)-1-[(2S)-2-[(di-tert-butoxycarbonyl)amino]-3-methoxy-3-oxopropyl]-4,5-dimethoxybenzene was dissolved in 2.5 mL dry acetonitrile. A saturated solution of potassium (4-methoxylphenyl)trifluoroborate (153.4 mg, 0.72 mmol) in 6 mL dry acetonitrile was added to the reaction mixture followed by trimethylsilyl trifluoroacetate (133.4 mg, 0.72 mmol) solution in 1 mL dry acetonitrile. Acetonitrile was then removed in vacuo and dichloromethane (3×5 mL) were used to extract the remaining yellow oil. The combined dichloromethane solutions were washed (3×12 mL) with aqueous acetate buffer (NaOAc: HOAc=0.5 M: 0.5 M, pH=5) and dried over sodium sulfate. Dichloromethane was removed in vacuo to yield a pale yellow oil. Dichloromethane was removed in vacuo to yield a pale yellow oil. The oil was dissolved in 3 mL dry acetonitrile and poured into a 3 mL aqueous solution of sodium hexafluorophosphate (1 g, 6 mmol) precipitating the diaryliodonium hexafluorophosphate salt. The mixture was extracted with dichloromethane (3×6 mL) and the combined organic layers were dried over sodium sulfate, and the solvent was removed under reduced pressure. Minimum amount of ethyl acetate was used to rinse off the brown color. Remained oil (200 mg, mmol) was dissolved in a mixture of dichloromethane (2.5 mL) and ethyl acetate (2.5 mL). This solution was transferred to a 20 mL borosilicate glass vial. Pentane (15 mL) was carefully layered on top of the previous solution. Colorless needles formed at the solution interface; these were collected after 20 h. The needles were subjected to a second round of recrystallization using the identical conditions (dichloromethane (2.5 mL), ethyl acetate (2.5 mL), pentane (15 mL) layering, 20 h in dark) to yield colorless needles of [2-[(2S)-2-[(Di-tert-butoxycarbonyl)amino]-3-methoxy-3-oxopropyl]-4,5-dimethoxyphenyl]-(4′-methoxyphenyl)iodonium hexafluorophosphate (120 mg). This compound was dissolved in 1 mL acetonitrile/water (9:1 by volume) solution and slowly passed down an Amberlite IRA-400 ion exchange column (triflate counterion). (The column was prepared for ion exchange by treating the commercially obtained Amberlite IRA-400 (Cl) resin with saturated sodium triflate solution and washing with 10 column volumes of distilled water.) [2-[(2S)-2-[(Di-tert-butoxycarbonyl)amino]-3-methoxy-3-oxopropyl]-4,5-dimethoxyphenyl]-(4′-methoxyphenyl)iodonium triflate (120 mg, 0.14 mmol) was collected and dried under dynamic vacuum for 20 h. The salt was dissolved in a mixture of dichloromethane (3 mL) and ethyl acetate (3 mL). This solution was transferred to a 50 mL borosilicate glass Schlenk tube. Pentane (20 mL) was carefully layered on top of the previous dichloromethane solution. The tube was capped and the sealed container was shielded from ambient light with aluminum foil. Colorless needles formed at the solution interface; these were collected after 48 h to yield colorless needles of [2-[(2S)-2-[(Di-tert-butoxycarbonyl)amino]-3-methoxy-3-oxopropyl]-4,5-dimethoxyphenyl]-(4′-methoxyphenyl)iodonium triflate (90 mg, 0.11 mmol). The crystals were dried under vacuum and stored in a 40° C. freezer under N₂. ¹H NMR (CD₂Cl₂, 400 MHz, 25° C.): δ 7.94 (d, J=8.8 Hz, H2′/H6′, 2H), 7.30 (s, H6, 1H), 6.99 (d, J=8.8 Hz, H3′/H5′, 2H), 6.93 (s, H3, 1H), 5.10 (dd, J₁=7.4 Hz, J₂=7.3 Hz, CH, 1H), 3.85 (s, —OCH₃, 3H), 3.84 (s, —OCH₃, 3H), 3.76 (s, —OCH₃, 3H), 3.74 (s, —COOCH₃, 3H), 3.62 (dd, J₁=14.3 Hz, J₂=7.3 Hz, —CH₂, 1H), 3.39 (dd, J₁=14.3 Hz, J₂=7.4 Hz, —CH₂, 1H), 1.44 (s, Boc, 18H); ¹³C NMR (CD₂Cl₂, 400 MHz, 25° C.): δ 171.0 (C═O), 163.7 (C4′), 153.5 (C═O), 152.7 (C4), 150.8 (C5), 137.5 (C2′/C6′), 134.4 (C2), 118.8 (C6), 118.6 (C3′/C5′), 114.6 (C3), 107.6 (C1), 102.7 (C1′), 84.8 (3° C. on Boc), 58.9 (α-C), 57.1 (4-OCH₃), 56.6 (5-OCH₃), 56.4 (4′-OCH₃), 53.4 (COOCH₃), 39.9 ((3-c), 28.2 (1° C. on Boc); ¹⁹F NMR (CD₃CN, 400 MHz, 25° C.): δ −79.3 (s, 3F); HRMS (HRFAB): calcd. for C₂₉H₃₉INO₉ [M−OTf]⁺ 672.1669, 673.1703 found.

### Example 9

### [2-[(2S)-2-[(Di-tert-butoxycarbonyl)amino]-3-methoxy-3-oxopropyl]-4,5-dimethoxyphenyl]-[4′-(3,3-dimethylbutoxy)phenyl]iodonium hexafluorophosphate

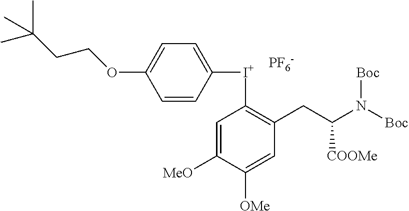

(65%). ¹H NMR (CD₃CN, 400 MHz, 25° C.): δ 7.96 (d, J=9.1 Hz, 2H), 7.41 (s, 1H), 7.04 (d, J=9.1 Hz, 2H), 6.95 (s, 1H), 5.09 (dd, J₁=9.3 Hz, J₂=5.8 Hz, 1H), 4.10 (t, J=7.2 Hz, 2H), 3.82 (s, 3H), 3.84 (s, 3H), 3.76 (s, 3H), 3.75 (s, 3H), 3.58 (dd, J₁=14.7 Hz, J₂=5.8 Hz, 1H), 3.39 (dd, J₁=14.7 Hz, J₂=5.8 Hz, 1H), 1.70 (t, J=7.2 Hz, 2H), 1.38 (s, 18H), 0.97 (s, 9H); ¹³C NMR (CD₃CN, 100 MHz, 25° C.) δ 171.2, 163.8, 153.9, 153.2, 151.1, 138.4, 135.0, 119.7, 115.6, 107.4, 102.4, 85.0, 67.4, 59.3, 57.2, 56.8, 53.5, 42.7, 39.5, 30.4, 29.9, 28.1; ¹⁹F NMR (CD₃CN, 400 MHz, 25° C.): δ −72.9 (d, J=706.2 Hz, 6F). HRMS: (HREI) calcd. for C₃₄H₄₉INO₉PF₆ [M−PF₆+Na]⁺ 742.6703 found 742.2457.

General Procedure for One-Pot Syntheses of Diaryliodonium Salts from Aryl Iodides

In a N₂ charged glove box, 0.5 mmol of an aryl iodide was dissolved in 3 mL dry acetonitrile. Trimethylsilyl acetate (165 mg, 1.25 mmol) was added to the solution followed by a solution of F-TEDA-BF₄ (220 mg, 0.65 mmol) in 3 mL dry acetonitrile. The reaction mixture was allowed to stand at room temperature for 3-8 h. A saturated solution of potassium (4-methoxylphenyl)trifluoroborate (117.2 mg, 0.55 mmol) in 6 mL dry acetonitrile was added to the reaction mixture. Acetonitrile was then removed in vacuo and 3×3 mL dichloromethane were used to extract the remaining yellow oil. The combined dichloromethane solutions were washed (4×6 mL) with aqueous acetate buffer (NaOAc: HOAc=0.5 M: 0.5 M, pH=5) and dried over sodium sulfate. The dichloromethane was removed in vacuo to yield the crude product, which was purified by silica gel chromatography and/or crystallization. After recrystallization, the obtained acetate salts were subject to ion exchange to either the hexafluorophosphate or triflate salts. Typically, the acetate salt was dissolved in minimum amount of acetonitrile/water (9:1 by volume) solution and slowly passed down an Amberlite IRA-400 ion exchange column (triflate or hexafluorophosphates counterion). (The column was prepared for ion exchange by treating the commercially obtained Amberlite IRA-400 (Cl) resin with saturated sodium triflate or sodium hexafluorophosphate solution and washing with 10 column volumes of distilled water.) The triflate or hexafluorophosphates salts were collected and dried under dynamic vacuum for 20 h and submitted to recrystallization by layering in mixed solvent systems (dichloromethane and pentane or dichloromethane, ethyl acetate and pentane).

### Example 10

### Bis(4-methoxyphenyl)iodonium hexafluorophosphate

Recrystallization in a mixture of diethyl ether/dichloromethane gave 391 mg of bis(4-methoxyphenyl)iodonium hexafluorophosphate (80.5%). ¹H NMR (CD₃CN, 400 MHz, 25° C.): δ 7.973 (d, J=9.1 Hz, 4H, H2/H2′/H6/H6′), 7.046 (d, J=9.1 Hz, 4H, H3/H3′/H5/H5′), 3.833 (s, 6H, OMe); ¹³C NMR (CD₃CN, 100 MHz, 25° C.) δ 164.61 (C4/C4′), 138.55 (C2/C2′/C6/C6′), 119.42 (C3/C3′/C5/C5′), 103.36 (C1/C1′), 57.06 (OMe); ¹⁹F NMR (CD₃CN, 376 MHz, 25° C.) δ −72.833 (d, 1JP-F=707.3 Hz, PF₆—); HRMS (HRFAB): calcd. for C₁₄H₁₄O₂I [M−PF₆]+341.0038 found 341.0036.

### Example 11

### (3,4-Dimethoxyphenyl)(4′-methoxyphenyl)iodonium hexafluorophosphate

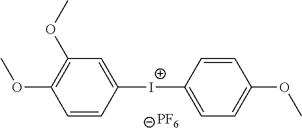

Recrystallization with diethyl ether/dichloromethane gave 370 mg (71.7%) of (3,4-dimethoxyphenyl)(4′-methoxyphenyl)iodonium hexafluorophosphate. ¹H NMR (CD₃CN, 400 MHz, 25° C.): δ7.986 (d, J=9.1 Hz, 2H, H2′/H6′), 7.647 (dd, J1=8.9 Hz, J2=2.2 Hz, 1H, H6), 7.558 (d, J=2.2 Hz, 1H, H2), 7.049 (d, J=9.1 Hz, 2H, H3′/H5′), 7.022 (d, J=8.9 Hz, 1H, H5), 1543.845 (s, 3H, 3-OMe), 3.843 (s, 3H, 4′-OMe), 3.834 (s, 3H, 4-OMe); ¹³C NMR (CD₃CN, 100 MHz, 25° C.) δ 164.58 (C4′), 154.62 (C4), 152.50 (C3), 138.49 (C2′/C6′), 130.65 (C6), 119.38 (C2), 119.13 (C3′/C5′), 115.52 (C5), 103.37 (C1), 102.64 (C1′), 57.49 (3-OMe), 57.14 (4′-OMe), 57.05 (4-OMe); ¹⁹F NMR (CD₃CN, 376 MHz, 25° C.) 6-72.786 (d, 1JP-F=705.8 Hz, PF₆—); HRMS (HRFAB): calcd. for C₁₅H₁₆O₃I [M−PF₆]+371.0144 found 371.0156.

### Example 12

### (2-Methoxyphenyl)(4′-methoxyphenyl)iodonium hexafluorophosphate

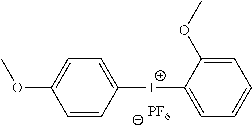

Recrystallization from a mixture of diethyl ether/dichloromethane gave 405 mg (83.3%) of (2-methoxyphenyl)(4′-methoxyphenyl)iodonium hexafluorophosphate. ¹H NMR (CD₃CN, 400 MHz, 25° C.): δ7.988 (d, J=9.2 Hz, 2H, H2′/H6′), 7.878 (d, J=8.4 Hz, 1H, H6), 7.659 (td, J1=8.4 Hz, 155 J2=1.3 Hz, 1H, H4), 7.232 (dd, J1=8.4 Hz, J2=1.3 Hz, 1H, H5), 7.063 (td, J1=8.4 Hz, J2=1.3 Hz, 1H, H3), 7.051 (d, J=9.2, 2H, H3′/H5′), 3.970 (s, 3H, 2-OMe), 3.841 (s, 3H, 4′-OMe); ¹³C NMR (CD₃CN, 100 MHz, 25° C.) δ 164.73 (C4′), 157.90 (C2), 139.52 (C2′/C6′), 137.08 (C4), 136.79 (C6), 125.36 (C3), 119.44 (C3′/C5′), 114.70 (C5), 104.69 (C1), 100.92 (C1′), 58.40 (2-OMe), 57.06 (4′-OMe); ¹⁹F NMR (CD₃CN, 376 MHz, 25° C.) δ −72.675 (d, 1JP-F=706.2 Hz, PF₆—); HRMS (HRFAB): calcd. For C₁₄H₁₄O₂I [M−PF₆]+341.0038 found 341.0035.

### Example 13

### (4,5-Dimethoxy-2-methylphenyl)(4′-methoxyphenyl)iodonium hexafluorophosphate

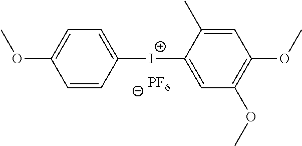

Recrystallization from a mixture of diethyl ether/dichloromethane to give 397 mg (75%) of (4,5-dimethoxy-2-methylphenyl)(4-methoxyphenyl)iodonium hexafluorophosphate. ¹H NMR (CD₃CN, 400 MHz, 25° C.): δ7.939 (d, J=9.2 Hz, 2H, H2′/H6′), 7.593 (s, 1H, H6), 7.055 (d, J=9.2 Hz, 2H, H3′/H5′), 7.026 (s, 1H, H5), 3.835 (s, 6H, 3/4′-OMe), 3.828 (s, 3H, 4-OMe), 2.550 (s, 3H, 2-Me); ¹³C NMR (CD₃CN, 100 MHz, 25° C.) δ 164.45 (C4′), 154.63 (C4), 150.46 (C5), 138.28 (C2′/C6′), 136.71 (C2), 120.59 (C6), 119.41 (C3′/C5′), 115.28 (C3), 107.01 (C1), 102.58 (C1′), 57.51 (3-OMe), 57.14 (4′-OMe), 57.04 (4-OMe); ¹⁹F NMR (CD₃CN, 376 MHz, 25° C.) δ −72.735 (d, 1JP-F=706.9 Hz, PF₆—); HRMS (HRFAB): calcd. For C₁₆H₁₈O₃I [M−PF₆]+3385.0301 found 3385.0313

### Example 14

### Phenyl(4-methoxyphenyl)iodonium hexafluorophosphate

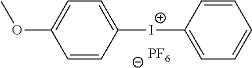

Recrystallization from a mixture of diethyl ether/dichloromethane gave 355 mg (77.9%) of phenyl(4-methoxyphenyl)iodonium hexafluorophosphate. ¹H NMR (CD₃CN, 400 MHz, 25° C.): δ8.022 (d, J=7.6 Hz, 2H, H2/H6), 8.011 (d, J=9.4 Hz, 2H, H2′/H6′), 7.701 (t, J=7.6 Hz, 1H, H4), 7.734 (t, J=7.6 Hz, 2H, H3/H5), 7.063 (d, J=9.4 Hz, 2H, H3′/H5′), 3.839 (s, 6H, OMe); ¹³C NMR (CD₃CN, 100 MHz, 25° C.) δ 164.77 (C4′), 139.04 (C2′/C6′), 136.22 (C2/C6), 134.27 (C4), 133.77 (C3/C5), 119.58 (C3′/C5′), 115.29 (C1), 102.50 (C1′), 57.09 (OMe); ¹⁹F NMR (CD₃CN, 376 MHz, 25° C.) δ −72.754 (d, 1JP-F=707.7 Hz, PF₆—); HRMS (HRFAB): calcd. for C₁₃H₁₂OI [M−PF₆]+310.9925 found 310.9932.

### Example 15

### (3-(Trifluoromethyl)phenyl)(4′-methoxyphenyl)iodonium hexafluorophosphate

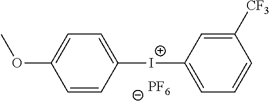

Recrystallization from a mixture of diethyl ether/dichloromethane gave 503 mg (96.1%) of (3-(trifluoromethyl)phenyl)(4′-methoxyphenyl)iodonium hexafluorophosphate. ¹H NMR (CD₃CN, 400 MHz, 25° C.): δ8.384 (s, 1H, H2), 8.266 (d, J=8.1 Hz, 1H, H6), 8.056 (d, J=9.2 Hz, 2H, H2′/H6′), 7.996 (d, J=8.1 Hz, 1H, H4), 7.716 (t, J=8.1 Hz, 1H, H5), 7.083 (d, J=9.2, 2H, H3′/H5′), 3.847 (s, 3H, 4′-OMe); ¹³C NMR (CD₃CN, 100 MHz, 25° C.) δ 164.99 (C4′), 139.99 (C6), 139.38 (C2′/C6′), 134.44 (C5), 134.281 (q, J=33.6 Hz, C3), 133.08 (q, J=3.7 Hz, C2), 133.05 (q, J=3.7 Hz, C4), 124.11 (q, J=272.8 Hz, CF3), 119.71 (C3′/C5′), 114.83 (C1), 102.54 (C1′), 57.13 (4′-OMe); ¹⁹F NMR (CD₃CN, 376 MHz, 25° C.) δ −63.420 (J1(F—C)=272.8 Hz, J2(F—C)=33.6 Hz, CF3), −72.625 (d, J1(P—F)=707.1 Hz, PF₆—); HRMS (HRFAB): calcd. for C₁₄H₁₁OIF₃ [M−PF₆]+378.9807 found 378.9817.

### Example 16

### (3-Cyanophenyl)(4′-methoxyphenyl)iodonium hexafluorophosphate

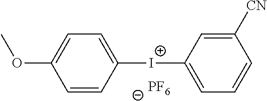

Recrystallization from a mixture of diethyl ether/dichloromethane gave 354 mg (73.7%) of (3-cyanophenyl)(4′-methoxyphenyl)iodonium hexafluorophosphate. ¹H NMR (CD₃CN, 400 MHz, 25° C.): δ 8.389 (t, J=1.6 Hz, 1H, H2), 8.273 (dd, J1=8.2 Hz, J2=1.6 Hz, 1H, H6), 8.038 (d, J=9.4 Hz, 2H, H2′/H6′), 8.017 (dd, J1=8.2 Hz, J2=1.6 Hz, 1H, H4), 7.665 (t, J=8.2 Hz, 1H, H5), 7.082 (d, J=9.4, 2H, H3′/H5′), 3.850 (s, 3H, 4′-OMe); ¹³C NMR (CD₃CN, 100 MHz, 25° C.) δ 165.04 (C4′), 140.40 (C6), 139.50 (C2), 139.47 (C2′/C6′), 137.79 (C5), 134.13 (C4), 119.75 (C3′/C5′), 117.63 (C3), 116.75 (CN), 114.53 (C1), 102.56 (C1′), 57.16 (4′-0Me); ¹⁹F NMR (CD₃CN, 376 MHz, 25° C.) δ −72.675 (d, 1JP-F=707.5 Hz, PF₆—); HRMS (HRFAB): calcd. for C₁₄H₁₁NOI [M−PF₆]+335.9885 found 335.9876.

### Example 17

### (S)-(4-(3-((4-(tert-butoxycarbonyl)morpholin-2-yl)methoxy)pyridine-2-yloxy)-2-fluorophenyl)(4-methoxyphenyl)iodonium hexafluorophosphate

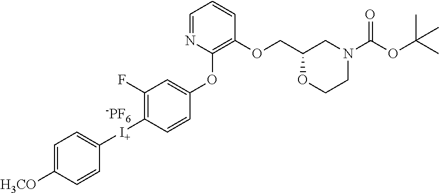 [SMILES: COc1ccc([I+]c2ccc(Oc3ncccc3OC[C@@H]3CN(C(=O)OC(C)(C)C)CCO3)cc2F)cc1 ; "-PF6"]

This compound was obtained by slow evaporation of an acetone/hexane solution. Filtration afforded (S)-(4-(3-((4-(tert-butoxycarbonyl)morpholin-2-yl)methoxy)pyridine-2-yloxy)-2-fluorophenyl)(4-methoxyphenyl)iodonium hexafluorophosphate (0.023 g, 68%) as an off-white amorphous solid. ¹⁹F NMR (CD₃CN) 376 MHz δ −96.02 (m, 1F), δ −72.89 (d, J=703.1 Hz, 6F). ¹H NMR (CD₃CN) 400 MHz δ 1.425 (s, 9H), δ 2.499 (s, 1H), δ 2.815 (s, 1H), δ 3.389 (td, J₁=2.8 Hz, J₂=11.6 Hz, 1H), δ 3.581 (m, 1H), δ 3.737 (m, 2H), δ 3.844 (s, 3H), δ 4.019 (m, 2H), δ 7.054 (dd, J₁=2.7 Hz, J₂=8.8 Hz, 1H), δ 7.054 (d, J=9.2 Hz, 2H), δ 7.134 (dd, J₁=4.8 Hz, J₂=8.0 Hz, 1H), δ 7.238 (dd, J₁=4.7 Hz, J₂=8.2 Hz, 1H), δ 7.489 (dd, J₁=1.6 Hz, J₂=8.2 Hz, 1H), δ 7.810 (dd, J₁=1.6, J₂=4.9 Hz, 1H). δ 8.023 (d, J=9.2 Hz, 2H), δ 8.081 (dd, J₁=6.95 Hz, J₂=8.97 Hz, 1H). ¹³C NMR (CD₃CN) 125 MHz δ 28.93, 45.23, 45.84, 57.09, 67.29, 70.72, 74.51, 80.97, 94.42, 103.35, 108.93, 119.52, 123.71, 124.08, 138.98, 139.08, 139.86, 139.88, 146.04, 155.93, 162.31, 163.07, 164.75. HRMS (HRFAB) calcd. for C₂₈H₃₁FIN₂O₆ [M+H]⁺ 637.1204. found 637.1206.

### Example 18

### (5-(4-((3R,4R)-4-(ethoxycarbonyl)-1-oxo-2-propyl-1,2,3,4-tetrahydroisoquinolin-3-yl)phenoxy)-2-fluorophenyl)(4-methoxyphenyl)iodonium hexafluorophosphate

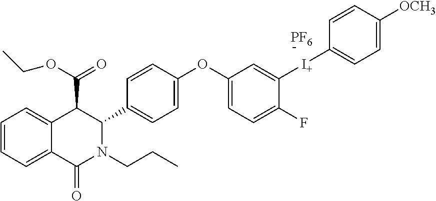 [SMILES: CCCN1C(=O)c2ccccc2[C@@H](C(=O)OCC)[C@@H]1c1ccc(Oc2ccc(F)c([I+]c3ccc(OC)cc3)c2)cc1 ; "PF6"]

This material was obtained by evaporation of an acetone/hexane solution. Filtration afforded (5-(4-((3R,4R)-4-(ethoxycarbonyl)-1-oxo-2-propyl-1,2,3,4-tetrahydroisoquinolin-3-yl)phenoxy)-2-fluorophenyl)(4-methoxyphenyl)iodonium hexafluorophosphate (15.5 mg, 33.7%) as an off-white amorphous solid. ¹⁹F NMR (CD₃CN) 376 MHz δ −106.18 (m, F), δ −72.98 (d, J=707 Hz, PF₆). ¹H NMR (CD₃CN) 400 MHz δ 0.8790 (t, J=7.2 Hz, 3H), δ 1.203 (t, J=7.2 Hz, 2H), δ 1.602 (m, 2H), δ 2.755 (ddd, J=5.2, 8.8, 13.7 Hz, 1H), δ 3.839 (s, 3H), δ 3.989 (ddd, J=7.1, 8.8, 13.4 Hz, 1H), δ 4.065 (d, J=1.7 Hz, 1H), δ 4.141 (quar., J=7.2 Hz, 1H), δ 4.144 (quar., J=7.2 Hz, 1H), δ 5.352 (d, J=1.7 Hz, 1H), δ 6.821 (d, J=8.8 Hz, 2H), δ 7.005 (d, J=9.2 Hz, 2H), δ 7.083 (d, J=8.8 Hz, 2H), δ 7.175 (m, 1H), δ 7.225 (m, 1H), δ 7.406 (m, 1H), δ 7.425 (m, 2H), δ 7.622 (dd, J=1.1, 3.0 Hz, 1H), δ 7.948 (d, J=9.2 Hz, 2H), δ 8.011 (m, 1H).

### Example 19

### (3-Cyano-5-((2-methylthiazol-4-yl)ethynyl)phenyl)(4-methoxyphenyl)iodonium hexafluorophosphate

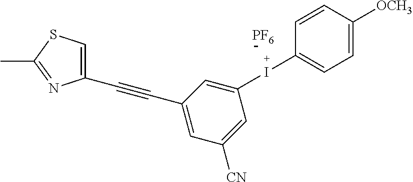

Recrystallization from acetone/hexane yielded 0.070 g (40%) of a colorless solid. ¹H NMR (CD₃CN) 400 MHz δ 2.684 (s, 3H), δ 3.858 (s, 3H), δ 7.0945 (d, J=9.2, 2H), δ 7.701 (s, 1H), δ 8.057 (d, J=9.2, 2H), δ 8.153 (t, J=1.6 Hz, 1H), δ 8.357 (t, J=1.6 Hz, 1H), δ 8.416 (t, J=1.6 Hz, 1H). ¹⁹F NMR (CD₃CN) 376 MHz δ −72.56 (d, J=748 Hz, PF₆). ¹³C NMR (CD₃CN) 150 MHz δ 19.37, δ 56.93, δ 84.61, δ 89.84, δ 102.36, δ 114.03, δ 116.72, δ 116.73, δ 119.58 δ 127.31, δ 128.08, δ 135.79, δ 138.63, δ 139.36, δ 139.93, δ 142.10, δ 164.91, δ 168.04. HRMS (positive mode) obsd mass (M+H)⁺ 456.9867; calcd mass (C₂₀H₁₄N₂OSI+H)⁺, 456.9872.

### Example 20

### (2-methoxy-5-(2-(4-methoxyphenyl)propan-2-yl)phenyl)(4-methoxyphenyl)iodonium hexafluorophosphate

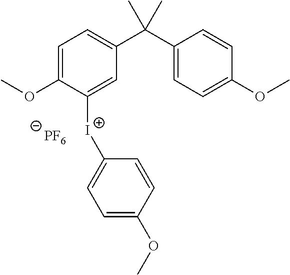

The initial ion exchange yielded a light brown oil. The oil was dissolved in 3 mL of a 1:1 solution of ethyl acetate:dichloromethane and added to a 20 mL vial. Pentane was carefully layered over the ethyl acetate:dichloromethane mixture until the vial was full. The vial was sealed and protected from the light. After 3 days, the crystallized product was collected by vacuum filtration to give (2-methoxy-5-(2-(4-methoxyphenyl)propan-2-yl)phenyl)(4-methoxyphenyl)iodonium hexafluorophosphate as colorless crystalline needles; yield 0.30 g (52%). ¹H NMR (CD₃CN) 400 MHz δ 1.619 (s, 6H), δ 3.762 (s, 3H), δ 3.854 (s, 3H), δ 3.920 (s, 3H), δ 6.798 (d, J=8.2 Hz, 2H), δ 6.982 (d, J=8.4 Hz, 2H), δ 7.095 (d, J=8.4 Hz, 2H), δ 7.112 (d, J=8.4 Hz, 1H), δ 7.471 (dd, J₁=8.2 Hz, J₂=2.8 Hz, 1H), 87.620 (d, J=2.8 Hz, 1H), δ 7.897 (d, J=8.4 Hz, 2H).

### Example 21

### (N,N-di-(t-butoxycarbonyl)-2-((4,5-dimethoxyphenethylamine dicarbonate)(4-methoxyphenyl)iodonium hexafluorophosphate

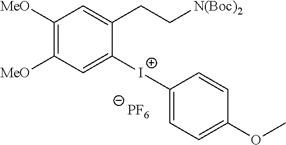

The pasty solid was dissolved in 3 mL dichloromethane and 7 mL of hexanes was layered on top, and this mixture was sealed in a vial protected from light. After the solid had crystallized, it was collected by vacuum filtration to afford (N,N-di-(t-butoxycarbonyl)-2-(4,5-dimethoxyphethylamine dicarbonate)(4-methoxyphenyl)iodonium hexafluorophosphate as a white amorphous solid; yield 0.49 g (65.2%) ¹H NMR (CD₃CN) 400 MHz δ 1.44 (s, 18H), δ 3.10 (t, J=7.16 Hz, 2H), δ 3.80 (t, J=7.16 Hz, 2H), δ 3.82 (s, 3H), δ 3.83 (s, 3H), δ 3.84 (s, 3H), δ 6.95 (s, 1H), δ 7.04 (d, J=9.01 Hz, 2H), δ 7.56 (s, 1H), δ 8.01 (d, J=9.01 Hz, 2H). ¹³C NMR (CD₃CN) 100 MHz δ 28.3, 38.3, 47.4, 56.8, 56.9, 57.3, 83.8, 107.0, 115.2, 119.1, 120.3, 136.6, 138.2, 151.0, 153.8, 154.2, 164.3. ¹⁹F NMR (CD₃CN) 400 MHz δ −72.9 (d, J=707.0 Hz, 6F). HRMS: (HREI) calcd. for C₂₇H₃₇O₇NIPF₆ [M−PF₆+Na]⁺ 614.9165. found.

### Example 22

### (N,N-di-(t-butoxycarbonyl)-2-(4,5-dimethoxyphenethylamine dicarbonate)(4-(3,3-dimethylbutoxyphenyl))iodonium hexafluorophosphate

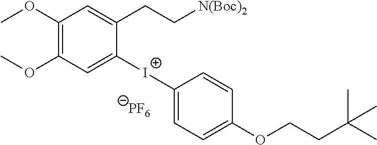

The pasty solid was recrystallized by dissolving the solid in 3 mL dichloromethane and layering 7 mL of hexanes and sealing the contents in a vial, protected from light. After the solid had crystallized, it was collected by vacuum filtration to afford (N,N-di-(t-butoxycarbonyl)-2-(4,5-dimethoxyphenethylamine dicarbonate)(4-(3,3-dimethylbutoxyphenyl))iodonium hexafluorophosphate as a white amorphous solid; yield 0.49 g (65.2%) ¹H NMR (CD₃CN) 400 MHz δ 0.968 (s, 9H), δ 1.440 (s, 18H), δ 1.692 (t, J=7.2 Hz, 2H), δ 3.100 (t, J=7.2 Hz, 2H), δ 3.795 (t, J=7.2 Hz, 2H), δ 3.815 (s, 3H), δ 3.843 (s, 3H), δ 4.093 (t, J=7.2 Hz, 2H), δ 6.954 (s, 1H), δ 7.024 (d, J=8.4 Hz, 2H), δ 7.544 (s, 1H). δ 7.990 (d, J=8.4 Hz, 2H). ¹³C NMR (CD₃CN) 100 MHz δ 28.60, 30.23, 30.71, 38.63, 43.05, 47.72, 57.13, 57.63, 67.61, 84.10, 103.07, 107.39, 115.45, 119.83, 120.68, 136.84, 138.45, 151.16, 154.01, 154.39, 163.90. ¹⁹F NMR (CD₃CN) 376 MHz δ −79.36.

### Example 23

### (3-Cyano-5-(pyridine-2-ylethynyl)phenyl)(4-methoxyphenyl)iodonium hexafluorophosphate

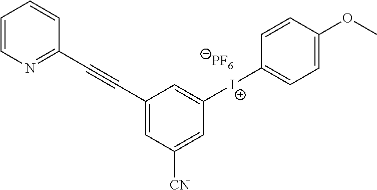 [SMILES: COc1ccc([I+]c2cc(C#N)cc(C#Cc3ccccn3)c2)cc1 ; "PF6"]

The crude filtered product was dissolved in CH₂Cl₂ to remove it from the filter and the solvent was evaporated. The colorless solid was recrystallized from CH₂Cl₂/heptanes to give a colorless, crystalline solid. (14.6 mg, 50%). ¹H NMR (300 MHz, CD₃CN) 6=8.63 (d, 1H, J=4.8 Hz), 8.49 (d, 1H, J=1.2 Hz), 8.40 (s, 1H), 8.21 (d, 1H, J=0.8 Hz), 8.01 (d, 2H, J=9.2 Hz), 7.90 (t, 1H, J=7.6 Hz), 7.68 (d, 1H, J=7.6 Hz), 7.48 (t, 1H, J=6.2 Hz), 7.10 (d, 2H, J=9.2 Hz), 3.86 (s, 3H); ¹³C NMR (75 MHz, CD₃CN) 6=150.51, 141.36, 139.12, 138.22, 137.92, 136.81, 127.89, 124.35, 118.44, 117.30, 115.64, 55.84; ¹⁹F NMR (282 MHz, CD₃CN): −72.96 (d, 6 F, J=705 Hz); HR-FAB MS: (M−PF₆)+437.0149 m/z (calcd for C₂₁H₁₄IN₂O, 437.0145).

### Example 24

### (3-cyano-5-((6-methylpyridin-2-yl)ethynyl)phenyl)(4-methoxyphenyl)iodonium hexafluorophosphate

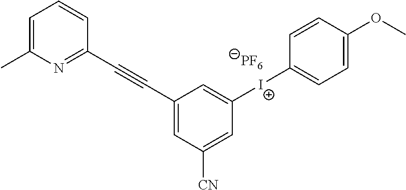

The crude product was recrystallized from CH₂Cl₂/heptanes to give a colorless, crystalline solid (12.5 mg, 50%). ¹H NMR (400 MHz, CD₃CN): 6=8.47 (s, 1H), 8.39 (s, 1H), 8.20 (s, 1H), 8.07 (d, 2H, J=8.1 Hz), 7.72 (t, 1H, J=8.0 Hz), 7.44 (d, 1H, J=8.0 Hz), 7.29 (d, 1H, J=8.0 Hz), 7.10 (d, 2H, J=9.2 Hz), 3.86 (s, 3H), 2.52 (s, 3H). ¹³C NMR (100 MHz, CD₃CN): 6=163.82, 159.59, 141.36, 140.57, 139.16, 138.29, 137.89, 137.10, 126.78, 125.09, 124.06, 118.49, 115.66, 112.93, 101.28, 93.33, 82.99, 55.85, 23.46; ¹⁹F (376 MHz, CD₃CN) δ=−72.79 (d, 6 F, 703.1 Hz); HR-FAB MS: (M−PF₆)⁺ 451.0299 m/z (calcd for C₂₂H₁₆IN₂O, 451.03).

### Example 25

### (S)-3-(3,4-dimethoxyphenyl)-1-methoxy-1-oxopropan-2-amine

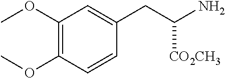 [SMILES: COC(=O)[C@@H](N)Cc1ccc(OC)c(OC)c1]

3,4-dimethoxy-L-phenylalanine (100.0 g, 0.44 mol) was added to 1.3 L of methanol and the solution was cooled to 0° C. with an ice-water bath. Thionyl chloride (48 mL, 0.66 mol) was added slowly to the chilled solution. The ice bath was removed and the reaction mixture was heated at reflux for 10 hours. The solution was allowed to cool to room temperature and the methanol was removed by rotary evaporation. The oily residue was dissolved in 250 mL of deionized water, and the resulting solution was brought to pH 12 with saturated aqueous sodium carbonate. The aqueous solution was extracted with dichloromethane (5×300 mL) and the combined organic extracts were dried with sodium sulfate, filtered, and evaporated to yield (S)-3-(3,4-dimethoxyphenyl)-1-methoxy-1-oxopropan-2-amine (106 g, quant.) as a light yellow oil.

### Example 26

### (S)-3-(2-iodo-4,5-dimethoxyphenyl)-1-methoxy-1-oxopropan-2-amine

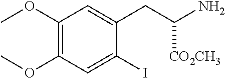 [SMILES: COC(=O)[C@@H](N)Cc1cc(OC)c(OC)cc1I]

Trifluoroacetic acid (39 mL, 0.502 mmol) was added to a stirred solution of (S)-3-(3,4-dimethoxyphenyl)-1-methoxy-1-oxopropan-2-amine (60.0 g, 0.251 mol) in 2 L of acetonitrile. N-iodosuccinimide (56.5 g, 0.251 mol) was added in portions over 20 minutes to the stirred reaction mixture, and the 3 L flask round bottom flask was shielded with aluminum foil. After 18 hours, the acetonitrile was removed and the remaining solid was dissolved in deionized water. This solution was treated with saturated aqueous sodium bisulfite until the purple color disappeared. The pH was adjusted to 12 using a saturated aqueous potassium carbonate and the solution was extracted with dichloromethane (3×200 mL). The combined organic extracts were dried over sodium sulfate and the solvent was removed by rotary evaporation yield (S)-3-(2-iodo-4,5-dimethoxyphenyl)-1-methoxy-1-oxopropan-2-amine (77.8 g, 85%) as a pale yellow oil. ¹H NMR (CDCl₃) 400 MHz δ 1.63 (s, 2H), δ 2.87 (dd, J₁=11.3 Hz, J₂=14.0 Hz, 1H), δ 3.15 (dd, J₁=11.3 Hz, J₂=14.0 Hz, 1H), δ 3.71 (s, 3H), 3.81 (dd, J₁=11.3 Hz, J₂=14.0 Hz, 1H), δ 3.85 (s, 6H), δ 6.72 (s, 1H), δ 7.20 (s, 1H).

### Example 27

### (S)-3-(2-Iodo-4,5-dimethoxyphenyl)-1-methoxy-1-oxopropan-2-amine hydrochloride

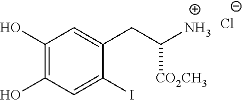 [SMILES: [Cl-] ; COC(=O)[C@@H]([NH3+])Cc1cc(O)c(O)cc1I]

Under N₂, (S)-3-(2-iodo-4,5-dimethoxyphenyl)-1-methoxy-1-oxopropan-2-amine was dissolved in dry, distilled dichloromethane. The solution was chilled to 0° C. and boron tribromide was added dropwise to the vigorously stirred reaction mixture. Upon complete addition of boron tribromide, the solution was stirred at 0° C. for an additional 30 minutes. After 30 minutes, the crude reaction mixture was cautiously poured onto 30 grams of ice. The aqueous solution was separated and washed three times with dichloromethane. The aqueous layer was brought to pH 2 by the careful addition of NaHCO₃, saturated with sodium chloride, and extracted (4×100 mL) with ethyl acetate. The ethyl acetate layers were combined, dried over sodium sulfate, and the solvent was removed by rotary evaporation to yield the product as a colorless amorphous solid. ¹H NMR (d₆-acetone) 400 MHz δ 2.85 (dd, dd, J₁=11.3 Hz, J₂=14.0 Hz, 1H), δ 2.94 (dd, J₁=11.3 Hz, J₂=14.0 Hz, 1H), δ 3.57 (s, 3H), δ 3.83 (t, J=7.1 Hz, 1H), δ 6.53 (s, 1H), δ 6.83 (s, 2H), δ 6.86 (s, 1H).

### Example 28

### Methyl (S)-2-((tert-butoxycarbonylamino)-3-(4,5-dihydroxy-2-iodophenyl)propanoate

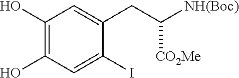

(S)-3-(2-iodo-4,5-dimethoxyphenyl)-1-methoxy-1-oxopropan-2-amine hydrochloride (0.5 g) was dissolved in 5 mL of dry dimethylformamide, and triethylamine (0.3 mL, 1.5 eq) was added, followed by solid tert-butyl dicarbonate (0.29 g, 0.99 eq). The solution was heated to 60° C. and allowed to stir for 18 hours. The reaction mixture was cooled to room temperature and DMF was removed by azeotropic distillation with toluene under reduced pressure. Upon complete removal of the solvent, the oily residue was dissolved in ethyl acetate and washed with acetate buffer (3×15 mL) and deionized water (3×10 mL). The organic layer was dried with sodium sulfate, filtered, and removed by rotary evaporation to yield a brown solid. The brown solid was chromatographed on silica using an ethyl acetate:hexane solvent gradient (0-25%-50%) to yield the product as a colorless solid. ¹H NMR (d₆-acetone) 400 MHz δ 1.35 (s, 9H), δ 2.89 (dd, J₁=11.3 Hz, J₂=14.0 Hz, 1H), δ 3.12 (dd, J₁=11.3 Hz, J₂=14.0 Hz, 1H), δ 3.67 (s, 3H), δ 4.43 (dd, J₁=11.3 Hz, J₂=14.0 Hz, 1H), δ 6.20 (d, 6.8 Hz, 1H), δ 6.84 (s, 1H), δ 7.28 (s, 1H), δ 8.19 (s, 2H).

### Example 29

### 2-(2-iodo-4,5-dimethoxyphenyl)ethanamine

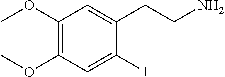

To a solution of N-Iodosuccinamide (8.3 g, 37 mmol) in 70 mL of acetonitrile was added 2-(3,4-dimethoxyphenyl)ethanamine (3.04 g, 16.8 mmol) and trifluoroacetic acid (5.4 mL, 53 mmol). The reaction was stirred in the dark for 17 hours at room temperature. The acetonitrile was removed under reduced pressure and the remaining oil was dissolved in 80 mL of water and treated with a saturated aqueous solution of sodium bisulfite until all iodine was quenched. This solution was adjusted to pH 10 with aqueous KOH, precipitating a light yellow solid. The solid was collected by vacuum filtration to give 2-(2-iodo-4,5-dimethoxyphenyl)ethanamine (4.18 g, 81.0%). ¹H NMR (CDCl₃) 400 MHz δ 2.562 (s, 2H), δ 2.797 (t, J=6.8 Hz, 2H), δ 2.910 (t, J=6.8 Hz, 2H), δ 3.814, (s, 3H), δ 3.828 (s, 3H), δ 6.732 (s, 1H), δ 7.188 (s, 1H). ¹³C NMR (CDCl₃) 100 MHz δ 42.48, 43.94, 56.06, 56.26, 88.36, 112.78, 121.83, 134.73, 148.13, 149.41

### Example 30

### N-t-butoxycarbonyl-2-(2-iodo-4,5-dihydroxyphenyl)ethanamine

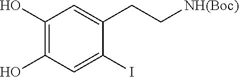

Under an inert atmosphere, 2-(2-iodo-4,5-dimethoxyphenyl)ethanamine (18.3 g, 59.6 mmol) was dissolved in 230 mL of dry, distilled dichloromethane. The reaction mixture was cooled to −78° C. and boron tribromide (11.3 mL, 119 mmol) was added dropwise to the reaction mixture. The cooling bath was removed from the reaction flask, and the mixture was allowed to warm to room temperature and stirred for 18 hours. After 18 hours, the reaction mixture was cooled to 0° C. and quenched with 100 mL of ice water. The aqueous layer was removed and the organic layer was extracted with deionized water (3×25 mL). The aqueous layer was neutralized to pH 6 by addition of solid sodium bicarbonate. THF (150 mL) was added to the aqueous layer and the solution was stirred vigorously to avoid bilayer formation of the solvents. An additional 50 mL aliquot of saturated aqueous sodium bicarbonate was added to the reaction mixture, followed by a 1 M solution of Boc-anhydride in THF (12.88 g of Boc-anhydride in 60 mL of THF). The mixture was allowed to stir for 2 hours before the THF layer was removed and the aqueous layer was extracted with ethyl acetate (3×50 mL). The organic layers were combined, dried with sodium sulfate, and solvents were removed in vacuo to give a light brown oil. The oil was chromatographed through a 2″ silica plug using a gradient of ethyl acetate/hexanes (0-25%-50%). Removal of the organic solvents in vacuo yielded the product (11.3 g, 50%) as a colorless solid. ¹H NMR (d₆-acetone) 400 MHz δ 1.40 (s, 9H), δ 2.76 (t, J=7.0 Hz, 2H), δ (quartet, J=6.1 Hz, 2H), δ 6.05 (s, 1H), δ 6.80 (s, 1H), δ 7.24 (s, 1H), δ 8.08 (s, 2H).

### Example 31

### N-(t-butoxycarbonyl)-2-(2-iodo-4,5-bis(ethoxymethoxy)phenyl)ethanamine

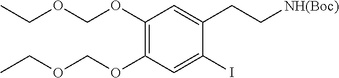

Under an inert atmosphere, N-t-butoxycarbonyl-2-(2-iodo-4,5-dihydroxyphenyl)ethanamine (5.0 g, 13.2 mmol) was dissolved in 35 mL of dry, distilled THF. The solution was chilled to 0° C. and diisopropylethylamine (5.8 mL, 33.0 mmol) was added by syringe, and the reaction mixture was allowed to stir for 5 minutes. Ethoxymethyl chloride (3.1 mL, 33.0 mmol) was added dropwise by syringe. After the addition of EOMCl was completed, the cooling bath was removed and the solution was allowed to warm to room temperature. The reaction mixture was then heated to reflux and allowed to stir for 18 hours. After 18 hours, the reaction mixture was allowed to cool to room temperature and the mixture was quenched with a 50 mL aliquot of ice-water. The THF was separated and the aqueous layer was extracted with ethyl acetate (2×40 mL). The organic fractions were combined and were extracted (3×50 mL) with an aqueous solution containing 10% potassium carbonate. The combined organic layers were washed (2×40 mL) with sodium chloride, dried over sodium sulfate, filtered, and the solvents were removed in vacuo to yield N-(t-butoxycarbonyl)-6-iodo-3,4-bis-(ethoxymethoxy)phenethylamine (5.4 g, 82%) as a colorless oil. ¹H NMR (CDCl₃) 400 MHz δ 1.25 (t, J=7.4 Hz, 3H), δ 1.26 (t, J=7.4 Hz, 3H), δ 1.45 (s, 9H), δ 2.85 (t, J=7.0 Hz, 2H), δ 3.34 (quartet, J=6.2 Hz, 2H), δ 3.76 (quartet, J=7.1 Hz, 2H), δ 3.77 (quartet, J=7.1 Hz, 2H), δ 4.59 (s, 1H), δ 5.23 (s, 2H), δ 5.24 (s, 2H), δ 7.04 (s, 1H), δ 7.58 (s, 1H).

### Example 32

### 2-[2-[(di-tert-butoxycarbonyl)amino]ethyl]-4,5-bis(ethoxymethoxy)iodobenzene

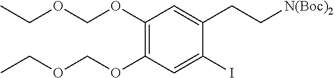

N-(t-butoxycarbonyl)-2-(2-iodo-4,5-bis(ethoxymethoxy)phenyl)ethanamine (4.5 g, 9.1 mmol) was dissolved in 90 mL of acetonitrile. Triethylamine (10 mL, 72.8 mmol), dimethylaminopyridine (1.11 g, 9.1 mmol), and Boc anhydride (2.97 g, 14 mmol) were added to the reaction mixture and the solution was stirred at room temperature for 24 hours. After 24 hours, deactivated silica was added to the solution and the solvent was removed in vacuo. After the silica was completely dry, the crude contents were loaded onto a deactivated silica gel column. The mixture was then chromatographed (R_{f}=0.34) using an ethyl acetate/hexanes gradient (0-6%-15%) to yield a light yellow oil as the product. ¹H NMR (CDCl₃) 400 MHz δ 1.21 (t, J=7.1 Hz, 3H), δ 1.23 (t, J=7.1 Hz, 3H), δ 1.47 (s, 18H), δ 2.95 (t, J=7.2 Hz, 2H), δ 3.73 (quartet, J=7.1 Hz, 2H), δ 3.74 (quartet, J=7.1 Hz, 2H), δ 3.80 (t, J=7.2 Hz, 2H), δ 5.22 (s, 2H), δ 5.23 (s, 2H), δ 7.03 (s, 1H), δ 7.56 (s, 1H).

Silica gel was deactivated in the following manner: A 5% triethylamine/hexanes solution was prepared and silica gel was added until a viscous slurry was obtained. The silica gel was then filtered by vacuum filtration and washed with hexanes.

### Example 33

### 2-(Diacetoxyiodo)-1-[2-[(di-tert-butoxycarbonyl)amino]ethyl]-4,5-bis(ethoxymethoxy)benzene

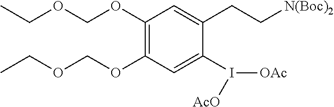

In a N₂ charged glove box, 0.51 g 2-[2-[(di-tert-butoxycarbonyl)amino]ethyl]-4,5-bis(ethoxymethoxy)iodobenzene was dissolved in 5 mL of dry acetonitrile and transferred to a 20 mL high density polyethylene vial with trimethylsilyl acetate (330 mg, 2.5 mmol) and the mixture was stirred at room temperature. Next, a freshly prepared solution of F-TEDA-BF₄ (439 mg, 1.30 mmol) in 8 mL dry acetonitrile was added dropwise to the stirring mixture with a glass pipette. The reaction mixture was then allowed to stir at room temperature for 5 hours before it was transferred to a 100 mL round bottom flask and the solvent was removed by rotary evaporation. The oily residue was washed with dichloromethane (3×10 mL), leaving behind the colorless precipitated salts which remained in the flask. The combined dichloromethane extracts were washed (4×20 mL) with aqueous acetate buffer (NaOAc: HOAc=0.5 M: 0.5 M, pH=5) and dried over sodium sulfate. Removal of the solvent by rotary evaporation yielded a pale yellow oil which was dried under dynamic vacuum overnight to yield 2-(Diacetoxyiodo)-1-[2-[(di-tert-butoxycarbonyl)amino]ethyl]-4,5-di(ethoxymethoxy)benzene.

### Example 34

### 2-(Diacetoxyiodo)-1-[(2S)-2-[(di-tert-butoxycarbonyl)amino]-3-methoxy-3-oxopropyl]-4,5-bis(ethoxymethoxy)benzene

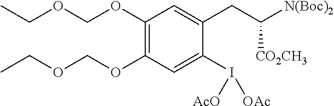

In a N₂ charged glove box, 1.13 g of 2-[(2S)-2-[(di-tertbutoxycarbonyl)amino]-3-methoxy-3-oxopropyl]-4,5-dimethoxyiodobenzene was dissolved in 10 mL of dry acetonitrile and transferred to a 20 mL high density polyethylene vial with trimethylsilyl acetate (660 mg, 5 mmol). A solution of F-TEDA-BF₄ (878 mg, 2.60 mmol) in 16 mL of dry acetonitrile was added dropwise to the reaction mixture, and the solution was allowed to stand at room temperature for 8 h before it was transferred to a 100 mL round bottom flask. The flask was removed from the glove box and the solvent was removed by rotary evaporation. The oily residue was washed with dichloromethane (3×10 mL), leaving behind the colorless precipitated salts which remained in the flask. The combined dichloromethane extracts were washed (4×20 mL) with aqueous acetate buffer (NaOAc: HOAc=0.5 M: 0.5 M, pH=5) and dried over sodium sulfate. Removal of the solvent by rotary evaporation yielded a pale yellow oil which was dried under dynamic vacuum overnight to yield 2-(diacetoxyiodo)-1-[(2S)-2-[(di-tertbutoxycarbonyl)amino]-3-methoxy-3-oxopropyl]-4,5-bis(ethoxymethoxy)benzene.

### Example 35

### 4-Iodo-L-phenylalanine

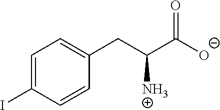 [SMILES: [NH3+][C@@H](Cc1ccc(I)cc1)C(=O)[O-]]

In a 250 mL round bottom flask equipped with a magnetic stir bar, concentrated sulfuric acid (18 mL, 337 mmol) was added (dropwise) to a solution of L-phenylalanine (25 g, 151 mmol) in 140 mL of acetic acid. Iodine (15.3 g, 60.2 mmol) was added to the reaction flask in one portion, followed by cautious addition of sodium iodate (6.3 g, 32.0 mmol). The flask was placed in a silicon oil bath, and the reaction mixture was stirred at 70° C. for 20 hours. After 20 hours, a 1.0 g portion of sodium periodate was added to the solution. After 25 hours, another 1.0 g portion of sodium periodate was added to the reaction mixture. After the sodium periodate addition at 25 hours there was a visible color change from crimson to orange. (The progress of the reaction was monitored by TLC (16:3:2.5, MEK:AcOH:H₂O, R_{f}=0.5)). After 25 hours, the solution was cooled to room temperature and the solvent was removed by rotary evaporation to give an orange viscous oil. The oil was diluted with 200 mL of deionized water and washed with diethyl ether (2×100 mL) and dichloromethane (2×100 mL). The aqueous layer was passed through activated carbon, passed through a 0.2 μm PTFE membrane filter, and neutralized to pH 7 with 3 M NaOH. A colorless precipitate formed upon neutralization. The precipitate was filtered by vacuum and dissolved in 160 mL of boiling acetic acid. After the solution cooled to room temperature over 1.5 hours, large, pale yellow crystals formed. The crystals were filtered by vacuum and washed with small portions of ice-cold acetic acid and ice-cold ethanol. The colorless solid was transferred to a tared round bottom flask and dried under dynamic high vacuum overnight to yield 4-iodo-L-phenylalanine in 45% yield. ¹H NMR (D₂O) 400 MHz δ 3.19 (dd, J₁=11.3 Hz, J₂=14.0 Hz, 1H), δ 3.30 (dd, J₁=11.3 Hz, J₂=14.0 Hz, 1H), δ 4.34 (dd, J₁=5.9 Hz, J₂=7.6 Hz, 1H), δ 7.10 (d, J=8.4 Hz, 2H), δ 7.78 (d, J=8.4 Hz, 2H).

### Example 36

### 4-Iodo-L-phenylalanine methyl ester

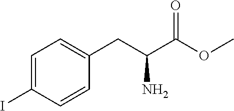

In a 1 L round bottom flask 4-Iodo-L-phenylalanine (20 g, 68.8 mmol) was dissolved in 690 mL of methanol. Thionyl chloride (10.0 mL, 68.8 mmol) was added dropwise by syringe, and the mixture was heated at reflux for 8 hrs. After 8 hours, methanol was removed under reduced pressure leaving behind a colorless solid, which was subjected to dynamic high vacuum for 6 hrs. The product was dissolved in a saturated sodium carbonate solution, and extracted with dichloromethane (3×50 mL). The organic extracts were combined, dried with sodium sulfate, and evaporated under reduced pressure to yield 14.9 g (quant.) of the product as an orange viscous oil. ¹H NMR (D2O) 400 MHz δ 3.22 (dd, J₁=11.3 Hz, J₂=14.0 Hz, 1H), δ 3.32 (dd, J₁=11.3 Hz, J₂=14.0 Hz, 1H), δ 3.85 (s, 3H), 4.45 (t, J=6.7 Hz, 1H), δ 7.09 (d, J=8.4 Hz, 2H), 87.81 (d, J=8.4 Hz, 2H).

### Example 37

### Methyl (S)-2-(tert-butoxycarbonyl)amino)-3-(4-iodophenyl)propanoate

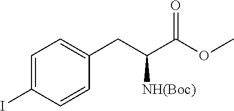

In a 250 mL round bottom flask fitted with a magnetic stir bar, 4-iodo-L-phenylalanine methyl ester (5.0 g, 16.4 mmol) was dissolved in 30 mL of tetrahydrofuran and the reaction flask was chilled to 0° C. in an ice bath. Saturated sodium bicarbonate (30 mL) was added to the flask and the reaction was stirred vigorously to minimize the formation of a bilayer. A 1 M solution of di-tert-butyl dicarbonate (4.3 g, 19.7 mmol) in tetrahydrofuran was added to the reaction flask slowly. The ice bath was removed and the reaction was stirred at room temperature for 2 hrs. After 2 hours, the mixture was poured into a separatory funnel. The tetrahydrofuran layer was removed, and the aqueous layer was extracted with ethyl acetate (2×50 mL). The organic fractions were combined and washed with 5% HCl (2×20 mL), deionized water (2×20 mL), and saturated sodium chloride (2×20 mL). The organic layers were dried with sodium sulfate, filtered, and the solvent was evaporated under reduced pressure to yield the product as a light yellow solid. The solid was carried to the next step without further purification. ¹H NMR (CDCl₃) 400 MHz δ 1.42 (s, 9H), δ 2.97 (dd, J₁=11.3 Hz, J₂=14.0 Hz, 1H), δ 3.07 (dd, J₁=11.3 Hz, J₂=14.0 Hz, 1H), δ 3.72 (s, 3H), δ 4.56 (quartet, J=7.1 Hz, 1H), δ 4.98 (d, J=7.4 Hz, 1H), δ 6.87 (d, J=8.4 Hz, 2H), δ 7.61 (d, J=8.4 Hz, 2H).

### Example 38

### Methyl (S)-2-(di-tert-butoxycarbonyl)amino)-3-(4-iodophenyl)propanoate

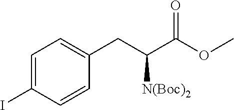

In a 250 mL round bottom flask fitted with a magnetic stir bar, methyl (S)-2-(tert-butoxycarbonyl)amino)-3-(4-iodophenyl)propanoate (6.7 g, 16.6 mmol) was dissolved in 170 mL of acetonitrile. Triethylamine (14 mL, 99.6 mmol) was added to the reaction flask, followed by 4-dimethylaminopyridine (0.41 g, 3.3 mmol), and di-tert-butyl dicarbonate (5.4 g, 25 mmol). This mixture was stirred at room temperature for 20 hours before the acetonitrile was removed by rotary evaporation leaving behind a dark red oil. The oil was dissolved in 100 mL of dichloromethane and the organic layer was washed with deionized water (3×40 mL) and brine (1×40 mL). The dichloromethane was dried with sodium sulfate, filtered, and evaporated to give a light brown oil. The oil was chromatographed on a silica column, which prior to chromatography was treated with a 10% solution of triethylamine/hexanes, then washed with 3 column-volumes of hexanes. Chromatographic separation (R_{f}=0.38, 4:1 ethylacetate:hexanes) of the product using a gradient of ethyl acetate/hexanes (2%-10%-20%) followed by subsequent removal of the solvents in vacuo yielded the product (6.5 g, 78.4%) as a colorless oil. ¹H NMR (CDCl₃) 400 MHz δ 1.41 (s, 18H), δ 3.16 (dd, J₁=11.3 Hz, J₂=14.0 Hz, 1H), δ 3.37 (dd, J₁=11.3 Hz, J₂=14.0 Hz, 1H), δ 3.74 (s, 3H), δ 5.11 (quartet, J=5.1 Hz, 1H), δ 6.94 (d, J=8.4 Hz, 2H), δ 7.59 (d, J=8.4 Hz, 2H).

### Example 39

### 4-(((S)-2-(di-tert-butoxycarbonyl)amino)-3-oxo-3-methoxypropyl)phenyl) (bis-acetoxy)-λ³-iodane

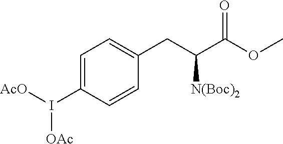

In a N₂ charged glove box, methyl (S)-2-(di-tert-butoxycarbonyl)amino)-3-(4-iodophenyl)propanoate (6.4 g, 12.6 mmol) was dissolved in 63 mL of dry, distilled acetonitrile in a polyethylene container. To the same container was added trimethylsilyl acetate (4.2 g, 31.4 mmol) and the reaction mixture was stirred. In a separate plastic flask, SelectFluor® was dissolved in 103 mL of dry, distilled acetonitrile and the Selectfluor® mixture was added dropwise to the stirred phenylalanine/trimethylsilyl acetate mixture, and the solution was allowed to stir for 8 hours. After 8 hours, the acetonitrile was removed under reduced pressure to yield a colorless solid. The solid was washed with dichloromethane (3×50 mL) and the organic fractions were combined. The combined organic extracts were washed with aqueous acetate buffer (4×40 mL) (NaOAc: HOAc; 0.5M:0.5M; pH=5), and dried over sodium sulfate. The dichloromethane was removed under reduced pressure to afford a yellow oil, which was treated with 40 mL of pentanes and sonicated until the salt solidified. The pentane was decanted off and the colorless solid was placed under high dynamic vacuum for 5 hours. The colorless solid was then carried forward to the next step without further purification. ¹H NMR (CD₃CN) 400 MHz δ 1.40 (s, 18H), δ 1.93 (s, 6H), δ 3.29 (dd, J₁=11.3 Hz, J₂=14.0 Hz, 1H), δ 3.48 (dd, J₁=11.3 Hz, J₂=14.0 Hz, 1H), δ 3.75 (s, 3H), δ 5.25 (dd, J₁=11.3 Hz, J₂=14.0 Hz, 1H), δ 7.38 (d, J=8.4 Hz, 2H), δ 8.07 (d, J=8.4 Hz, 2H).

### Example 40

### [(4-methoxyphenyl)((4-(S)-2-(di-tert-butoxycarbonyl)amino)-3-oxo-3-methoxypropyl)phenyl)(trifluoromethanesulfonyl)-λ³-iodane

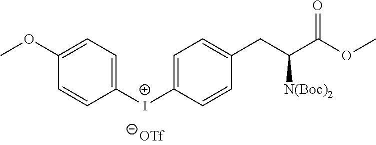

In a N₂ charged glove 4-(((S)-2-(di-tert-butoxycarbonyl)amino)-3-oxo-3-methoxypropyl)phenyl)(bis-acetoxy)-λ³-iodane (1.0 g, 1.6 mmol) was dissolved in 5.6 mL of dry, distilled acetonitrile. In a separate flask potassium (4-methoxyphenyl)trifluoroborate (0.34 g, 1.6 mmol) was dissolved in 13 mL of dry, distilled acetonitrile and was subsequently added to the hypervalent iodine solution. Next, trimethylsilyl trifluoroacetate (0.29 g, 1.6 mmol) was added dropwise to the reaction vial while stirring. After 10 minutes at room temperature the solvent was removed under reduced pressure to yield an oil. The oil was dissolved in 20 mL of dichloromethane and the organic layer was washed with aqueous acetate buffer (3×12 mL) (NaOAc: HOAc; 0.5 M: 0.5 M; pH=5) and evaporated to yield a light yellow solid. The solid was dissolved in 4 mL of dry acetonitrile and an aqueous solution of sodium hexafluorophosphate (1.0 g in 4 mL deionized water) was added to the reaction flask and the solution was stirred for 3 minutes. The resulting precipitate was extracted with dichloromethane (3×20 mL), and the organic extracts were combined, dried over sodium sulfate, and evaporated to provide a colorless solid. This material was dissolved in 3 mL of an acetonitrile/water (90:10) solution and passed through an IRA-400 resin (previously loaded with trifluoromethanesulfonate) with an additional 25 mL of acetonitrile/water (90:10). The solvent was removed under reduced pressure to give a colorless oil. ¹H NMR (CD₃CN) 400 MHz δ 1.21 (s, 18H), δ 3.21 (dd, J₁=11.3 Hz, J₂=14.0 Hz, 1H), δ 3.42 (dd, J₁=11.3 Hz, J₂=14.0 Hz, 1H), δ 3.69 (s, 3H), δ 3.83 (s, 3H), δ 5.16 (dd, J₁=4.9 Hz, J₂=10.9 Hz, 1H), 87.05 (d, J=8.4 Hz, 2H), δ 7.33 (d, J=8.4 Hz, 2H), δ 7.96 (d, J=8.4 Hz, 2H), δ 8.02 (d, J=8.4 Hz, 2H).

### Example 41

### N-(3-iodobenzyl)maleimide

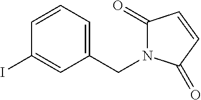 [SMILES: O=C1C=CC(=O)N1Cc1cccc(I)c1]

DIAD (12 mmol, 2.43 g, 2.40 mL, 1.2 eq.) was added over the course of one hour to a solution of 3-iodobenzyl alcohol (10 mmol, 2.34 g, 1.0 eq.), PPh₃ (11 mmol, 2.88 g, 1.1 eq.), and maleimide (11 mmol, 1.07 g, 1.1 eq.) in 100 mL of THF. After the resulting yellow solution was stirred overnight, the solvent was removed and the residue was purified by column chromatography on silica gel (hexanes:ethyl acetate=1:5, R_{f}=0.3) and washed with hexane to obtain 1.79 g (57%) of product as a white solid. ¹H NMR (CD₃CN, 400 MHz): δ 7.64 (d, J=1.6 Hz, 1H), 7.63 (d, J=9.6 Hz, 1H), 7.27 (d, J=7.6 Hz, 1H), 7.09 (t, J=7.6 Hz, 1H), 6.78 (s, 6H), 4.56 (s, 2H).

### Example 42

### [3-((2,5-dioxo-2,5-dihydro-1H-pyrrol-1-yl)methyl)phenyl]-(4′-methoxyphenyl)iodonium triflate

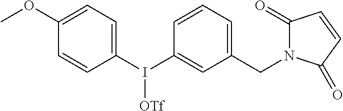 [SMILES: COc1ccc(I(OS(=O)(=O)C(F)(F)F)c2cccc(CN3C(=O)C=CC3=O)c2)cc1]

In a N₂ charged glovebox, a solution of TMSOAc (10.4 mmol, 1.37 g, 2.6 eq.) in 50 mL of dry CH₃CN was added dropwise to a solution of Selectfluor™ (5.2 mmol, 1.84 g, 1.3 eq.) in 50 mL of dry CH₃CN. The resulting colorless mixture was then added dropwise to a solution of N-(3-iodobenzyl)maleimide (4 mmol, 1.25 g, 1.0 eq.) in dry CH₃CN (150 mL). After the resulting solution was stirred at room temperature for one day, potassium 4-methoxyphenyltrifluoroborate (856 mg, 4 mmol, 1.0 equiv.) was added. Immediately thereafter, a solution of TMSOTf (764 mg, 3.4 mmol, 0.8 eq.) in 50.0 mL of dry CH₃CN was added in a dropwise fashion, and the mixture was allowed to stand at room temperature for 30 min. The acetonitrile was removed under reduced pressure. Deionized water (200 mL) was added to the remaining solid and the mixture was extracted (3×50 mL) with CH₂Cl₂. The combined organic layers were washed with water (50 mL) and the obtained water layer was extracted (50 mL×2) with CH₂CH₂ again. The combined organic extracts were dried over sodium sulfate, filtered, and the solvent was removed by rotary evaporation. This compound was dissolved in 1 mL acetonitrile/water (9:1 by volume) solution and slowly passed down an Amberlite IRA-400 ion exchange column (triflate counterion). After removal of the solvents under reduced pressure, the purified iodonium triflate product (1.06 g, 47%) was obtained by washing the colorless residue with EtOAc to remove any organic impurities. ¹H NMR (CD₃CN, 400 MHz): δ 7.98 (d, J=8.4 Hz, 2H), 7.90 (d, J=8.4 Hz, 2H), 7.86 (s, 1H), 7.58 (d, J=7.2 Hz, 1H), 7.47 (t, J=8.0 Hz, 1H), 7.07 (d, J=8.4 Hz, 2H), 6.83 (s, 2H), 4.64 (s, 2H), 3.85 (s, 3H); ¹⁹F NMR (CD₃CN, 376 MHz): δ −79.3 (s, 3F).

### Example 43

### N-(4-iodobenzyl)maleimide

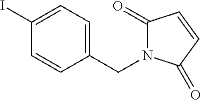 [SMILES: O=C1C=CC(=O)N1Cc1ccc(I)cc1]

This compound was prepared starting with 4-iodobenzyl alcohol using the identical procedure described in example 41 on a 10 mmol scale. Silica gel chromatography (hexanes:ethyl acetate=1:5, R_{f}=0.3), yielded the title compound (2.0 g of product, 64%) ¹H NMR (C₆D₆, 400 MHz): δ 7.35 (d, J=8.0 Hz, 2H), 6.81 (d, J=8.0 Hz, 2H), 5.61 (s, 2H), 4.13 (s, 2H).

### Example 44

### [4-((2,5-dioxo-2,5-dihydro-1H-pyrrol-1-yl)methyl)phenyl]-(4′-methoxyphenyl)iodonium triflate

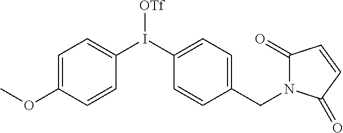 [SMILES: COc1ccc(I(OS(=O)(=O)C(F)(F)F)c2ccc(CN3C(=O)C=CC3=O)cc2)cc1]

This compound was prepared from N-(4-iodobenzyl)maleimide using the same procedure that is described in example 42. A (3 mmol scale reaction yielded 910 mg of product, (53%). ¹H NMR (CD₃CN, 400 MHz): δ 7.99 (d, J=9.2 Hz, 2H), 7.97 (d, J=8.8 Hz, 2H), 7.40 (d, J=8.4 Hz, 2H), 7.05 (d, J=9.2 Hz, 2H), 6.80 (s, 2H), 4.67 (s, 2H), 3.84 (s, 3H); ¹⁹F NMR (CD₃CN, 376 MHz): δ −79.3 (s, 3F).

### Example 45

### 4-(4-iodobenzyl)benzoic acid

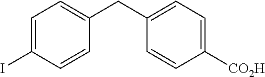 [SMILES: O=C(O)c1ccc(Cc2ccc(I)cc2)cc1]

In a 500 mL round bottom flask that was shielded from light with aluminum foil, a stirred solution of 4-benzylbenzoic acid (1.06 g, 5 mmol, 1.0 eq.), NIS (1.24 g, 5.5 mmol, 1.1 eq.) and Yb(OTf)₃ (310 mg, 0.50 mmol, 0.1 eq.) in CH₃CN (100 mL) was heated to 75-80° C. for 12 hours. After 12 h, a supplementary portion of NIS (0.56 g, 2.5 mmol, 0.5 eq.) was added to drive the reaction to completion. After an additional hour, the solvent was removed by rotary evaporation, and the residue was partitioned between water and ethyl acetate. The mixture was extracted (3×50 mL) with ethyl acetate and the combined organic extracts were washed with water, dried over MgSO₄, and filtered. The solvent was removed by rotary evaporation, and the residue was purified by flash chromatography on silica gel (hexanes:ethyl acetate=1:1, Rf=0.2) to give 4-(4-iodobenzyl)benzoic acid as a white solid (1.28 g, 76%). ¹H NMR (CD₃CN, 400 MHz): δ 7.91 (d, J=8.0 Hz, 2H), 7.65 (d, J=8.4 Hz, 2H), 7.31 (d, J=8.4 Hz, 2H), 7.03 (d, J=8.4 Hz, 2H), 3.99 (s, 2H).

### Example 46

### 2,5-dioxopyrrolidin-1-yl 4-(4-iodobenzyl)benzoate

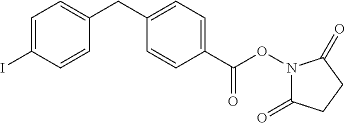 [SMILES: O=C(ON1C(=O)CCC1=O)c1ccc(Cc2ccc(I)cc2)cc1]

4-(4-Iodobenzyl)benzoic acid (3.8 mmol, 1.28 g, 1.0 eq.) and N-hydroxysuccinimide (5.7 mmol, 0.66 g, 1.5 eq) were dissolved in anhydrous CH₂Cl₂ (20 mL). The mixture was cooled to 0° C. before N,N′-dicyclohexylcarbodiimide (DCC, 5.7 mmol, 1.18 g, 1.5 eq) dissolved in 10 mL CH₂Cl₂ was added in a dropwise fashion. The mixture was stirred for 12 hours at room temperature and filtered to remove precipitated N,N′-dicyclohexylurea. The residue was washed with additional CH₂Cl₂, and the combined filtrate was evaporated in vacuo. The residue was purified by column chromatography (hexanes: ethyl acetate=1:5, Rf=0.6). Recrystallization with isopropanol or toluene/hexane afforded the title compound as a colorless solid (0.60 g, 36%). Recrystallization with isopropanol or toluene/hexane afforded the title compound as a colorless solid. ¹H NMR (CD₃CN, 400 MHz): δ 8.04 (d, J=8.4 Hz, 2H), 7.67 (d, J=8.4 Hz, 2H), 7.42 (d, J=8.4 Hz, 2H), 7.04 (d, J=8.4 Hz, 2H), 4.05 (s, 2H), 2.83 (s, 4H).

### Example 47

### [4-(4-(((2,5-dioxopyrrolidin-1-yl)oxy)carbonyl)benzyl)phenyl]-]-(4′-methoxyphenyl)iodonium triflate

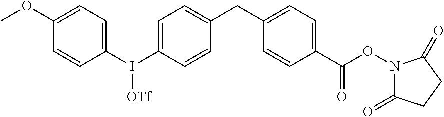 [SMILES: COc1ccc(I(OS(=O)(=O)C(F)(F)F)c2ccc(Cc3ccc(C(=O)ON4C(=O)CCC4=O)cc3)cc2)cc1]

In a N₂ charged glovebox, a solution of TMSOAc (3.90 mmol, 516 mg, 2.6 eq.) in 20 mL of dry CH₃CN was added dropwise to a solution of Selectfluor™ (1.95 mmol, 691 mg, 1.3 eq.) in 20 mL of dry CH₃CN. The resulting colorless mixture was then added slowly (dropwise) to a solution of 2,5-dioxopyrrolidin-1-yl 4-(4-iodobenzyl)benzoate (1.5 mmol, 653 mg, 1.0 eq.) in 40 mL of dry CH₃CN. The mixture was stirred at room temperature for 2 days before potassium 4-methoxyphenyltrifluoroborate (320 mg, 1.5 mmol, 1.0 equiv.) was added. Immediately thereafter, a solution of TMSOTf (267 mg, 1.2 mmol, 0.8 eq.) in 20.0 mL of dry CH₃CN was added slowly (dropwise), and the mixture was allowed to stand at room temperature for 30 minutes. The acetonitrile was removed by rotary evaporation, 100 mL of deionized water was added, and the mixture was extracted (3×30 mL) with CH₂Cl₂. The combined organic extracts were washed with water (50 mL) and the aqueous layer was extracted (2×50 mL) with CH₂Cl₂ again. The combined organic extracts were dried over sodium sulfate, filtered, and concentrated in vacuo. The residue was washed with methy t-butyl ether (MTBE). This compound was dissolved in 1 mL acetonitrile/water (9:1 by volume) solution and slowly passed down an Amberlite IRA-400 ion exchange column (triflate counterion). After removal of the solvents under reduced pressure, the purified iodonium triflate product was obtained by washing the colorless residue with pentane to remove any organic impurities (540 mg, 52%). ¹H NMR (CD₃CN, 400 MHz): δ 8.05 (d, J=8.0 Hz, 2H), 7.99 (d, J=9.2 Hz, 2H), 7.95 (d, J=8.4 Hz, 2H), 7.42 (d, J=8.4 Hz, 2H), 7.38 (d, J=8.4 Hz, 2H), 7.05 (d, J=9.2 Hz, 2H), 4.16 (s, 2H), 3.83 (s, 3H), 2.84 (s, 4H); ¹⁹F NMR (CD₃CN, 376 MHz): δ −79.3 (s, 3F).

1. A process for making a compound of Formula I: 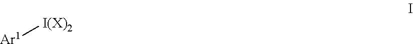
comprising:
treating a compound of Formula II:
Ar¹—I II
with a tetravalent silicon moiety having at least one X group bound to Si; and (1-chloromethyl-4-fluoro-1,4-diazoniabicyclo[2.2.2]octane)bis(tetrafluoroborate), (1-fluoro-4-methyl-1,4-diazoniabicyclo[2.2.2]octane)bis(tetrafluoroborate), or optionally substituted N-fluoropyridinium tetrafluoroborate;
wherein:
each X is, independently, a ligand that is a conjugate base of an acid HX, wherein HX has a pKa of less than or equal to 12; and
Ar¹ is optionally substituted aryl or heteroaryl, wherein Ar¹ does not have unprotected protic groups.

2. The process of claim 1, wherein the process is carried out in the absence of added acid.

3-7. (canceled)

8. The process of claim 1, wherein each X is, independently, a ligand that is a conjugate base of an acid HX, wherein HX has a pKa of less than or equal to 5.

9. (canceled)

10. The process of claim 1, wherein the tetravalent silicon moiety is (R¹)₃Si—X, wherein each R¹ is, independently, C₁₋₁₂ alkyl or aryl.

11-13. (canceled)

14. The process of claim 1, wherein the process utilizes 2 equivalents or more of the tetravalent silicon moiety for 1 equivalent of the compound of Formula II.

15. (canceled)

16. The process of claim 14, wherein the tetravalent silicon moiety is (R¹)₃Si—X, wherein each R¹ is, independently, C₁₋₁₂ alkyl or aryl.

17-18. (canceled)

19. The process of claim 1, further comprising converting the compound of Formula I to a compound of Formula III: 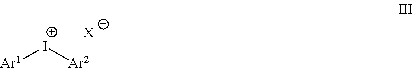
wherein Ar² is an optionally substituted aryl or heteroaryl.

20. The process of claim 19, wherein said converting comprises reacting the compound of Formula I with a compound of Formula IV:
Ar²-M¹ IV
wherein M¹ is a borate, stannane, silane, magnesium moiety, or zinc moiety.

21. The process of claim 20, wherein M¹ is Sn(R^{x})₃, Si(R^{y})₃, B(OR^{z})₂, or B(X²)₃M²; wherein:
each R^{x} is, independently, C₁₋₆ alkyl;
each R^{y} is, independently, C₁₋₆ alkyl;
each R^{z} is, independently, OH or C₁₋₆ alkoxy; or
two R^{z} groups, taken together with the oxygen atoms to which they are attached and the boron atom to which the oxygen atoms are attached, form a 5- to 6-membered heterocyclic ring, which is optionally substituted with 1, 2, 3, or 4 C₁₋₄ alkyl groups;
each X² is, independently, halo; and
M² is a counterion.

22-23. (canceled)

24. The process of claim 21, wherein the process is carried out in the presence of a catalyst.

25. (canceled)

26. The process of claim 19, further comprising subjecting the compound of Formula III to ion-exchange in order to form a compound of Formula V: 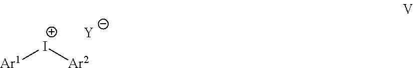
wherein Y is a counterion that is different than X.

27-28. (canceled)

29. A process of forming a compound of Formula III: 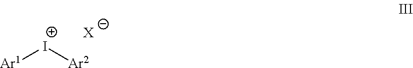
comprising:
(a) treating a compound of Formula II:
Ar¹—I II
with more than 2 equivalents of (R¹)₃Si—X; and less than 2 equivalents of (1-chloro methyl-4-fluoro-1,4-diazoniabicyclo[2.2.2]octane)bis(tetrafluoroborate) or (1-fluoro-4-methyl-1,4-diazoniabicyclo[2.2.2]octane)bis(tetrafluoroborate) in the absence of added acid to form a compound of Formula I: 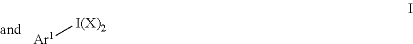
(b) reacting the compound of Formula I with Ar²BF₃M² in the presence of a catalyst to form a compound of Formula III: wherein:
Each X is, independently, a ligand that is a conjugate base of an acid HX, wherein HX has a pKa of less than or equal to 12;
Ar¹ is optionally substituted aryl or heteroaryl, wherein Ar¹ does not have unprotected protic groups;
Ar² is an optionally substituted aryl or heteroaryl;
each R¹ is, independently, C₁₋₄ alkyl; and
M² is a cation.

30-34. (canceled)

35. The process of claim 1, wherein:
Ar¹ is aryl or heteroaryl, which is optionally substituted by one or more groups independently selected from halo, cyano, nitro, C₁₋₁₆ alkyl, C₁₋₆haloalkyl, C₂₋₁₆ alkenyl, C₂₋₁₆ alkynyl, C₁₋₆ alkoxy, C₃₋₁₄ cycloalkyl, C₃₋₁₄ cycloalkyl-C₁₋₄-alkyl, C₂₋₁₄ heterocycloalkyl, C₂₋₁₄ heterocycloalkyl-C₁₋₄-alkyl, C₆₋₁₄ aryl, C₆₋₁₄ aryl-C₁₋₄-alkyl, C₁₋₁₄ heteroaryl, C₁₋₁₄ heteroaryl-C₁₋₄-alkyl, —S(═O)R^{a}, —S(═O)₂R^{a}, —S(═O)₂NR^{g}R^{h}, —C(C═O)R^{b}, —C(C═O)NR^{g}R^{h}, —OC(═O)R^{a}, —OC(═O)NR^{g}R^{h}, —NR^{k}C(═O)R^{a}, —NR^{k}C(═O)OR^{b}, —NR^{k}C(═O)NR^{g}NR^{h}, —NR^{k}S(═O)₂R^{a}, —NR^{k}S(═O)₂NR^{g}R^{h}, C(═NRⁱ)NR^{g}R^{h}, NR^{k}C(═NRⁱ)NR^{g}R^{h}, —OR^{e}, —SR^{d}, —S(═O)₂OR^{e}, —C(C═O)OR^{f}, and —NR^{g}R^{h}; wherein said C₁₋₆ alkyl, C₁₋₆haloalkyl, C₂₋₆ alkenyl, C₂₋₆ alkynyl, C₁₋₆ alkoxy, C₃₋₁₄ cycloalkyl, C₃₋₁₄ cycloalkyl-C₁₋₄-alkyl, C₂₋₁₄ heterocycloalkyl, C₂₋₁₄ heterocycloalkyl-C₁₋₄-alkyl, C₆₋₁₄ aryl, C₆₋₁₄ aryl-C₁₋₄-alkyl, C₁₋₁₄heteroaryl, and C₁₋₁₄ heteroaryl-C₁₋₄-alkyl are each optionally substituted by one or more independently selected R² groups;
each Rⁱ is independently selected from H, C₁₋₆ alkyl, CN, C₁₋₆ alkoxy, or C(O)C₁₋₆ alkyl;
each R^{a} is independently selected from H, C₁₋₆ alkyl, C₁₋₆haloalkyl, C₂₋₆ alkenyl, C₂₋₆ alkynyl, aryl, C₃₋₁₀ cycloalkyl, C₃₋₁₀ cycloalkyl-C₁₋₄-alkyl, C₂₋₁₀heterocycloalkyl, C₂₋₁₀heterocycloalkyl-C₁₋₄-alkyl, C₆₋₁₀ aryl, C₆₋₁₀ aryl-C₁₋₄-alkyl, C₁₋₁₀heteroaryl, wherein said C₁₋₆ alkyl, C₁₋₆haloalkyl, C₂₋₆ alkenyl, C₂₋₆ alkynyl, C₃₋₁₀ cycloalkyl, C₃₋₁₀ cycloalkyl-C₁₋₄-alkyl, C₂₋₁₀ heterocycloalkyl, C₂₋₁₀ heterocycloalkyl-C₁₋₄-alkyl, C₆₋₁₀ aryl, C₆₋₁₀ aryl-C₁₋₄-alkyl, C₁₋₁₀ heteroaryl, and C₁₋₁₀ heteroaryl-C₁₋₄-alkyl are each optionally substituted by one or more independently selected R² groups;
each R^{b} is independently selected from C₁₋₆ alkyl, C₁₋₆haloalkyl, C₂₋₆ alkenyl, C₂₋₆ alkynyl, C₃₋₁₀ cycloalkyl, C₃₋₁₀ cycloalkyl-C₁₋₄-alkyl, C₂₋₁₀ heterocycloalkyl, C₂₋₁₀ heterocycloalkyl-C₁₋₄-alkyl, C₆₋₁₀ aryl, C₆₋₁₀ aryl-C₁₋₄-alkyl, C₁₋₁₀heteroaryl, wherein said C₁₋₆ alkyl, C₁₋₆haloalkyl, C₂₋₆ alkenyl, C₂₋₆ alkynyl, aryl, C₃₋₁₀ cycloalkyl, C₃₋₁₀ cycloalkyl-C₁₋₄-alkyl, C₂₋₁₀ heterocycloalkyl, C₂₋₁₀ heterocycloalkyl-C₁₋₄-alkyl, C₆₋₁₀ aryl, C₆₋₁₀ aryl-C₁₋₄-alkyl, C₁₋₁₀ heteroaryl, and C₁₋₁₀ heteroaryl-C₁₋₄-alkyl are each optionally substituted by one or more independently selected R² groups;
each R^{c} is independently selected from a protecting group, C₁₋₆ alkyl, C₁₋₆haloalkyl, C₂₋₆ alkenyl, C₂₋₆ alkynyl, C₃₋₁₀ cyclo alkyl, C₃₋₁₀ cycloalkyl-C₁₋₄-alkyl, C₂₋₁₀ heterocycloalkyl, C₂₋₁₀ heterocycloalkyl-C₁₋₄-alkyl, C₆₋₁₀ aryl, C₆₋₁₀ aryl-C₁₋₄-alkyl, C₁₋₁₀heteroaryl, wherein said C₁₋₆ alkyl, C₁₋₆haloalkyl, C₂₋₆ alkenyl, C₂₋₆ alkynyl, aryl, C₃₋₁₀ cycloalkyl, C₃₋₁₀ cycloalkyl-C₁₋₄-alkyl, C₂₋₁₀ heterocycloalkyl, C₂₋₁₀ heterocycloalkyl-C₁₋₄-alkyl, C₆₋₁₀ aryl, C₆₋₁₀ aryl-C₁₋₄-alkyl, C₁₋₁₀ heteroaryl, and C₁₋₁₀ heteroaryl-C₁₋₄-alkyl are each optionally substituted by one or more independently selected R² groups;
each R^{d} is independently selected from a protecting group, C₁₋₆ alkyl, C₁₋₆haloalkyl, C₂₋₆ alkenyl, C₂₋₆ alkynyl, C₃₋₁₀ cyclo alkyl, C₃₋₁₀ cycloalkyl-C₁₋₄-alkyl, C₂₋₁₀ heterocycloalkyl, C₂₋₁₀ heterocycloalkyl-C₁₋₄-alkyl, C₆₋₁₀ aryl, C₆₋₁₀ aryl-C₁₋₄-alkyl, C₁₋₁₀heteroaryl, wherein said C₁₋₆ alkyl, C₁₋₆haloalkyl, C₂₋₆ alkenyl, C₂₋₆ alkynyl, aryl, C₃₋₁₀ cycloalkyl, C₃₋₁₀ cycloalkyl-C₁₋₄-alkyl, C₂₋₁₀ heterocycloalkyl, C₂₋₁₀ heterocycloalkyl-C₁₋₄-alkyl, C₆₋₁₀ aryl, C₆₋₁₀ aryl-C₁₋₄-alkyl, C₁₋₁₀ heteroaryl, and C₁₋₁₀ heteroaryl-C₁₋₄-alkyl are each optionally substituted by one or more independently selected R² groups;
each R^{e} is independently selected from a protecting group, C₁₋₆ alkyl, C₁₋₆haloalkyl, C₂₋₆ alkenyl, C₂₋₆ alkynyl, C₃₋₁₀ cyclo alkyl, C₃₋₁₀ cycloalkyl-C₁₋₄-alkyl, C₂₋₁₀ heterocycloalkyl, C₂₋₁₀ heterocycloalkyl-C₁₋₄-alkyl, C₆₋₁₀ aryl, C₆₋₁₀ aryl-C₁₋₄-alkyl, C₁₋₁₀heteroaryl, wherein said C₁₋₆ alkyl, C₁₋₆ haloalkyl, C₂₋₆ alkenyl, C₂₋₆ alkynyl, aryl, C₃₋₁₀ cycloalkyl, C₃₋₁₀ cycloalkyl-C₁₋₄-alkyl, C₂₋₁₀ heterocycloalkyl, C₂₋₁₀ heterocycloalkyl-C₁₋₄-alkyl, C₆₋₁₀ aryl, C₆₋₁₀ aryl-C₁₋₄-alkyl, C₁₋₁₀ heteroaryl, and C₁₋₁₀ heteroaryl-C₁₋₄-alkyl are each optionally substituted by one or more independently selected R² groups;
each R^{f} is independently selected from a protecting group, C₁₋₆ alkyl, C₁₋₆ haloalkyl, C₂₋₆ alkenyl, C₂₋₆ alkynyl, C₃₋₁₀ cyclo alkyl, C₃₋₁₀ cycloalkyl-C₁₋₄-alkyl, C₂₋₁₀ heterocycloalkyl, C₂₋₁₀ heterocycloalkyl-C₁₋₄-alkyl, C₆₋₁₀ aryl, C₆₋₁₀ aryl-C₁₋₄-alkyl, C₁₋₁₀ heteroaryl, wherein said C₁₋₆ alkyl, C₁₋₆ haloalkyl, C₂₋₆ alkenyl, C₂₋₆ alkynyl, aryl, C₃₋₁₀ cycloalkyl, C₃₋₁₀ cycloalkyl-C₁₋₄-alkyl, C₂₋₁₀ heterocycloalkyl, C₂₋₁₀ heterocycloalkyl-C₁₋₄-alkyl, C₆₋₁₀ aryl, C₆₋₁₀ aryl-C₁₋₄-alkyl, C₁₋₁₀ heteroaryl, and C₁₋₁₀ heteroaryl-C₁₋₄-alkyl are each optionally substituted by one or more independently selected R² groups;
each R^{k}, R^{g} and R^{h} is independently selected from a protecting group, C₁₋₆ alkyl, C₁₋₆ haloalkyl, C₂₋₆ alkenyl, C₂₋₆ alkynyl, C₃₋₁₀ cycloalkyl, C₃₋₁₀ cycloalkyl-C₁₋₄-alkyl, C₂₋₁₀ heterocycloalkyl, C₂₋₁₀ heterocycloalkyl-C₁₋₄-alkyl, C₆₋₁₀ aryl, C₆₋₁₀ aryl-C₁₋₄-alkyl, C₁₋₁₀ heteroaryl, wherein said C₁₋₆ alkyl, C₁₋₆ haloalkyl, C₂₋₆ alkenyl, C₂₋₆ alkynyl, aryl, C₃₋₁₀ cycloalkyl, C₃₋₁₀ cycloalkyl-C₁₋₄-alkyl, C₂₋₁₀ heterocycloalkyl, C₂₋₁₀ heterocycloalkyl-C₁₋₄-alkyl, C₆₋₁₀ aryl, C₆₋₁₀ aryl-C₁₋₄-alkyl, C₁₋₁₀ heteroaryl, and C₁₋₁₀ heteroaryl-C₁₋₄-alkyl are each optionally substituted by one or more independently selected R² groups;
or alternatively, R^{k} and R^{a}, taken together with the atoms to which they are attached, form a heterocycloalkyl or heteroaryl ring, which is optionally substituted by one or more R² groups;
or alternatively, R^{k} and R^{b}, taken together with the atoms to which they are attached, form a heterocycloalkyl or heteroaryl ring, which is optionally substituted by one or more R² groups;
or alternatively, R^{k} and R^{g}, taken together with the atoms to which they are attached, form a heterocycloalkyl or heteroaryl ring, which is optionally substituted by one or more R² groups;
or alternatively, R^{g} and R^{h}, taken together with the nitrogen atom to which they are attached, form a heterocycloalkyl or heteroaryl ring, which is optionally substituted by one or more R⁴ groups;
each R² is independently selected from halo, cyano, nitro, C₁₋₆ alkyl, C₁₋₆ haloalkyl, C₁₋₆ haloalkyl, C₂₋₆ alkenyl, C₂₋₆ alkynyl, C₁₋₆ alkoxy, C₃₋₁₀ cycloalkyl, C₃₋₁₀ cycloalkyl-C₁₋₄-alkyl, C₂₋₁₀ heterocycloalkyl, C₂₋₁₀ heterocycloalkyl-C₁₋₄-alkyl, C₆₋₁₀ aryl, C₆₋₁₀ aryl-C₁₋₄-alkyl, C₁₋₁₀ heteroaryl, C₁₋₁₀heteroaryl-C₁₋₄-alkyl, —S(═O)R^{a1}, —S(═O)₂R^{a1}, —S(═O)₂NR^{g1}R^{h1}, —C(C═O)R^{b1}, —C(C═O)NR^{g1}R^{h1}, —OC(═O)R^{a1}, —OC(═O)NR^{g1}R^{h1}, —NR^{k1}C(═O)R^{a1}, —NR^{k1}C(═O)OR^{b1}, —NR^{k1}C(═O)NR^{g1}NR^{h1}, —NR^{k1}S(═O)₂R^{a1}, —NR^{k1}S(═O)₂NR^{g1}R^{h1}, C(═NRⁱ)NR^{g1}R^{h1}, NR^{k1}C(═NRⁱ)NR^{g1}R^{h1}, —OR^{c1}, —SR^{d1}, —S(═O)₂OR^{e1}, —C(═O)OR^{f1}, and —NR^{g1}R^{h1}; wherein said C₁₋₆ alkyl, C₁₋₆ haloalkyl, C₂₋₆ alkenyl, C₂₋₆ alkynyl, C₁₋₆ alkoxy, C₃₋₁₀ cycloalkyl, C₃₋₁₀ cycloalkyl-C₁₋₄-alkyl, C₂₋₁₀ heterocycloalkyl, C₂₋₁₀ heterocycloalkyl-C₁₋₄-alkyl, C₆₋₁₀ aryl, C₆₋₁₀ aryl-C₁₋₄-alkyl, C₁₋₁₀heteroaryl, and C₁₋₁₀ heteroaryl-C₁₋₄-alkyl are each optionally substituted by one or more independently selected R³ groups;
each R^{a1} is independently selected from H, C₁₋₆ alkyl, C₁₋₆ haloalkyl, C₂₋₆ alkenyl, C₂₋₆ alkynyl, C₃₋₁₀ cycloalkyl, C₃₋₁₀ cycloalkyl-C₁₋₄-alkyl, C₂₋₁₀ heterocycloalkyl, C₂₋₁₀ heterocycloalkyl-C₁₋₄-alkyl, C₆₋₁₀ aryl, C₆₋₁₀ aryl-C₁₋₄-alkyl, C₁₋₁₀ heteroaryl, wherein said C₁₋₆ alkyl, C₁₋₆ haloalkyl, C₂₋₆ alkenyl, C₂₋₆ alkynyl, C₃₋₁₀ cycloalkyl, C₃₋₁₀ cycloalkyl-C₁₋₄-alkyl, C₂₋₁₀ heterocycloalkyl, C₂₋₁₀ heterocycloalkyl-C₁₋₄-alkyl, C₆₋₁₀ aryl, C₆₋₁₀ aryl-C₁₋₄-alkyl, C₁₋₁₀ heteroaryl, and C₁₋₁₀ heteroaryl-C₁₋₄-alkyl are each optionally substituted by one or more independently selected R³ groups;
each R^{b1} is independently selected from C₁₋₆ alkyl, C₁₋₆ haloalkyl, C₂₋₆ alkenyl, C₂₋₆ alkynyl, C₃₋₁₀ cycloalkyl, C₃₋₁₀ cycloalkyl-C₁₋₄-alkyl, C₂₋₁₀ heterocycloalkyl, C₂₋₁₀ heterocycloalkyl-C₁₋₄-alkyl, C₆₋₁₀ aryl, C₆₋₁₀ aryl-C₁₋₄-alkyl, C₁₋₁₀ heteroaryl, wherein said C₁₋₆ alkyl, C₁₋₆ haloalkyl, C₂₋₆ alkenyl, C₂₋₆ alkynyl, C₃₋₁₀ cycloalkyl, C₃₋₁₀ cycloalkyl-C₁₋₄-alkyl, C₂₋₁₀ heterocycloalkyl, C₂₋₁₀ heterocycloalkyl-C₁₋₄-alkyl, C₆₋₁₀ aryl, C₆₋₁₀ aryl-C₁₋₄-alkyl, C₁₋₁₀heteroaryl, and C₁₋₁₀ heteroaryl-C₁₋₄-alkyl are each optionally substituted by one or more independently selected R³ groups;
each R^{c1} is independently selected from a protecting group, C₁₋₆ alkyl, C₁₋₆ haloalkyl, C₂₋₆ alkenyl, C₂₋₆ alkynyl, C₃₋₁₀ cycloalkyl, C₃₋₁₀ cycloalkyl-C₁₋₄-alkyl, C₂₋₁₀ heterocycloalkyl, C₂₋₁₀ heterocycloalkyl-C₁₋₄-alkyl, C₆₋₁₀ aryl, C₆₋₁₀ aryl-C₁₋₄-alkyl, C₁₋₁₀heteroaryl, wherein said C₁₋₆ alkyl, C₁₋₆ haloalkyl, C₂₋₆ alkenyl, C₂₋₆ alkynyl, C₃₋₁₀ cycloalkyl, C₃₋₁₀ cycloalkyl-C₁₋₄-alkyl, C₂₋₁₀ heterocycloalkyl, C₂₋₁₀ heterocycloalkyl-C₁₋₄-alkyl, C₆₋₁₀ aryl, C₆₋₁₀ aryl-C₁₋₄-alkyl, C₁₋₁₀ heteroaryl, and C₁₋₁₀ heteroaryl-C₁₋₄-alkyl are each optionally substituted by one or more independently selected R³ groups;
each R^{d1} is independently selected from a protecting group, C₁₋₆ alkyl, C₁₋₆ haloalkyl, C₂₋₆ alkenyl, C₂₋₆ alkynyl, C₃₋₁₀ cycloalkyl, C₃₋₁₀ cycloalkyl-C₁₋₄-alkyl, C₂₋₁₀ heterocycloalkyl, C₂₋₁₀ heterocycloalkyl-C₁₋₄-alkyl, C₆₋₁₀ aryl, C₆₋₁₀ aryl-C₁₋₄-alkyl, C₁₋₁₀heteroaryl, wherein said C₁₋₆ alkyl, C₁₋₆ haloalkyl, C₂₋₆ alkenyl, C₂₋₆ alkynyl, C₃₋₁₀ cycloalkyl, C₃₋₁₀ cycloalkyl-C₁₋₄-alkyl, C₂₋₁₀ heterocycloalkyl, C₂₋₁₀ heterocycloalkyl-C₁₋₄-alkyl, C₆₋₁₀ aryl, C₆₋₁₀ aryl-C₁₋₄-alkyl, C₁₋₁₀ heteroaryl, and C₁₋₁₀ heteroaryl-C₁₋₄-alkyl are each optionally substituted by one or more independently selected R³ groups;
each R^{e1} is independently selected from a protecting group, C₁₋₆ alkyl, C₁₋₆ haloalkyl, C₂₋₆ alkenyl, C₂₋₆ alkynyl, C₃₋₁₀ cyclo alkyl, C₃₋₁₀ cycloalkyl-C₁₋₄-alkyl, C₂₋₁₀ heterocycloalkyl, C₂₋₁₀ heterocycloalkyl-C₁₋₄-alkyl, C₆₋₁₀ aryl, C₆₋₁₀ aryl-C₁₋₄-alkyl, C₁₋₁₀ heteroaryl, wherein said C₁₋₆ alkyl, C₁₋₆ haloalkyl, C₂₋₆ alkenyl, C₂₋₆ alkynyl, C₃₋₁₀ cycloalkyl, C₃₋₁₀ cycloalkyl-C₁₋₄-alkyl, C₂₋₁₀ heterocycloalkyl, C₂₋₁₀ heterocycloalkyl-C₁₋₄-alkyl, C₆₋₁₀ aryl, C₆₋₁₀ aryl-C₁₋₄-alkyl, C₁₋₁₀ heteroaryl, and C₁₋₁₀ heteroaryl-C₁₋₄-alkyl are each optionally substituted by one or more independently selected R³ groups;
each R^{f1} is independently selected from a protecting group, C₁₋₆ alkyl, C₁₋₆ haloalkyl, C₂₋₆ alkenyl, C₂₋₆ alkynyl, C₃₋₁₀ cyclo alkyl, C₃₋₁₀ cycloalkyl-C₁₋₄-alkyl, C₂₋₁₀ heterocycloalkyl, C₂₋₁₀ heterocycloalkyl-C₁₋₄-alkyl, C₆₋₁₀ aryl, C₆₋₁₀ aryl-C₁₋₄-alkyl, C₁₋₁₀ heteroaryl, wherein said C₁₋₆ alkyl, C₁₋₆ haloalkyl, C₂₋₆ alkenyl, C₂₋₆ alkynyl, C₃₋₁₀ cycloalkyl, C₃₋₁₀ cycloalkyl-C₁₋₄-alkyl, C₂₋₁₀ heterocycloalkyl, C₂₋₁₀ heterocycloalkyl-C₁₋₄-alkyl, C₆₋₁₀ aryl, C₆₋₁₀ aryl-C₁₋₄-alkyl, C₁₋₁₀ heteroaryl, and C₁₋₁₀ heteroaryl-C₁₋₄-alkyl are each optionally substituted by one or more independently selected R³ groups;
each R^{k1}, R^{g1} and R^{h2} is independently selected from a protecting group, C₁₋₆ alkyl, C₁₋₆ haloalkyl, C₂₋₆ alkenyl, C₂₋₆ alkynyl, C₃₋₁₀ cycloalkyl, C₃₋₁₀ cycloalkyl-C₁₋₄-alkyl, C₂₋₁₀ heterocycloalkyl, C₂₋₁₀ heterocycloalkyl-C₁₋₄-alkyl, C₆₋₁₀ aryl, C₆₋₁₀ aryl-C₁₋₄-alkyl, C₁₋₁₀ heteroaryl, wherein said C₁₋₆ alkyl, C₁₋₆ haloalkyl, C₂₋₆ alkenyl, C₂₋₆ alkynyl, C₃₋₁₀ cycloalkyl, C₃₋₁₀ cycloalkyl-C₁₋₄-alkyl, C₂₋₁₀ heterocycloalkyl, C₂₋₁₀ heterocycloalkyl-C₁₋₄-alkyl, C₆₋₁₀ aryl, C₆₋₁₀ aryl-C₁₋₄-alkyl, C₁₋₁₀heteroaryl, and C₁₋₁₀ heteroaryl-C₁₋₄-alkyl are each optionally substituted by one or more independently selected R³ groups;
or alternatively, R^{k1} and R^{a1}, taken together with the atoms to which they are attached, form a heterocycloalkyl or heteroaryl ring, which is optionally substituted by one or more R³ groups;
or alternatively, R^{k1} and R^{b1}, taken together with the atoms to which they are attached, form a heterocycloalkyl or heteroaryl ring, which is optionally substituted by one or more R³ groups;
or alternatively, R^{k1} and R^{g1}, taken together with the atoms to which they are attached, form a heterocycloalkyl or heteroaryl ring, which is optionally substituted by one or more R³ groups;
or alternatively, R^{g1} and R^{h1}, taken together with the nitrogen atom to which they are attached, form a heterocycloalkyl or heteroaryl ring, which is optionally substituted by one or more R³ groups;
each R³ is independently selected from halo, cyano, nitro, C₁₋₆ alkyl, C₁₋₆ haloalkyl, C₁₋₆ haloalkyl, C₂₋₆ alkenyl, C₂₋₆ alkynyl, C₁₋₆ alkoxy, C₃₋₁₀ cycloalkyl, C₃₋₁₀ cycloalkyl-C₁₋₄-alkyl, C₂₋₁₀ heterocycloalkyl, C₂₋₁₀ heterocycloalkyl-C₁₋₄-alkyl, C₆₋₁₀ aryl, C₆₋₁₀ aryl-C₁₋₄-alkyl, C₁₋₁₀ heteroaryl, C₁₋₁₀heteroaryl-C₁₋₄-alkyl, —S(═O)R^{a2}, —S(═O)₂R^{a2}, —S(═O)₂NR^{g2}R^{h2}, —C(C═O)R^{b2}, —C(C═O)NR^{g2}R^{h2}, —OC(═O)R^{a2}, —OC(═O)NR^{g2}R^{h2}, —NR^{k2}C(═O)R^{a2}, —NR^{k2}C(═O)OR^{b2}, —NR^{k2}C(═O)NR^{g2}NR^{h2}, —NR^{k2}S(═O)₂R^{a2}, —NR^{k2}S(═O)₂NR^{g2}R^{h2}, C(═NRⁱ)NR^{g2}R^{h2}, NR^{k2}C(═NRⁱ)NR^{g2}R^{h2}, —OR^{e2}, —SR^{d2}, —S(═O)₂OR^{e2}, —C(═O)OR^{f2}, and —NR^{g2}R^{h2}; wherein said C₁₋₆ alkyl, C₁₋₆ haloalkyl, C₂₋₆ alkenyl, C₂₋₆ alkynyl, C₁₋₆ alkoxy, C₃₋₁₀ cycloalkyl, C₃₋₁₀ cycloalkyl-C₁₋₄-alkyl, C₂₋₁₀ heterocycloalkyl, C₂₋₁₀ heterocycloalkyl-C₁₋₄-alkyl, C₆₋₁₀ aryl, C₆₋₁₀ aryl-C₁₋₄-alkyl, C₁₋₁₀heteroaryl, and C₁₋₁₀ heteroaryl-C₁₋₄-alkyl are each optionally substituted by one or more independently selected R⁴ groups;
each R^{a2} is independently selected from H, C₁₋₆ alkyl, C₁₋₆ haloalkyl, C₂₋₆ alkenyl, C₂₋₆ alkynyl, C₃₋₁₀ cycloalkyl, C₃₋₁₀ cycloalkyl-C₁₋₄-alkyl, C₂₋₁₀ heterocycloalkyl, C₂₋₁₀ heterocycloalkyl-C₁₋₄-alkyl, C₆₋₁₀ aryl, C₆₋₁₀ aryl-C₁₋₄-alkyl, C₁₋₁₀ heteroaryl, wherein said C₁₋₆ alkyl, C₁₋₆ haloalkyl, C₂₋₆ alkenyl, C₂₋₆ alkynyl, C₃₋₁₀ cycloalkyl, C₃₋₁₀ cycloalkyl-C₁₋₄-alkyl, C₂₋₁₀ heterocycloalkyl, C₂₋₁₀ heterocycloalkyl-C₁₋₄-alkyl, C₆₋₁₀ aryl, C₆₋₁₀ aryl-C₁₋₄-alkyl, C₁₋₁₀ heteroaryl, and C₁₋₁₀ heteroaryl-C₁₋₄-alkyl are each optionally substituted by one or more independently selected R⁴ groups;
each R^{b2} is independently selected from C₁₋₆ alkyl, C₁₋₆ haloalkyl, C₂₋₆ alkenyl, C₂₋₆ alkynyl, C₃₋₁₀ cycloalkyl, C₃₋₁₀ cycloalkyl-C₁₋₄-alkyl, C₂₋₁₀ heterocycloalkyl, C₂₋₁₀ heterocycloalkyl-C₁₋₄-alkyl, C₆₋₁₀ aryl, C₆₋₁₀ aryl-C₁₋₄-alkyl, C₁₋₁₀ heteroaryl, wherein said C₁₋₆ alkyl, C₁₋₆ haloalkyl, C₂₋₆ alkenyl, C₂₋₆ alkynyl, C₃₋₁₀ cyclo alkyl, C₃₋₁₀ cycloalkyl-C₁₋₄-alkyl, C₂₋₁₀ heterocycloalkyl, C₂₋₁₀ heterocycloalkyl-C₁₋₄-alkyl, C₆₋₁₀ aryl, C₆₋₁₀ aryl-C₁₋₄-alkyl, C₁₋₁₀heteroaryl, and C₁₋₁₀ heteroaryl-C₁₋₄-alkyl are each optionally substituted by one or more independently selected R⁴ groups;
each R^{e2} is independently selected from a protecting group, C₁₋₆ alkyl, C₁₋₆ haloalkyl, C₂₋₆ alkenyl, C₂₋₆ alkynyl, C₃₋₁₀ cyclo alkyl, C₃₋₁₀ cycloalkyl-C₁₋₄-alkyl, C₂₋₁₀ heterocycloalkyl, C₂₋₁₀ heterocycloalkyl-C₁₋₄-alkyl, C₆₋₁₀ aryl, C₆₋₁₀ aryl-C₁₋₄-alkyl, C₁₋₁₀ heteroaryl, wherein said C₁₋₆ alkyl, C₁₋₆ haloalkyl, C₂₋₆ alkenyl, C₂₋₆ alkynyl, C₃₋₁₀ cycloalkyl, C₃₋₁₀ cycloalkyl-C₁₋₄-alkyl, C₂₋₁₀ heterocycloalkyl, C₂₋₁₀ heterocycloalkyl-C₁₋₄-alkyl, C₆₋₁₀ aryl, C₆₋₁₀ aryl-C₁₋₄-alkyl, C₁₋₁₀ heteroaryl, and C₁₋₁₀ heteroaryl-C₁₋₄-alkyl are each optionally substituted by one or more independently selected R⁴ groups;
each R^{d2} is independently selected from a protecting group, C₁₋₆ alkyl, C₁₋₆ haloalkyl, C₂₋₆ alkenyl, C₂₋₆ alkynyl, C₃₋₁₀ cyclo alkyl, C₃₋₁₀ cycloalkyl-C₁₋₄-alkyl, C₂₋₁₀ heterocycloalkyl, C₂₋₁₀ heterocycloalkyl-C₁₋₄-alkyl, C₆₋₁₀ aryl, C₆₋₁₀ aryl-C₁₋₄-alkyl, C₁₋₁₀ heteroaryl, wherein said C₁₋₆ alkyl, C₁₋₆ haloalkyl, C₂₋₆ alkenyl, C₂₋₆ alkynyl, C₃₋₁₀ cycloalkyl, C₃₋₁₀ cycloalkyl-C₁₋₄-alkyl, C₂₋₁₀ heterocycloalkyl, C₂₋₁₀ heterocycloalkyl-C₁₋₄-alkyl, C₆₋₁₀ aryl, C₆₋₁₀ aryl-C₁₋₄-alkyl, C₁₋₁₀ heteroaryl, and C₁₋₁₀ heteroaryl-C₁₋₄-alkyl are each optionally substituted by one or more independently selected R⁴ groups;
each R^{e2} is independently selected from a protecting group, C₁₋₆ alkyl, C₁₋₆ haloalkyl, C₂₋₆ alkenyl, C₂₋₆ alkynyl, C₃₋₁₀ cyclo alkyl, C₃₋₁₀ cycloalkyl-C₁₋₄-alkyl, C₂₋₁₀ heterocycloalkyl, C₂₋₁₀ heterocycloalkyl-C₁₋₄-alkyl, C₆₋₁₀ aryl, C₆₋₁₀ aryl-C₁₋₄-alkyl, C₁₋₁₀ heteroaryl, wherein said C₁₋₆ alkyl, C₁₋₆ haloalkyl, C₂₋₆ alkenyl, C₂₋₆ alkynyl, C₃₋₁₀ cycloalkyl, C₃₋₁₀ cycloalkyl-C₁₋₄-alkyl, C₂₋₁₀ heterocycloalkyl, C₂₋₁₀ heterocycloalkyl-C₁₋₄-alkyl, C₆₋₁₀ aryl, C₆₋₁₀ aryl-C₁₋₄-alkyl, C₁₋₁₀ heteroaryl, and C₁₋₁₀ heteroaryl-C₁₋₄-alkyl are each optionally substituted by one or more independently selected R⁴ groups;
each R^{f2} is independently selected from a protecting group, C₁₋₆ alkyl, C₁₋₆ haloalkyl, C₂₋₆ alkenyl, C₂₋₆ alkynyl, C₃₋₁₀ cyclo alkyl, C₃₋₁₀ cycloalkyl-C₁₋₄-alkyl, C₂₋₁₀ heterocycloalkyl, C₂₋₁₀ heterocycloalkyl-C₁₋₄-alkyl, C₆₋₁₀ aryl, C₆₋₁₀ aryl-C₁₋₄-alkyl, C₁₋₁₀heteroaryl, wherein said C₁₋₆ alkyl, C₁₋₆ haloalkyl, C₂₋₆ alkenyl, C₂₋₆ alkynyl, C₃₋₁₀ cycloalkyl, C₃₋₁₀ cycloalkyl-C₁₋₄-alkyl, C₂₋₁₀ heterocycloalkyl, C₂₋₁₀ heterocycloalkyl-C₁₋₄-alkyl, C₆₋₁₀ aryl, C₆₋₁₀ aryl-C₁₋₄-alkyl, C₁₋₁₀ heteroaryl, and C₁₋₁₀ heteroaryl-C₁₋₄-alkyl are each optionally substituted by one or more independently selected R⁴ groups;
each R^{k2}, R^{g2} and R^{h2} is independently selected from a protecting group, C₁₋₆ alkyl, C₁₋₆ haloalkyl, C₂₋₆ alkenyl, C₂₋₆ alkynyl, C₃₋₁₀ cycloalkyl, C₃₋₁₀ cycloalkyl-C₁₋₄-alkyl, C₂₋₁₀ heterocycloalkyl, C₂₋₁₀ heterocycloalkyl-C₁₋₄-alkyl, C₆₋₁₀ aryl, C₆₋₁₀ aryl-C₁₋₄-alkyl, C₁₋₁₀ heteroaryl, wherein said C₁₋₆ alkyl, C₁₋₆ haloalkyl, C₂₋₆ alkenyl, C₂₋₆ alkynyl, C₃₋₁₀ cycloalkyl, C₃₋₁₀ cycloalkyl-C₁₋₄-alkyl, C₂₋₁₀ heterocycloalkyl, C₂₋₁₀ heterocycloalkyl-C₁₄-alkyl, C₆₋₁₀ aryl, C₆₋₁₀ aryl-C₁₋₄-alkyl, C₁₋₁₀ heteroaryl, and C₁₋₁₀ heteroaryl-C₁₋₄-alkyl are each optionally substituted by one or more independently selected R⁴ groups;
or alternatively, R^{k2} and R^{a2}, taken together with the atoms to which they are attached, form a heterocycloalkyl or heteroaryl ring, which is optionally substituted by one or more R⁴ groups;
or alternatively, R^{k2} and R^{b2}, taken together with the atoms to which they are attached, form a heterocycloalkyl or heteroaryl ring, which is optionally substituted by one or more R⁴ groups;
or alternatively, R^{k2} and R^{g2}, taken together with the atoms to which they are attached, form a heterocycloalkyl or heteroaryl ring, which is optionally substituted by one or more R⁴ groups;
or alternatively, R^{g2} and R^{h2}, taken together with the nitrogen atom to which they are attached, form a heterocycloalkyl or heteroaryl ring, which is optionally substituted by one or more R⁴ groups;
each R⁴ is independently selected from halo, cyano, nitro, C₁₋₆ alkyl, C₁₋₆ haloalkyl, C₁₋₆ alkyl-NR^{4a}—C₁₋₆ alkylene, C₁₋₆ alkyl-O—C₁₋₆ alkylene, C₂₋₆ alkenyl, C₂₋₆ alkynyl, C₃₋₁₀ cycloalkyl, C₃₋₁₀ cycloalkyl-C₁₋₄-alkyl, C₂₋₁₀ heterocycloalkyl, C₂₋₁₀heterocycloalkyl-C₁₋₄-alkyl, C₆₋₁₀ aryl, C₆₋₁₀ aryl-C₁₋₄-alkyl, C₁₋₁₀heteroaryl, hydroxy, C₁₋₆ alkoxy, C₁₋₆ haloalkoxy, C₁₋₆ alkylthio, C₁₋₆ alkylsulfinyl, C₁₋₆ alkylsulfonyl, carbamyl, C₁₋₆ alkylcarbamyl, di(C₁₋₆alkyl)carbamyl, carboxy, amino, C₁₋₆ alkylamino, di-C₁₋₆ alkylamino, C₁₋₆ alkylcarbonyl, C₁₋₆ alkoxycarbonyl, C₁₋₆ alkylcarbonyloxy, C₁₋₆ alkylcarbonylamino, C₁₋₆ alkylsulfonylamino, amino sulfonyl, C₁₋₆ alkylamino sulfonyl, di(C₁₋₆ alkyl)aminosulfonyl, amino sulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆alkyl)aminocarbonylamino; wherein said C₁₋₆ alkyl, C₁₋₆ haloalkyl, C₂₋₆ alkenyl, C₂₋₆ alkynyl, C₁₋₆ alkoxy, C₁₋₆ alkyl-NR^{4a}—C₁₋₆ alkylene, C₁₋₆ alkyl-O—C₁₋₆ alkylene, C₃₋₁₀ cyclo alkyl, C₃₋₁₀ cycloalkyl-C₁₋₄-alkyl, C₂₋₁₀ heterocycloalkyl, C₂₋₁₀ heterocycloalkyl-C₁₋₄-alkyl, C₆₋₁₀ aryl, C₆₋₁₀ aryl-C₁₋₄-alkyl, C₁₋₁₀ heteroaryl are each optionally substituted by one or more groups selected from halo, cyano, nitro, C₁₋₆ alkyl, C₁₋₆ haloalkyl, C₂₋₆ alkenyl, C₂₋₆ alkynyl, C₁₋₆alkoxy, hydroxy, C₁₋₆alkoxy, C₁₋₆haloalkoxy, C₁₋₆alkylthio, C₁₋₆ alkylsulfinyl, C₁₋₆ alkylsulfonyl, carbamyl, C₁₋₆ alkylcarbamyl, di(C₁₋₆alkyl)carbamyl, carboxy, amino, C₁₋₆ alkylamino, di-C₁₋₆ alkylamino, C₁₋₆alkylcarbonyl, C₁₋₆alkoxycarbonyl, C₁₋₆ alkylcarbonyloxy, C₁₋₆ alkylcarbonylamino, C₁₋₆alkylsulfonylamino, amino sulfonyl, C₁₋₆ alkylamino sulfonyl, di(C₁₋₆alkyl)aminosulfonyl, amino sulfonylamino, C₁₋₆ alkylaminosulfonylamino, di(C₁₋₆alkyl)aminosulfonylamino, aminocarbonylamino, C₁₋₆ alkylaminocarbonylamino, di(C₁₋₆alkyl)aminocarbonylamino, and C₃₋₁₀ cycloalkyl-C₁₋₄-alkyl, C₂₋₁₀ heterocycloalkyl, C₂₋₁₀ heterocycloalkyl-C₁₋₄-alkyl, C₆₋₁₀ aryl, C₆₋₁₀ aryl-C₁₋₄-alkyl, C₁₋₁₀ heteroaryl; and
each R^{4a} is independently selected from H and C₁₋₆ alkyl;
provided that each hydrogen atom in which is directly attached to a nitrogen atom, sulfur atom, or oxygen atom in any of the aforementioned groups is replaced by a protecting group.

36. The process of claim 1, wherein the compound of Formula II is selected from: 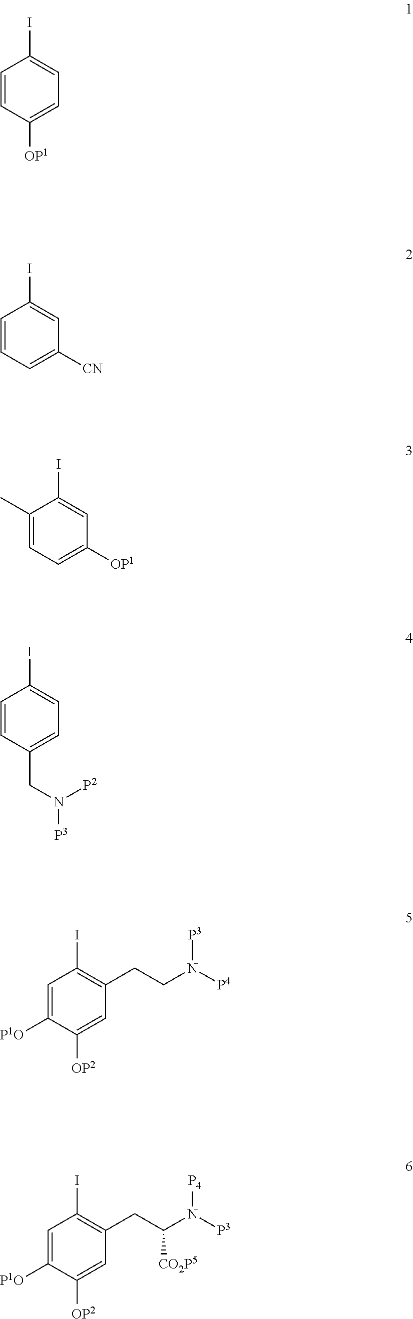 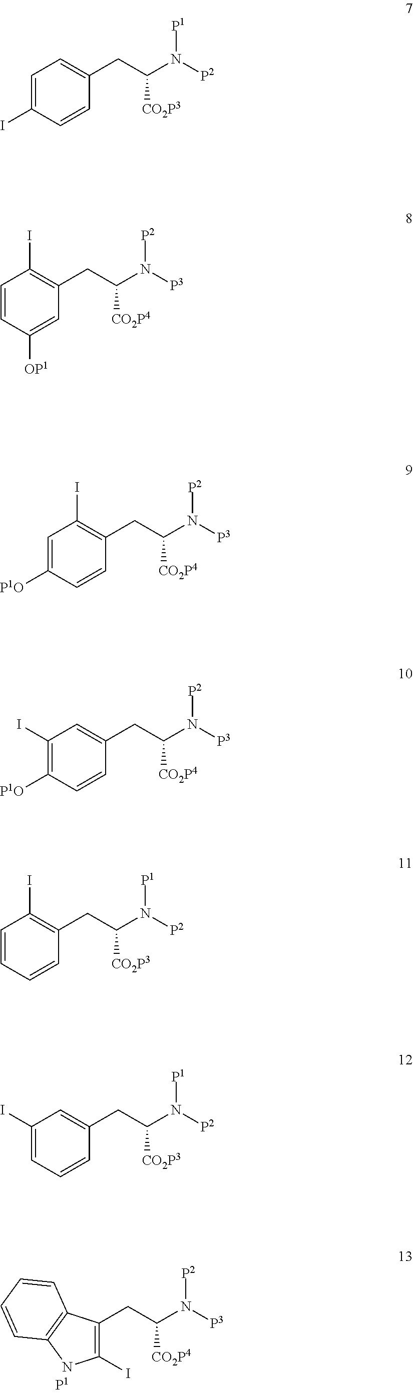 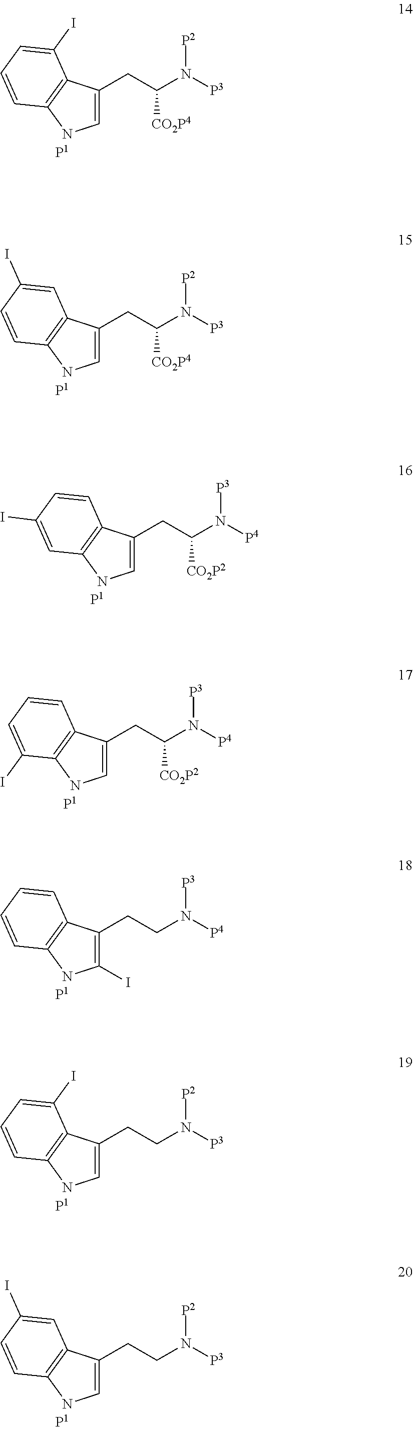 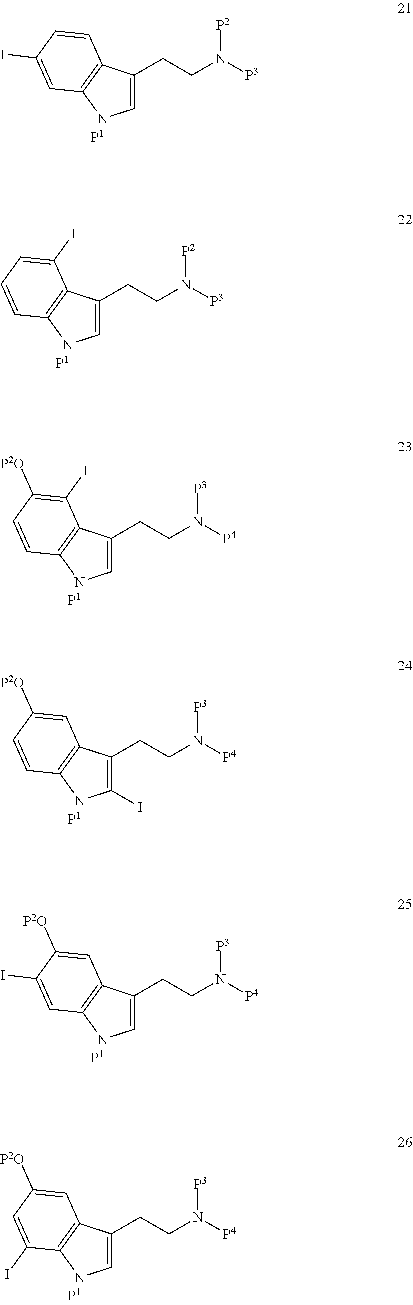 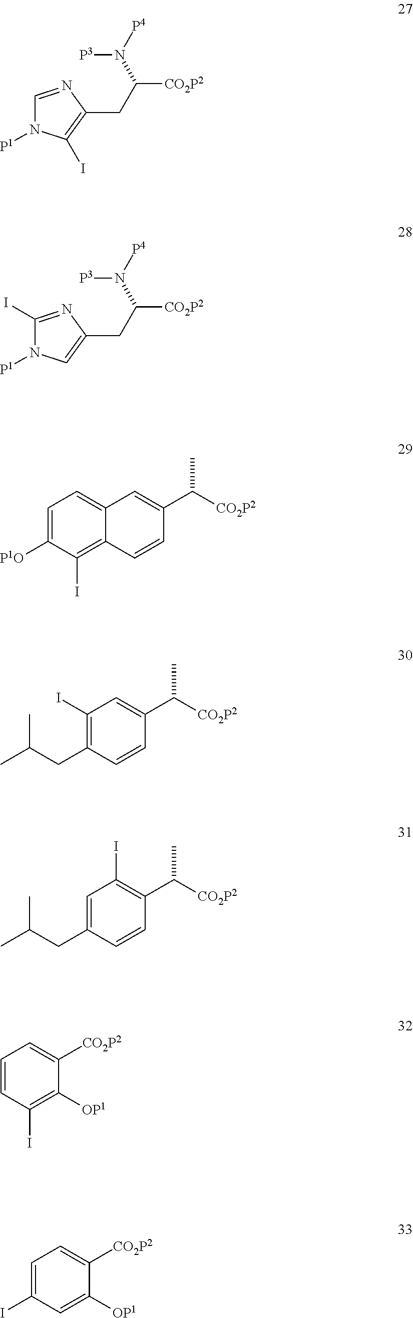 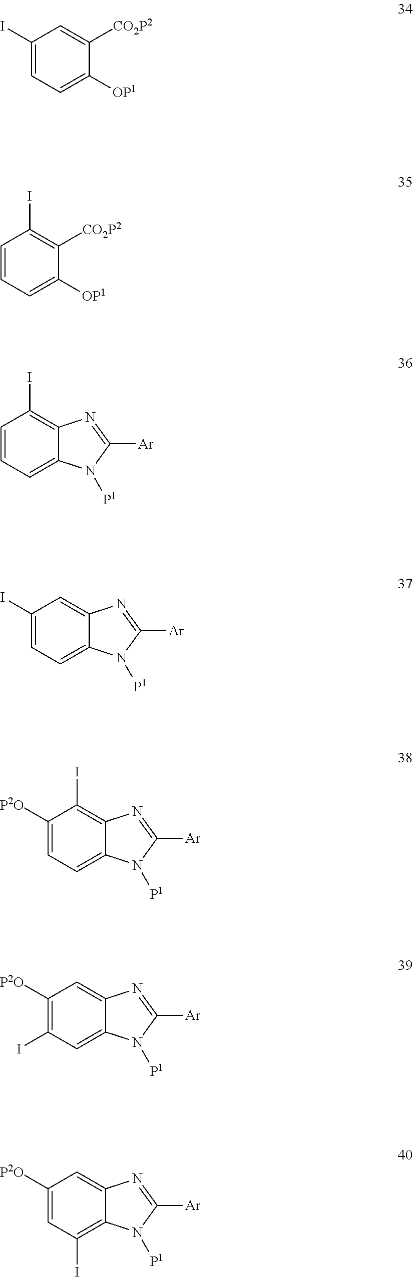 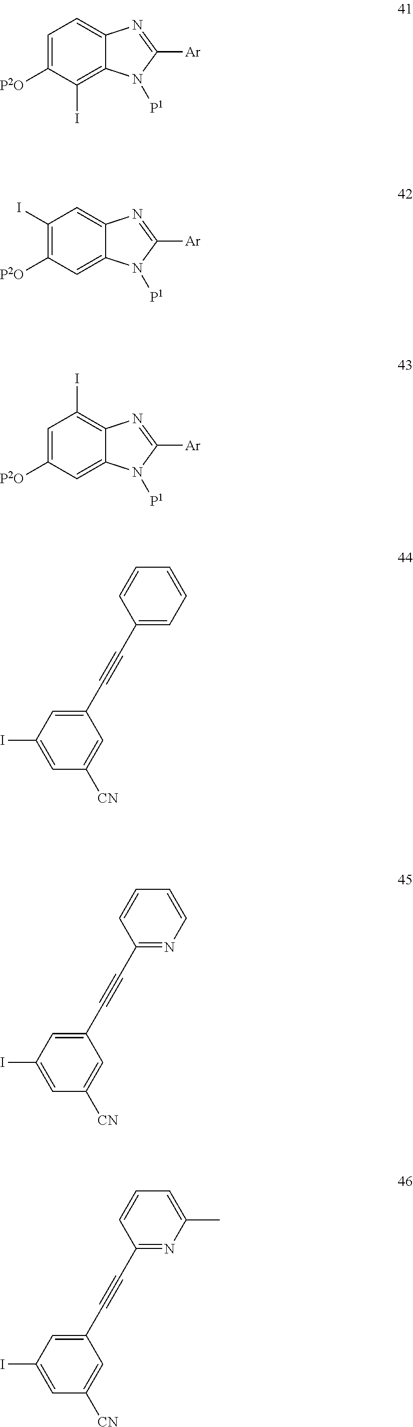 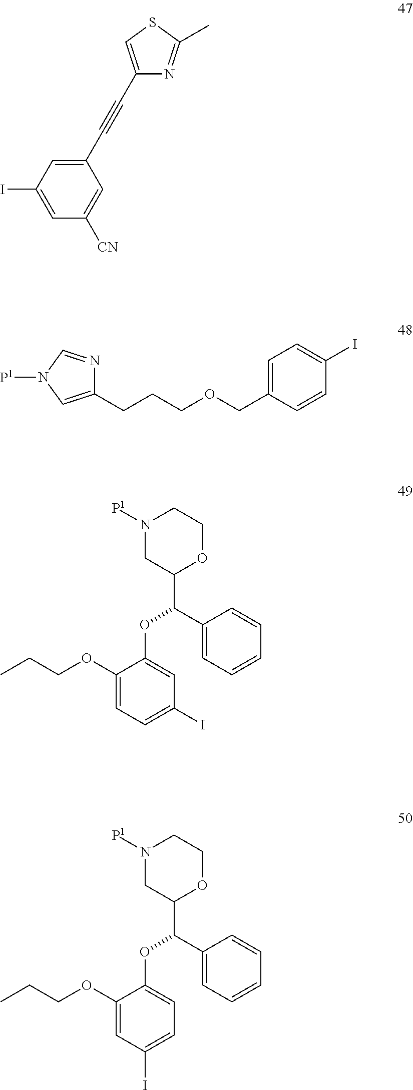 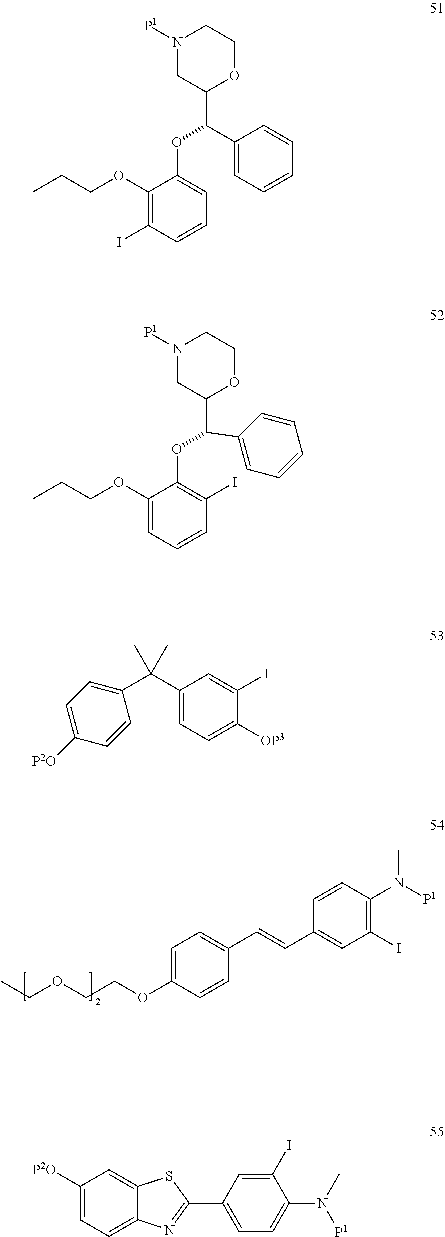 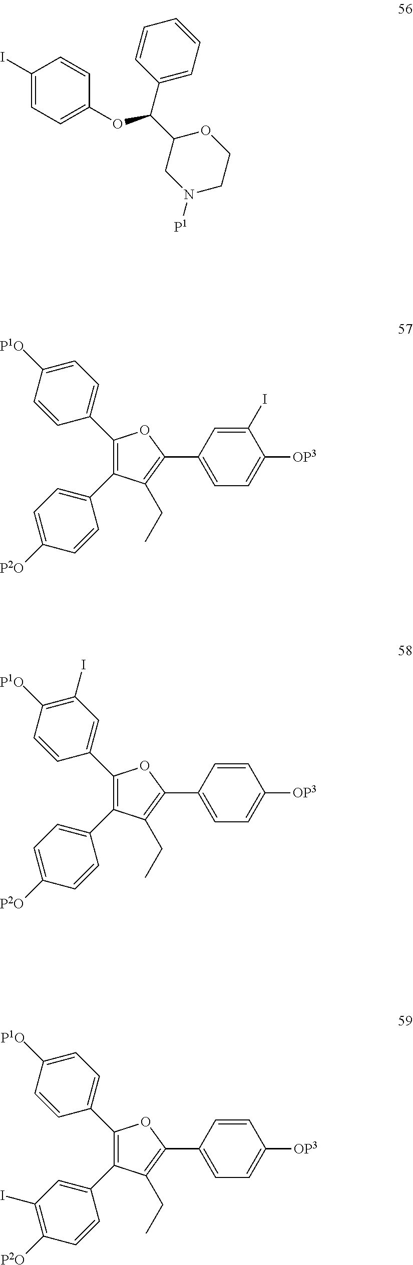 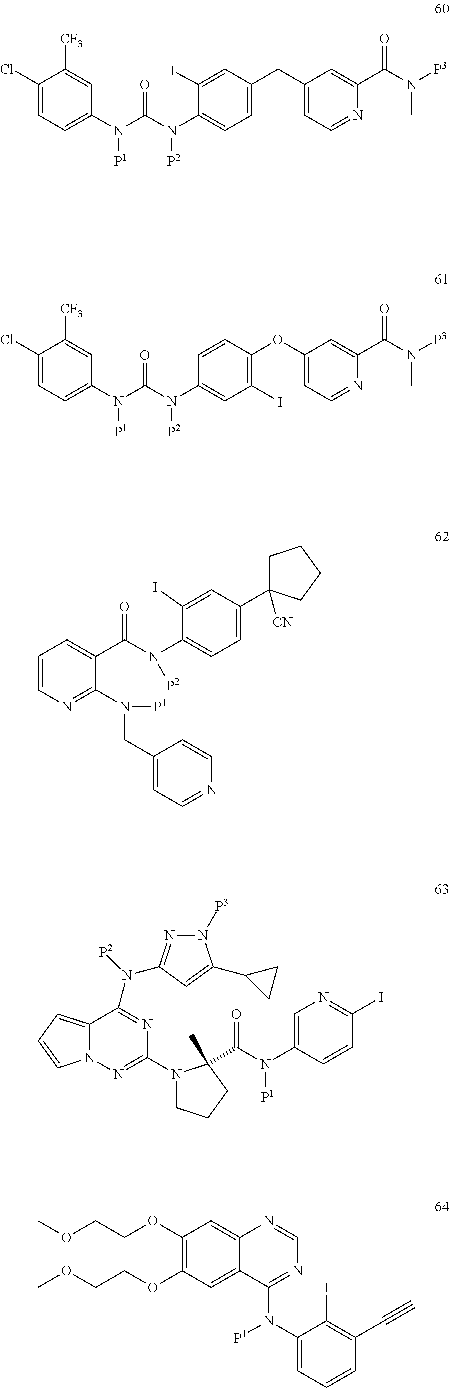 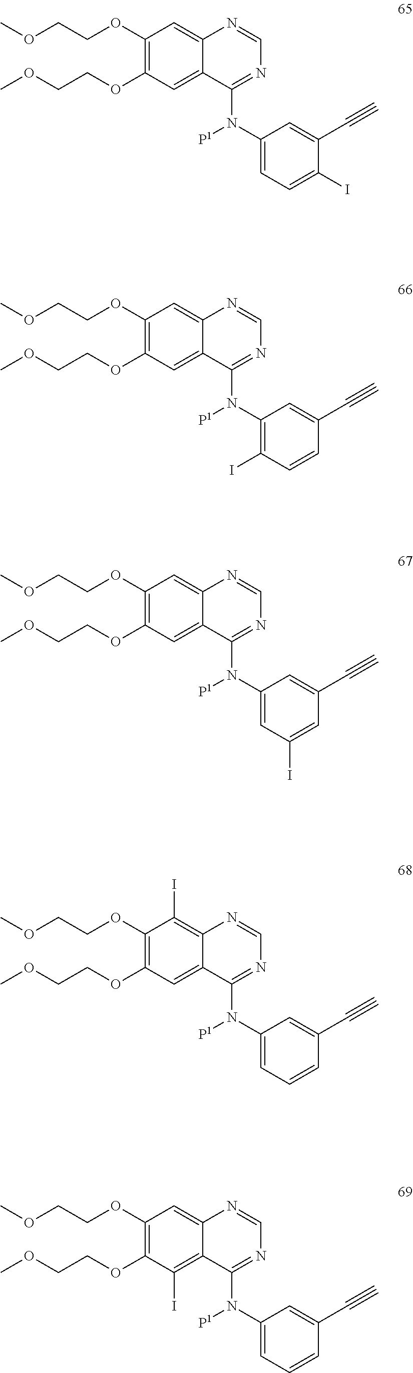 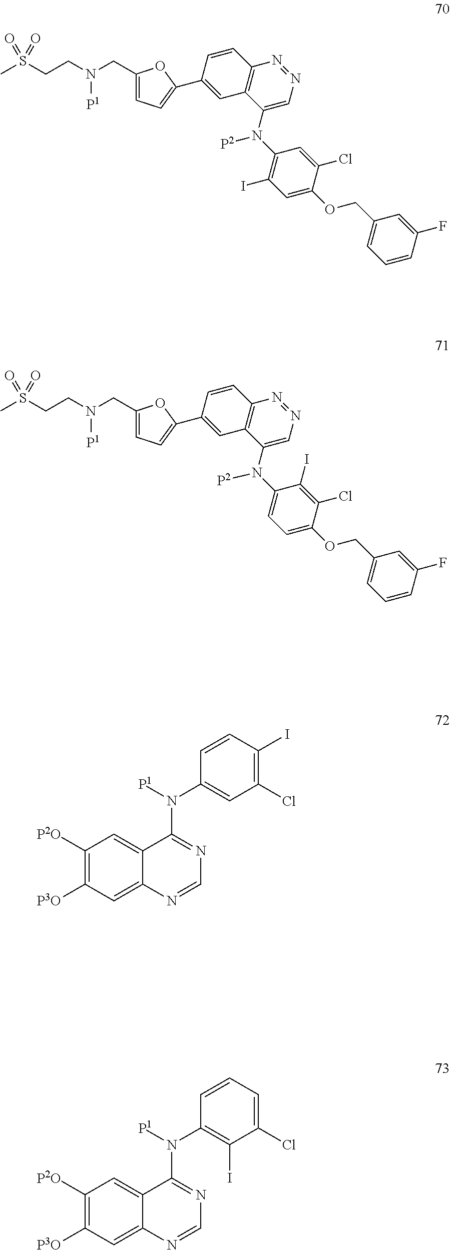 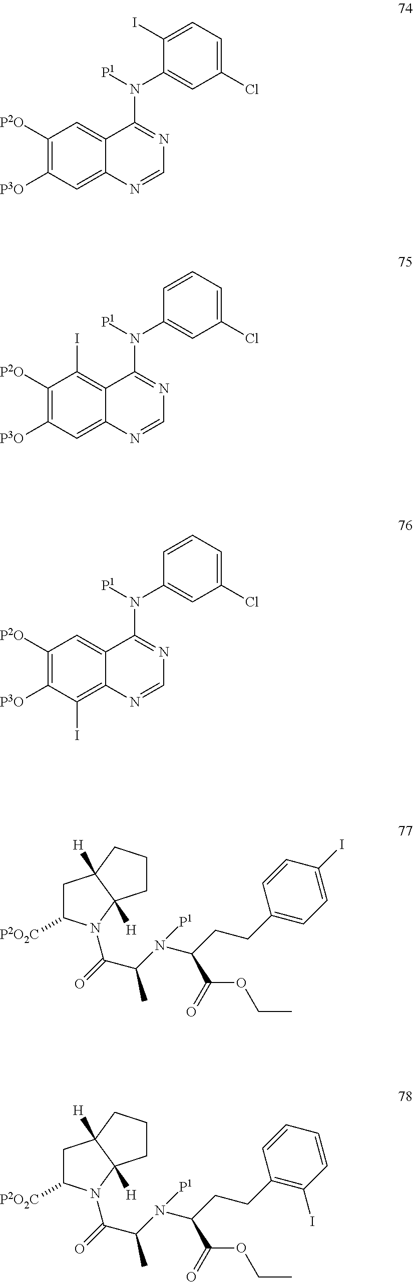 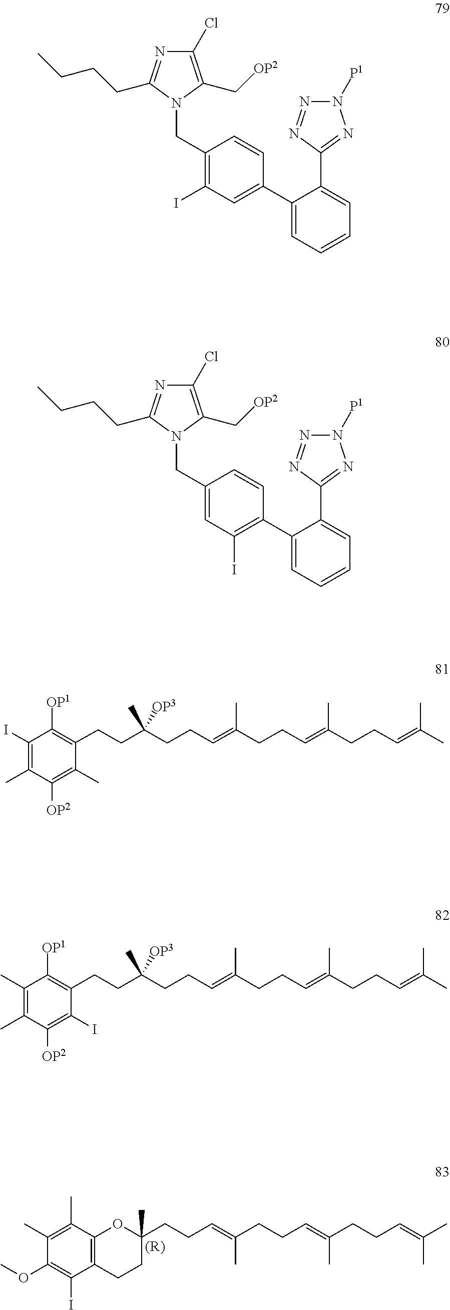 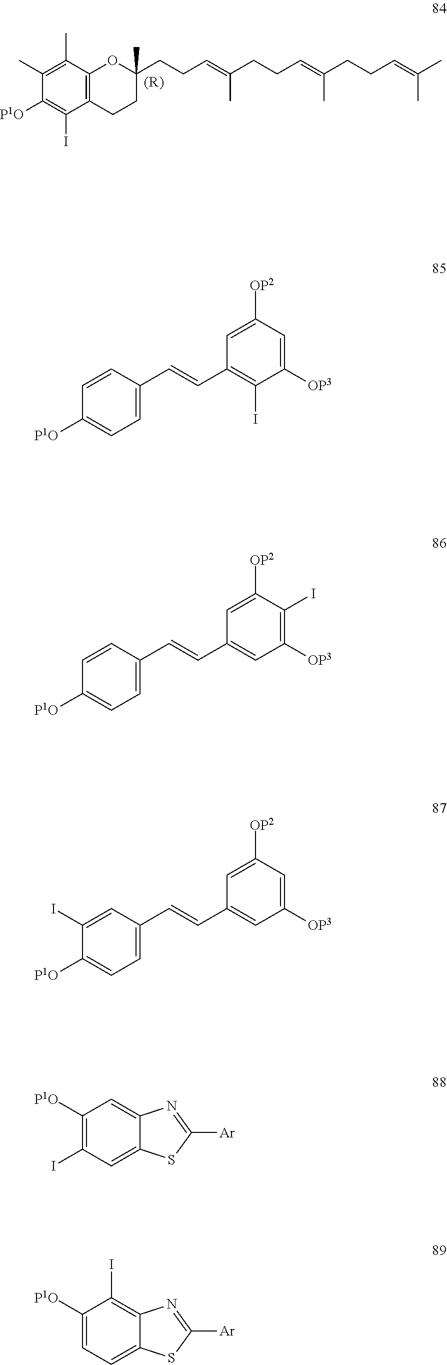 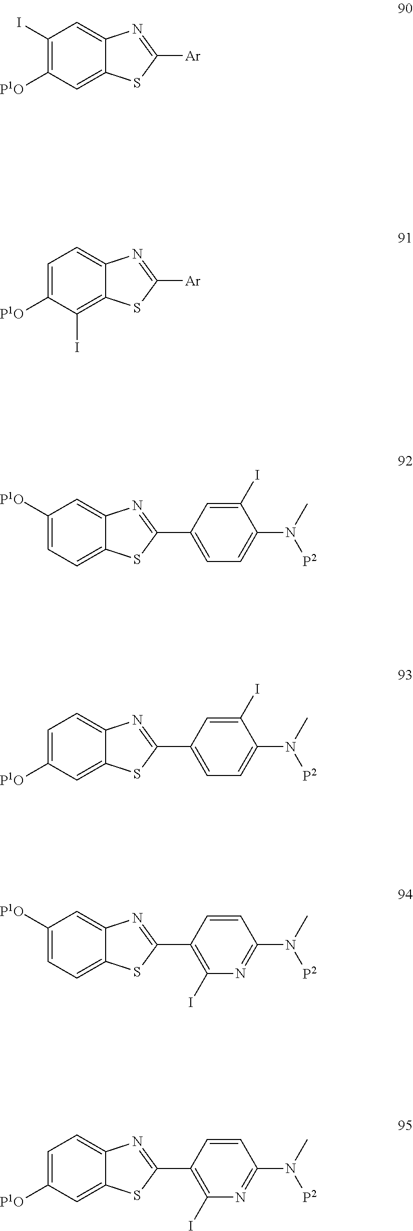 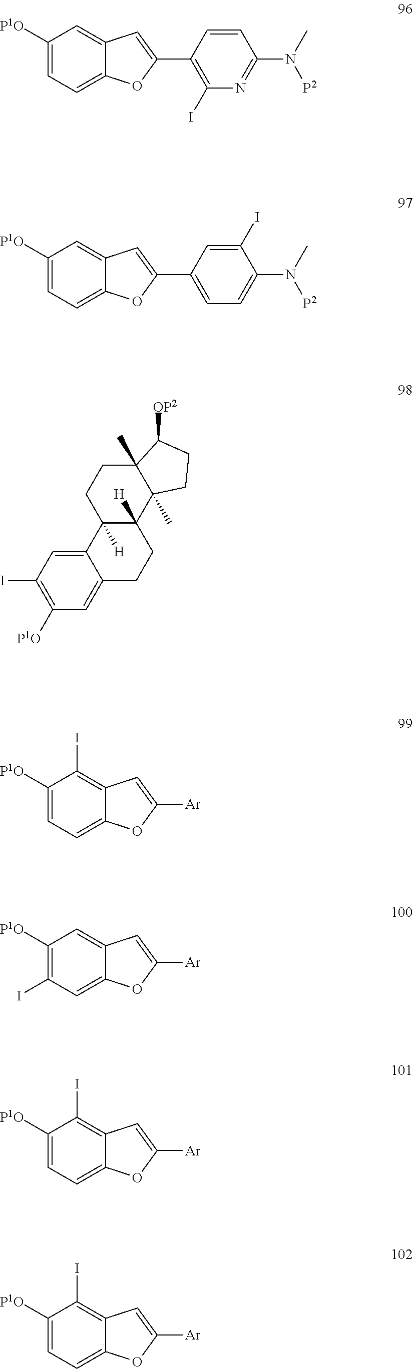 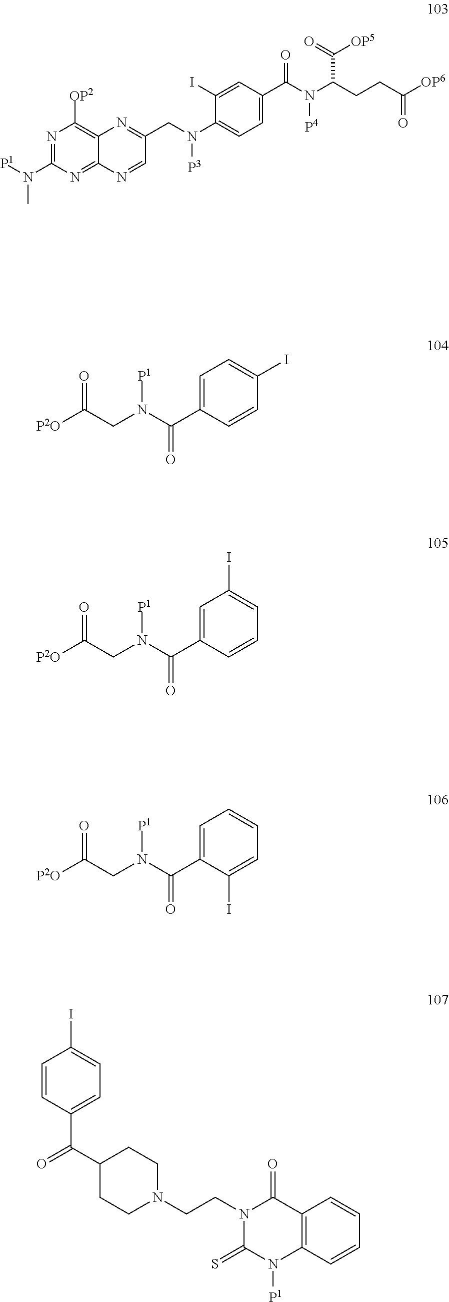 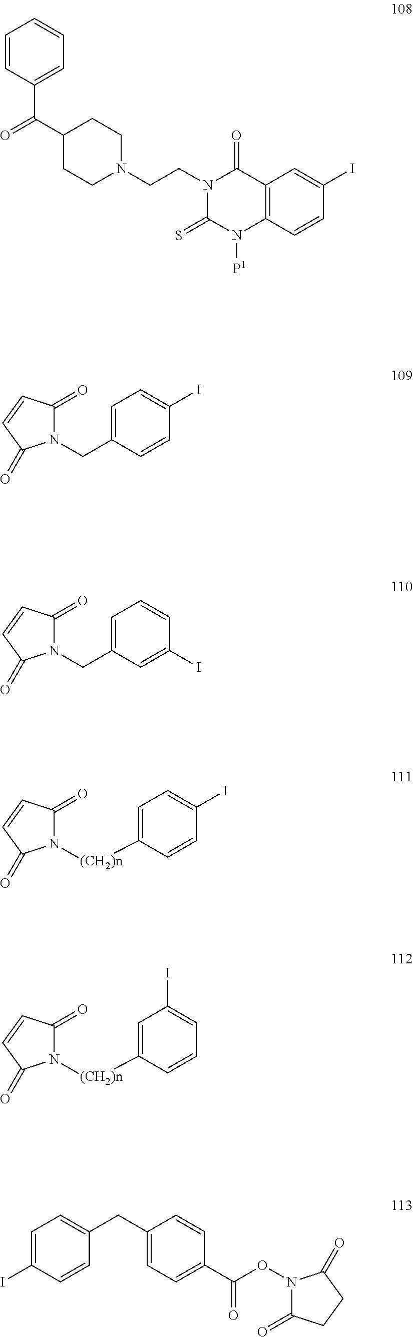
wherein Ar is an optionally substituted aryl or heteroaryl, wherein Ar does not have unprotected protic groups; and P¹, P², P³, P⁴, P⁵, and P⁶ are each, independently, protecting groups.

37. The process of claim 1, wherein Ar¹ is: 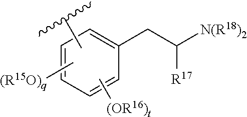
wherein;
q is 0 or 1;
t is 0 or 1;
R¹⁵ and R¹⁶ are each, independently, an acid labile protecting group;
R¹⁷ is selected from hydrogen and C(O)₂R¹⁹;
R¹⁸ in each occurrence is independently selected from hydrogen and t-butoxycarbonyl; and
R¹⁹ is selected from hydrogen, methyl, and t-butyl.

38-40. (canceled)

41. The process of claim 1, wherein Ar¹ is: 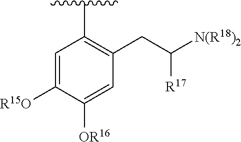
wherein;
R¹⁵ and R¹⁶ are ethoxymethyl;
R¹⁷ is selected from hydrogen and C(O)₂R¹⁹;
R¹⁸ in each occurrence is independently selected from hydrogen and t-butoxycarbonyl; and
R¹⁹ is selected from hydrogen, methyl, and t-butyl.

42. The process of claim 1, wherein Ar¹ is: 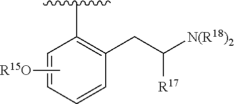
wherein;
R¹⁵ is alkoxymethyl;
R¹⁷ is selected from hydrogen and C(O)₂R¹⁹;
R¹⁸ in each occurrence is independently selected from hydrogen and t-butoxycarbonyl; and
R¹⁹ is selected from hydrogen, methyl, and t-butyl.

43. A compound of Formula III, selected from the group consisting of: 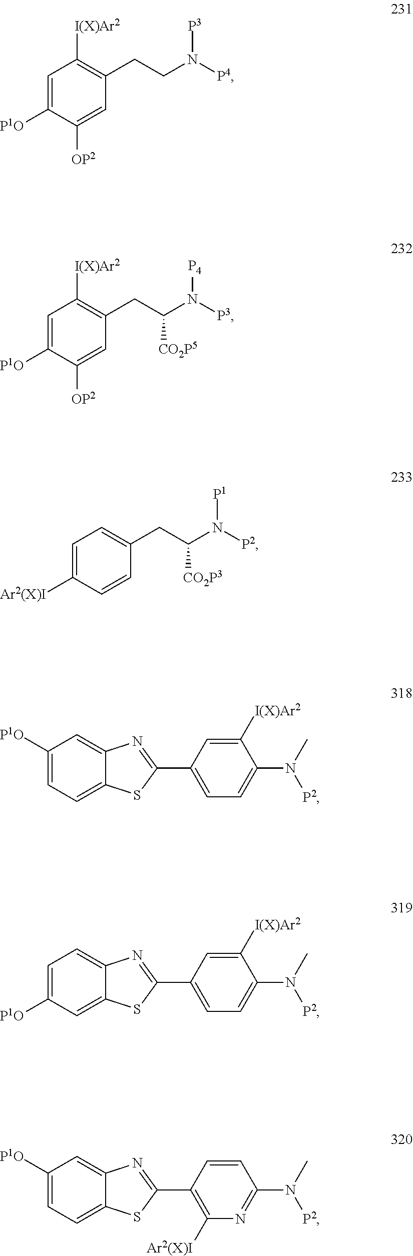 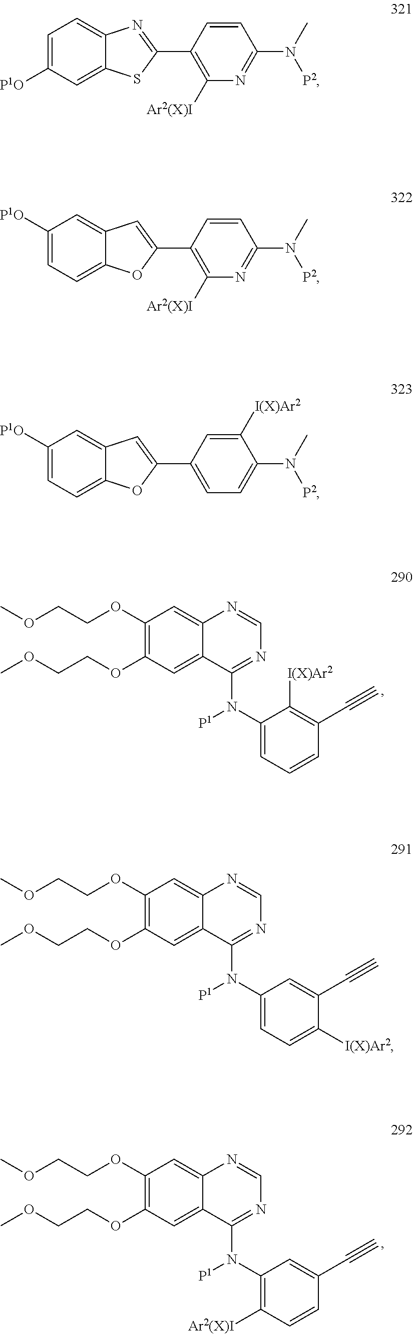 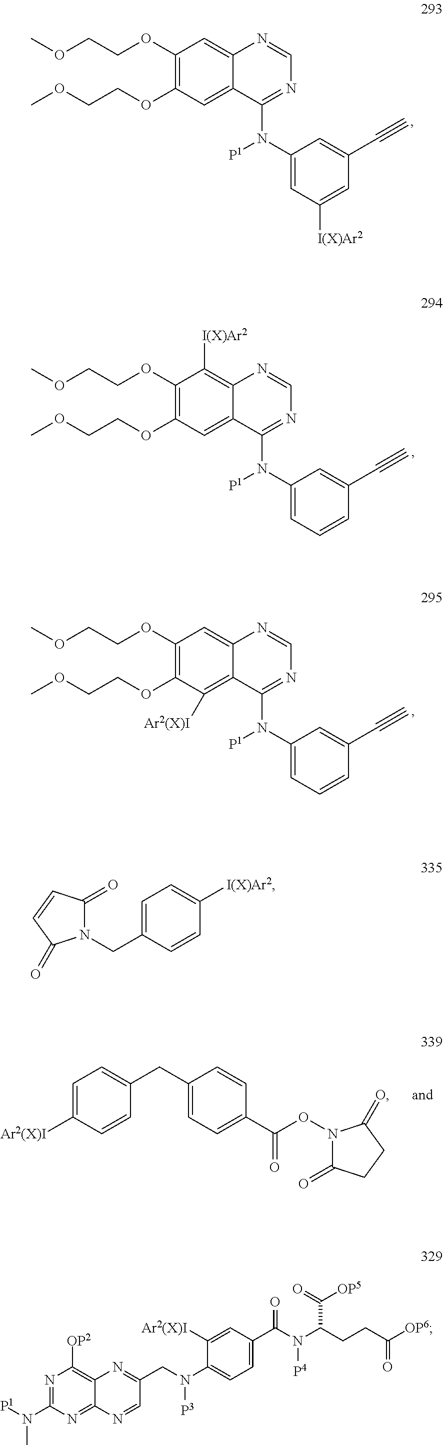
wherein Ar² is an optionally substituted aryl or heteroaryl;
X is a ligand that is a conjugate base of an acid HX, wherein HX has a pKa of less than or equal to 5; and
P¹, P², P³, P⁴, P⁵ and P⁶ are each, independently, protecting groups.

44. (canceled)

45. A compound of Formula I, selected from the group consisting of: 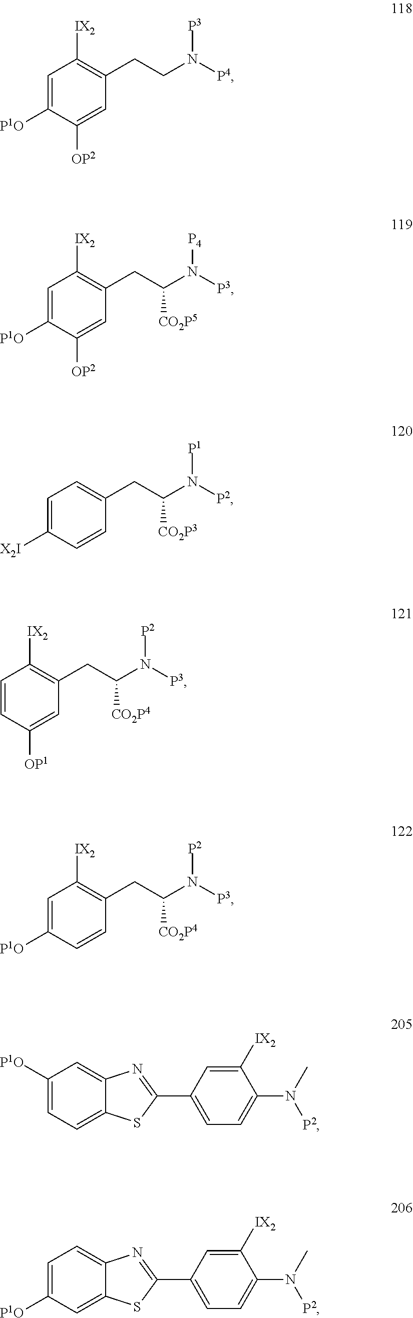 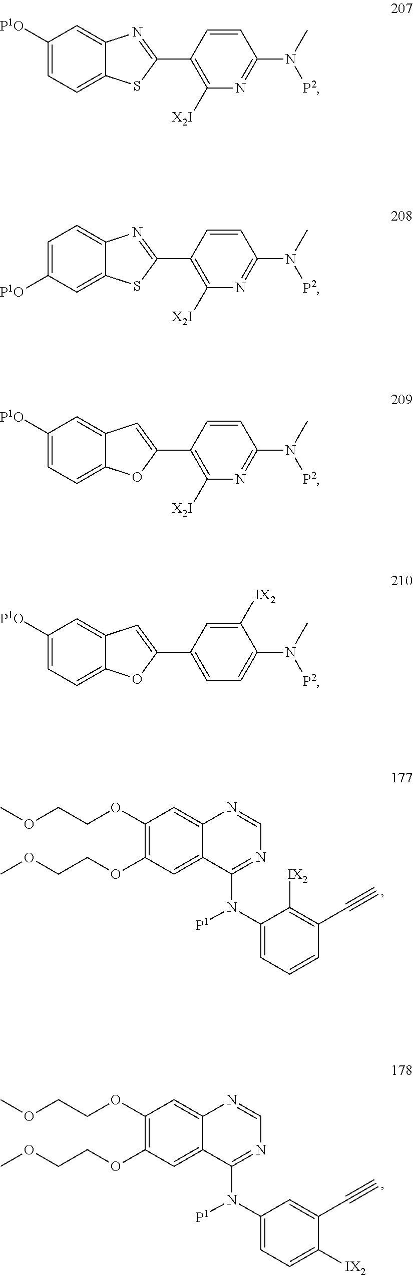 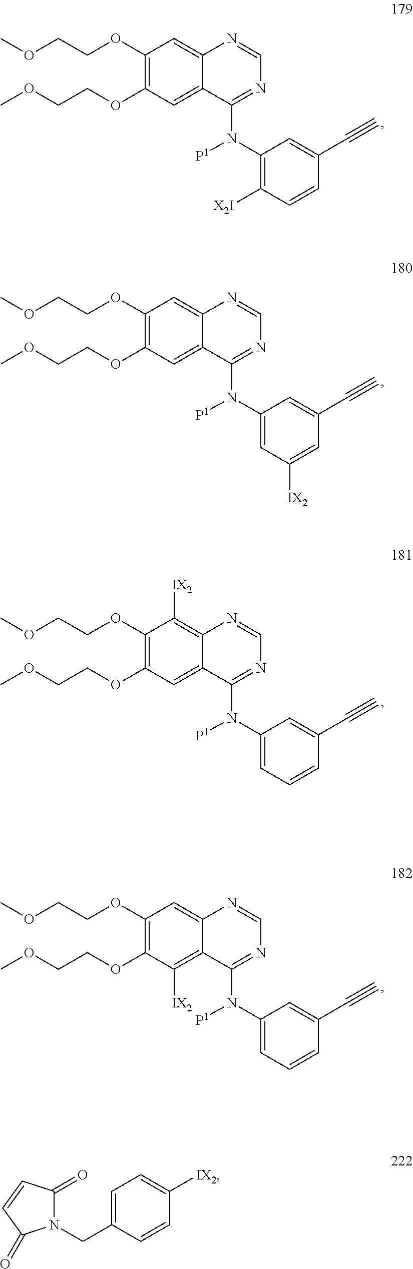 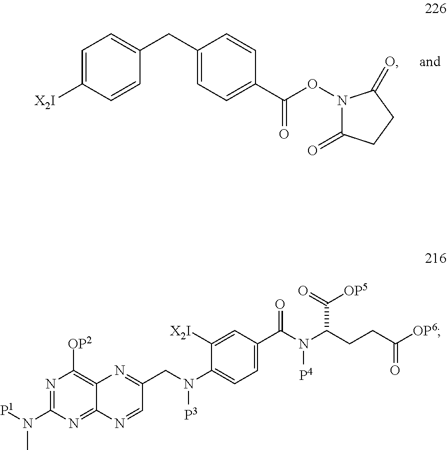
wherein
X is a ligand that is a conjugate base of an acid HX, wherein HX has a pKa of less than or equal to 5; and
P¹, P², P³, P⁴, P⁵ and P⁶ are each, independently, protecting groups.

46-58. (canceled)

59. A process for making a compound of Formula VI:
Ar¹—W VI
comprising reacting a compound of Formula V: 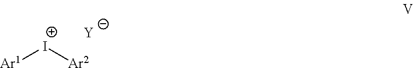
with a compound MW, in a polar solvent, such that a compound of Formula VI is formed;
wherein:
M is a counter ion;
Ar¹ and Ar² are independently, optionally substituted aryl or heteroaryl;
Y is an anion for which the pKa of the conjugate acid H—Y is less than about 1; and
W is selected from the group consisting of fluorine, iodine, radioactive isotopes of fluorine and iodine, and astatine.
